SEQUENCE LISTING (83 sequences)

SEQ ID NO: 1 - DNA, 34 bp, synthetic construct
  misc_feature 1..34: note = Description of Artificial Sequence: Synthetic oligonucleotide
  misc_feature 1..34: note = LoxP site
  source 1..34: mol_type = other DNA
ataacttcgtataatgtatgctatacgaagttat

SEQ ID NO: 2 - DNA, 1686 bp, synthetic construct
  misc_feature 1..1686: note = Description of Artificial Sequence: Synthetic polynucleotide
  misc_feature 1..1686: note = pCAGG promoter
  source 1..1686: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggtta

SEQ ID NO: 3 - DNA, 21 bp, synthetic construct
  misc_feature 1..21: note = Description of Artificial Sequence: Synthetic oligonucleotide
  misc_feature 1..21: note = NLS used in Intein
  source 1..21: mol_type = other DNA
cccaagaagaagaggaaggtg

SEQ ID NO: 4 - protein, 7 aa, synthetic construct
  REGION 1..7: note = Description of Artificial Sequence: Synthetic peptide
  REGION 1..7: note = NLS used in Intein
PKKKRKV

SEQ ID NO: 5 - DNA, 123 bp, synthetic construct
  misc_feature 1..123: note = Description of Artificial Sequence: Synthetic polynucleotide
  misc_feature 1..123: note = nCre 19-59
  source 1..123: mol_type = other DNA
acgagtgatgaggttcgcaagaacctgatggacatgttcagggatcgccaggcgttttctgagcatacctggaaaatgcttctgtccgtttgccggtcgtgggcggcatggtgcaagttgaat

SEQ ID NO: 6 - protein, 41 aa, synthetic construct
  REGION 1..41: note = Description of Artificial Sequence: Synthetic polypeptide
  REGION 1..41: note = nCre 19-59
TSDEVRKNLMDMFRDRQAFSEHTWKMLLSVCRSWAAWCKLN

SEQ ID NO: 7 - DNA, 387 bp, synthetic construct
  misc_feature 1..387: note = Description of Artificial Sequence: Synthetic polynucleotide
  misc_feature 1..387: note = nIntein
  source 1..387: mol_type = other DNA
aaatttgcggaatattgcctcagttttggcaccgaaattttaaccgttgagtacggcccattgcccattggcaaaattgtgagtgaagaaattaattgttctgtgtacagtgttgatccagaagggagagtttacacccaggcgatcgcccaatggcatgaccggggagagcaggaagtattggaatatgaattggaagatggttcagtaatccgagctacctctgaccaccgctttttaaccaccgattatcaactgttggcgatcgaagaaatttttgctaggcaactggacttgttgactttagaaaatattaagcaaactgaagaagctcttgacaaccatcgtcttccctttccattacttgacgctgggacaattaaataa

SEQ ID NO: 8 - protein, 128 aa, synthetic construct
  REGION 1..128: note = Description of Artificial Sequence: Synthetic polypeptide
  REGION 1..128: note = nIntein
KFAEYCLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 9 - DNA, 123 bp, synthetic construct
  misc_feature 1..123: note = Description of Artificial Sequence: Synthetic polynucleotide
  misc_feature 1..123: note = cIntein
  source 1..123: mol_type = other DNA
atggttaaagttatcggtcgtcgttccctcggagtgcaaagaatatttgatattggtcttccccaagaccataattttctgctagccaatggggcgatcgccgccaattgttttaacaaatcc

SEQ ID NO: 10 - protein, 41 aa, synthetic construct
  REGION 1..41: note = Description of Artificial Sequence: Synthetic polypeptide
  REGION 1..41: note = cIntein
MVKVIGRRSLGVQRIFDIGLPQDHNFLLANGAIAANCFNKS

SEQ ID NO: 11 - DNA, 855 bp, synthetic construct
  misc_feature 1..855: note = Description of Artificial Sequence: Synthetic polynucleotide
  misc_feature 1..855: note = cCre 60-343
  source 1..855: mol_type = other DNA
aaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 12 - protein, 284 aa, synthetic construct
  REGION 1..284: note = Description of Artificial Sequence: Synthetic polypeptide
  REGION 1..284: note = cCre 60-343
NRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 13 - DNA, 66 bp, synthetic construct
  misc_feature 1..66: note = Description of Artificial Sequence: Synthetic oligonucleotide
  misc_feature 1..66: note = P2A
  source 1..66: mol_type = other DNA
ggcagcggcgccaccaacttcagcctgctgaagcaggccggcgacgtggaggagaaccccggcccc

SEQ ID NO: 14 - protein, 22 aa, synthetic construct
  REGION 1..22: note = Description of Artificial Sequence: Synthetic peptide
  REGION 1..22: note = P2A
GSGATNFSLLKQAGDVEENPGP

SEQ ID NO: 15 - DNA, 714 bp, synthetic construct
  misc_feature 1..714: note = Description of Artificial Sequence: Synthetic polynucleotide
  misc_feature 1..714: note = GFP
  source 1..714: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 16 - protein, 238 aa, synthetic construct
  REGION 1..238: note = Description of Artificial Sequence: Synthetic polypeptide
  REGION 1..238: note = GFP
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 17 - DNA, 63 bp, synthetic construct
  misc_feature 1..63: note = Description of Artificial Sequence: Synthetic oligonucleotide
  misc_feature 1..63: note = T2A
  source 1..63: mol_type = other DNA
ggcagtggagagggcagaggaagtctgctaacatgcggtgacgtcgaggagaatcctggccca

SEQ ID NO: 18 - protein, 21 aa, synthetic construct
  REGION 1..21: note = Description of Artificial Sequence: Synthetic peptide
  REGION 1..21: note = T2A
GSGEGRGSLLTCGDVEENPGP

SEQ ID NO: 19 - DNA, 156 bp, synthetic construct
  misc_feature 1..156: note = Description of Artificial Sequence: Synthetic polynucleotide
  misc_feature 1..156: note = 5'ssODN (with LoxP)
  source 1..156: mol_type = other DNA
tatgcagatgcctatgtttagatagtatggtagccccaatagctttaccaaaattcctcctataacttcgtataatgtatgctatacgaagttatatagtgaacataaatataacatttttttgtaagagcttctttattgcttctatctaaccca

SEQ ID NO: 20 - DNA, 156 bp, synthetic construct
  misc_feature 1..156: note = Description of Artificial Sequence: Synthetic polynucleotide
  misc_feature 1..156: note = 3'ssODN (With LoxP)
  source 1..156: mol_type = other DNA
tccactttcctctgacctgaactgagtgagaaactttgaggctgtgagttacgttcaagtcataacttcgtataatgtatgctatacgaagttattaaagggtgcacgtggaatgtgggtgtgcgaagcacatcaccgctgtagttattccattac

SEQ ID NO: 21 - DNA, 20 bp, synthetic construct
  misc_feature 1..20: note = Description of Artificial Sequence: Synthetic oligonucleotide
  misc_feature 1..20: note = sgRNA-5'ssODN
  misc_feature 1..20: note = ch2:34,397,674..34,397,693 white leghorn layer GRCg7w
  source 1..20: mol_type = other DNA
atatttatgttcactatagg

SEQ ID NO: 22 - DNA, 20 bp, synthetic construct
  misc_feature 1..20: note = Description of Artificial Sequence: Synthetic oligonucleotide
  misc_feature 1..20: note = sgRNA-3'ssODN
  misc_feature 1..20: note = ch2:34,392,740..34,392,759 white leghorn layer GRCg7w
  source 1..20: mol_type = other DNA
tgagttacgttcaagtctaa

SEQ ID NO: 23 - DNA, 1330 bp, synthetic construct
  misc_feature 1..1330: note = Description of Artificial Sequence: Synthetic polynucleotide
  misc_feature 1..1330: note = 5' HA of TV to the last exon of DAZL
  misc_feature 1..1330: note = ch2:34,384,285..34,385,615 white leghorn layer GRCg7w
  source 1..1330: mol_type = other DNA
gaattgtggaagaattgcagctactaaaaaggcagactgggtagcacttcccaccttactcagatttaggagtctcaagacatgtagatgggtgaaatttattattcttttctctgcttgcttttctggaaagctacctctatttggccagcagttggctctgaattcctttctttaaaggagacctgactgctctcctgtctgcctcttacacagctgtactggtgtgtcactgatagaaacgtttaccttgaaagtgacaagtgggggcgttgatacccattaacaacattaaagggaaaatttcaggaaacaagaaaactgagaggagttcagaatgaaatgtaagccctgtgacagactcacactgatggtagtagggtaactgatggagatgcatctataacaattctaacttttatttcagaagtctgtggacaggagcatacaaacagtagtatcttgtctgtttaaccctgaaaaccgtctgaggaacacctttgtatcacaagaagactacttcagggtatgtagtgaaagcaaaactaaatctctaaaaccagctgatcttaactttgttcatatacaggatgccatgaagacaaaggtttatctgtacttgcttgagattttactttttgtatgtgcattgagatgaatgactggttagctctcagttggttaatactcaatgaaaattgcagaaattgagttcagtggatttaagtgcattgatacaaggctgcataagaactgctgagcagtcagctattggatatttagagctaatcatccctcaggagcaatctgctgaaaggtaagcagattgcatcttgtaaacgagagagcagagttaaacgtttgtggtctttgctccacttaagtacccacagtggatgtctatctgtacttttggttaaaaaatagataatttctacaacagactgtagttaggtcagtacctggtcatcaggtattaatggaaagctttttgctgtgcattcaatcttcatagaggtctgctttccaattacattctctttgttttaagtataactttgaaactcaaatataaaagcctgctacttttttttgttctaaaaagacagatgtggactaggcacctgtactgtgaaaccagaatagagaagctgttgggcagatttgaagaggattgagtaatatagagaatgtcttgagatatttgtgaagcttttattgctttgttgcaattgttgtctagaaacatgggctttttctgttttttctcctattccaggagaggagggcgcatcactttaggaaaggaagagcagtgctcaaaagtgtt

SEQ ID NO: 24 - DNA, 1565 bp, synthetic construct
  misc_feature 1..1565: note = Description of Artificial Sequence: Synthetic polynucleotide
  misc_feature 1..1565: note = 3' HA of TV to the last exon of DAZL
  misc_feature 1..1565: note = ch2:34,382,718..34,384,284 white leghorn layer GRCg7w
  source 1..1565: mol_type = other DNA
tgatgaacaaagactttgaagtacataaatgtattactttgatgttcctacagttcagtttagtaagatgtgtagtaaaaagtgtaaccttgttcaaaaagttgcttcaagttgatgtttgtgttctgttttacctgttccagaatagctatttttgcttgagaagtttgaagttgtaagagttgaaatatttccaggttttattactagcttgcatgcttttcctgctaactaactgaaatgctaatcttaaggaatttatatggggaaggggaaaaaagaaaaacactttgtttggtacgtgtggattttcttctgagctttaaggtacagtttgttgcatgttaaaatttagttcttattaaaccacaactttaagttactaacgtcaaccagttacctcttgcagttcaaaagttgaagcagttccttgtccaagatggagtattttaaaactgagctcttaatcagtggaacagaagacgtcacggtgtaactcaactgaagccctttaagtcccggttctctttagactacctaatcaatgtctttgtttgctaacgacagtttatctatgtgaatcctaaaattcctatatgtaacttaagatgcaagaatgtaattagttacattggctgctcagtggagtatgacttttttttttactggattaattttagcaatacctgtatcttaaaattgtgagaaaatactgcatttaaaatatgcctaactttgtgatgcaatatgttaatcaaagaatacatgtaagcatattttaataataattatgtagattttagtcatgtattttgaaacaattaaaatttttaattttgacttaccttcccagtgtgagtgacatcctaatataatacttctaaatcttaagctgctttgagaaaggcatgcagcgtatttattgaaggaattgaagatttcttaccctacataagaattccagttaggacaagtttatagcaacaaactttcacatttgctgttagttctacctgcgattttgaaggagtacaactgagaacagcactcagtcttgtatgtgtgttgggtcctagtctgattcatttttcttattactacccttatacctcagtctccaagtaaaaaaggaaataactcctcctttgtagacgtgtatatgtgaatgaatagaatggcatgtcccacttcaaatgtctagaagtagatgttggtgaaacatgcaataagagctgagttgctctgtacctggatagtgggctgtaagatgcagcacaggagtgtcccaggactgttgttcaggagtaggagtcaggggagggaggcagggcttgggggatagcaataatagttggtgccgttctccataaaattactcagaagcaatgttctggcagcataaatcgacctaaagttgttaactttttctctgctcagaccgttccctagcactaaagatgctgtgtccttaagtgtgtttccatactatccatcaccatgtttttcccaacctattgctttaatagtattgttagtttgatatacttaacttcttccgcgtttcc

SEQ ID NO: 25 - DNA, 20 bp, synthetic construct
  misc_feature 1..20: note = Description of Artificial Sequence: Synthetic oligonucleotide
  misc_feature 1..20: note = TV-sgRNA
  misc_feature 1..20: note = ch2:34,384,310..34,384,329 white leghorn layer GRCg7w
  source 1..20: mol_type = other DNA
agggcgcatcacttcagaaa

SEQ ID NO: 26 - DNA, 1053 bp, synthetic construct
  misc_feature 1..1053: note = Description of Artificial Sequence: Synthetic polynucleotide
  misc_feature 1..1053: note = Cre rcombinaze including NLS
  source 1..1053: mol_type = other DNA
atggcacccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttgtgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctgcaagatggcgat

SEQ ID NO: 27 - protein, 351 aa, synthetic construct
  REGION 1..351: note = Description of Artificial Sequence: Synthetic polypeptide
  REGION 1..351: note = Cre rcombinaze including NLS
MAPKKKRKVSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVVRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLQDGD

SEQ ID NO: 28 - DNA, 1296 bp, synthetic construct
  misc_feature 1..1296: note = Description of Artificial Sequence: Synthetic polynucleotide
  misc_feature 1..1296: note = Flipase recombinase including NLS
  source 1..1296: mol_type = other DNA
atggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatc

SEQ ID NO: 29 - protein, 432 aa, synthetic construct
  REGION 1..432: note = Description of Artificial Sequence: Synthetic polypeptide
  REGION 1..432: note = Flipase recombinase including NLS
MAPKKKRKVMSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 30 - DNA, 48 bp, synthetic construct
  misc_feature 1..48: note = Description of Artificial Sequence: Synthetic oligonucleotide
  misc_feature 1..48: note = FRT
  source 1..48: mol_type = other DNA
gaagttcctattccgaagttcctattctctagaaagtataggaacttc

SEQ ID NO: 31 - DNA, 170 bp, synthetic construct
  misc_feature 1..170: note = Description of Artificial Sequence: Synthetic polynucleotide
  misc_feature 1..170: note = FRT-5'ssODN (with FRT)
  source 1..170: mol_type = other DNA
tatgcagatgcctatgtttagatagtatggtagccccaatagctttaccaaaattcctcctgaagttcctattccgaagttcctattctctagaaagtataggaacttcatagtgaacataaatataacatttttttgtaagagcttctttattgcttctatctaaccca

SEQ ID NO: 32 - DNA, 170 bp, synthetic construct
  misc_feature 1..170: note = Description of Artificial Sequence: Synthetic polynucleotide
  misc_feature 1..170: note = FRT-3'ssODN (With FRT)
  source 1..170: mol_type = other DNA
tccactttcctctgacctgaactgagtgagaaactttgaggctgtgagttacgttcaagtcgaagttcctattccgaagttcctattctctagaaagtataggaacttctaaagggtgcacgtggaatgtgggtgtgcgaagcacatcaccgctgtagttattccattac

SEQ ID NO: 33 - DNA, 23 bp, synthetic construct
  source 1..23: mol_type = other DNA; note = sgRNA; note = Dazl CRISPR1
atatttatgttcactataggagg

SEQ ID NO: 34 - DNA, 23 bp, synthetic construct
  source 1..23: mol_type = other DNA; note = sgRNA; note = Dazl CRISPR2
tgagttacgttcaagtctaaagg

SEQ ID NO: 35 - DNA, 156 bp, synthetic construct; identical to SEQ ID NO: 19
  source 1..156: mol_type = other DNA; note = ssODN; note = DAZL ssODN 1

SEQ ID NO: 36 - DNA, 156 bp, synthetic construct; identical to SEQ ID NO: 20
  source 1..156: mol_type = other DNA; note = ssODN; note = DAZL ssODN2

SEQ ID NO: 37 - DNA, 21 bp, synthetic construct
  source 1..21: mol_type = other DNA; note = Primers; note = F-Pre-D-ssODN1
tgctttagtgaaagatgtccc

SEQ ID NO: 38 - DNA, 23 bp, synthetic construct
  source 1..23: mol_type = other DNA; note = Primers; note = R-Pre-D-ssODN1
gttactttatttggcctaatacc

SEQ ID NO: 39 - DNA, 24 bp, synthetic construct
  source 1..24: mol_type = other DNA; note = Primers; note = F-Pre-D-ssODN2
tgtaatgtttgaagtgtgttttag

SEQ ID NO: 40 - DNA, 23 bp, synthetic construct
  source 1..23: mol_type = other DNA; note = Primers; note = R-Pre-D-ssODN2
gcacctcagaaaaaaagttttag

SEQ ID NO: 41 - DNA, 7189 bp, Gallus gallus
  source 1..7189: mol_type = genomic DNA; note = Whole Genomic Sequence; note = Wild type
agtgggagactgagtgagatgacctcctgaagttccctcctatctcagctattctatctacagaaatactgaactcttttcaagaggatgcctcaaaactatcttactatctaaaatatatatatatattttaatagtatttgtagcacctcttctaagacaggtatgttccaattaacagcaaacaggagataactgttccttaagaatgcttctagttagggccataatagaagtgaaaagagggcaactgtttccctcctcccccccctcccccccccaaaaaaagaaaaacaaacaaacaaaaaaccaacccaacctggtgtctgctctattttggcacaatgaaacaagcttagattcagcagctatcacaagcagatttactctttttgtgctctagaaatactggtactgatgtgttgccactcttgaaatcaaatatgaatctatttaattgaaaaagataagtatttttccttcactgttggattttattttcaaggttccagacatttgaaacaaaaatggaaaagagctgatacagtattgtagagcctcccttgtttttgtttgtttttcccccaagatgagggtggtgagacactgggacaggttgcctagaggctttgtgtgccccatcattagaagaattcatagatgggttggtcaatgctttgagcaacatgctttagtgaaagatgtccctgcccatggcaaggaggatggactagaatatctctaagggccctttccaacccatatcattcttgattctatttaagggacatttgttgcatgaaaacctatgcagatgcctatgtttagatagtatggtagccccaatagctttaccaaaattcctcctatagtgaacataaatataacatttttttgtaagagcttctttattgcttctatctaacccaactctgagtccaaatacatctagggcagcaagctattctggattattagtgtcagcatttaaaaagttgcacatttggtatgcttttaaaaacgataggggtattaggccaaataaagtaacatgtgagaaaacaggctgcctgttatatgccctccagttgatcagtttaactttgtacttttttttttccccagattaatcagtcttatacctttgaaatttatagaaagttgctaagctagcacagtcaagagtttgtcacagcatccataatgttcccttccccagaagggtctgaaaatctagatatattctcaagagaagaaaacaaaaagtatttaaaaccagcttccaaaggcagtggaagcaccttattaacatcgaaaaagaaaagcttccagagatatgtcccttctagaaagatcttaacagtctagtgagaaggtgttagaaaaggcattatggtgtaatggaaccagcctgctccagagtatccatataacatcggtgtgagaaaacattgccaaaacgttcagctttgcctcaaagtgggcaactctaaccaaagcctcctcaagcagtacaacctcactgggcatataacgcggctcttctgacaggaaggtgctaccactacgctctctgaacagcagggcctcaaacaatggccctacctaatctcctaacaaaggaaaaggttacggcacacatcgcccttcgtcttcagccctctcctgaaacacacaccgagggctgaactctcccacaccgcggcggtgcggtgcggtgcggtgcgggccggaccaggccaggccaggccaggccagcggggcgggtggggctttccccgccccttcccccggggtctacttaagaagagcggccgtcgaaaagaactgcctctttcgcacccctcttaaaaaagaaaagaaagaaaaaaaaaaaaaagacaaaaaaaatacaaacacaaaaaagtggggttctttagtgtctgttttcccaacactccaattgtttttgtcttgaaggcctcgtttgtttttaagtgtgcgggcgctgtcacagctccggggaacgatggtaagtcgggaccggggacaacgggggagcggagcggtggtcgggcgggtggagagcagggtgtgaggtgcgccgggccggcgggcagttggcggggcctcctgaagctcgcgtcgtgccgtggtggcagggcgggggacgggggagtcccgcagctgcggcgctgccgcctcacagcgccttgcggagactctgagaattactcccttttacaataggttctagaatcttacgtgtacggtatgaatttcagtgatactgcgagaaaatgataagaaaagaaggctgagacatgagttggtgttgtggtcagaggcgtggtgtcaggaggacttcgaggcaggagacgcgtcgtgccgcgagcggccattttgtgactccgtgcacagtcacggcccttctactccgccgcctccggtcggtgccccgaggtggcagttgcacccgctcggcgctggagcccggcgtgacgccttgccctgcgaccgcgggctctccatcagctccgtctattctcagccttttggcactccctcccctttcccttttcctaagcccgtccaggcccgtgacccgtttcccggcgccgagctgttgagccgcccgagcggaccgttatcacctcgggagctccggccccaccaggcggtttccggcagcgctccgcgggcccccagctgtggtcagcggggatgctgtccggccccgcgcgtgccccttccatgcaaagagaccttcgtcttcgagagttttctgcgtaatgtggtccccgttggggcgaagcctcgcgtcgctgggtgcgggtacagttctggttggtgctaaacgtgtgcagggaagggagcggtaaagcgttcctgtcgaggtgactgtgctctggtacacgagccggcagctgaaactaaactggttgcacacagtactgtttctcaagaaattctttattttcgggttttttttacgcacggtatctgctattctaacgctgacacctcagcaacttccaggcaggaccgggtagtggagtcgagcagcgctgctgtatggctccccctgccgcctccgggcgggcgcaacgccgcggagccgccgccatcttctccttccgcagcggctgccatggcaaccgagtgctcgtcgggaaggcgggtagcgggcgtgaatacggcgcagaatgccccttattttaaatgaaccccgtgtgagttcacggaggaagcagtctgagacatcaggaagcagtgcttgtgtggccgaaggaaaaagtgaaggttttcctcccctagacagagttgcagacatcactcaggcagttccacctcttttgtttaccaccccctgttgtaattaaggggttattctattttcctgccttgaagtcgatgctttgtgaaacaatgcagagagtaaattacttgatttaccttgtgataaaggaaaaagggctagggggaaagaaattcagcaggttgaaaaactgaataagctgtgagtctcttaacacctagaaattggttttcaaagttactttgtgagctgcagagacagacagagaagctttgcttaaggcagttttgaatggtctggtccctttagttggactcagtgatccttatgggtcctctccagctcggaatgttctatgattcagctgatgcttgagcttcatggtacgtaattcatccatttaagtgttgtttacagttttgggaattggaaatggagcaggctttggctactgattcagcagcagtgcagtttgcccgtagtatgcacagaagtaaggctgcattgggtagtaaaataacaatgacattttgtgctgataaaataagggtgatgcgttgcttgaattgacatgttttctgttgtagtactgaaggcttcaaaaataacttcgaaaggagaaagacattgtttcatgtcttcatactgtaatactttttcatcatgttcaccagtagctcctttttatgttctgagaaataagcatgttaatgcatctaaaagcttgtttttgatgttgaaaagtttgtcacactgcttaaagaacttacttgcccctttagatatttaacatgcagaagggttaaagaactgtgtaagaactgtattttctttgaaagttttattctgcatcattattgcgagaaactaatgatgtgcttctatggcagccaaggccgtttcttacctgcatgtcagcagtatctgtctgccgacccactttcttgtgctgattctggctctcctgggtgcatttgctgtgagctgcttcctgttgaaagttgccagaggatttgcacagccaagtgaatgctttctttgagcttcggtcactgaaacaggtccaaaaagagggaacagtagatgctaacaggtcagctcaaggttgctgtggagttggtctcatatttgagttaaaatgttctttttctggggatatggtcaaataagtggtctccagctctcctcttgtttccttcttgctgtgatgtgttctcatgctagaagtgtttacacaactgtggaatgagcttggatcagaggcagttgtgttggaaaaccacaacccattttctcctgtaaaatgttgggccttattccctttgcagtggtgtgaagagctgtcctgatacaataggaaggaagggagaagagtattctctatctgcgtacaacagaagtaatctgtgatacaatttgcagttggtagcatataattgatgaagtaaacagaattatatacaagaacatggtactggatgtgatggagccaggtacttcaaggtctagcttcattagatgctgttgaactgtgcttctcagactaggtcttttccatctccatttctgatggagatgaaacttaatctgaggtgtatctgatgcttatctgatagcacagtaagctgattctattgtctaaaataacagaatactaatctggtcaaaacttgctctcttgatgctgttcttcctcaaaaactaaatgtgaactgacagctgttggatcttctgcagaggaggtgtaagagaagagatggagcaaactgtctctgctttttaggggtagtctggtgttaatttggctcactcatgttgaaatcacacacgtggaaagctgagttctagttctggcgttacttaattgctggttgaatcaggccacatggacagtttggttcttgtaattttcttagtcccttaccggatgtccaatttgaatgaacattgatcacttgcaggtgcagtttaaataactctgtgttaaacgtctaatttgttacaatcacgagagcccatttttcaatgtaaattattcttaggtttcaaagtatcagtaacctcaactaaatcaaaagatctgcctatctgaaatagggataatgctacaccaggtgagctgcaaggaaaaggttattaatgttttgagatgtcttaatacagacaaatgagcacaaataaggtggtcaaagtagttgttttttttacaagccagataaagaatgacatgtacatagaaccattcattcagttgagaagatgtggaaccaaagtttcatccatctgaggtgtatttcagttcttgcaaatatctttgtgtaatgtttgaagtgtgttttagagtatggaacacgtcttggtgtcatcagcaacaagaaatgaaattgtgtggtctctgtgaacaaatgattccctaaataaacagtaatccagaatccactttcctctgacctgaactgagtgagaaactttgaggctgtgagttacgttcaagtctaaagggggcgcgtggaatgtgggtgtgcgaagcacatcaccgccgtagttattccattacaccatgtagatatgtgcagcgcactcttaagatcctgcttcggtgtgtgccactcagtgacaagatcagtcgttcatatttctcttgtagttaaattgactaaaactttttttctgaggtgcacataaagtgaatattctaccaagacggtgtcatctacctcacagctaaattgtagtataactgtaccatcgttccaaggaatttcataagctttactcacttcttgagcatcacaggcttttgatcaggaaaatggagttcattcatcggataatcaattcacagcgtagaacttaagaatttcttgcttgcatctaaaaggaattgtgtaaaaatttgcgatgaataatttcggggttctcattgtaagttgggtagtagcaacaatggttgtgaagcttccagtcaggcaaggctgcttagtgtagcctagcttgagtcttgatctcaaagaagagtaggttcaaacttgtccatttgagtgttccatttgcagtaagtacttcctttaagtaattggaaagatgctattgatacttatggtctcaaactgtatcaataggaagatggaggcctacctagtagtgtattacaatgtgactgaaatatttctgtttaaccttttcagtctgcaaatgcggaagcccagtgtggaactatctcagaggataatacccattcgtcaacaacctgccaaggatatgttttaccagaaggaaaaatcatgccaaatacagtctttgttggtggaattgatataagggtatttatgtactttcaatggttttaaactacatatgacacgctgtagtgggaaagaaataagaattttaacttctggagggcttttttttaattgggtctttactgatcttgaaataatgcattatggtaagagaactttgaaacaaacaaaagagattttcctggaatattaggagatgtgtttaaaaatggtacttgttgctttaaaacaattgtaaccgtttactgtgtctgtgaagtagttcaagacttggtttcttttagatgaatgaagcagaaattcggagttactttgaacaatatggtactgtgaaggaggtgaaaataatcactgacagaactggtgtttccaaagggtgagcagaatgtcattagttactgcttttgtagatgtaattctaacataaatgatgtctgttgttaagtagttggtcacttaccatgcttaagcctttcaaattgagatgaattaaagtgaaacatgtaaaactcgactgatcttaaatctgtataatcttacaattgagaactggacatattttatggtgtacctgttcagggaaaat

SEQ ID NO: 42 - DNA, 398 bp, synthetic construct
  source 1..398: mol_type = other DNA; note = PCR product sequence; note = Verified sequence of PCR product ssODN1
tgctttagtgaaagatgtccctgcccatggcaaggaggatggactagaatatctctaagggccctttccaacccatatcattcttgattctatttaagggacatttgttgcatgaaaacctatgcagatgcctatgtttagatagtatggtagccccaatagctttaccaaaattcctcctataacttcgtataatgtatgctatacgaagttatatagtgaacataaatataacatttttttgtaagagcttctttattgcttctatctaacccaactctgagtccaaatacatctagggcagcaagctattctggattattagtgtcagcatttaaaaagttgcacatttggtatgcttttaaaaacgataggggtattaggccaaataaagtaac

SEQ ID NO: 43 - DNA, 398 bp, synthetic construct
  source 1..398: mol_type = other DNA; note = PCR product sequence; note = Verified sequence of PCR product ssODN2
tgtaatgtttgaagtgtgttttagagtacggaacacgtcttggtgtcatcagcaacaagaaatgaaattgtgtgtgatctctgtgaacaaatgattccctaaataaacagtaatccagaatccactttcctctgacctgaactgagtgagaaactttgaggctgtgagttacgttcaagtcataacttcgtataatgtatgctatacgaagttattaaagggtgcacgtggaatgtgggtgtgcgaagcacatcaccgctgtagttattccattacaccatgtagatatgtgcagtgcactcttaagatcctgcttcggtgtgtgccactcagtgacaagatcagtcgttcatatttctcttgtagttaaattgactaaaactttttttctgaggtgc

SEQ ID NO: 44 - DNA, 8655 bp, synthetic construct
  source 1..8655: mol_type = other DNA; note = Whole Genomic Sequence; note = With LoxP insertions
agtgggagactgagtgagatgacctcctgaagttccctcctatctcagctattctatctacagaaatactgaactcttttcaagaggatgcctcaaaactatcttactatctaaaatatatatatatattttaatagtatttgtagcacctcttctaagacaggtatgttccaattaacagcaaacaggagataactgttccttaagaatgcttctagttagggccataatagaagtgaaaagagggcaactgtttccctcctcccccccctcccccccccaaaaaaagaaaaacaaacaaacaaaaaaccaacccaacctggtgtctgctctattttggcacaatgaaacaagcttagattcagcagctatcacaagcagatttactctttttgtgctctagaaatactggtactgatgtgttgccactcttgaaatcaaatatgaatctatttaattgaaaaagataagtatttttccttcactgttggattttattttcaaggttccagacatttgaaacaaaaatggaaaagagctgatacagtattgtagagcctcccttgtttttgtttgtttttcccccaagatgagggtggtgagacactgggacaggttgcctagaggctttgtgtgccccatcattagaagaattcatagatgggttggtcaatgctttgagcaacatgctttagtgaaagatgtccctgcccatggcaaggaggatggactagaatatctctaagggccctttccaacccatatcattcttgattctatttaagggacatttgttgcatgaaaaccgatagtatggtagccccaatagctttaccaaaattcctcctataacttcgtataatgtatgctatacgaagttatatagtgaacataaatataacatttttttgtaagagcttctttattgcttctatctaacccaactctgagtccaaatacatctagggcagcaagctattctggattattagtgtcagcatttaaaaagttgcacatttggtatgcttttaaaaacgataggggtattaggccaaataaagtaacatgtgagaaaacaggctgcctgttatatgccctccagttgatcagtttaactttgtacttttttttttccccagattaatcagtcttatacctttgaaatttatagaaagttgctaagctagcacagtcaagagtttgtcacagcatccataatgttcccttccccagaagggtctgaaaatctagatatattctcaagagaagaaaacaaaaagtatttaaaaccagcttccaaaggcagtggaagcaccttattaacatcgaaaaagaaaagcttccagagatatgtcccttctagaaagatcttaacagtctagtgagaaggtgttagaaaaggcattatggtgtaatggaaccagcctgctccagagtatccatataacatcggtgtgagaaaacattgccaaaacgttcagctttgcctcaaagtgggcaactctaaccaaagcctcctcaagcagtacaacctcactgggcatataacgcggctcttctgacaggaaggtgctaccactacgctctctgaacagcagggcctcaaacaatggccctacctaatctcctaacaaaggaaaaggttacggcacacatcgcccttcgtcttcagccctctcctgaaacacacaccgagggctgaactctcccacaccgcggcggtgcggtgcggtgcggtgcgggccggaccaggccaggccaggccaggccagcggggcgggtggggctttccccgccccttcccccggggtctacttaagaagagcggccgtcgaaaagaactgcctctttcgcacccctcttaaaaaagaaaagaaagaaaaaaaaaaaaaagacaaaaaaaatacaaacacaaaaaagtggggttctttagtgtctgttttcccaacactccaattgtttttgtcttgaaggcctcgtttgtttttaagtgtgcgggcgctgtcacagctccggggaacgatggtaagtcgggaccggggacaacgggggagcggagcggtggtcgggcgggtggagagcagggtgtgaggtgcgccgggccggcgggcagttggcggggcctcctgaagctcgcgtcgtgccgtggtggcagggcgggggacgggggagtcccgcagctgcggcgctgccgcctcacagcgccttgcggagactctgagaattactcccttttacaataggttctagaatcttacgtgtacggtatgaatttcagtgatactgcgagaaaatgataagaaaagaaggctgagacatgagttggtgttgtggtcagaggcgtggtgtcaggaggacttcgaggcaggagacgcgtcgtgccgcgagcggccattttgtgactccgtgcacagtcacggcccttctactccgccgcctccggtcggtgccccgaggtggcagttgcacccgctcggcgctggagcccggcgtgacgccttgccctgcgaccgcgggctctccatcagctccgtctattctcagccttttggcactccctcccctttcccttttcctaagcccgtccaggcccgtgacccgtttcccggcgccgagctgttgagccgcccgagcggaccgttatcacctcgggagctccggccccaccaggcggtttccggcagcgctccgcgggcccccagctgtggtcagcggggatgctgtccggccccgcgcgtgccccttccatgcaaagagaccttcgtcttcgagagttttctgcgtaatgtggtccccgttggggcgaagcctcgcgtcgctgggtgcgggtacagttctggttggtgctaaacgtgtgcagggaagggagcggtaaagcgttcctgtcgaggtgactgtgctctggtacacgagccggcagctgaaactaaactggttgcacacagtactgtttctcaagaaattctttattttcgggttttttttacgcacggtatctgctattctaacgctgacacctcagcaacttccaggcaggaccgggtagtggagtcgagcagcgctgctgtatggctccccctgccgcctccgggcgggcgcaacgccgcggagccgccgccatcttctccttccgcagcggctgccatggcaaccgagtgctcgtcgggaaggcgggtagcgggcgtgaatacggcgcagaatgccccttattttaaatgaaccccgtgtgagttcacggaggaagcagtctgagacatcaggaagcagtgcttgtgtggccgaaggaaaaagtgaaggttttcctcccctagacagagttgcagacatcactcaggcagttccacctcttttgtttaccaccccctgttgtaattaaggggttattctattttcctgccttgaagtcgatgctttgtgaaacaatgcagagagtaaattacttgatttaccttgtgataaaggaaaaagggctagggggaaagaaattcagcaggttgaaaaactgaataagctgtgagtctcttaacacctagaaattggttttcaaagttactttgtgagctgcagagacagacagagaagctttgcttaaggcagttttgaatggtctggtccctttagttggactcagtgatccttatgggtcctctccagctcggaatgttctatgattcagctgatgcttgagcttcatggtacgtaattcatccatttaagtgttgtttacagttttgggaattggaaatggagcaggctttggctactgattcagcagcagtgcagtttgcccgtagtatgcacagaagtaaggctgcattgggtagtaaaataacaatgacattttgtgctgataaaataagggtgatgcgttgcttgaattgacatgttttctgttgtagtactgaaggcttcaaaaataacttcgaaaggagaaagacattgtttcatgtcttcatactgtaatactttttcatcatgttcaccagtagctcctttttatgttctgagaaataagcatgttaatgcatctaaaagcttgtttttgatgttgaaaagtttgtcacactgcttaaagaacttacttgcccctttagatatttaacatgcagaagggttaaagaactgtgtaagaactgtattttctttgaaagttttattctgcatcattattgcgagaaactaatgatgtgcttctatggcagccaaggccgtttcttacctgcatgtcagcagtatctgtctgccgacccactttcttgtgctgattctggctctcctgggtgcatttgctgtgagctgcttcctgttgaaagttgccagaggatttgcacagccaagtgaatgctttctttgagcttcggtcactgaaacaggtccaaaaagagggaacagtagatgctaacaggtcagctcaaggttgctgtggagttggtctcatatttgagttaaaatgttctttttctggggatatggtcaaataagtggtctccagctctcctcttgtttccttcttgctgtgatgtgttctcatgctagaagtgtttacacaactgtggaatgagcttggatcagaggcagttgtgttggaaaaccacaacccattttctcctgtaaaatgttgggccttattccctttgcagtggtgtgaagagctgtcctgatacaataggaaggaagggagaagagtattctctatctgcgtacaacagaagtaatctgtgatacaatttgcagttggtagcatataattgatgaagtaaacagaattatatacaagaacatggtactggatgtgatggagccaggtacttcaaggtctagcttcattagatgctgttgaactgtgcttctcagactaggtcttttccatctccatttctgatggagatgaaacttaatctgaggtgtatctgatgcttatctgatagcacagtaagctgattctattgtctaaaataacagaatactaatctggtcaaaacttgctctcttgatgctgttcttcctcaaaaactaaatgtgaactgacagctgttggatcttctgcagaggaggtgtaagagaagagatggagcaaactgtctctgctttttaggggtagtctggtgttaatttggctcactcatgttgaaatcacacacgtggaaagctgagttctagttctggcgttacttaattgctggttgaatcaggccacatggacagtttggttcttgtaattttcttagtcccttaccggatgtccaatttgaatgaacattgatcacttgcaggtgcagtttaaataactctgtgttaaacgtctaatttgttacaatcacgagagcccatttttcaatgtaaattattcttaggtttcaaagtatcagtaacctcaactaaatcaaaagatctgcctatctgaaatagggataatgctacaccaggtgagctgcaaggaaaaggttattaatgttttgagatgtcttaatacagacaaatgagcacaaataaggtggtcaaagtagttgttttttttacaagccagataaagaatgacatgtacatagaaccattcattcagttgagaagatgtggaaccaaagtttcatccatctgaggtgtatttcagttcttgcaaatatctttgtgtaatgtttgaagtgtgttttagagtatggaacacgtcttggtgtcatcagcaacaagaaatgaaattgtgtggtctctgtgaacaaatgattccctaaataaacagtaatccagaatccactttcctctgacctgaactgagtgagaaactttgaggctgtgagttacgttcaagtcataacttcgtataatgtatgctatacgaagttattaaagggtgcacgtggaatgtgggtgtgcgaagcacatcaccgctgtagttattccattacaccatgtagatatgtgcagcgcactcttaagatcctgcttcggtgtgtgccactcagtgacaagatcagtcgttcatatttctcttgtagttaaattgactaaaactttttttctgaggtgcacataaagtgaatattctaccaagacggtgtcatctacctcacagctaaattgtagtataactgtaccatcgttccaaggaatttcataagctttactcacttcttgagcatcacaggcttttgatcaggaaaatggagttcattcatcggataatcaattcacagcgtagaacttaagaatttcttgcttgcatctaaaaggaattgtgtaaaaatttgcgatgaataatttcggggttctcattgtaagttgggtagtagcaacaatggttgtgaagcttccagtcaggcaaggctgcttagtgtagcctagcttgagtcttgatctcaaagaagagtaggttcaaacttgtccatttgagtgttccatttgcagtaagtacttcctttaagtaattggaaagatgctattgatacttatggtctcaaactgtatcaataggaagatggaggcctacctagtagtgtattacaatgtgactgaaatatttctgtttaaccttttcagtctgcaaatgcggaagcccagtgtggaactatctcagaggataatacccattcgtcaacaacctgccaaggatatgttttaccagaaggaaaaatcatgccaaatacagtctttgttggtggaattgatataagggtatttatgtactttcaatggttttaaactacatatgacacgctgtagtgggaaagaaataagaattttaacttctggagggcttttttttaattgggtctttactgatcttgaaataatgcattatggtaagagaactttgaaacaaacaaaagagattttcctggaatattaggagatgtgtttaaaaatggtacttgttgctttaaaacaattgtaaccgtttactgtgtctgtgaagtagttcaagacttggtttcttttagatgaatgaagcagaaattcggagttactttgaacaatatggtactgtgaaggaggtgaaaataatcactgacagaactggtgtttccaaagggtgagcagaatgtcattagttactgcttttgtagatgtaattctaacataaatgatgtctgttgttaagtagttggtcacttaccatgcttaagcctttcaaattgagatgaattaaagtgaaacatgtaaaactcgactgatcttaaatctgtataatcttacaattgagaactggacatattttatggtgtacctgttcagggaaaatagtgttaatgtccctcaaccagtgggagcaaagcataaaacttagtggatgcttgtggggactgtttcacaggctgaaattttgactttatgatggccatagcaattaagcagccatcagtgtagtaccactaatgtaattgagacagggagtagactttcattggggcagttggactgcagtcttttgttgctcaggggtaacttagaggcaatcaaactgtttcaggtggtgagtgaaacttaaaggatggtagaaagctagagacattcccattggatatgtagaaagtactctgatctgtagtgaagaacttaagtgaagatgcctaggactctgcccagttgagttcagaggaagctctcccagctttgaaattagacttgctttgcgaggaagacttcacctctaaagatgcaccaattgttttctctgagcaggttccaaaaagtagcattttttttttcatagacacatatagtaacgagctgaaaatactgagcttaatgtctcttgcctggtctttgtggtgaattctaatgtgtgattagcaagcatatgttctgattattgataaattgctgtatgtcaatcaatggaatactctactgcggttctgagaattgtctccaatattaaggcttaaataaacaagaggtagtgagataaattgaaaacctcttttgggatcgcttcctccaatagtgtaattattcctgtagttcctcctttcattcaaacctctgcaggaagtacagaatttagtacatactaattgaaggagcttttggctttctgatgctactaatattaacagtagtactcacttgagtaatttaaatgagagaatattgaatctggcatttaattcctttcatttggcccagtgtgctgtcagtcagcagcaaatgtactttcatgctgaattatatattaatgtcctgttaatatcagttaatgttctttttactgttttagtttttttaaaaagaactaacagcggtcaaaaaatgaaaatgtagtatttgaataatattttttttcttttcaggtatggatttgtttcattcctggacaatgtggatgttcaaaagatagtagaagtaagctctttatgtcttaagttgtcagaagaaccttctgtatgaaggttgtaggtgtggttaggggataccagtcccaactgagaaaataaaaaagactagaagtgccccaaagtaaacttgcttaaatattgttgtgatttaccccagcagattgggaagtaccatgtactattttcctcactgcactccttcccagtggtatgggaagagaaaaaacaaaaaccccaaaaaaacaccacaaagtagagcttgtaagttgagatgaaaactatttaccaagagaataggaagaggaaaaatgggtaacggggt

SEQ ID NO: 45 - DNA, 20 bp, synthetic construct
  source 1..20: mol_type = other DNA; note = sgRNA; note = DAZL cr1 End
agggcgcatcacttcagaaa

SEQ ID NO: 46 - DNA, 1330 bp, synthetic construct
  source 1..1330: mol_type = other DNA; note = DAZL Cre-Intein targeting vectors; note = 5' homology arm
gaattgtggaagaattgcagctactaaaaaggcagactgggtagcacttcccaccttactcagatttaggagtctcaagacatgtagatgggtgaaatttattattcttttctctgcttgcttttctggaaagctacctctatttggccagcagttggctctgaattcctttctttaaaggagacctgactgctctcctgtctgcctcttacacagctgtactggtgtgtcactgatagaaacgtttaccttgaaagtgacaagtgggggcgttgatacccattaacaacattaaagggaaaatttcaggaaacaagaaaactgagaggagttcagaatgaaatgtaagccctgtgacagactcacactgatggtagtagggtaactgatggagatgcatctataacaattctaacttttatttcagaagtctgtggacaggagcatacaaacagtagtatcttgtctgtttaaccctgaaaaccgtctgaggaacacctttgtatcacaagaagactacttcagggtatgtagtgaaagcaaaactaaatctctaaaaccagctgatcttaactttgttcatatacaggatgccatgaagacaaaggtttatctgtacttgcttgagattttactttttgtatgtgcattgagatgaatgactggttagctctcagttggttaatactcaatgaaaattgcagaaattgagttcagtggatttaagtgcattgatacaaggctgcataagaactgctgagcagtcagctattggatatttagagctaatcatccctcaggagcaatctgctgaaaggtaagcagattgcatcttgtaaacgagagagcagagttaaacgtttgtggtctttgctccacttaagtacccacagtggatgtctatctgtacttttggttaaaaaatagataatttctacaacagactgtagttaggtcagtacctggtcatcaggtattaatggaaagctttttgctgtgcattcaatcttcatagaggtctgctttccaattacattctctttgttttaagtataactttgaaactcaaatataaaagcctgctacttttttttgttctaaaaagacagatgtggactaggcacctgtactgtgaaaccagaatagagaagctgttgggcagatttgaagaggattgagtaatatagagaatgtcttgagatatttgtgaagcttttattgctttgttgcaattgttgtctagaaacatgggctttttctgttttttctcctattccaggagaggagggcgcatcacttcagaaaaggaagagcagtgctcaaaagtgtt

SEQ ID NO: 47 - DNA, 1780 bp, synthetic construct
  source 1..1780: mol_type = other DNA; note = DAZL Cre-Intein targeting vectors; note = 3' homology arm
tgatgaacaaagactttgaagtacataaatgtattactttgatgttcctacagttcagtttagtaagatgtgtagtaaaaagtgtaaccttgttcaaaaagttgcttcaagttgatgtttgtgttctgttttacctgttccagaatagctatttttgcttgagaagtttgaagttgtaagagttgaaatatttccaggttttattactagcttgcatgcttttcctgctaactaactgaaatgctaatcttaaggaatttatatggggaaggggaaaaaagaaaaacactttgtttggtatgtgtggattttcttctgagctttaaggtacagtttgttgcatgttaaaatttagttcttattaaaccacaactttaagttactaacgtcaaccagttacctcttgcagttcaaaagttgaagcagttccttgtccaagatggagtattttaaaactgagctcttaatcagtggaacagaagacgtcacggtgtaactcaactgaagccctttaagtcccggttctctttagactacctaatcaatgtctttgtttgctaacgacagttcatctatgtgaatcctaaaattcctatatgtaacttaagatgcaagaatgtaattagttacattggctgctcagtggagtatgacttttttttttactggattaattttagcaatacctgtatcttaaaattgtgagaaaatactgcatttaaaatatgcctaactttgtgatgcaatatgttaatcaaagaatacatgtaagcatattttaataataattatgtagattttagtcatgtattttgaaacaattaaaatttttaattttgacttaccttcccagtgtgagtgacatcctaatataatacttctaaatcttaagctgctttgagaagggcatgcagcgtatttattgaaggaattgaagatttcttaccctacataagaattccagttaggacaagtttatagcaacaaactttcacatttgctgttagttctacctgcgattttgaaggagtacaactgagaacagcactcagtcttgtatgtgtgttgggtcctagtctgattcatttttcttattactacccttatacctcagtctccaagtaaaaaaggaaataactcctcctttgtagacgtgtatatgtgaatgaatagaatggcatgtcccacttcaaatgtctagaagtagatgttggtgaaacatgcaataagagctgagttgctctggacctggatagtgggctgtaagatgcagcacaggagtgtcccaggactgttgttcaggagtaggagtcaggggagggaggcagggcttgggggatagcaataatagttggtgccgttctccataaaattactcagaagcaatgttctggcagcataaatcgacctaaagttgttaactttttctctgctcagaccgttccctatcactaaagatgctgtgtccttaagtgtgtttccatactatctatcaccatgtttttcccaacctattgctttaatagtattgttagtttgatatacttaacttcttccgcgtttccagcttctgtgtaaatggttttcaatggtacacaattaaacttcacatacaagtaatctggagttttctgagtacaaggaccctttccttcgcaactttaagtacctgagcagcagccgacttggattgttacatctgtgatgtcagtttattgataaaggtaattgtttcttcgcaaatgcagaattcattttggcttaatgttctagttggctt

SEQ ID NO: 48 - DNA, 21 bp, synthetic construct
  source 1..21: mol_type = other DNA; note = DAZL NLS-nCre-nIntein targeting vector; note = NLS
cccaagaagaagaggaaggtg

SEQ ID NO: 49 - DNA, 125 bp, synthetic construct
  source 1..125: mol_type = other DNA; note = DAZL NLS-nCre-nIntein targeting vector; note = n-Cre recombinase
acgagtgatgaggttcgcaagaacctgatggacatgttcagggatcgccaggcgttttctgagcatacctggaaaatgcttctgtccgtttgccggtcgtgggcggcatggtgcaagttgaataa

SEQ ID NO: 50 - DNA, 382 bp, synthetic construct
  source 1..382: mol_type = other DNA; note = DAZL NLS-nCre-nIntein targeting vector; note = n-Intein
atttgcggaatattgcctcagttttggcaccgaaattttaaccgttgagtacggcccattgcccattggcaaaattgtgagtgaagaaattaattgttctgtgtacagtgttgatccagaagggagagtttacacccaggcgatcgcccaatggcatgaccggggagagcaggaagtattggaatatgaattggaagatggttcagtaatccgagctacctctgaccaccgctttttaaccaccgattatcaactgttggcgatcgaagaaatttttgctaggcaactggacttgttgactttagaaaatattaagcaaactgaagaagctcttgacaaccatcgtcttccctttccattacttgacgctgggacaattaaa

SEQ ID NO: 51 - DNA, 852 bp, synthetic construct
  source 1..852: mol_type = other DNA; note = DAZL cCre-cIntein targeting vector; note = c-Cre recombinase
aaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctgcaagatggcgat

SEQ ID NO: 52 - DNA, 120 bp, synthetic construct
  source 1..120: mol_type = other DNA; note = DAZL cCre-cIntein targeting vector; note = c-Intein
gttaaagttatcggtcgtcgttccctcggagtgcaaagaatatttgatattggtcttccccaagaccataattttctgctagccaatggggcgatcgccgccaattgttttaacaaatcc

SEQ ID NO: 53 - DNA, 66 bp, synthetic construct; identical to SEQ ID NO: 13
  source 1..66: mol_type = other DNA; note = DAZL Cre-Intein targeting vectors; note = P2A

SEQ ID NO: 54 - DNA, 63 bp, synthetic construct; identical to SEQ ID NO: 17
  source 1..63: mol_type = other DNA; note = DAZL Cre-Intein targeting vectors; note = T2A

SEQ ID NO: 55 - DNA, 714 bp, synthetic construct; identical to SEQ ID NO: 15
  source 1..714: mol_type = other DNA; note = DAZL Cre-Intein targeting vectors; note = GFP

SEQ ID NO: 56 - DNA, 21 bp, synthetic construct
  source 1..21: mol_type = other DNA; note = Primer A; note = F-DAZL-ATGCodonNest
gaattgtggaagaattgcagc

SEQ ID NO: 57 - DNA, 20 bp, synthetic construct
  source 1..20: mol_type = other DNA; note = Primer B; note = F_DAZL_EndSeq1
ggatgccatgaagacaaagg

SEQ ID NO: 58 - DNA, 21 bp, synthetic construct
  source 1..21: mol_type = other DNA; note = Primer C; note = R_DAZL_EndSeq1
tgcagccttgtatcaatgcac

SEQ ID NO: 59 - DNA, 21 bp, synthetic construct
  source 1..21: mol_type = other DNA; note = Primer D; note = F_DAZL_EndSeq2
aggaagagcagtgctcaaaag

SEQ ID NO: 60 - DNA, 18 bp, synthetic construct
  source 1..18: mol_type = other DNA; note = Primer E; note = R-magP2A
ggggccggggttctcctc

SEQ ID NO: 61 - DNA, 20 bp, synthetic construct
  source 1..20: mol_type = other DNA; note = Primer F; note = Fw-PBRAGE-EGFP
agatccgccacaacatcgag

SEQ ID NO: 62 - DNA, 20 bp, synthetic construct
  source 1..20: mol_type = other DNA; note = Primer G; note = Rv-PBRAGE-EGFP
tgctcaggtagtggttgtcg

SEQ ID NO: 63 - DNA, 20 bp, synthetic construct
  source 1..20: mol_type = other DNA; note = Primer H; note = F_DAZL_EndSeq3
tggctgctcagtggagtatg

SEQ ID NO: 64 - DNA, 22 bp, synthetic construct
  source 1..22: mol_type = other DNA; note = Primer I; note = R-Pre-D3HA-Nest
ccttgtactcagaaaactccag

SEQ ID NO: 65 - DNA, 22 bp, synthetic construct
  source 1..22: mol_type = other DNA; note = Primer J; note = R-Pre-D3HA
aagccaactagaacattaagcc

SEQ ID NO: 66 - DNA, 3209 bp, Gallus gallus
  source 1..3209: mol_type = genomic DNA; note = Genomic Sequence; note = Wild type
gaaggctatcttgcttccttgaaactaataatacctagtttgtagtgtttagggatgtgtaaaatgtgttgatgtcaagattctagttagtctgataagaattgtggaagaattgcagctactaaaaaggcagactgggtagcacttcccaccttactcagatttaggagtctcaagacatgtagatgggtgaaatttattattcttttctctgcttgcttttgtggaaagctacctctatttggccagcagttggctctgaattcctttctttaaaggagacctgactgctctcctgtctgcctcttacacagttgtactggtgtgtcactgatagaaacgtttaccttgaaagtgacaagtgggggcgttgatacccattaacaacattaaagggaaaatttcaggaagcaagaaaactgagaggagttcagaatgaaatgtaagccctgtgacagactcacactgatggtagtagggtaactgatggagatgcatctataacaattctaacttttatttcagaagtctgtggacaggagcatacaaacagtagtatcctgtctgtttaaccctgaaaaccgtctgaggaacacctttgtatcacaagaagactacttcagggtatgtagtgaaagcaaaactaaatctctaaaaccagctgatcttaactttgttcatatacaggatgccatgaagacaaaggtttatctgtacttgcttgagattttaatttttgtatgtgcattgagatgaatgactggttagctctcagttggttaatactcaatgaaaattgcagaaattgagttcagtggatttaagtgcattgatacaaggctgcataagaactgctgagcagtcagctattggatatttagagctaatcatccctcaggagcaatctgctgaaaggtaagcagattgcatcttgtaaacgagagagcagagttaaacgtttgtggtctttgctccacttaagtacccactgtggatgtctatctgtacttttggttaaaaaatagataatttctacaacagactgtagttaggtcagtacctggtcatcaggtattaatggaaagctttttgctgtgcattcaatcttcatagaggtctgctttccaattacattctctttgttttaagtataacattgaaactcaaatataaaagcctgctacttttttttgttctaaaaagacagatgtggactaggcacctgtactgtgaaaccagaatagagaagctgttgggcagatttgaagaggattgagtaatatagagaatgtcttgagatatttgtgaagcttttattgctttgttgcgattgttgtctagaaacatgggctttttctgttttttctcctattccaggagaggagggcgcatcacttcagaaaaggaagagcagtgctcaaaagtgtttgatgaacaaagactttgaagtacataaatgtattactttgatgttcctacagttcacgtttagtaagatgtgtagtaaaaagtgtaaccttgttcaaaaagttgcttcaagttgatgtttgtgttctgttttacctgttccagaatagctatttttgcttgagaagtttgaagttgtaagagttgaaatatttccaggttttattactagcttgcatgcttttcctgctaactaactgaaatgctaatcttaaggaatttatatggggaaggggaaaaaagaaaaacactttgtttggtatgtgtggattttcttctgagctttaaggtacagtttgttgcatgttaaaatttagttcttattaaaccacaactttaagttactaacgtcaaccagttacctcttgcagttcaaaagttgaagcagttccttgtccaagatggagtattttaaaactgagctcttaatcagtggaacagaagacgtcacggtgtaactcaactgaagccctttaagtcccggttctctttagactacctaatcaatgtctttgtttgctaacgacagttcatctatgtgaatcctaaaattcctatatgtaacttaagatgcaagaatgtaattagttacattggctgctcagtggagtatgacttttttttttactggattaattttagcaatacctgtatcttaaaattgtgagaaaatactgcatttaaaatatgcctaactttgtgatgcaatatgttaatcaaagaatacatgtaagcatattttaataataattatgtagattttagtcatgtattttgaaacaattaaaatttttaattttgacttaccttcccagtgtgagtgacatcctaatataatacttctaaatcttaagctgctttgagaagggcatgcagcgtatttattgaaggaattgaagatttcttaccctacataagaattccagttaggacaagtttatagcaacaaactttcacatttgctgttagttctacctgcgattttgaaggagtacaactgagaacagcactcagtcttgtatgtgtgttgggtcctagtctgattcatttttcttattactacccttatacctcagtctccaagtaaaaaaggaaataactcctcctttgtagacgtgtatatgtgaatgaatagaatggcatgtcccacttcaaatgtctagaagtagatgttggtgaaacatgcaataagagctgagttgctctggacctggatagtgggctgtaagatgcagcacaggagtgtcccaggactgttgttcaggagtaggagtcaggggagggaggcagggcttgggggatagcaataatagttggtgccgttctccataaaattactcagaagcaatgttctggcagcataaatcgacctaaagttgttaactttttctctgctcagaccgttccctatcactaaagatgctgtgtccttaagtgtgtttccatactatctatcaccatgtttttcccaacctattgctttaatagtattgttagtttgatatacttaacttcttccgcgtttccagcttctgtgtaaatggttttcaatggtacacaattaaacttcacatacaagtaatctggagttttctgagtacaaggaccctttccttcgcaactttaagtacctgagcagcagccgacttggattgttacatctgtgatgtcagtttattgataaaggtaattgtttcttcgcaaatgcagaattcattttggcttaatgttctagttggctt

SEQ ID NO: 67 - DNA, 4581 bp, synthetic construct
  source 1..4581: mol_type = other DNA; note = Genomic Sequence; note = NLS-nCre-nIntein kockin to DAZL
gaaggctatcttgcttccttgaaactaataatacctagtttgtagtgtttagggatgtgtaaaatgtgtttatgtcaagattttagttagtctgataagaattgtggaagaattgcagctactaaaaaggcagactgggtagcacttcccaccttactcagatttaggagtctcaagacatgtagatgggtgaaatttattattcttttctctgcttgcttttgtggaaagctacctctatttggccagcagttggctctgaattcctttctttaaaggagacctgactgctctcctgtctgcctcttacacagctgtactggtgtgtcactgatagaaacgtttaccttgaaagtgacaagtgggggcgttgatacccattaacaacattaaagggaaaatttcaggaaacaagaaaactgagaggagttcagaatgaaatgtaagccctgtgacagactcacactgatggtagtagggtaactgatggagatgcatctataacaattctaacttttatttcagaagtctgtggacaggagcatacaaacagtagtatcttgtctgtttaaccctgaaaaccgtctgaggaacacctttgtatcacaagaagactacttcagggtatgtagtgaaagcaaaactaaatctctaaaaccagctgatcttaactttgttcatatacaggatgccatgaagacaaaggtttatctgtacttgcttgagattttactttttgtatgtgcattgagatgaatgactggttagctctcagttggttaatactcaatgaaaattgcagaaattgagttcagtggatttaagtgcattgatacaaggctgcataagaactgctgagcagtcagctattggatatttagagctaatcatccctcaggagcaatctgctgaaaggtaagcagattgcatcttgtaaacgagagagcagagttaaacgtttgtggtctttgctccacttaagtacccactgtggatgtctatctgtacttttggttaaaaaatagataatttctacaacagactgtagttaggtcagtacctggtcatcaggtattaatggaaagctttttgctgtgcattcaatcttcatagaggtctgctttccaattacattctctttgttttaagtataactttgaaactcaaatataaaagcctgctacttttttttgttctaaaaagacagatgtggactaggcacctgtactgtgaaaccagaatagagaagctgttgggcagatttgaagaggattgagtaatatagagaatgtcttgagatatttgtgaagcttttattgctttgttgcaattgttgtctagaaacatgggctttttctgttttttctcctattccaggagaggagggcgcatcactttaggaaaggaagagcagtgctcaaaagtgttggcagcggcgccaccaacttcagcctgctgaagcaggccggcgacgtggaggagaaccccggcccccccaagaagaagaggaaggtgacgagtgatgaggttcgcaagaacctgatggacatgttcagggatcgccaggcgttttctgagcatacctggaaaatgcttctgtccgtttgccggtcgtgggcggcatggtgcaagttgaataaatttgcggaatattgcctcagttttggcaccgaaattttaaccgttgagtacggcccattgcccattggcaaaattgtgagtgaagaaattaattgttctgtgtacagtgttgatccagaagggagagtttacacccaggcgatcgcccaatggcatgaccggggagagcaggaagtattggaatatgaattggaagatggttcagtaatccgagctacctctgaccaccgctttttaaccaccgattatcaactgttggcgatcgaagaaatttttgctaggcaactggacttgttgactttagaaaatattaagcaaactgaagaagctcttgacaaccatcgtcttccctttccattacttgacgctgggacaattaaaggcagtggagagggcagaggaagtctgctaacatgcggtgacgtcgaggagaatcctggcccagtgagcaagggcgaggagctgttcaccggggtgttgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgatgaacaaagactttgaagtacataaatgtattactttgatgttcctacagttcagtttagtaagatgtgtagtaaaaagtgtaaccttgttcaaaaagttgcttcaagttgatgtttgtgttctgttttacctgttccagaatagctatttttgcttgagaagtttgaagttgtaagagttgaaatatttccaggttttattactagcttgcatgcttttcctgctaactaactgaaatgctaatcttaaggaatttatatggggaaggggaaaaaagaaaaacactttgtttggtatgtgtggattttcttctgagctttaaggtacagtttgttgcatgttaaaatttagttcttattaaaccacaactttaagttactaacgtcaaccagttacctcttgcagttcaaaagttgaagcagttccttgtccaagatggagtattttaaaactgagctcttaatcagtggaacagaagacgtcacggtgtaactcaactgaagccctttaagtcccggttctctttagactacctaatcaatgtctttgtttgctaacgacagttcatctatgtgaatcctaaaattcctatatgtaacttaagatgcaagaatgtaattagttacattggctgctcagtggagtatgacttttttttttactggattaattttagcaatacctgtatcttaaaattgtgagaaaatactgcatttaaaatatgcctaactttgtgatgcaatatgttaatcaaagaatacatgtaagcatattttaataataattatgtagattttagtcatgtattttgaaacaattaaaatttttaattttgacttaccttcccagtgtgagtgacatcctaatataatacttctaaatcttaagctgctttgagaagggcatgcagcatatttattgaaggaattgaagatttcttaccctacataagaattccagttaggacaagtttatagcaacaaacttttatgtttgctgttagttcctacctgcaattttgaaggagtacaactgagaacagcactcagtcttgtatgtgtgttgggtcctagtctgattcatttttcttattactacccttatacctcagtctccaagtaaaaaaggaaataactcctcctttgtagacgtgtatatgtgaatgaatagaatggcatgtcccacttcaaatgtctagaagtagatgttggtgaaacatgcaataagagctgagttgctctggacctggatagtgggctgtaagatgcagcacaggagtgtcccaggactgttgttcaggagtaggagtcaggggagggaggcagggcttgggggatagcaataatagttggtgccgttctccataaaattactcagaagcaatgttctggcagcataaatcgacctaaagttgttaacttttttctctgctcagactgttccctatcactaaagatgctgtgtccttaagtgtgtttccatactatctatcaccatgtttttcccaacctattgctttaatagtattgttagtttgatatacttaacttcttccgcgtttccagcttctgtgtaaatggttttcaatggtacacaattaaacttcacatacaagtaatctggagttttctgagtacaaggaccctttccttcgcaactttaagtacctgagcagcagccgacttggattgttacatctgtgatgtcagtttattgataaaggtaattgtttcttcgcaaatgcagaattcattttggcttaatgttctagttggctt

SEQ ID NO: 68 - DNA, 5023 bp, synthetic construct
  source 1..5023: mol_type = other DNA; note = Genomic Sequence; note = cCre-cIntein knockin to DAZL
gaaggctatcttgcttccttgaaactaataatacctagtttgtagtgtttagggatgtgtaaaatgtgtttatgtcaagattttagttagtctgataagaattgtggaagaattgcagctactaaaaaggcagactgggtagcacttcccaccttactcagatttaggagtctcaagacatgtagatgggtgaaatttattattcttttctctgcttgcttttctggaaagctacctctatttggccagcagttggctctgaattcctttctttaaaggagacctgactgctctcctgtctgcctcttacacagctgtactggtgtgtcactgatagaaacgtttaccttgaaagtgacaagtgggggcgttgatacccattaacaacattaaagggaaaatttcaggaaacaagaaaactgagaggagttcagaatgaaatgtaagccctgtgacagactcacactgatggtagtagggtaactgatggagatgcatctataacaattctaacttttatttcagaagtctgtggacaggagcatacaaacagtagtatcttgtctgtttaaccctgaaaaccgtctgaggaacacctttgtatcacaagaagactacttcagggtatgtagtgaaagcaaaactaaatctctaaaaccagctgatcttaactttgttcatatacaggatgccatgaagacaaaggtttatctgtacttgcttgagattttactttttgtatgtgcattgagatgaatgactggttagctctcagttggttaatactcaatgaaaattgcagaaattgagttcagtggatttaagtgcattgatacaaggctgcataagaactgctgagcagtcagctattggatatttagagctaatcatccctcaggagcaatctgctgaaaggtaagcagattgcatcttgtaaacgagagagcagagttaaacgtttgtggtctttgctccacttaagtacccacagtggatgtctatctgtacttttggttaaaaaatagataatttctacaacagactgtagttaggtcagtacctggtcatcaggtattaatggaaagctttttgctgtgcattcaatcttcatagaggtctgctttccaattacattctctttgttttaagtataactttgaaactcaaatataaaagcctgctacttttttttgttctaaaaagacagatgtggactaggcacctgtactgtgaaaccagaatagagaagctgttgggcagatttgaagaggattgagtaatatagagaatgtcttgagatatttgtgaagcttttattgctttgttgcaattgttgtctagaaacatgggctttttctgttttttctcctattccaggagaggagggcgcatcacttcagaaaaggaagagcagtgctcaaaagtgttggcagcggcgccaccaacttcagcctgctgaagcaggccggcgacgtggaggagaaccccggccccgttaaagttatcggtcgtcgttccctcggagtgcaaagaatatttgatattggtcttccccaagaccataattttctgctagccaatggggcgatcgccgccaattgttttaacaaatccaaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctgcaagatggcgatggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgatgaacaaagactttgaagtacataaatgtattactttgatgttcctacagttcagtttagtaagatgtgtagtaaaaagtgtaaccttgttcaaaaagttgcttcaagttgatgtttgtgttctgttttacctgttccagaatagctatttttgcttgagaagtttgaagttgtaagagttgaaatatttccaggttttattactagcttgcatgcttttcctgctaactaactgaaatgctaatcttaaggaatttatatggggaaggggaaaaaagaaaaacactttgtttggtacgtgtggattttcttctgagctttaaggtacagtttgttgcatgttaaaatttagttcttattaaaccacaactttaagttactaacgtcaaccagttacctcttgcagttcaaaagttgaagcagttccttgtccaagatggagtattttaaaactgagctcttaatcagtggaacagaagacgtcacggtgtaactcaactgaagccctttaagtcccggttctctttagactacctaatcaatgtctttgtttgctaacgacagtttatctatgtgaatcctaaaattcctatatgtaacttaagatgcaagaatgtaattagttacattggctgctcagtggagtatgacttttttttttactggattaattttagcaatacctgtatcttaaaattgtgagaaaatactgcatttaaaatatgcctaactttgtgatgcaatatgttaatcaaagaatacatgtaagcatattttaataataattatgtagattttagtcatgtattttgaaacaattaaaatttttaattttgacttaccttcccagtgtgagtgacatcctaatataatacttctaaatcttaagctgctttgagaaaggcatgcagcgtatttattgaaggaattgaagatttcttaccctacataagaattccagttaggacaagtttatagcaacaaactttcacatttgctgttagttctacctgcgattttgaaggagtacaactgagaacagcactcagtcttgtatgtgtgttgggtcctagtctgattcatttttcttattactacccttatacctcagtctccaagtaaaaaaggaaataactcctcctttgtagacgtgtatatgtgaatgaatagaatggcatgtcccacttcaaatgtctagaagtagatgttggtgaaacatgcaataagagctgagttgctctgtacctggatagtgggctgtaagatgcagcacaggagtgtcccaggactgttgttcaggagtaggagtcaggggagggaggcagggcttgggggatagcaataatagttggtgccgttctccataaaattactcagaagcaatgttctggcagcataaatcgacctaaagttgttaactttttctctgctcagaccgttccctagcactaaagatgctgtgtccttaagtgtgtttccatactatccatcaccatgtttttcccaacctattgctttaatagtattgttagtttgatatacttaacttcttccgcgtttccagcttctgtgtaaatggttttcaatggtacacaattaaacttcacatacaagtaatctggagttttctgagtacaaggaccctttccttcgcaactttaagtacctgagcagcagccgacttggattgttacatctgtgatgtcagtttattgataaaggtaattgtttcttcgcaaatgcagaattcattttggcttaatgttctagttggctt

SEQ ID NO: 69 - DNA, 1056 bp, synthetic construct
  source 1..1056: mol_type = other DNA; note = cre recombinase expression plasmid; note = cre recombinase
atggcacccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttgtgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctgcaagatggcgattag

SEQ ID NO: 70 - protein, 7 aa, synthetic construct
  source 1..7: note = NLS AA
PKKKRKV

SEQ ID NO: 71 - protein, 41 aa, synthetic construct; identical to SEQ ID NO: 6
  source 1..41: note = n-Cre recombinase AA

SEQ ID NO: 72 - protein, 127 aa, synthetic construct
  source 1..127: note = n-Intein AA
FAEYCLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 73 - protein, 284 aa, synthetic construct
  source 1..284: note = c-Cre recombinase AA
NRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLQDGD

SEQ ID NO: 74 - protein, 40 aa, synthetic construct
  source 1..40: note = c-Intein AA
VKVIGRRSLGVQRIFDIGLPQDHNFLLANGAIAANCFNKS

SEQ ID NO: 75 - protein, 22 aa, synthetic construct
  source 1..22: note = P2A AA
GSGATNFSLLKQAGDVEENPGP

SEQ ID NO: 76 - protein, 21 aa, synthetic construct
  source 1..21: note = T2A AA
GSGEGRGSLLTCGDVEENPGP

SEQ ID NO: 77 - protein, 238 aa, synthetic construct; identical to SEQ ID NO: 16
  source 1..238: note = GFP AA

SEQ ID NO: 78 - protein, 457 aa, synthetic construct
  source 1..457: note = construct; note = nCre-nIntein: P2A-NLS-nCre-nIntein-T2A-GFP
GSGATNFSLLKQAGDVEENPGPPKKKRKVTSDEVRKNLMDMFRDRQAFSEHTWKMLLSVCRSWAAWCKLNKFAEYCLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIKGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 79 - protein, 605 aa, synthetic construct
  source 1..605: note = construct; note = cCre-cIntein: P2A-cIntein-cCre-T2A-GFP
GSGATNFSLLKQAGDVEENPGPVKVIGRRSLGVQRIFDIGLPQDHNFLLANGAIAANCFNKSNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLQDGDGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 80 - DNA, 5894 bp, synthetic construct
  source 1..5894: mol_type = other DNA; note = cre recombinase expression plasmid; note = pCAG-cre recombinase full plasmid sequence (GenBank: AF334827.1; Cloning Vector pTurbo-Cre)
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggcttgatcgaagcttgcccaccatggcacccaagaagaagaggaaggtgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttgtgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctgcaagatggcgattagaagctttcgatcaagccgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattg

SEQ ID NO: 81 - protein, 286 aa, synthetic construct
  source 1..286: note = cre recombinase expression plasmid AA; note = pCAG cre recombinase full plasmid sequence translation (GenBank: AF334827.1; Cloning Vector pTurbo-Cre AA)
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 82 - DNA, 5571 bp, synthetic construct
  source 1..5571: mol_type = other DNA; note = Complete plasmid seq including backbone; note = SpyTag Expression vector: pCAG promoter driving expression of the following elements: Flag tag-NLS-nCre-nGFP-SpyTag
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgcccttgtgtcagtcgacgccaccatggattacaaggatgacgacgataagagcccgggcgtgcccaagaagaagaggaaagtcacctctgatgaagtcaggaagaacctgatggacatgttcagggacaggcaggccttctctgaacacacctggaagatgctcctgtctgtgtgcagatcctgggctgcctggtgcaagctgaactccggaggaggcggaatgagcaagggcgaggagctgttcaccggcgtggtgcccatcctggtggagctggacggcgacgtgaacggccacaagttcagcgtgagaggcgagggcgagggcgacgccaccatcggcaagctgaccctgaagttcatctgcaccaccggcaagctgcccgtgccctggcccaccctggtgaccaccctgacctacggcgtgcagtgcttcagcagataccccgaccacatgaagagacacgacttcttcaagagcgccatgcccgagggctacgtgcaagagagaaccatcagcttcaaggacgacggcaagtacaagaccagagccgtggtgaagttcgagggcgacaccctggtgaacagaatcgagctgaagggcaccgacttcaaggaggacggcaacatcctgggccacaagctggagtacaacttcaacagccacaacgtgtacatcaccgccgacaagggaggaggtggctccggaggaggtggctccgtgcctactatcgtgatggtggacgcctacaagcgttacaagtgagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattg

SEQ ID NO: 83 - DNA, 6415 bp, synthetic construct
  source 1..6415: mol_type = other DNA; note = Complete plasmid seq including backbone; note = SpyCatcher Expression vector: pCAG promoter driving expression of the following elements: Myc Tag-NLS-SpyCatcher-cGFP-cCre
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcacctgcacatgggacgtcgacctgaggtaattataacccgggccctatatatggatcggctagcgccaccatggagcagaaactcatctctgaagaggatctgagcccgggcgtgcccaagaagaagaggaaagtcggcgccatggtaaccaccttatcaggtttatcaggtgagcaaggtccgtccggtgatatgacaactgaagaagatagtgctacccatattaaattctcaaaacgtgatgaggacggccgtgagttagctggtgcaactatggagttgcgtgattcatctggtaaaactattagtacatggatttcagatggacatgtgaaggatttctacctgtatccaggaaaatatacatttgtcgaaaccgcagcaccagacggttatgaggtagcaactgctattacctttacagttaatgagcaaggtcaggttactgtaaatggcgaagcaactaaaggtgacgctcatactggaggaggtggctccggaggaggtggctcccagaagaacggcatcaaggccaacttcaccgtgagacacaacgtggaggacggcagcgtgcagctggccgaccactaccagcagaacacccccatcggcgacggccccgtgctgctgcccgacaaccactacctgagcacccagaccgtgctgagcaaggaccccaacgagaagagagaccacatggtgctgcacgagtacgtgaacgccgccggcatcaccggcggtggcggatccaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaagatggggactgagcggccgccaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtcgacattgattattg

(19) United States
(12) Patent Application Publication
(10) Pub. No.: US 2025/0380675 A1
(43) Pub. Date: Dec. 18, 2025

(54) Totally Sterile Population Of Avian Embryos, Production And Uses Thereof

(71) Applicant: THE STATE OF ISRAEL, MINISTRY OF AGRICULTURE & RURAL DEVELOPMENT, AGRICULTURAL R, Rishon LeZion (IL)

(72) Inventors: Yuval CINNAMON, Jerusalem (IL); Enbal BEN-TAL COHEN, Rehovot (IL)

(21) Appl. No.: 18/878,966

(22) PCT Filed: Jun. 28, 2023
(86) PCT No.: PCT/IL2023/050665

§ 371 Date: Dec. 26, 2024

Related U.S. Application Data
(60) Provisional application No. 63/356,139, filed on Jun. 28, 2022.

(51) Int. Cl. A01K 67/0275; C12N 9/12; C12N 9/22; C12N 15/11

(52) U.S. Cl. CPC A01K 67/0275; C12N 9/1241; C12N 9/226; C12N 15/11; A01K 2217/07; A01K 2227/30; C12N 2310/20

(57) ABSTRACT

The present disclosure relates to deoxyribonucleic acid (DNA) editing agents, and their use in preparing genetically modified cells and birds. The present disclosure further relates to fertile genetically modified avians and genetically modified avian primordial germ cells (PGCs) for producing sterile genetically modified avians (birds) that can serve as surrogate hosts for donor PGCs. The present disclosure further relates to methods for producing fertile avian strains that can produce a population of embryos and offspring, in both sexes, all of which are sterile and viable, and further relates to their subsequent use as sterile surrogate hosts for donor PGCs.

### SEQUENCE LISTING STATEMENT

The instant application contains a Sequence Listing which has been submitted electronically in XML format and is hereby incorporated by reference in its entirety. Said XML copy, created on Jun. 27, 2023, is named P-611396-PC_SL.xml and is 181.3 Kilobytes in size.

### FIELD OF INTEREST

The present disclosure relates to deoxyribonucleic acid (DNA) editing agents and their use in preparing genetically modified cells and birds. The present disclosure further relates to genetically modified avian primordial germ cells (PGCs) and fertile genetically modified avians (birds) for producing sterile genetically modified avians that can serve as surrogate hosts for donor PGCs. The present disclosure further relates to methods for producing avian strains that can produce viable totally sterile populations of avian embryos and offspring, in both sexes, and for their subsequent use as surrogate hosts for donor PGCs.

### BACKGROUND

The poultry industry accounts for over one-third of dietary protein production worldwide. With respect to chickens, one of the most popular poultry species, this figure originates mainly from two sources, table eggs from layer-type hens and broiler meat. Increase in world population and predicted demand for food by 2050, requires adaptation to food production to sustain the next generations, in an economical and affordable manner. It is estimated that >65 billion broilers are slaughtered annually worldwide. Notably, this is >10 times more than all the rest of the livestock animals altogether and demand for broiler meat is constantly growing worldwide. Significant increase in broiler egg production per hen would lower the numbers of required hens, thereby improving productivity, animal welfare issues and contribute for worldwide sustainability. Moreover, the production cost and footprint per egg will decrease.

Within the last 60 years of genetic selection, the broiler and layer have become extremely distinguished breeds, exploiting the best performances according to market's needs. While the modern broiler breeder hen produces ˜120-140 eggs per year, the modern layer-type breeder hen can lay more than 340 eggs, for significantly less feed per egg. Moreover, while commercial layer-type breeder hens will reach sexual maturity and start laying eggs at the age of 16-18 weeks and can lay eggs until >72 weeks of age, the commercial broiler breeder will start laying eggs at the age of 26-28 weeks until reaching 60-62 weeks of age. These figures demonstrate the tremendous advantage of the layer-type hen in terms of efficiency and reproduction.

One problem within the poultry industry is that male chicks, as non-layers, are an inevitable byproduct of the industry, and thus, they are manually sorted and culled using labor-intensive techniques. Sex determination in chickens is based on combinatorial segregation of the sex chromosomes Z and W in the hen's gametes. Each male chick harbors a Z chromosome which segregates from its mother hen and a second Z chromosome from its father.

Chickens and other avians (birds) reproduce by eggs, which, in most species, are fertilized internally in the female and then coated with a shell prior to laying. The avian embryo then incubates in the egg externally until hatching. Typically, one or both parents will participate in the incubation process.

The gametes in adults (sperm in males and eggs in females) arise from a unique population of embryonic cells called Primordial Germ Cells (PGCs). In chickens, the first PGCs are identified in the center of the embryonic blastoderm at oviposition. Within the first 24 hours (h) of incubation, the PGCs migrate to an extra embryonic region—the Germinal Crescent, at the anterior side of the embryo. As the blood system develops, the PGCs migrate through the blood circulation and colonize the Germinal Ridge, the anlage of the embryonic gonads. In chickens, two symmetrical embryonic gonads are formed for both sexes and by the ninth day of incubation, in females, the right gonad regresses, while the left gonad develops into a single ovary. In males, both embryonic gonads develop into the testes. Reaching sexual maturity, the PGCs give rise to gametes—ovulating eggs and sperm in females and males, respectively. Since PGCs are not somatic cells, they have no role other than hereditary. If PGCs fail to form, migrate, or differentiate into functional gametes, the organism cannot reproduce, rendering it sterile. Thus, ablating PGCs or affecting their ability to migrate to the gonads and differentiate into gametes results in sterility.

At early stages of embryogenesis, PGCs relocate to the embryonic gonads through the bloodstream, where they can be collected from, and returned to.

In addition, PGCs can readily undergo various types of genetic transformation, including, but not limited to, gene silencing, gene misexpression, gene overexpression, and transgenesis, whereas foreign DNA elements, which otherwise do not exist in the genome, can be inserted in a random or targeted manner into the genome. These modifications can improve, amongst other traits, agricultural performance, health, disease resistance, resilience to various stress conditions, and behavioral characteristics, and they can also be used to introduce traits which do not naturally exist in chickens.

Moreover, cryopreservation of chicken gametes has presented a long-standing challenge, because the huge, ovulated egg cannot successfully be restored from cryopreservation, inseminated, and restored back to the infundibulum of the oviduct, and sperm cryopreservation is highly unreliable. For many decades, poultry breeding companies created thousands of invaluable genetic colonies having diverse genetic backgrounds.

Because gamete cryopreservation is not reliable and efficiently feasible in chickens, these flocks must be kept alive. Even when they are not regularly in use, the vast majority of the colonies are kept merely for genetic records, backup in case of a catastrophe, and genetic diversity preservation. This situation imposes vast economic losses and harms livestock welfare. Additionally, numerous endangered chicken breeds, non-commercial and wild breeds cannot be cryopreserved, thus increasing the chance of losing potential genetic diversity and increasing breed extinctions.

Unlike gamete cryopreservation, cryopreservation of PGCs is readily feasible. PGCs can be collected at various embryonic stages starting from the freshly laid egg, the germinal crescent, the bloodstream, or directly from the gonads. When sufficient quantities of PGCs are obtained, either by direct collection (e.g., from the gonads) or following culturing, PGCs can be cryopreserved in liquid nitrogen for many years.

Generating genome edited chicken breeds is a multi-step process. Following genomic transformation, the genome-modified PGCs are currently injected to a surrogate recipient host embryo alongside to its endogenous PGCs, thereby giving rise to “chimera,” and the two populations of PGCs colonize the gonad. The ratio between the endogenous and modified PGCs in the gonads, and their potential to give rise to functional gametes, is reflected in the germline transmission, which is variable. Namely, in the case of males, in this example, this will be the ratio between modified and wild-type sperm cells, in a given semen sample, that can fertilize eggs. Low germline transmission ratio results in months of laborious screening for founder chicks which originate from modified PGCs. Alternatively, modification can be achieved by injecting viruses into the blastoderm, but this method is highly inefficient and inaccurate.

Clearly, by definition, sterile chickens cannot breed, therefore healthy and fertile heterozygotes, which carry a single mutated allele, are required to preserve the trait, and by crossing heterozygotes, null sterile embryos are obtained. Where the mutation or ablation of the gene does not have an impact on viability, the heterozygous-heterozygous cross will produce homozygous null sterile embryos, but only in a Mendelian ratio of 1:4.

It would be desirable to have compositions and methods for producing modified PGCs and for obtaining fertile genetically modified birds therefrom, followed by subsequent breeding of sterile surrogate birds from the genetically modified fertile birds. It would also be desirable to have compositions and methods for transforming sterile surrogate host birds in order to have them, upon sexual maturity, produce gametes originating from a selected genetic background of interest.

### SUMMARY

The compositions and methods provided herein are directed to the ability to collect PGCs, culture them and back transplant donor PGCs to host chimera embryos that will hatch and grow to sexual maturity. Upon sexual maturity the host avians will produce gametes originating from the genetic background of the transplanted PGCs.

In some aspects, disclosed herein is a deoxyribonucleic acid (DNA) editing system comprising: (a) a first agent comprising a first exogenous polynucleotide, the first exogenous polynucleotide comprising: (i) a first promoter; and (ii) a first element of interest operably linked to the promoter, the first element of interest encoding a first protein moiety of interest, wherein a first genetically modified avian having a genome comprising the first agent retains fertility and viability while producing a primordial germ cell (PGC) having a genome comprising the first agent without impairing viability of the PGC; and (b) a second agent comprising a second exogenous polynucleotide, the second exogenous polynucleotide comprising: (i) a second promoter; and (ii) a second element of interest operably linked to the promoter, the second element of interest encoding a second protein moiety of interest, wherein a second genetically modified avian having a genome comprising the second agent retains fertility and viability while producing a primordial germ cell (PGC) having a genome comprising the second agent without impairing viability of the PGC, the first agent and the second agent, when co-expressed in a genetically modified progeny avian embryo or progeny avian, wherein the genetically modified progeny avian embryo or progeny avian is a progeny of a first genetically modified avian arising from the first genetically modified PGC comprising the first agent and an opposite-gendered second genetically modified avian arising from the second genetically modified PGC comprising the second agent, inducing sterility or inhibiting fertility in the genetically modified progeny avian embryo or progeny avian without impairing viability.

In another aspect, provided herein is a primordial germ cell (PGC) system comprising a first genetically modified avian primordial germ cell (PGC) and a second genetically modified avian primordial germ cell (PGC): (a) the first genetically modified avian PGC comprising a first agent, the first agent comprising a first exogenous polynucleotide, the first exogenous polynucleotide comprising: (i) a first promoter; and (ii) a first element of interest operably linked to the promoter, the first element of interest encoding a first protein moiety of interest, wherein a first genetically modified avian having a genome comprising the first agent retains fertility and viability while producing a primordial germ cell (PGC) having a genome comprising the first agent without impairing viability of the PGC; and (b) the second genetically modified avian PGC comprising a second agent, the second agent comprising a second exogenous polynucleotide, the second exogenous polynucleotide comprising: (i) a second promoter; and (ii) a second element of interest operably linked to the promoter, the second element of interest encoding a second protein moiety of interest, wherein a second genetically modified avian having a genome comprising the second agent retains fertility and viability while producing a primordial germ cell (PGC) having a genome comprising the second agent without impairing viability of the PGC, the first agent and the second agent, when co-expressed in a genetically modified progeny avian embryo or progeny avian, wherein the genetically modified progeny avian embryo or progeny avian is a progeny of a first genetically modified avian arising from the first genetically modified PGC comprising the first agent and an opposite-gendered second genetically modified avian arising from the second genetically modified PGC comprising the second agent, inducing sterility or inhibiting fertility in the genetically modified progeny avian embryo or progeny avian without impairing viability.

In other aspects, provided herein is a sterile avian breeding system comprising a first genetically modified avian and a second genetically modified avian having an opposite sex to the first genetically modified avian: (a) the first genetically modified avian comprising a first genetically modified avian PGC comprising a first agent, the first agent comprising a first exogenous polynucleotide, the first exogenous polynucleotide comprising: (i) a first promoter; and (ii) a first element of interest operably linked to the promoter, the first element of interest encoding a first protein moiety of interest; and (b) the second genetically modified avian comprising a second genetically modified avian PGC comprising a second agent, the second agent comprising a second exogenous polynucleotide, the second exogenous polynucleotide comprising: (i) a second promoter; and (ii) a second element of interest operably linked to the promoter, the second element of interest encoding a second protein moiety of interest, the first agent and the second agent, when co-expressed in a genetically modified progeny avian embryo or progeny avian, wherein the genetically modified progeny avian embryo or progeny avian is a progeny of the first genetically modified avian arising from the first genetically modified PGC comprising the first agent and an opposite-gendered second genetically modified avian arising from the second genetically modified PGC comprising the second agent, inducing sterility or inhibiting fertility in the genetically modified progeny avian embryo or progeny avian without impairing viability.

In other aspects, provided herein is a method for producing a sterile genetically modified avian or a population of sterile genetically modified avians from two fertile independently genetically modified avians, the method comprising: (a) obtaining a first primordial germ cell (PGC) from an avian; (b) integrating into a chromosome of interest in the first PGC a first agent, the first agent comprising a first exogenous polynucleotide, the first exogenous polynucleotide comprising: (i) a first promoter; and (ii) a first element of interest operably linked to the promoter, the first element of interest encoding a first protein moiety of interest, wherein the first genetically modified avian having a genome comprising the first agent retains fertility and viability while producing a primordial germ cell (PGC) having a genome comprising the first agent without impairing viability of the PGC; (c) producing first pure PGC colonies comprising the first agent; (d) transplanting a first pure PGC colony into a male chick embryo to produce a first chimera male chick embryo and transplanting a first pure PGC colony into a female chick embryo to produce a first chimera female chick embryo; (e) producing a population of first chimera founder adult avians by hatching and rearing the first chimera founder chicks to sexual maturity, by mating first chimera founder chicks at adulthood to produce first chimera offspring, or both; (f) screening the first population of chimera founder adult avians to verify homozygosity for the first agent; (g) obtaining a second primordial germ cell (PGC) from an avian; (h) integrating into a chromosome of interest in the second PGC a second agent, the first agent comprising a second exogenous polynucleotide, the second exogenous polynucleotide comprising: (i) a second promoter; and (ii) a second element of interest operably linked to the promoter, the second element of interest encoding a second protein moiety of interest, wherein the second genetically modified avian having a genome comprising the second agent retains fertility and viability while producing a primordial germ cell (PGC) having a genome comprising the second agent without impairing viability of the PGC; (h) producing second pure PGC colonies comprising the second agent; (i) transplanting a second pure PGC colony into a male chick embryo to produce a second chimera male chick embryo and transplanting a second pure PGC colony into a female chick embryo to produce a second chimera female chick embryo; (i) producing a population of second chimera founder adult avians by hatching and rearing the second chimera founder chicks to sexual maturity, by mating chimera founder chicks at adulthood to produce offspring, or both; (j) screening the second population of chimera founder adult avians to verify homozygosity for the second agent; (k) selecting a male homozygous for the first agent from the first population of adult avians; (l) selecting a female homozygous for the second agent from the second population of adult avians; and (m) breeding the male adult avian from the first population with the female adult avian from the second population to produce a population of one or more sterile genetically modified progeny avian embryos, the first agent and the second agent, when co-expressed in the one or more genetically modified progeny avian embryos, inducing sterility or inhibiting fertility in the one or more genetically modified progeny avian embryos without impairing viability.

### BRIEF DESCRIPTION OF THE DRAWINGS

The patent or application file contains at least one drawing executed in color. Copies of this patent or patent application publication with color drawings will be provided by the Office upon request and payment of the necessary fees.

The subject matter regarded as the totally sterile population of avian embryos, products and uses thereof is particularly pointed out and distinctly claimed in the concluding portion of the specification. The totally sterile population of avian embryos, products and uses, however, both as to organization and method of operation, together with objects, features, and advantages thereof, may best be understood by reference to the following detailed description when read with the accompanying drawings in which:

FIG. 1 is a schematic depicting the overall method for producing a surrogate hen from a sterile chick. Primordial germ cells (PGCs) are injected into a sterile chick embryo. At maturity, the resulting chicken becomes a surrogate parent to PGC-derived progeny.

FIGS. 2A-2B describe a general method for generating a population of sterile embryos, e.g., while maintaining founder populations. FIG. 2A is a schematic depicting a general method for generating a population of sterile embryos. Two breeds are created, Breed A and Breed B, and the genetic background of them is represented by an illustration of a chromosome in blue and red. In each breed, there is a semi-inactivating element (SIE), depicted in Breed A (top left) and Breed B (top right) in yellow and green ovals, respectively. In each of the breeds there is a copy of an active fertility-required gene (FRG), depicted in white, thus, within itself, each breed is fertile. The SIEs can be a binary mechanism that directly inactivate the FRGs, and/or and binary mechanism that controls wild-type or exogenous sterility-inducing factors (SIFs). By crossing Breed A with Breed B, the offspring Breed AB (bottom) arises. Breed AB contains the two SIE copies, one from each parent. Having these two elements together, will either deactivate the FRG or activate the SIF (yellow arrow). The inactivated FRG, depicted in the black oval, renders the organisms of Breed AB sterile. FIG. 2B is a flowchart depicting an exemplary method for generating a population of sterile embryos, along with their subsequent use as surrogates.

FIGS. 3A-F are schematics depicting two exemplary methods (Intein and SpyTag/SpyCatcher based split Cre systems) for generating a population of sterile embryos, while maintaining founder populations. FIGS. 3A-C depict an exemplary Intein system. FIG. 3A is a schematic representation of the first SIE on Breed A, comprising a promoter depicted in blue, followed by a Nuclear Localization Signal (NLS, SEQ ID, SEQ ID NO: 3 & SEQ ID NO: 4) depicted in purple, followed by the 19-59 amino acids of the Cre enzyme (nCre, SEQ ID NO: 5 & SEQ ID NO: 6) depicted in yellow, followed by the N-terminal part of the Intein (nIntein, SEQ ID NO: 7 & SEQ ID NO: 8) depicted in red. FIG. 3B is a schematic representation depicting how these elements are fused in an open reading frame of the second SIE on Breed B, comprising a promoter depicted in blue, followed by the C-terminal part of the Intein (cIntein, SEQ ID NO: 9 & SEQ ID NO: 10) depicted in red, followed by the 60-343 amino acids of the Cre recombinase enzyme (cCre, SEQ ID NO: 11 & SEQ ID NO: 12) depicted in yellow. FIG. 3C is a schematic representation of the outcomes from crossing Breed A and Breed B to yield Breed AB. When the nCre-nIntein and the cIntein-cCre are co-expressed, the Intein peptides dimerize and cleave the Intein out (red circle), leaving the NLS and the two parts of Cre covalently bound and active. FIGS. 3D-F depict an exemplary SpyTag/SpyCatcher system. FIG. 3D is a schematic representation of the first SIE transgene on Breed A, comprising a promoter depicted in blue, followed by a NLS depicted in purple, followed by the nCre (SEQ ID NO: 5 & SEQ ID NO: 6) depicted in yellow, followed by the 1-155 amino acids of the Green Fluorescent Protein (nGFP) depicted in green, followed by SpyTag sequence depicted in bordeaux. In some embodiments, SEQ ID NO: 82 sets forth the nucleotide sequences of the expression vector incorporating the first SIE transgene. FIG. 3E is a schematic representation of the second SIE transgene on Breed B, comprising a promoter depicted in blue, followed by a NLS, followed by the SpyCatcher sequence depicted in bordeaux, followed by the 156-238 amino acids of the Green Fluorescent Protein (cGFP), followed by the cCre (SEQ ID NO: 11 & SEQ ID NO: 12) depicted in yellow. In some embodiments, SEQ ID NO: 83 sets forth the nucleotide sequences of the expression vector incorporating the second SIE transgene. FIG. 3F is a schematic representation of the outcomes from crossing Breed A with Breed B. When the NLS-nCre-nGFP-SpyTag and the NLS-SpyCatcher-cGFP-cCre are co-expressed, the Spy-Tag/SpyCatcher dimerize and covalently bind. This unites the two inactive parts of the GFP and of the Cre, rendering the fused protein to become fluorescent and the Cre enzyme, active.

FIGS. 4A-C are schematics depicting an exemplary method uniting a binary regulatory mechanism and a strategy to deactivate a fertility-required gene (FRG) FIG. 4A is a schematic representation of the genomic region of deleted in azoospermia-like (DAZL) gene (ch2:34,383,439.34,400,982; white leghorn layer GRCg7w). The gene is depicted in a black line, the locations of the exons are in blue boxes, and the first and last exons are marked with red and pink arrows, respectively. The genomic regions flanking the DAZL locus are depicted in a dashed line. The location of the sequences corresponding to the single-guide ribonucleic acids (sgRNAs) of the CRISPR sites, for integration of the single-stranded oligodeoxynucleotides (ssODNs) (5′ssODN and 3′ ssODN, respectively, pink boxes) are marked by the hash sign (#). The location of the sequences corresponding to the sgRNAs of the TV CRISPR sites, for integration of the TVs, is marked by an asterisk (*). The genomic regions corresponding to the 5′ and 3′ homology arms (HA), of the TV are marked by dark green boxes. FIG. 4B is a schematic representation of the 5′HA-P2A-NLS-nCre-nIntein-T2A-GFP-3′HA targeting vector. The P2A self-cleaving peptide (orange box) is fused in-frame to the DNA sequence coding for the last amino acid of DAZL. This is followed by a NLS (dark purple box), nCre (yellow box), nIntein (red box), T2A (light blue box), and GFP (light green box). The entire cassette is flanked by the 5′HA and 3′HA (dark green boxes). FIG. 4C is a schematic representation of the 5′HA-P2A-cIntein-cCre-T2A-GFP-3′HA targeting vector. The P2A self-cleaving peptide (orange box) is fused in-frame to the DNA sequence coding for the last amino acid of DAZL. This is followed by the cIntein (red box), cCre (yellow box), T2A (light blue box), and GFP (light green box). The entire cassette is flanked by the 5′HA and 3′HA (dark green boxes).

FIGS. 5A-D are schematics depicting an exemplary method of generating two fertile breeds that, when crossed, give rise to sterile embryos, using two different DNA recombinase enzymes, Cre and flippase (Flp). Two breeds are created: Breed A (FIGS. 5A-5B) and Breed B (FIGS. 5C-5D). FIG. 5A depicts a schematic representation of the genomic region of DAZL (ch2:34,383,439.34,400,982; white leghorn layer GRCg7w) of Breed A. The gene is depicted in a black line, the locations of the exons are in blue, and the first and last exons are marked with red and pink arrows, respectively. The genomic regions flanking the DAZL locus are depicted in a dashed line. The location of the sequences corresponding to the sgRNAs of the CRISPR sites, for integration of the ssODNs (5′ssODN and 3′ ssODN, respectively, pink boxes) are marked by the hash sign (#). The locations of the sequences corresponding to the sgRNAs of the TV CRISPR sites, for integration of the TV (shown in FIG. 5B) is marked by an asterisk (*). The genomic regions corresponding to the 5′ and 3′ homology arms (HA), of the TV are marked by dark green boxes. FIG. 5B is a schematic representation of the 5′HA-P2A-NLS-Flp-T2A-GFP-3′HA targeting vector of Breed A. The P2A self-cleaving peptide (orange box) is fused in-frame to the DNA sequence coding for the last amino acid of DAZL. This is followed by a NLS (dark purple box), the coding sequence of the Flp enzyme (red box), T2A (light blue box), and GFP (light green box). The entire cassette is flanked by the 5′HA and 3′HA (dark green boxes). FIG. 5C is a schematic representation of the genomic region of DAZL (ch2:34,383,439.34,400,982; white leghorn layer GRCg7w) of Breed B. The gene is depicted in a black line, the locations of the exons are in blue, and the first and last exons are marked with red and pink arrows, respectively. The genomic regions flanking the DAZL locus are depicted in a dashed line. The location of the sequences corresponding to the sgRNAs of the CRISPR sites, for integration of the FRT sequences, using ssODNs (FRT-5′ssODN and FRT-3′ ssODN, respectively, pink boxes) are marked by the hash sign (#). The locations of the sequences corresponding to the sgRNAs of the TV CRISPR sites, for integration of the TV (shown in FIG. 5D) is marked by an asterisk (*). The genomic regions corresponding to the 5′ and 3′ homology arms (HA), of the TV are marked by dark green boxes. FIG. 5D is a schematic representation of the 5′HA-P2A-NLS-Cre-T2A-GFP-3′HA targeting vector of Breed B. The P2A self-cleaving peptide (orange box) is fused in-frame to the DNA sequence coding for the last amino acid of DAZL. This is followed by a NLS (dark purple box), the coding sequence of the Cre enzyme (yellow box), T2A (light blue box), and GFP (light green box). The entire cassette is flanked by the 5′HA and 3′HA (dark green boxes).

FIGS. 6A-6B depict an exemplary strategy to integrate the two LoxP sites, using CRISPR-mediated homologous recombination of ssODNs, and results of the same. FIG. 6A is a schematic depicting the strategy to knock out the DAZL gene via the insertion of two LoxP sites that flank the promoter region on exon1 in primordial germ cells' (PGCs) genome. To ensure precise integration of the LoxP sites at the desired location, two single-stranded oligodeoxynucleotides (ssODN) were planned. ssODN1 (SEQ ID NO: 35) was designed for integration upstream, and ssODN2 (SEQ ID NO: 36) downstream, of exon 1. Each ssODN comprised 60 base pair (bp) homology arms on either side of the LoxP sequence, resulting in a total oligonucleotide sequence length of 156 bp. Homologous recombination was facilitated using CRISPR-mediated double-strand breaks (DSBs) at each site, achieved with CRISPR1 (SEQ ID NO: 33) and CRISPR2 (SEQ ID NO: 34) sgRNAs, respectively. (CRISPR1 sgRNA and CRISPR2 sgRNA are RNA molecules having RNA sequences corresponding to the DNA sequences of SEQ ID NO: 33 and SEQ ID NO: 34, respectively.) Primers flanking each ssODN integration site (A: F-Pre-D-ssODN1 [SEQ ID NO: 37]; B: R-Pre-D-ssODN1 [SEQ ID NO: 38]; C: F-Pre-D-ssODN2 [SEQ ID NO: 39]; D: R-Pre-D-ssODN2 [SEQ ID NO: 40]) were designed to validate the integration by performing PCR amplification of the region containing the inserted LoxP sequence. ssODN1 homology arms are shown (red) upstream of the promoter, while ssODN2 homology arms are shown (green) downstream of exon 1. FIG. 6B is a photograph depicting PCR analysis, via gel electrophoresis (marker in left lane), of LoxP integration. Extracted PGCs genomic DNA was used as template for PCR amplification with the following primers: A—Fwd-ssODN1 (SEQ ID NO: 37), B—Rev-ssODN1 (SEQ ID NO: 38), C—Fwd-ssODN2 (SEQ ID NO: 39), D—Rev-ssODN2 (SEQ ID NO: 40), as depicted in FIG. 6A. Genomic DNA from unmodified (i.e., not modified with LoxP as described herein) GMO (UM; WT) PGCs was used as a positive control. The expected size of the unmodified product is 364 bp. Upon LoxP integration, the predicted size increases to 398 bp for both ssODN1 and ssODN2 integrations. The PCR products of both ssODN1 and ssODN2 were sequenced-verified (SEQ ID NO: 42 and SEQ ID NO: 43, respectively). Notably, the successful homologous recombination of both ssODNs occurred on both alleles, as confirmed by PCR, as evidenced by the appearance of a single band (Homo) and sequencing. As a result, the primordial germ cells (PGCs) generated in this process are homozygous for the insertion of the LoxP sites. Lanes: 1—Marker; 2-3—PCR product of ssODN1 (2—unmodified GMO; 3—homologous recombination); 4-5—PCR product of ssODN2 (4—unmodified GMO; 5—homologous recombination). The WT band in each lane is the WT PCR product of unmodified PGCs.

FIGS. 7A-7C depict an exemplary strategy for targeting vectors for integration of Cre-Intein fusion proteins into the reading frame of the DAZL the gene. FIG. 7A is a schematic representation of targeting vectors (TV). The two vectors have the same 5′ and 3′ homology arms (HA; SEQ ID NO: 46 and SEQ ID NO: 47, respectively), directing the integration to the DAZL locus. The fusion proteins, NLS-nCre-nIntein (SEQ ID NOS: 70-72, encoded by SEQ ID NOS: 48-50, respectively; see FIG. 7A, left) or cCre-cIntein (SEQ ID NOS: 73-73, encoded by SEQ ID NOS: 51-52, respectively; see FIG. 7A, right) are fused in-frame to the C-terminus of the DAZL coding sequence by P2A (SEQ ID NO: 53, encoding SEQ ID NO: 75). Following them is the T2A peptide (SEQ ID NO: 54, encoding SEQ ID NO: 76) fused to the reporter gene GFP (SEQ ID NO: 55; encoding SEQ ID NO: 77). The nCre-nIntein TV (left) comprises P2A-NLS-nCre-nIntein-T2A-GFP (encoding SEQ ID NO: 78), and the cCre-cIntein TV (right) comprises P2A-cIntein-cCre-T2A-GFP (encoding SEQ ID NO: 79). The CRISPR-mediated homologous recombination is done using a specific sgRNA sequence (SEQ ID NO: 45). (The specific sgRNA sequence is an RNA molecule having an RNA sequence corresponding to the DNA sequence of SEQ ID NO: 45.) The last two nucleic acids in the DAZL NLS-nCre-nIntein TV (nCre recombinase) (SEQ ID NO: 49) are for creating an in-frame link with the first amino acid of the DAZL NLS-nCre-nIntein TV (nIntein) (SEQ ID NO: 50). This codon enters a linking lysine (K) amino acid. The sgRNA sequence may include a protospacer adjacent motif (PAM) sequence, which is a short nucleotide sequence (usually 2-6 bp) enabling the Cas nuclease in a CRISPR system to cut, e.g., added to the 3′-end of the sgRNA (e.g., “AGG”) at the 3′-end of the sgRNA. The primers that were used for sequencing and PCR analysis are depicted as A-J (SEQ ID NOS: 56-65, respectively; A—F-DAZL-ATGCodon; B—F_DAZL_EndSeq1; C=R_DAZL_EndSeq1; D=F_DAZL_EndSeq2; E—R-magP2A; F—Fw-PBRAGE-EGFP; G—Rv-PGRAGE-EGFP; H—F_DAZL_EndSeq3; I—R-Pre-D3HA-Nest; J—R-Pre-D3HA). On-site integration was validated with primers A and G (SEQ ID NOS: 56 and 62, respectively), as well as F and J (SEQ ID NOS: 61 and 65, respectively). Primers A and J (SEQ ID NOS: 56 and 65, respectively) are located outside of the homology arms, while primers G and F (SEQ ID NOS: 62 and 61, respectively) are located inside the insert. For the correct integration of the NLS-nCre-nIntein vector (see SEQ ID NOS: 48-50), the expected PCR product between primers A and G (SEQ ID NOS: 56 and 62, respectively) is 2692 bp, to confirm correct integration of the 5′HA (SEQ ID NO: 46). The expected PCR product, upon correct integration, between primers F and J (SEQ ID NOS: 61 and 65, respectively) is 2001 bp, to confirm correct integration of the 3′HA (SEQ ID NO: 47). For the correct integration of the cCre-cIntein vector (see SEQ ID NOS: 51-52), the expected PCR product between primers A and G (SEQ ID NOS: 56 and 62, respectively) is 3136 bp, to confirm correct integration of the 5′HA (SEQ ID NO: 46) and the expected PCR product, upon correct integration, between primers F and J (SEQ ID NOS: 61 and 65, respectively) is 1998 bp, to confirm correct integration of the 3′HA (SEQ ID NO: 47). FIG. 7B is a photograph depicting PCR analysis of transformed PGCs colonies. Either the NLS-nCre-nIntein targeting vector (depicted in short as cInt-cCre), or the targeting vector cCre-cIntein (depicted in short as cInt-cCre) were integrated to the genome of PGCs. Extracted genomic DNA for each colony was used as template for PCR as described above. The PCR analysis confirmed the predicted products length, confirming that both targeting vectors were correctly integrated. The primers A-J (SEQ ID NOS: 56-65, respectively) were used for sequencing of unmodified PGCs genome (SEQ ID NO: 66) for control, and to validate the correct integration of the NLS-nCre-nIntein TV and cCre-cIntein TV into DAZL to yield the NLS-nCre-nIntein knocking to DAZL (SEQ ID NO: 67) and cCre-cIntein knocking to DAZL (SEQ ID NO: 68). Lanes: 1—Marker; 2-3—DAZL TV cInt-cCre (2-5′HA; 3-3′HA); 4-5—DAZL TV nCre-nInt (4-5′HA; 5-3′HA). FIG. 7C is a series of photographs depicting two pure PGCs lines, which underwent homologous recombination—mediated integration with the targeting vectors shown in FIG. 7A. Each colony is presented in brightfield and fluorescence (GFP), left to right, respectively. The colony on the left (the two left panels) has the nCre-nIntein integration. The colony on the right has the cCre-cIntein integration. These two colonies correspond to the targeting vectors in FIG. 7A.

It will be appreciated that for simplicity and clarity of illustration, elements shown in the FIGURES have not necessarily been drawn to scale. For example, the dimensions of some of the elements may be exaggerated relative to other elements for clarity. Further, where considered appropriate, reference numerals may be repeated among the FIGURES to indicate corresponding or analogous elements.

### DETAILED DESCRIPTION

In the following detailed description, numerous specific details are set forth in order to provide a thorough understanding of the totally sterile population of avian embryos, products and uses disclosed herein. However, it will be understood by those skilled in the art that the present sterile avian embryos, products and uses may be practiced without these specific details. In other instances, well-known methods, procedures, and components have not been described in detail so as not to obscure the present description of sterile avian embryos, products and uses disclosed herein.

Before explaining at least one embodiment of the disclosure in detail, it is to be understood that the disclosure is not necessarily limited in its application to the details set forth in the following description or exemplified by the Examples. The disclosure encompasses other embodiments or can be practiced or carried out in various ways.

Sterile avians are beneficial for several applications and their usefulness is relevant to both poultry (e.g., layer and broiler chickens) and game industries, as well as for research and for wild avian breed and species conservation. Provided herein are fertile genetically modified avians and genetically modified avian primordial germ cells (PGCs) for producing sterile genetically modified avians (birds) that can serve as surrogate hosts for donor PGCs. Also provided herein are deoxyribonucleic acid (DNA) editing systems and methods for producing fertile avian strains that can produce viable sterile embryos and offspring, in both sexes, and for their subsequent use as surrogate hosts for donor PGCs.

Reaching sexual maturity, the primordial germ cells (PGCs) give rise to gametes-ovulating eggs and sperm in females and males, respectively. Because PGCs are not somatic cells, they have no role other than hereditary. If PGCs fail to form, migrate, or differentiate into functional gametes, the organism cannot reproduce, rendering it sterile. In the absence of PGCs, no eggs or sperm are formed, which renders the organism sterile.

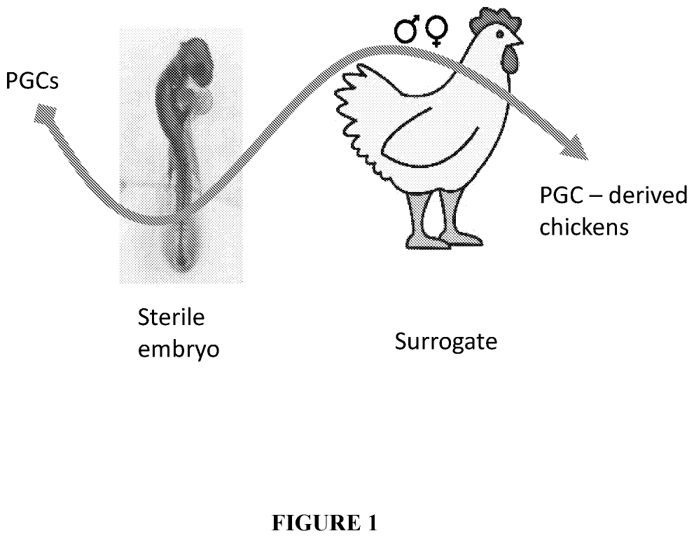

PGCs with desirable traits can then be implanted into sterile embryonic avians (e.g., sterile layer chick embryos can be implanted with PGCs for broiler chickens, thereby enable mature layer surrogate chickens to produce broiler chickens as offspring), as shown in FIG. 1.

Thus, ablating PGCs or affecting their ability to differentiate to functional gametes results in sterility. Generating a genetically modified sterile chicken can be obtained by multiple approaches. In one embodiment, the method comprises creating one or more genetic deleterious manipulations of genes (e.g., knocking-out), which are required for PGC survival, migration, and gamete formation. Collectively, these genes are referred to as Fertility-Required Genes (FRGs). In another embodiment, the method comprises creating sterile chickens by exploiting the unique expression pattern of one or more FRGs to activate one or more exogenous elements affecting the ability of PGCs to form gametes or PGC survival. In some embodiments, the embryos and birds, for example but not limited to chickens, described and produced herein are totally sterile. In some embodiments, produced herein are totally sterile populations of avian embryos. In some embodiments, produced herein are totally sterile populations of chicken embryos.

FRG have an isolated role in PGCs, thus affecting them will have no maladaptive effects on somatic cells, or the wellbeing of the sterile organism. FRG can be autosomal with two alleles or located on the sex chromosomes—W & Z, with one allele, in females. Generating deleterious modification in FRG, affecting all functional alleles, results in a sterile organism that by definition cannot breed and propagate the modification to the next generation. Therefore, in case of a recessive modification, the organism should be heterozygote with respect to the modification, or should have at least one FRG allele with sufficient activity to support fertility in order to propagate the modification throughout generations. If the deleteriously-modified FRG is located on an autosomal chromosome, by crossing between two heterozygote individuals, due to Mendelian segregation, 25% of the embryos will be homozygote for the modification and will be sterile. In the instance in which the deleteriously-modified FRG is located on the Z sex chromosome, only the males (having two Z chromosomes) will be fertile and will be able to propagate the modification to the next generations, but the females, which receive modified FRG on the Z chromosome and do not have an intact FRG copy, are sterile. In this case, 50% of the females (25% of the total embryos) will be sterile. In addition, in the situation in which the FRG have a dominant effect, requiring two intact copies of the FRG, no sterile chicken could be obtained.

Being a unique cell population, several FRGs are specifically expressed in PGCs. Thus, the molecular mechanisms that regulate specific expression in PGCs can be harnessed and utilized to express exogenous regulating elements (i.e., foreign regulating elements). These elements can affect the ability to form functional gametes or affect PGCs survivability. In both cases, the resulting organism will be sterile. Examples of such elements include, but are not limited to, toxins that will induce PGCs death, elements that induce programmed-cell death, genomic modifiers that will deactivate FRGs, or elements that inhibit the expression or activity of FRGs (e.g., small interfering ribonucleic acid(s) [siRNA]), or dominant-negative forms of FRGs. Collectively, these wild-type or exogenous Sterility-Inducing Factors are referred hereinafter as SIFs.

In certain embodiments, the initial PGCs or avians (e.g., the avians from which the PGCs are harvested) or the genetic material of either are wild-type (WT) PGCs, avians, or genetic material, respectively. In certain embodiments, wild-type (WT) PGCs, avians, or genetic material comprise PGCs, avians, or genetic material, respectively, of a strain, gene/genetic material, or characteristic prevailing in natural conditions, as distinct from an atypical mutant form or type. In certain embodiments, wild-type (WT) PGCs, avians, or genetic material comprise PGCs, avians, or genetic material as found in their respective natural, non-mutated form(s). In certain embodiments, the wild type (WT) is the phenotype, genotype, or gene that predominates in a natural population of organisms or strain of organisms in contrast to that of natural or laboratory mutant forms, or it is a cell or organism or strain displaying the wild type. In certain embodiments, the wild type (WT) is the gene, characteristic, or phenotype that is typical or that occurs most frequently in the natural population. In certain embodiments, the wild type (WT) is the standard or norm of allele and is the most prevalent phenotype or genotype among a particular population. In certain embodiments, the wild type (WT) is the typical form or phenotype or genotype of a species or organism resulting from a natural breeding population.

With regard to the natural conditions of commercial chickens available today, a s skilled artisan would appreciate that commercial chickens underwent huge genomic and genetic changes throughout the years using selection. Thus, while these chickens do not present “natural” characteristics, they are considered as WT. As used throughout, the term “wild-type” or “WT” may encompass any genetic state of an avian, for example a commercial chicken, prior to the modifications described herein. In certain embodiments, however, the wild type (WT) may also refer to a genetically modified organism (GMO) or a cell derived from a genetically modified organism (e.g., a PGC derived from a GMO), or a cell that has been previously genetically modified (e.g., a PGC) that has not yet been further modified according to the methods, vectors, system, etc., described herein. In certain embodiments, the modifications of the methods, vectors, system, etc., described herein, are made to a genetically modified organism (GMO) or cell (e.g., a genetically modified PGC or a PGC derived from a GMO) or genetic material from either of these, and wild-type (WT) comprises the GMO, cell, or genetic material prior to further modification by the methods, vectors, system, etc., described herein (e.g., the baseline or unmodified GMO, cell, or genetic material). For example, wild type (WT) can include, e.g., genomic DNA from an unmodified (i.e., not modified with LoxP, Cre, Intein, or any of the other modifications as described herein) GMO.

Provided herein is a genetic solution to ensure, e.g., three things. The first is to allow a sterility-inducing genetic mechanism to propagate throughout generations without inducing sterility on a fertile genetic background. The second is to ensure that by genetic crosses, the sterility-inducing genetic mechanism will be activated in all the resulting embryos, rendering all of them sterile. In some embodiments of the population of avian embryos produced using the methods described and exemplified herein, the population of embryos are totally sterile. In some embodiments, the methods described and exemplified herein, produce a population of sterile embryos that has 100% sterility (all the embryos are sterile). Producing embryos with 100% sterility provides an improvement and advantage over methods that result in 25% Mendelian rate of success for sterility, wherein only a quarter of the embryos produced are sterile. The third is the ability to use FRGs with a dominant effect.

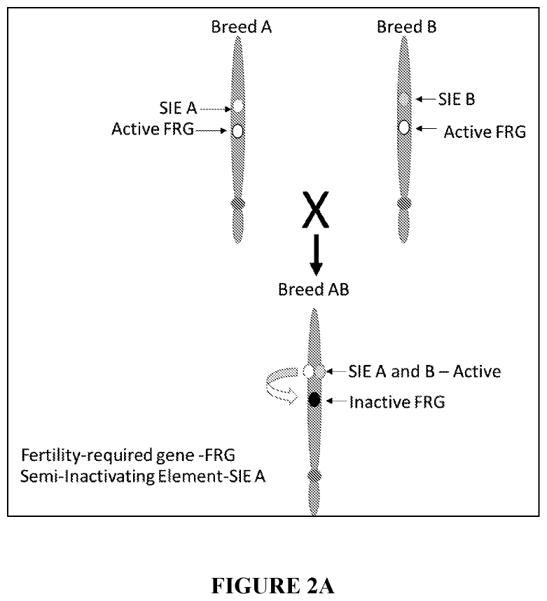

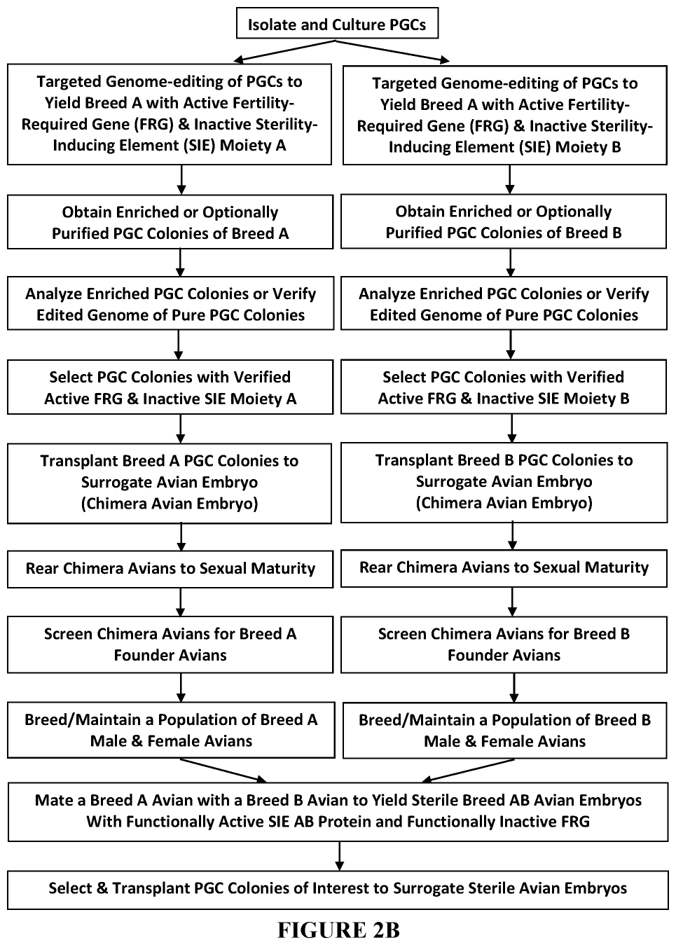

Provided herein is a solution based on a general approach in which two separate breeds are created, each breed having one semi-inactivating element (SIE) that, by itself, has no activity. By crossing between the two breeds, each of the resulting embryos will receive two SIEs, one from each parent. The two SIEs lead to a deleterious effect on the activity of FRGs. Having only inactivated FRGs, the embryo will develop into a sterile organism. Alternatively, two SIEs will activate the SIF which will affect the survival of the PGCs or will interrupt their function. This is a binary-based activation process in which two separate breeds are created, each having one SIE, and by crossing between the two breeds, the resulting embryos receive a copy of each element, which, dimerized, covalently bound, or otherwise taken together, become active and lead to deleterious mutation in fertility-required genes (FRG) or to active the SIF, which will affect the survival of the PGCs or will interrupt their function, as shown in FIGS. 2A-2B.

Provided herein are sterile gene-edited or genetically modified avians and gene-edited or genetically modified avian primordial germ cells (PGCs) for producing sterile genetically modified avians (birds) that can serve as surrogate hosts for donor PGCs. Also provided herein are deoxyribonucleic acid (DNA) editing systems and methods for producing avian strains that can produce viable sterile embryos and offspring, in both sexes, and for their subsequent use as surrogate hosts for donor PGCs.

In some aspects, disclosed herein is a deoxyribonucleic acid (DNA) editing system comprising: (a) a first agent comprising a first exogenous polynucleotide, the first exogenous polynucleotide comprising: (i) a first promoter; and (ii) a first element of interest operably linked to the promoter, the first element of interest encoding a first protein moiety of interest, wherein a first genetically modified avian having a genome comprising the first agent retains fertility and viability while producing a primordial germ cell (PGC) having a genome comprising the first agent without impairing viability of the PGC; and (b) a second agent comprising a second exogenous polynucleotide, the second exogenous polynucleotide comprising: (i) a second promoter; and (ii) a second element of interest operably linked to the promoter, the second element of interest encoding a second protein moiety of interest, wherein a second genetically modified avian having a genome comprising the second agent retains fertility and viability while producing a primordial germ cell (PGC) having a genome comprising the second agent without impairing viability of the PGC, the first agent and the second agent, when co-expressed in a genetically modified progeny avian embryo or progeny avian, wherein the genetically modified progeny avian embryo or progeny avian is a progeny of a first genetically modified avian arising from the first genetically modified PGC comprising the first agent and an opposite-gendered second genetically modified avian arising from the second genetically modified PGC comprising the second agent, inducing sterility or inhibiting fertility in the genetically modified progeny avian embryo or progeny avian without impairing viability.

As used throughout, the term “opposite-gendered second genetically modified avian” encompasses the situation wherein the second genetically modified avian has an opposite gender compared with the first genetically modified avian.

In some embodiments, the first promoter, the second promoter, or both comprises: (a) a promoter specific to a primordial germ cell (PGC); (b) a tissue-specific promoter; or (c) a ubiquitous promoter.

In some embodiments, the first protein moiety of interest is a functionally inactive first protein moiety of interest and the second protein moiety of interest is a functionally inactive second protein moiety of interest, wherein: (a) the functionally inactive first protein moiety of interest and the functionally inactive second protein moiety of interest, dimerized or bound covalently, comprise at least one functionally active protein of interest or fragment thereof, wherein: (i) the functionally active protein of interest or fragment thereof comprises a genomic modifier, the genomic modifier targeting a gene of interest (GOI) or fragment thereof on a chromosome, the GOI modified to introduce one or more target sites specific to the genomic modifier, and the GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active gene of interest (GOI), the modified trait inducing sterility or inhibiting fertility in the functionally modified and genetically modified progeny avian embryo or progeny avian without impairing viability; (ii) the functionally active protein of interest or fragment thereof comprises a toxin inducing cell death of a PGC, either without impairing viability of somatic cells or limited to benign somatic cell loss or both, in the functionally modified and genetically modified progeny avian embryo or progeny avian, and inducing sterility or inhibiting fertility in the functionally modified and genetically modified progeny avian embryo or progeny avian without impairing viability; or (iii) a combination thereof, (b) the functionally inactive first protein moiety of interest and the functionally inactive second protein moiety of interest, when co-expressed, comprise at least one functionally active protein of interest or fragment thereof, the co-expression of which inducing cell death of a PGC without impairing viability of somatic cells in the functionally modified and genetically modified progeny avian embryo or progeny avian and inducing sterility or inhibiting fertility in the functionally modified and genetically modified avian without impairing viability; or (c) a combination of any of the above.

In some embodiments, the gene of interest (GOI) sequence or fragment thereof has (a) an isolated function specific to a PGC, or (b) a function specific to gametogenesis, meiosis, gamete maturation, gamete function, or gamete fertilization in the genetically modified avian, wherein deletion, disruption, or functional modification of the gene of interest (GOI) reduces or inhibits survival, maturation, or differentiation of a PGC or the specific gametogenesis, gamete maturation, or gamete functional modification reduces or inhibits gametogenesis, meiosis, gamete function, or gamete fertilization in an avian. In some embodiments, the gene of interest (GOI) sequence or fragment thereof comprises a deleted in azoospermia-like (DAZL) gene, a deleted in azoospermia 1 (DAZ1) gene, a zona pellucida binding protein 12 (ZPBP1/2) gene, a cyclin-dependent kinases regulatory subunit 2 (CKS2; CDC28 Protein Kinase Regulatory Subunit 2) gene, a spermatogenesis associated 16 (SPATA16) gene, a DEAD-box helicase 4 (DDX4) gene, a serine/threonine-protein phosphatase PP1-gamma catalytic subunit (PPP1CC) gene, an Izumo sperm-egg fusion 1 (IZUMO1) gene, a synaptonemal complex central element protein 1 (SYCE1) gene, a YTH domain-containing 2 (YTHDC2) gene, a Meiosis Specific With Coiled-Coil Domain (MEIOC) gene, a septin-4 (SEPT4) gene, a stromal antigen 3 (STAG3) gene, a Nanos C2HC-type zinc finger 3 (NANOS3) gene, or a combination of any of these.

In some embodiments, the genomic modifier comprises a site-specific recombinase enzyme or functionally active fragment thereof. In some embodiments, the site-specific recombinase comprises a tyrosine recombinase or a serine recombinase. In some embodiments, the tyrosine recombinase comprises a Cre recombinase (Cre), a Dre recombinase (Dre), a flippase recombinase (Flp), or a Vika recombinase (Vika), and the target site comprises a recombinase recognition site, the recombinase recognition site comprising, respectively, a Lox site (Lox), a Rox site (Rox), a FRT site (FRT), or a Vox site (Vox). In some embodiments, the tyrosine recombinase comprises Cre and the recombinase recognition site comprises a locus of X-over P1 site (LoxP) (locus of X-over P site). In some embodiments, the tyrosine recombinase comprises Flp and the recombinase recognition site comprises FRT.

In certain embodiments, the functionally inactive first Cre protein moiety of interest has a sequence at least 95% identical to SEQ ID NO: 6 or SEQ ID NO: 71, and the functionally inactive second Cre protein moiety of interest has a sequence at least 95% identical to SEQ ID NO: 12 or to SEQ ID NO: 73. In certain embodiments, the functionally inactive first Cre protein moiety of interest comprises the sequence of SEQ ID NO: 6 or SEQ ID NO: 71, and the functionally inactive second Cre protein moiety of interest comprises the sequence of SEQ ID NO: 12 or to SEQ ID NO: 73.

In some embodiments, the first exogenous polynucleotide further encodes a first intein moiety operably linked to the first promoter and to the first element of interest encoding the functionally inactive first protein moiety of interest; and the second exogenous polynucleotide further encodes a second intein moiety distinct from the first intein moiety and operably linked to the second promoter and to the second element of interest encoding the functionally inactive second protein moiety of interest, wherein the first intein moiety and the second intein moiety, dimerized or covalently bound, comprise a functionally active intein or fragment thereof, the functionally active intein or fragment thereof splicing the functionally inactive first protein moiety of interest and the functionally inactive second protein moiety of interest to produce the functionally active protein of interest or fragment thereof. In some embodiments, the first intein moiety and the second intein moiety are dimerized (i.e., form a dimer). In some embodiments, the first intein moiety and the second intein moiety are covalently bound. In some embodiments, the first intein moiety is a functionally inactive intein moiety and the second intein moiety is a functionally inactive intein moiety.

In certain embodiments, the functionally inactive first intein moiety has a sequence at least 95% identical to SEQ ID NO: 8 or SEQ ID NO: 72, and the functionally inactive second intein moiety has a sequence at least 95% identical to SEQ ID NO: 10 or SEQ ID NO: 74. In certain embodiments, the functionally inactive first intein moiety comprises the sequence of SEQ ID NO: 8 or SEQ ID NO: 72, and the functionally inactive second intein moiety comprises the sequence of SEQ ID NO: 10 or SEQ ID NO: 74.

In some embodiments, the first exogenous polynucleotide further encodes a first conjugating element operably linked to the first promoter and to the first element of interest encoding the functionally inactive first protein moiety of interest; and the second exogenous polynucleotide further encodes a second conjugating element operably linked to the second promoter and to the second element of interest encoding the functionally inactive second protein moiety of interest, the first conjugating element conjugating to the second conjugating element to produce the functionally active protein of interest or fragment thereof. In some embodiments, the first conjugating element comprises SpyTag, and the second conjugating element comprises SpyCatcher.

In some embodiments, the first conjugating element comprises the first element of interest and the second conjugating element comprises the second element of interest. In some embodiments, (a) the first conjugating element comprises a functionally inactive first moiety of a recombinase and the second conjugating element comprises a functionally inactive second moiety of the recombinase, the second moiety of the recombinase distinct from the first moiety of the recombinase, the first conjugating element conjugating to the second conjugating element to produce the functionally active recombinase or fragment thereof, (b) the first conjugating element comprises a functionally inactive first moiety of a CRISPR protein and the second conjugating element comprises a functionally inactive second moiety of the CRISPR protein, the second moiety of the CRISPR protein distinct from the first moiety of the CRISPR protein, the first conjugating element conjugating to the second conjugating element to produce the functionally active CRISPR protein or fragment thereof, or (c) a combination thereof.

In some embodiments, the first exogenous polynucleotide further encodes a first functionally inactive marker moiety operably linked to the first promoter, the first conjugating element, and the first protein moiety of interest and the second exogenous polynucleotide further encodes a second functionally inactive marker moiety operably linked to the second promoter, the second conjugating element, and the second protein moiety of interest, the first conjugating element conjugating to the second conjugating element to produce a functionally active marker or fragment thereof.

In some embodiments, the first exogenous polynucleotide encodes a functionally active marker operably linked to the first protein moiety of interest; the second exogenous polynucleotide encodes a functionally active marker operably linked to the second protein moiety of interest; or a combination thereof. In some embodiments, the marker encoded by the first exogenous polynucleotide is distinct from the marker encoded by the second exogenous polynucleotide. In some embodiments, the marker is a fluorescent protein, a luminescent protein, or a chromoprotein.

In some embodiments, the first exogenous polynucleotide encodes a nuclear localization signal (NLS) operably linked to the first protein moiety of interest; the second exogenous polynucleotide encodes a nuclear localization signal (NLS) operably linked to the second protein moiety of interest; or a combination thereof. In some embodiments, the NLS is encoded by SEQ ID NO: 3 or SEQ ID NO: 48. In some embodiments, the NLS has a sequence at least 95% identical to SEQ ID NO: 4 or SEQ ID NO: 70. In some embodiments, the NLS has a sequence comprising SEQ ID NO: 4 or SEQ ID NO: 70.

In some embodiments, the first exogenous polynucleotide further comprises one or more self-cleaving peptides operably linked to the first protein moiety of interest and the second exogenous polynucleotide further comprises one or more self-cleaving peptides operably linked to the second protein moiety of interest. In some embodiments, the self-cleaving peptide comprises a P2A peptide. In some embodiments, the self-cleaving peptide comprises a T2A peptide. In some embodiments, the P2A peptide is encoded by SEQ ID NO: 13 or SEQ ID NO: 53. In some embodiments, the P2A peptide has a sequence at least 95% identical to SEQ ID NO: 14 or SEQ ID NO: 75. In some embodiments, the P2A peptide has a sequence comprising SEQ ID NO: 14 or SEQ ID NO: 75. In some embodiments, the T2A peptide is encoded by SEQ ID NO: 17 or SEQ ID NO: 54. In some embodiments, the T2A peptide has a sequence at least 95% identical to SEQ ID NO: 18 or SEQ ID NO: 76. In some embodiments, the T2A peptide has a sequence comprising SEQ ID NO: 18 or SEQ ID NO: 76.

In some embodiments, the first exogenous polynucleotide further comprises a first 5′ homology arm (HA) and a first 3′ homology arm (HA), said first 5′ HA and said first 3′ HA specific for a first insertion site of interest on the avian genome and wherein the second exogenous polynucleotide further comprises a second 5′ homology arm (HA) and a second 3′ homology arm (HA), said second 5′ HA and said second 3′ HA specific for a second insertion site of interest on the avian genome, wherein the first 5′ HA has a nucleotide sequence that is substantially homologous to the 5′ region flanking a first gene of interest (GOI) in a first chromosome of interest and the first 3′ HA has a nucleotide sequence that is substantially homologous to the 3′ region flanking the first GOI in the first chromosome of interest; the second 5′ HA has a nucleotide sequence that is substantially homologous to the 5′ region flanking a second GOI in a second chromosome of interest and the second 3′ HA, or both has a nucleotide sequence that is substantially homologous to the 3′ region flanking the second GOI in the second chromosome of interest; or both. In some embodiments, the first GOI and the second GOI are the same GOI and the first chromosome of interest and the second chromosome of interest are the same chromosome of interest.

In some embodiments, the gene of interest (GOI) or a fragment thereof comprises the Deleted in Azoospermia-Like (DAZL) gene or a fragment thereof and the first 5′ HA, the second 5′ HA, or both has a sequence at least 80% identical to SEQ ID NO: 23 or SEQ ID NO: 46, and the first 3′ HA, the second 3′ HA, or both has a sequence at least 80% identical to SEQ ID NO: 24 or SEQ ID NO: 47. In some embodiments, the gene of interest (GOI) or a fragment thereof comprises the Deleted in Azoospermia-Like (DAZL) gene or a fragment thereof and the first 5′ HA, the second 5′ HA, or both has a sequence at least 95% identical to SEQ ID NO: 23 or SEQ ID NO: 46, and the first 3′ HA, the second 3′ HA, or both has a sequence at least 95% identical to SEQ ID NO: 24 or SEQ ID NO: 47. In some embodiments, the gene of interest (GOI) or a fragment thereof comprises the Deleted in Azoospermia-Like (DAZL) gene or a fragment thereof and the first 5′ HA, the second 5′ HA, or both has a sequence 100% identical to SEQ ID NO: 23 or SEQ ID NO: 46, and the first 3′ HA, the second 3′ HA, or both has a sequence 100% identical to SEQ ID NO: 24 or SEQ ID NO: 47. In some embodiments, the gene of interest (GOI) or a fragment thereof comprises the Deleted in Azoospermia-Like (DAZL) gene or a fragment thereof and the first 5′ HA, the second 5′ HA, or both comprises the sequence of SEQ ID NO: 23 or SEQ ID NO: 46, and the first 3′ HA, the second 3′ HA, or both comprises the sequence of SEQ ID NO: 24 or SEQ ID NO: 47

In certain embodiments, the genomic modifier comprises a Cre recombinase (Cre) protein or functionally active fragment thereof, and the first exogenous polynucleotide further encodes a functionally inactive first intein moiety operably linked to the first promoter and to the first element of interest encoding a functionally inactive first Cre protein moiety of interest; and the second exogenous polynucleotide further encodes a functionally inactive second intein moiety distinct from the functionally inactive first intein moiety and operably linked to the second promoter and to the second element of interest encoding a functionally inactive second Cre protein moiety of interest, distinct from the first Cre protein moiety of interest, wherein the functionally inactive first CRE moiety or the functionally inactive second CRE moiety is fused in-frame to a nuclear localizing signal (NLS), wherein the functionally inactive first Cre moiety and the functionally inactive second Cre moiety, dimerized or bound covalently, comprise a functionally active Cre or functionally active fragment thereof, and wherein the functionally inactive first intein moiety and the functionally inactive second intein moiety, dimerized or bound covalently, comprise a functionally active intein or fragment thereof, the functionally active intein or fragment thereof splicing the functionally inactive first protein moiety of interest and the functionally inactive second protein moiety of interest to produce the functionally active protein of interest or fragment thereof

In some embodiments, the first exogenous polynucleotide, the second exogenous polynucleotide, or both further comprises at least one self-cleaving peptide. Examples of self-cleaving peptides include, but are not limited to, P2A and T2A.

In certain embodiments, the DNA editing system further comprises a first P2A moiety, a second P2A protein moiety, a first T2A protein moiety, a second T2A protein moiety, a first marker moiety, and a second marker moiety, the first agent comprising the first P2A moiety, the nuclear localizing signal (NLS), the functionally inactive first Cre moiety, the functionally inactive first intein moiety, the first T2A moiety, and the first marker moiety, wherein the first P2A moiety is operably linked 5′ to the nuclear localizing signal (NLS), the NLS is operably linked 5′ to the functionally inactive first Cre moiety, the functionally inactive first Cre moiety is operably linked 5′ to the functionally inactive first intein moiety, the functionally inactive first intein moiety is operably linked 5′ to the first T2A moiety, and the first T2A moiety is operably linked 5′ to the first marker moiety; and the second agent comprising the second P2A protein moiety, the functionally inactive second intein moiety, the functionally inactive second Cre moiety, a second T2A moiety, and a second marker moiety, wherein the second P2A protein moiety is operably linked 5′ to the functionally inactive second intein moiety, the functionally inactive second intein moiety is operably linked 5′ to the functionally inactive second Cre moiety, the functionally inactive second Cre moiety is operably linked 5′ to the second T2A moiety, and the second T2A moiety is operably linked 5′ to the second marker moiety.

In some embodiments of the DNA editing system, T2A and P2A are interchangeable. While T2A and P2A may in certain cases be interchangeable, the order of the other elements cannot be changed. For example, but not limited to this example, the order of P2A followed by NLS cannot be changed.

In some embodiments, the NLS is encoded by SEQ ID NO: 3 or SEQ ID NO: 48. In some embodiments, the NLS has a sequence at least 95% identical to SEQ ID NO: 4 or SEQ ID NO: 70. In some embodiments, the NLS has a sequence comprising SEQ ID NO: 4 or SEQ ID NO: 70.

In certain embodiments, the first agent has a sequence at least 80% identical to SEQ ID NO: 78 and the second agent has a sequence at least 80% identical to SEQ ID NO: 79 In certain embodiments, the first agent has a sequence at least 85% identical to SEQ ID NO: 78 and the second agent has a sequence at least 85% identical to SEQ ID NO: 79. In certain embodiments, the first agent has a sequence at least 90% identical to SEQ ID NO: 78 and the second agent has a sequence at least 90% identical to SEQ ID NO: 79. In certain embodiments, the first agent has a sequence at least 95% identical to SEQ ID NO: 78 and the second agent has a sequence at least 95% identical to SEQ ID NO: 79. In certain embodiments, the first agent has a sequence at least 96% identical to SEQ ID NO: 78 and the second agent has a sequence at least 96% identical to SEQ ID NO: 79. In certain embodiments, the first agent has a sequence at least 97% identical to SEQ ID NO: 78 and the second agent has a sequence at least 97% identical to SEQ ID NO: 79. In certain embodiments, the first agent has a sequence at least 98% identical to SEQ ID NO: 78 and the second agent has a sequence at least 98% identical to SEQ ID NO: 79. In certain embodiments, the first agent has a sequence at least 99% identical to SEQ ID NO: 78 and the second agent has a sequence at least 99% identical to SEQ ID NO: 79. In certain embodiments, the first agent has a sequence 100% identical to SEQ ID NO: 78 and the second agent has a sequence 100% identical to SEQ ID NO: 79. In certain embodiments, the first agent has a sequence comprising SEQ ID NO: 78 and the second agent has a sequence comprising SEQ ID NO: 79.

In some embodiments, the first agent further comprising a first 5′HA and a first 3′HA, wherein the first 5′ HA is operably linked 5′ to the first P2A moiety and the first marker moiety is operably linked 5′ to the first 3′ HA; and the second agent further comprising a second 5′ HA and a second 3′HA, wherein the first 5′ HA is operably linked 5′ to the first P2A moiety, the first T2A moiety is operably linked 5′ to the first 3′ HA, the second 5′ HA is operably linked 5′ to the second P2A moiety, and the second marker moiety is operably linked to the second 3′ HA.

In certain embodiments, the gene of interest (GOI) or a fragment thereof comprises the Deleted in AZoospermia-Like (DAZL) gene or a fragment thereof and wherein the first 5′ HA, the second 5′ HA, or both has a sequence at least 95% identical to SEQ ID NO: 23 or SEQ ID NO: 46, and the first 3′ HA, the second 3′ HA, or both has a sequence at least 95% identical to SEQ ID NO: 24 or SEQ ID NO: 47. In certain embodiments, the gene of interest (GOI) or a fragment thereof comprises the Deleted in AZoospermia-Like (DAZL) gene or a fragment thereof and wherein the first 5′ HA, the second 5′ HA, or both has a sequence comprising SEQ ID NO: 23 or SEQ ID NO: 46, and the first 3′ HA, the second 3′ HA, or both has a sequence comprising SEQ ID NO: 24 or SEQ ID NO: 47.

In some embodiments, the target site comprising a recombinase recognition site comprising a LoxP site (LoxP), the LoxP integrated into the GOI or its promoter region by homologous recombination of one or more single-stranded oligodeoxynucleotide (ssODN) moieties and one or more single-guide ribonucleic acid (sgRNA) moieties, wherein recognition of the LoxP site by Cre recombinase deletes, disrupts, or functionally modifies the GOI and reduces or inhibits survival, maturation, or differentiation of a PGC, or the specific gametogenesis, gamete maturation, or gamete functional modification reduces or inhibits gametogenesis, meiosis, gamete function, or gamete fertilization in an avian. In some embodiments, the LoxP site has a sequence being at least 95% identical to SEQ ID NO: 1. In some embodiments, the LoxP site comprises SEQ ID NO: 1.

In some embodiments, the target site comprising a recombinase recognition site comprising a FRT site (FRT), the FRT integrated into the GOI or its promoter region by homologous recombination of one or more single-stranded oligodeoxynucleotide (ssODN) moieties and one or more single-guide ribonucleic acid (sgRNA) moieties, wherein recognition of the FRT site by flippase (Flp) recombinase deletes, disrupts, or functionally modifies the GOI and reduces or inhibits survival, maturation, or differentiation of a PGC, or the specific gametogenesis, gamete maturation, or gamete functional modification reduces or inhibits gametogenesis, meiosis, gamete function, or gamete fertilization in an avian. In some embodiments, the FRT site has a sequence at least 95% identical to SEQ ID NO: 30. In some embodiments, the FRT site comprises SEQ ID NO: 30.

In certain embodiments, the one or more single-guide ribonucleic acid (sgRNA) moieties has a ribonucleic acid sequence corresponding to a deoxyribonucleic acid sequence at least 95% identical to SEQ ID NO: 33 and the one or more single-stranded oligodeoxynucleotide (ssODN) moieties has a sequence at least 95% identical to SEQ ID NO: 35; wherein the one or more single-guide ribonucleic acid (sgRNA) moieties has a ribonucleic acid sequence corresponding to a deoxyribonucleic acid sequence at least 95% identical to SEQ ID NO: 34 and the one or more single-stranded oligodeoxynucleotide (ssODN) moieties has a sequence at least 95% identical to SEQ ID NO: 36, respectively; wherein the one or more sgRNA moieties has a ribonucleic acid sequence corresponding to a deoxyribonucleic acid sequence at least 95% identical to SEQ ID NO: 21 and LoxP flanked by ssODN moieties has a sequence at least 95% identical to SEQ ID NO: 19; wherein the one or more sgRNA moieties has a ribonucleic acid sequence corresponding to a deoxyribonucleic acid sequence at least 95% identical to SEQ ID NO: 22 and LoxP flanked by ssODNs has a sequence at least 95% identical to SEQ ID NO: 20; or a combination of any of these. In certain embodiments, the one or more single-guide ribonucleic acid (sgRNA) moieties has a ribonucleic acid sequence corresponding to a deoxyribonucleic acid sequence comprising SEQ ID NO: 33 and the one or more single-stranded oligodeoxynucleotide (ssODN) moieties has a sequence comprising SEQ ID NO: 35; wherein the one or more single-guide ribonucleic acid (sgRNA) moieties has a ribonucleic acid sequence corresponding to a deoxyribonucleic acid sequence comprising SEQ ID NO: 34 and the one or more single-stranded oligodeoxynucleotide (ssODN) moieties has a sequence comprising SEQ ID NO: 36, respectively; wherein the one or more sgRNA moieties has a ribonucleic acid sequence corresponding to a deoxyribonucleic acid sequence comprising SEQ ID NO: 21 and LoxP flanked by ssODN moieties has a sequence comprising SEQ ID NO: 19; wherein the one or more sgRNA moieties has a ribonucleic acid sequence corresponding to a deoxyribonucleic acid sequence comprising SEQ ID NO: 22 and LoxP flanked by ssODNs has a sequence comprising SEQ ID NO: 20; or a combination of any of these. In some embodiments, at least one of the one or more ssODN moieties further comprising a protospacer adjacent motif (PAM) sequence moiety. In some embodiments, the PAM sequence moiety comprising 5′-AAG-3′ operably linked to the 3′-end of the at least one of the one or more ssODN moieties.

In another aspect, provided herein is a genomic modifying vector pair comprising: (a) a first vector comprising a first promoter operably linked to a first exogenous polynucleotide encoding a first nuclear localization signal (NLS), a functionally inactive first protein moiety of interest, and a first intein moiety; and (b) a second vector comprising a second promoter operably linked to a second exogenous polynucleotide encoding a second nuclear localization signal (NLS), a functionally inactive second protein moiety of interest, and a second intein moiety, wherein: (i) the functionally inactive first protein moiety of interest and the functionally inactive second protein moiety of interest, dimerized or bound covalently, comprise at least one functionally active protein of interest or fragment thereof, wherein: (a) the functionally active protein of interest or fragment thereof comprises a genomic modifier, the genomic modifier targeting a gene of interest (GOI) or fragment thereof on a chromosome, the GOI modified to introduce one or more target sites specific to the genomic modifier, and the GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active gene of interest (GOI), the modified trait inducing sterility or inhibiting fertility in the functionally modified and genetically modified progeny avian embryo or progeny avian without impairing viability; (b) the functionally active protein of interest or fragment thereof comprises a toxin inducing cell death of a PGC, either without impairing viability of somatic cells or limited to benign somatic cell loss or both, in the functionally modified and genetically modified progeny avian embryo or progeny avian, and inducing sterility or inhibiting fertility in the functionally modified and genetically modified progeny avian embryo or progeny avian without impairing viability; or (c) a combination thereof, (ii) the functionally inactive first protein moiety of interest and the functionally inactive second protein moiety of interest, when co-expressed, comprise at least one functionally active protein of interest or fragment thereof, the co-expression of which inducing cell death of a PGC without impairing viability of somatic cells in the functionally modified and genetically modified progeny avian embryo or progeny avian and inducing sterility or inhibiting fertility in the functionally modified and genetically modified avian without impairing viability; or (c) a combination of any of the above.

In some embodiments, the genomic modifier comprises a recombinase. In some embodiments, (a) the target site comprises a Lox site; and (b) the first exogenous polynucleotide encodes a protein comprising SEQ ID NO: 4, SEQ ID NO: 6, and SEQ ID NO: 8 and the second exogenous polynucleotide encodes a protein comprising SEQ ID NO: 4, SEQ ID NO: 12, and SEQ ID NO: 10; the first exogenous polynucleotide encodes a protein comprising SEQ ID NO: 70, SEQ ID NO: 71, and SEQ ID NO: 72, and the second exogenous polynucleotide encodes a protein comprising SEQ ID NO: 70, SEQ ID NO: 73, and SEQ ID NO: 74; or the first exogenous polynucleotide encodes a protein comprising SEQ ID NO: 78 and the second exogenous polynucleotide encodes a protein comprising SEQ ID NO: 79.

In further embodiments, the first protein moiety of interest comprises a functionally active first genomic modifier and the second protein moiety of interest comprising a functionally active second genomic modifier, wherein: (a) the functionally active second genomic modifier targeting a first gene of interest (GOI) or fragment thereof on a chromosome, the first GOI or fragment thereof modified to introduce one or more target sites specific to the second genomic modifier, and the first GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active first gene of interest (GOI), the modified trait inducing sterility or inhibiting fertility in the functionally modified and genetically modified progeny avian embryo or progeny avian without impairing viability; and (b) the functionally active first genomic modifier targeting a second gene of interest (GOI) or fragment thereof on a chromosome, the second GOI or fragment thereof modified to introduce one or more target sites specific to the second genomic modifier, and the second GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active second gene of interest, the modified trait inducing sterility or inhibiting fertility in the functionally modified and genetically modified progeny avian embryo or progeny avian without impairing viability.

In some embodiments, the gene of interest (GOI) sequence or fragment thereof has (a) an isolated function specific to a PGC, or (b) a function specific to gametogenesis, meiosis, gamete maturation, gamete function, or gamete fertilization in the genetically modified avian, wherein deletion, disruption, or functional modification of the gene reduces or inhibits survival, maturation, or differentiation of a PGC or the specific gametogenesis, gamete maturation, or gamete functional modification reduces or inhibits gametogenesis, meiosis, gamete function, or gamete fertilization in an avian. In some embodiments, the first gene of interest (GOI) sequence or fragment thereof, the second gene of interest (GOI) sequence or fragment thereof, or both comprise a deleted in azoospermia-like (DAZL) gene, a deleted in azoospermia 1 (DAZ1) gene, a zona pellucida binding protein 1/2 (ZPBP1/2) gene, a cyclin-dependent kinases regulatory subunit 2 (CKS2; CDC28 Protein Kinase Regulatory Subunit 2) gene, a spermatogenesis associated 16 (SPATA16) gene, a DEAD-box helicase 4 (DDX4) gene, a serine/threonine-protein phosphatase PP1-gamma catalytic subunit (PPP1CC) gene, an Izumo sperm-egg fusion 1 (IZUMO1) gene, a synaptonemal complex central element protein 1 (SYCE1) gene, a YTH domain-containing 2 (YTHDC2) gene, a Meiosis Specific With Coiled-Coil Domain (MEIOC) gene, a septin-4 (SEPT4) gene, a stromal antigen 3 (STAG3) gene, a Nanos C2HC-type zinc finger 3 (NANOS3) gene, or a combination of any of these. In some embodiments, the first gene of interest (GOI) and the second GOI comprise the same GOI. In some embodiments, the fragment of the first gene of interest (GOI) and the fragment of the second GOI are distinct fragments of the same GOI.

In some embodiments, the first genomic modifier, the second genomic modifier, or both independently comprise a non-identical site-specific recombinase enzyme or functionally active fragment thereof. In some embodiments, the site-specific recombinase comprises a tyrosine recombinase or a serine recombinase. In some embodiments, the tyrosine recombinase comprises a Cre recombinase (Cre), a Dre recombinase (Dre), a flippase recombinase (Flp), or a Vika recombinase (Vika), and the target site comprises a recombinase recognition site, the recombinase recognition site comprising, respectively, a Lox site (Lox), a Rox site (Rox), a FRT site (FRT), or a Vox site (Vox). In some embodiments, the tyrosine recombinase comprises Cre and the recombinase recognition site comprises a locus of X-over P1 site (LoxP). In some embodiments, the tyrosine recombinase comprises Flp and the recombinase recognition site comprises FRT.

In some embodiments, the first exogenous polynucleotide encodes a functionally active marker operably linked to the first protein moiety of interest; the second exogenous polynucleotide encodes a functionally active marker operably linked to the second protein moiety of interest; or a combination thereof. In some embodiments, the marker encoded by the first exogenous polynucleotide is distinct from the marker encoded by the second exogenous polynucleotide. In some embodiments, the marker is a fluorescent protein, a luminescent protein, or a chromoprotein.

In some embodiments, the first exogenous polynucleotide encodes a nuclear localization signal (NLS) operably linked to the first protein moiety of interest; the second exogenous polynucleotide encodes a nuclear localization signal (NLS) operably linked to the second protein moiety of interest; or a combination thereof.

In some embodiments, the first exogenous polynucleotide further comprises one or more self-cleaving peptides operably linked to the first protein moiety of interest and wherein the second exogenous polynucleotide further comprises one or more self-cleaving peptides operably linked to the second protein moiety of interest.

In some embodiments, the first exogenous polynucleotide further comprises a first 5′ homology arm (HA) and a first 3′ homology arm (HA), said first 5′ HA and said first 3′ HA specific for a first insertion site of interest on the avian genome and wherein the second exogenous polynucleotide further comprises a second 5′ homology arm (HA) and a second 3′ homology arm (HA), said second 5′ HA and said second 3′ HA specific for a second insertion site of interest on the avian genome, wherein the first 5′ HA has a nucleotide sequence that is substantially homologous to the 5′ region flanking a first gene of interest (GOI) in a first chromosome of interest and the first 3′ HA has a nucleotide sequence that is substantially homologous to the 3′ region flanking the first GOI in the first chromosome of interest; the second 5′ HA has a nucleotide sequence that is substantially homologous to the 5′ region flanking a second GOI in a second chromosome of interest and the second 3′ HA, or both has a nucleotide sequence that is substantially homologous to the 3′ region flanking the second GOI in the second chromosome of interest; or both. In some embodiments, the first GOI, prior to modification with the one or more target sites or to the deletion, disruption, or functional modification of the GOI, and the second GOI, prior to modification with the one or more target sites or to the deletion, disruption, or functional modification of the GOI, are the same GOI and the first chromosome of interest and the second chromosome of interest are the same chromosome of interest.

In certain embodiments, (a) the first genomic modifier comprises a functionally active tyrosine recombinase comprising Flipase (Flp), operably linked to a nuclear localizing signal (NLS), and the first GOI comprises a recombinase recognition site, the recombinase recognition site comprising LoxP; and the second genomic modifier comprises a functionally active tyrosine recombinase comprising Cre, operably linked to a nuclear localizing signal (NLS), and the second GOI comprises a recombinase recognition site, the recombinase recognition site comprising FRT. In certain embodiments, the first genomic modifier comprising Flp, operably linked to a NLS, has a sequence at least 95% identical to SEQ ID NO: 29; or wherein the second genomic modifier comprising Cre, operably linked to a NLS, has a sequence at least 95% identical to SEQ ID NO: 27. In certain embodiments, the first genomic modifier comprising Flp, operably linked to a NLS, has a sequence comprising SEQ ID NO: 29; or wherein the second genomic modifier comprising Cre, operably linked to a NLS, has a sequence comprising SEQ ID NO: 27.

In some embodiments, the first genomic modifier further comprises a first P2A moiety, a NLS, Flp, a first T2A moiety, and a first marker moiety, wherein the first P2A moiety is operably linked 5′ to the nuclear localizing signal (NLS), the NLS is operably linked 5′ to the Flp, the Flp is operably linked 5′ to the first T2A moiety, and the first T2A moiety is operably linked 5′ to the first marker moiety; and the second genomic modifier further comprises a second P2A moiety, a NLS, Cre, a second T2A moiety, and a second marker moiety, wherein the second P2A moiety is operably linked 5′ to the Cre, the Cre is operably linked 5′ to the second T2A moiety, and the second T2A moiety is operably linked 5′ to the second marker moiety. In some embodiments of a first genomic modifier and or a second genomic modifier, P2A and T2A are interchangeable.

In some embodiments, the first genomic modifier further comprising a first 5′HA and a first 3′HA, wherein the first 5′ HA is operably linked 5′ to the first P2A moiety and the first marker moiety is operably linked 5′ to the first 3′ HA; and the second genomic modifier further comprising a second 5′ HA and a second 3′HA, wherein the second 5′ HA is operably linked 5′ to the second P2A moiety, and the second T2A moiety is operably linked 5′ to the second 3′ HA.

In some embodiments, the gene of interest (GOI) or a fragment thereof comprises the Deleted in AZoospermia-Like (DAZL) gene or a fragment thereof.

In certain embodiments, (a) the first GOI comprising a recombinase recognition site comprises a LoxP site, the LoxP integrated into the GOI or its promoter region by homologous recombination of one or more single-stranded oligodeoxynucleotide (ssODN) moieties and one or more single-guide ribonucleic acid (sgRNA) moieties, wherein recognition of the LoxP site by a Cre recombinase deletes, disrupts, or functionally modifies the GOI and reduces or inhibits survival, maturation, or differentiation of a PGC, or the specific gametogenesis, gamete maturation, or gamete functional modification reduces or inhibits gametogenesis, meiosis, gamete function, or gamete fertilization in an avian, wherein the one or more single-guide ribonucleic acid (sgRNA) moieties has a ribonucleic acid sequence corresponding to a deoxyribonucleic acid sequence at least 95% identical to SEQ ID NO: 21 and the one or more single-stranded oligodeoxynucleotide (ssODN) moieties, with LoxP, has a sequence at least 95% identical to SEQ ID NO: 19; or wherein the one or more single-guide ribonucleic acid (sgRNA) moieties has a ribonucleic acid sequence corresponding to a deoxyribonucleic acid sequence at least 95% identical to SEQ ID NO: 22 and the one or more single-stranded oligodeoxynucleotide (ssODN) moieties, with LoxP, has a sequence at least 95% identical to SEQ ID NO: 20; or (b) the second GOI comprising a recombinase recognition site comprises a FRT site, the FRT integrated into the GOI or its promoter region by homologous recombination of one or more single-stranded oligodeoxynucleotide (ssODN) moieties and one or more single-guide ribonucleic acid (sgRNA) moieties, wherein recognition of the FRT site by flippase recombinase (Flp) deletes, disrupts, or functionally modifies the GOI and reduces or inhibits survival, maturation, or differentiation of a PGC, or the specific gametogenesis, gamete maturation, or gamete functional modification reduces or inhibits gametogenesis, meiosis, gamete function, or gamete fertilization in an avian, wherein the one or more single-guide ribonucleic acid (sgRNA) moieties has a ribonucleic acid sequence corresponding to a deoxyribonucleic acid sequence at least 95% identical to SEQ ID NO: 21 and the one or more single-stranded oligodeoxynucleotide (ssODN) moieties, with FRT, has a sequence at least 95% identical to SEQ ID NO: 31; or wherein the one or more single-guide ribonucleic acid (sgRNA) moieties has a ribonucleic acid sequence corresponding to a deoxyribonucleic acid sequence at least 95% identical to SEQ ID NO: 22 and the one or more single-stranded oligodeoxynucleotide (ssODN) moieties, with FRT, has a sequence at least 95% identical to SEQ ID NO: 32. In certain embodiments, at least one of the one or more ssODN moieties further comprising a protospacer adjacent motif (PAM) sequence moiety. In certain embodiments, the PAM sequence moiety comprising 5′-AAG-3′ operably linked to the 3′-end of the at least one of the one or more ssODN moieties.

In certain embodiments, (a) the first GOI comprising a recombinase recognition site comprises a LoxP site, the LoxP integrated into the GOI or its promoter region by homologous recombination of one or more single-stranded oligodeoxynucleotide (ssODN) moieties and one or more single-guide ribonucleic acid (sgRNA) moieties, wherein recognition of the LoxP site by a Cre recombinase deletes, disrupts, or functionally modifies the GOI and reduces or inhibits survival, maturation, or differentiation of a PGC, or the specific gametogenesis, gamete maturation, or gamete functional modification reduces or inhibits gametogenesis, meiosis, gamete function, or gamete fertilization in an avian, wherein the one or more single-guide ribonucleic acid (sgRNA) moieties has a ribonucleic acid sequence corresponding to a deoxyribonucleic acid sequence comprising SEQ ID NO: 21 and the one or more single-stranded oligodeoxynucleotide (ssODN) moieties, with LoxP, has a sequence comprising SEQ ID NO: 19; or wherein the one or more single-guide ribonucleic acid (sgRNA) moieties has a ribonucleic acid sequence corresponding to a deoxyribonucleic acid sequence comprising SEQ ID NO: 22 and the one or more single-stranded oligodeoxynucleotide (ssODN) moieties, with LoxP, has a sequence comprising SEQ ID NO: 20; or (b) the second GOI comprising a recombinase recognition site comprises a FRT site, the FRT integrated into the GOI or its promoter region by homologous recombination of one or more single-stranded oligodeoxynucleotide (ssODN) moieties and one or more single-guide ribonucleic acid (sgRNA) moieties, wherein recognition of the FRT site by flippase recombinase (Flp) deletes, disrupts, or functionally modifies the GOI and reduces or inhibits survival, maturation, or differentiation of a PGC, or the specific gametogenesis, gamete maturation, or gamete functional modification reduces or inhibits gametogenesis, meiosis, gamete function, or gamete fertilization in an avian, wherein the one or more single-guide ribonucleic acid (sgRNA) moieties has a ribonucleic acid sequence corresponding to a deoxyribonucleic acid sequence comprising SEQ ID NO: 21 and the one or more single-stranded oligodeoxynucleotide (ssODN) moieties, with FRT, has a sequence comprising SEQ ID NO: 31; or wherein the one or more single-guide ribonucleic acid (sgRNA) moieties has a ribonucleic acid sequence corresponding to a deoxyribonucleic acid sequence comprising SEQ ID NO: 22 and the one or more single-stranded oligodeoxynucleotide (ssODN) moieties, with FRT, has a sequence comprising SEQ ID NO: 32. In certain embodiments, at least one of the one or more ssODN moieties further comprising a protospacer adjacent motif (PAM) sequence moiety. In certain embodiments, the PAM sequence moiety comprising 5′-AAG-3′ operably linked to the 3′-end of the at least one of the one or more ssODN moieties.

In some embodiments, the gene of interest (GOI) or a fragment thereof comprises the Deleted in Azoospermia-Like (DAZL) gene or a fragment thereof and the first 5′ HA, the second 5′ HA, or both has a sequence at least 95% identical to SEQ ID NO: 46 or SEQ ID NO: 23 and the first 3′ HA, the second 3′ HA, or both has a sequence at least 95% identical to SEQ ID NO: 47 or SEQ ID NO: 24. In some embodiments, the gene of interest (GOI) or a fragment thereof comprises the Deleted in Azoospermia-Like (DAZL) gene or a fragment thereof and the first 5′ HA, the second 5′ HA, or both has a sequence comprising SEQ ID NO: 46 or SEQ ID NO: 23 and the first 3′ HA, the second 3′ HA, or both has a sequence comprising SEQ ID NO: 47 or SEQ ID NO: 24.

In some embodiments, the first exogenous polynucleotide, the second exogenous polynucleotide, or both further comprises at least one self-cleaving peptide.

In another aspect, provided herein is a primordial germ cell (PGC) system comprising a first genetically modified avian primordial germ cell (PGC) and a second genetically modified avian primordial germ cell (PGC): (a) the first genetically modified avian PGC comprising a first agent, the first agent comprising a first exogenous polynucleotide, the first exogenous polynucleotide comprising: (i) a first promoter; and (ii) a first element of interest operably linked to the promoter, the first element of interest encoding a first protein moiety of interest, wherein a first genetically modified avian having a genome comprising the first agent retains fertility and viability while producing a primordial germ cell (PGC) having a genome comprising the first agent without impairing viability of the PGC; and (b) the second genetically modified avian PGC comprising a second agent, the second agent comprising a second exogenous polynucleotide, the second exogenous polynucleotide comprising: (i) a second promoter; and (ii) a second element of interest operably linked to the promoter, the second element of interest encoding a second protein moiety of interest, wherein a second genetically modified avian having a genome comprising the second agent retains fertility and viability while producing a primordial germ cell (PGC) having a genome comprising the second agent without impairing viability of the PGC, the first agent and the second agent, when co-expressed in a genetically modified progeny avian embryo or progeny avian, wherein the genetically modified progeny avian embryo or progeny avian is a progeny of a first genetically modified avian arising from the first genetically modified PGC comprising the first agent and an opposite-gendered second genetically modified avian arising from the second genetically modified PGC comprising the second agent, inducing sterility or inhibiting fertility in the genetically modified progeny avian embryo or progeny avian without impairing viability.

In some embodiments, the first promoter, the second promoter, or both comprises: (a) a promoter specific to a primordial germ cell (PGC); (b) a tissue-specific promoter; or (c) a ubiquitous promoter.

In some embodiments, the first protein moiety of interest is a functionally inactive first protein moiety of interest and the second protein moiety of interest is a functionally inactive second protein moiety of interest, wherein: (a) the functionally inactive first protein moiety of interest and the functionally inactive second protein moiety of interest, dimerized or bound covalently, comprise at least one functionally active protein of interest or fragment thereof, wherein: (i) the functionally active protein of interest or fragment thereof comprises a genomic modifier, the genomic modifier targeting a gene of interest (GOI) or fragment thereof on a chromosome, the GOI modified to introduce one or more target sites specific to the genomic modifier, and the GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active gene of interest (GOI), the modified trait inducing sterility or inhibiting fertility in the functionally modified and genetically modified progeny avian embryo or progeny avian without impairing viability; (ii) the functionally active protein of interest or fragment thereof comprises a toxin inducing cell death of a PGC, either without impairing viability of somatic cells or limited to benign somatic cell loss or both, in the functionally modified and genetically modified progeny avian embryo or progeny avian, and inducing sterility or inhibiting fertility in the functionally modified and genetically modified progeny avian embryo or progeny avian without impairing viability; or (iii) a combination thereof, (b) the functionally inactive first protein moiety of interest and the functionally inactive second protein moiety of interest, when co-expressed, comprise at least one functionally active protein of interest or fragment thereof, the co-expression of which inducing cell death of a PGC without impairing viability of somatic cells in the functionally modified and genetically modified progeny avian embryo or progeny avian and inducing sterility or inhibiting fertility in the functionally modified and genetically modified avian without impairing viability; or (c) a combination of any of the above.

In some embodiments, the genomic modifier comprises a site-specific recombinase enzyme or functionally active fragment thereof.

In some embodiments, the gene of interest (GOI) sequence or fragment thereof has (a) an isolated function specific to a PGC, or (b) a function specific to gametogenesis, meiosis, gamete maturation, gamete function, or gamete fertilization in the genetically modified avian, wherein deletion, disruption, or functional modification of the gene reduces or inhibits survival, maturation, or differentiation of a PGC or the specific gametogenesis, gamete maturation, or gamete functional modification reduces or inhibits gametogenesis, meiosis, gamete function, or gamete fertilization in an avian.

In some embodiments, the gene of interest (GOI) sequence or fragment thereof comprises a deleted in azoospermia-like (DAZL) gene, a deleted in azoospermia 1 (DAZ1) gene, a zona pellucida binding protein 1/2 (ZPBP1/2) gene, a cyclin-dependent kinases regulatory subunit 2 (CKS2; CDC28 Protein Kinase Regulatory Subunit 2) gene, a spermatogenesis associated 16 (SPATA16) gene, a DEAD-box helicase 4 (DDX4) gene, a serine/threonine-protein phosphatase PP1-gamma catalytic subunit (PPP1CC) gene, an Izumo sperm-egg fusion 1 (IZUMO1) gene, a synaptonemal complex central element protein 1 (SYCE1) gene, a YTH domain-containing 2 (YTHDC2) gene, a Meiosis Specific With Coiled-Coil Domain (MEIOC) gene, a septin-4 (SEPT4) gene, a stromal antigen 3 (STAG3) gene, a Nanos C2HC-type zinc finger 3 (NANOS3) gene, or a combination of any of these.

In some embodiments, the first protein moiety of interest comprises a functionally active first genomic modifier and the second protein moiety of interest comprising a functionally active second genomic modifier, wherein: (a) the functionally active second genomic modifier targeting a first gene of interest (GOI) or fragment thereof on a chromosome, the first GOI or fragment thereof modified to introduce one or more target sites specific to the second genomic modifier, and the first GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active first gene of interest (GOI), the modified trait inducing sterility or inhibiting fertility in the functionally modified and genetically modified progeny avian embryo or progeny avian; and (b) the functionally active first genomic modifier targeting a second gene of interest (GOI) or fragment thereof on a chromosome, the second GOI or fragment thereof modified to introduce one or more target sites specific to the second genomic modifier, and the second GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active second gene of interest, the modified trait inducing sterility or inhibiting fertility in the functionally modified and genetically modified progeny avian embryo or progeny avian without impairing viability.

In some embodiments, the first genomic modifier, the second genomic modifier, or both comprises a site-specific recombinase enzyme or functionally active fragment thereof.

In some embodiments, the first gene of interest (GOI) sequence or fragment thereof, the second gene of interest (GOI) or fragment thereof, or both has (a) an isolated function specific to a PGC, or (b) a function specific to gametogenesis, meiosis, gamete maturation, gamete function, or gamete fertilization in the genetically modified avian, wherein deletion, disruption, or functional modification of the gene reduces or inhibits survival, maturation, or differentiation of a PGC or the specific gametogenesis, gamete maturation, or gamete functional modification reduces or inhibits gametogenesis, meiosis, gamete function, or gamete fertilization in an avian.

In some embodiments, the first gene of interest (GOI) sequence or fragment thereof, the second gene of interest (GOI) sequence or fragment thereof, or both comprise a deleted in azoospermia-like (DAZL) gene, a deleted in azoospermia 1 (DAZ1) gene, a zona pellucida binding protein 12 (ZPBP1/2) gene, a cyclin-dependent kinases regulatory subunit 2 (CKS2; CDC28 Protein Kinase Regulatory Subunit 2) gene, a spermatogenesis associated 16 (SPATA16) gene, a DEAD-box helicase 4 (DDX4) gene, a serine/threonine-protein phosphatase PP1-gamma catalytic subunit (PPP1CC) gene, an Izumo sperm-egg fusion 1 (IZUMO1) gene, a synaptonemal complex central element protein 1 (SYCE1) gene, a YTH domain-containing 2 (YTHDC2) gene, a Meiosis Specific With Coiled-Coil Domain (MEIOC) gene, a septin-4 (SEPT4) gene, a stromal antigen 3 (STAG3) gene, a Nanos C2HC-type zinc finger 3 (NANOS3) gene, or a combination of any of these.

In other aspects, provided herein is a sterile avian breeding system comprising a first genetically modified avian and a second genetically modified avian having an opposite sex to the first genetically modified avian: (a) the first genetically modified avian comprising a first genetically modified avian PGC comprising a first agent, the first agent comprising a first exogenous polynucleotide, the first exogenous polynucleotide comprising: (i) a first promoter; and (ii) a first element of interest operably linked to the promoter, the first element of interest encoding a first protein moiety of interest, and (b) the second genetically modified avian comprising a second genetically modified avian PGC comprising a second agent, the second agent comprising a second exogenous polynucleotide, the second exogenous polynucleotide comprising: (i) a second promoter; and (ii) a second element of interest operably linked to the promoter, the second element of interest encoding a second protein moiety of interest, the first agent and the second agent, when co-expressed in a genetically modified progeny avian embryo or progeny avian, wherein the genetically modified progeny avian embryo or progeny avian is a progeny of the first genetically modified avian arising from the first genetically modified PGC comprising the first agent and an opposite-gendered second genetically modified avian arising from the second genetically modified PGC comprising the second agent, inducing sterility or inhibiting fertility in the genetically modified progeny avian embryo or progeny avian without impairing viability.

In some embodiments, the first genetically modified avian having a genome comprising the first agent retains fertility and viability while producing a primordial germ cell (PGC) having a genome comprising the first agent without impairing viability of the PGC and the second genetically modified avian having a genome comprising the second agent retains fertility and viability while producing a primordial germ cell (PGC) having a genome comprising the second agent without impairing viability of the PGC.

In some embodiments, the first promoter, the second promoter, or both comprises: (a) a promoter specific to a primordial germ cell (PGC); (b) a tissue-specific promoter; or (c) a ubiquitous promoter.

In some embodiments, the first protein moiety of interest is a functionally inactive first protein moiety of interest and the second protein moiety of interest is a functionally inactive second protein moiety of interest, wherein: (a) the functionally inactive first protein moiety of interest and the functionally inactive second protein moiety of interest, dimerized or bound covalently, comprise at least one functionally active protein of interest or fragment thereof, wherein: (i) the functionally active protein of interest or fragment thereof comprises a genomic modifier, the genomic modifier targeting a gene of interest (GOI) or fragment thereof on a chromosome, the GOI modified to introduce one or more target sites specific to the genomic modifier, and the GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active gene of interest (GOI), the modified trait inducing sterility or inhibiting fertility in the functionally modified and genetically modified progeny avian embryo or progeny avian without impairing viability; (ii) the functionally active protein of interest or fragment thereof comprises a toxin inducing cell death of a PGC, either without impairing viability of somatic cells or limited to benign somatic cell loss or both, in the functionally modified and genetically modified progeny avian embryo or progeny avian, and inducing sterility or inhibiting fertility in the functionally modified and genetically modified progeny avian embryo or progeny avian without impairing viability; or (iii) a combination thereof, (b) the functionally inactive first protein moiety of interest and the functionally inactive second protein moiety of interest, when co-expressed, comprise at least one functionally active protein of interest or fragment thereof, the co-expression of which inducing cell death of a PGC without impairing viability of somatic cells in the functionally modified and genetically modified progeny avian embryo or progeny avian and inducing sterility or inhibiting fertility in the functionally modified and genetically modified avian without impairing viability; or (c) a combination of any of the above.

In some embodiments, the gene of interest (GOI) sequence or fragment thereof has (a) an isolated function specific to a PGC, or (b) a function specific to gametogenesis, meiosis, gamete maturation, gamete function, or gamete fertilization, in the genetically modified progeny avian embryo or progeny avian, wherein deletion, disruption, or functional modification of the gene reduces or inhibits survival, maturation, or differentiation of a PGC or the specific gametogenesis, gamete maturation, or gamete functional modification reduces or inhibits gametogenesis, meiosis, gamete function, or gamete fertilization in an avian.

In some embodiments, the gene of interest (GOI) sequence or fragment thereof comprising a deleted in azoospermia-like (DAZL) gene, a deleted in azoospermia 1 (DAZ1) gene, a zona pellucida binding protein 1/2 (ZPBP1/2) gene, a cyclin-dependent kinases regulatory subunit 2 (CKS2; CDC28 Protein Kinase Regulatory Subunit 2) gene, a spermatogenesis associated 16 (SPATA16) gene, a DEAD-box helicase 4 (DDX4) gene, a serine/threonine-protein phosphatase PP1-gamma catalytic subunit (PPP1CC) gene, an Izumo sperm-egg fusion 1 (IZUMO1) gene, a synaptonemal complex central element protein 1 (SYCE1) gene, a YTH domain-containing 2 (YTHDC2) gene, a Meiosis Specific With Coiled-Coil Domain (MEIOC) gene, a septin-4 (SEPT4) gene, a stromal antigen 3 (STAG3) gene, a Nanos C2HC-type zinc finger 3 (NANOS3) gene, or a combination of any of these.

In some embodiments, the genomic modifier comprising a site-specific recombinase enzyme or functionally active fragment thereof.

In some embodiments, the site-specific recombinase comprises a tyrosine recombinase or a serine recombinase. In some embodiments, the tyrosine recombinase comprises Cre, Dre, Flp, or Vika, and the target site comprises a recombinase recognition site, the recombinase recognition site comprising, respectively, Lox, Rox, FRT, or Vox. In some embodiments, the tyrosine recombinase comprises Cre and the recombinase recognition site comprises LoxP. In some embodiments, the tyrosine recombinase comprises Flp and the recombinase recognition site comprises FRT.

In some embodiments, the first exogenous polynucleotide further encodes a first intein moiety operably linked to the first promoter and to the first element of interest encoding the functionally inactive first protein moiety of interest; and the second exogenous polynucleotide further encodes a second intein moiety distinct from the first intein moiety and operably linked to the second promoter and to the second element of interest encoding the functionally inactive second protein moiety of interest, wherein the first intein moiety and the second intein moiety, dimerized or covalently bound, comprise a functionally active intein or fragment thereof, the functionally active intein or fragment thereof splicing the functionally inactive first protein moiety of interest and the functionally inactive second protein moiety of interest to produce the functionally active protein of interest or fragment thereof. In some embodiments, the first intein moiety is a functionally inactive intein moiety and the second intein moiety is a functionally inactive intein moiety.

In some embodiments, the first exogenous polynucleotide further encodes a first conjugating element operably linked to the first promoter and to the first element of interest encoding the functionally inactive first protein moiety of interest; and the second exogenous polynucleotide further encodes a second conjugating element operably linked to the second promoter and to the second element of interest encoding the functionally inactive second protein moiety of interest, the first conjugating element conjugating to the second conjugating element to produce the functionally active protein of interest or fragment thereof. In some embodiments, the first conjugating element comprises SpyTag and the second conjugating element comprises SpyCatcher.

In some embodiments, the first exogenous polynucleotide further encodes a first functionally inactive marker moiety operably linked to the first promoter, the first conjugating element, and the first protein moiety of interest and the second exogenous polynucleotide further encodes a second functionally inactive marker moiety operably linked to the second promoter, the second conjugating element, and the second protein moiety of interest, the first conjugating element conjugating to the second conjugating element to produce a functionally active marker or fragment thereof.

In some embodiments, the first exogenous polynucleotide encodes a functionally active marker operably linked to the first protein moiety of interest; the second exogenous polynucleotide encodes a functionally active marker operably linked to the second protein moiety of interest; or a combination thereof. In some embodiments, the marker encoded by the first exogenous polynucleotide is distinct from the marker encoded by the second exogenous polynucleotide. In some embodiments, the marker is a fluorescent protein, a luminescent protein, or a chromoprotein detectable in the cytoplasm.

In some embodiments, the first exogenous polynucleotide encodes a nuclear localization signal (NLS) operably linked to the first protein moiety of interest; the second exogenous polynucleotide encodes a nuclear localization signal (NLS) operably linked to the second protein moiety of interest; or a combination thereof.

In some embodiments, the first exogenous polynucleotide further comprises one or more self-cleaving peptides operably linked to the first protein moiety of interest and the second exogenous polynucleotide further comprises one or more self-cleaving peptides operably linked to the second protein moiety of interest.

In some embodiments, the first exogenous polynucleotide further comprises a first 5′ homology arm (HA) and a first 3′ homology arm (HA), said first 5′ HA and said first 3′ HA specific for a first insertion site of interest on the avian genome; and the second exogenous polynucleotide further comprises a second 5′ homology arm (HA) and a second 3′ homology arm (HA), said second 5′ HA and said second 3′ HA specific for a second insertion site of interest on the avian genome, wherein the first 5′ HA has a nucleotide sequence that is substantially homologous to the 5′ region flanking a first gene of interest (GOI) in a first chromosome of interest and the first 3′ HA has a nucleotide sequence that is substantially homologous to the 3′ region flanking the first GOI in the first chromosome of interest; the second 5′ HA has a nucleotide sequence that is substantially homologous to the 5′ region flanking a second GOI in a second chromosome of interest and the second 3′ HA, or both has a nucleotide sequence that is substantially homologous to the 3′ region flanking the second GOI in the second chromosome of interest; or both. In some embodiments, the first GOI and the second GOI are the same GOI and the first chromosome of interest and the second chromosome of interest are the same chromosome of interest.

In some embodiments, the gene of interest (GOI) or a fragment thereof comprises the Deleted in Azoospermia-Like (DAZL) gene or a fragment thereof and the first 5′ HA, the second 5′ HA, or both has a sequence at least 95% identical to SEQ ID NO: 46 or SEQ ID NO: 23 and the first 3′ HA, the second 3′ HA, or both has a sequence at least 95% identical to SEQ ID NO: 47 or SEQ ID NO: 24. In some embodiments, the gene of interest (GOI) or a fragment thereof comprises the Deleted in Azoospermia-Like (DAZL) gene or a fragment thereof and the first 5′ HA, the second 5′ HA, or both has a sequence comprising SEQ ID NO: 46 or SEQ ID NO: 23 and the first 3′ HA, the second 3′ HA, or both has a sequence comprising SEQ ID NO: 47 or SEQ ID NO: 24.

In some embodiments, the first exogenous polynucleotide, the second exogenous polynucleotide, or both further comprises at least one self-cleaving peptide.

In some embodiments, the first protein moiety of interest comprises a functionally active first genomic modifier and the second protein moiety of interest comprises a functionally active second genomic modifier, wherein: (a) the functionally active second genomic modifier targeting a first gene of interest (GOI) or fragment thereof on a chromosome, the first GOI or fragment thereof modified to introduce one or more target sites specific to the second genomic modifier, and the first GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active first gene of interest (GOI), the modified trait inducing sterility or inhibiting fertility in the functionally modified and genetically modified progeny avian embryo or progeny avian; and (b) the functionally active first genomic modifier targeting a second gene of interest (GOI) or fragment thereof on a chromosome, the second GOI or fragment thereof modified to introduce one or more target sites specific to the second genomic modifier, and the second GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active second gene of interest, the modified trait inducing sterility or inhibiting fertility in the functionally modified and genetically modified progeny avian embryo or progeny avian without impairing viability.

In some embodiments, the gene of interest (GOI) sequence or fragment thereof has (a) an isolated function specific to a PGC, or (b) a function specific to gametogenesis, meiosis, gamete maturation, gamete function, or gamete fertilization in the genetically modified avian, wherein deletion, disruption, or functional modification of the gene reduces or inhibits survival, maturation, or differentiation of a PGC or the specific gametogenesis, gamete maturation, or gamete functional modification reduces or inhibits gametogenesis, meiosis, gamete function, or gamete fertilization in an avian.

In some embodiments, the first gene of interest (GOI) sequence or fragment thereof and the second gene of interest (GOI) sequence or fragment thereof independently comprise a deleted in azoospermia-like (DAZL) gene, a deleted in azoospermia 1 (DAZ1) gene, a zona pellucida binding protein 1/2 (ZPBP1/2) gene, a cyclin-dependent kinases regulatory subunit 2 (CKS2; CDC28 Protein Kinase Regulatory Subunit 2) gene, a spermatogenesis associated 16 (SPATA16) gene, a DEAD-box helicase 4 (DDX4) gene, a serine/threonine-protein phosphatase PP1-gamma catalytic subunit (PPP1CC) gene, an Izumo sperm-egg fusion 1 (IZUMO1) gene, a synaptonemal complex central element protein 1 (SYCE1) gene, a YTH domain-containing 2 (YTHDC2) gene, a Meiosis Specific With Coiled-Coil Domain (MEIOC) gene, a septin-4 (SEPT4) gene, a stromal antigen 3 (STAG3) gene, a Nanos C2HC-type zinc finger 3 (NANOS3) gene, or a combination of any of these.

In some embodiments, the first gene of interest (GOI) and the second GOI comprising the same GOI. In some embodiments, the fragment of the first gene of interest (GOI) and the fragment of the second GOI are distinct fragments of the same GOI.

In some embodiments, the genomic modifier comprising a site-specific recombinase enzyme or functionally active fragment thereof. In some embodiments, the site-specific recombinase comprises a tyrosine recombinase or a serine recombinase. In some embodiments, the tyrosine recombinase comprises Cre, Dre, Flp, or Vika, and the target site comprises a recombinase recognition site, the recombinase recognition site comprising, respectively, Lox, Rox, FRT, or Vox. In some embodiments, the tyrosine recombinase comprises Cre, and the recombinase recognition site comprises LoxP. In some embodiments, the tyrosine recombinase comprises Flp, and the recombinase recognition site comprises FRT.

In some embodiments, the first exogenous polynucleotide encodes a functionally active marker operably linked to the first protein moiety of interest; the second exogenous polynucleotide encodes a functionally active marker operably linked to the second protein moiety of interest; or a combination thereof. In some embodiments, the marker encoded by the first exogenous polynucleotide is distinct from the marker encoded by the second exogenous polynucleotide. In some embodiments, the marker is a fluorescent protein, a luminescent protein, or a chromoprotein detectable in the cytoplasm.

In some embodiments, the first exogenous polynucleotide encodes a nuclear localization signal (NLS) operably linked to the first protein moiety of interest; the second exogenous polynucleotide encodes a nuclear localization signal (NLS) operably linked to the second protein moiety of interest; or a combination thereof.

In some embodiments, the first exogenous polynucleotide further comprises one or more self-cleaving peptides operably linked to the first protein moiety of interest; and the second exogenous polynucleotide further comprises one or more self-cleaving peptides operably linked to the second protein moiety of interest.

In some embodiments, the first exogenous polynucleotide further comprises a first 5′ homology arm (HA) and a first 3′ homology arm (HA), said first 5′ HA and said first 3′ HA specific for a first insertion site of interest on the avian genome; and the second exogenous polynucleotide further comprises a second 5′ homology arm (HA) and a second 3′ homology arm (HA), said second 5′ HA and said second 3′ HA specific for a second insertion site of interest on the avian genome, wherein the first 5′ HA has a nucleotide sequence that is substantially homologous to the 5′ region flanking a first gene of interest (GOI) in a first chromosome of interest and the first 3′ HA has a nucleotide sequence that is substantially homologous to the 3′ region flanking the first GOI in the first chromosome of interest; the second 5′ HA has a nucleotide sequence that is substantially homologous to the 5′ region flanking a second GOI in a second chromosome of interest and the second 3′ HA, or both has a nucleotide sequence that is substantially homologous to the 3′ region flanking the second GOI in the second chromosome of interest; or both. In some embodiments, the first GOI and the second GOI are the same GOI and the first chromosome of interest and the second chromosome of interest are the same chromosome of interest.

In some embodiments, the gene of interest (GOI) or a fragment thereof comprises the Deleted in Azoospermia-Like (DAZL) gene or a fragment thereof and the first 5′ HA, the second 5′ HA, or both has a sequence at least 95% identical to SEQ ID NO: 46 or SEQ ID NO: 23 and the first 3′ HA, the second 3′ HA, or both has a sequence at least 95% identical to SEQ ID NO: 47 or SEQ ID NO: 24. In some embodiments, the gene of interest (GOI) or a fragment thereof comprises the Deleted in Azoospermia-Like (DAZL) gene or a fragment thereof and the first 5′ HA, the second 5′ HA, or both has a sequence comprising SEQ ID NO: 46 or SEQ ID NO: 23 and the first 3′ HA, the second 3′ HA, or both has a sequence comprising SEQ ID NO: 47 or SEQ ID NO: 24.

In some embodiments, the first exogenous polynucleotide, the second exogenous polynucleotide, or both further comprises at least one self-cleaving peptide.

In some embodiments, the genetically modified avian PGC is derived from an avian of the Galliformes order, the Anseriformes order, the Otidiformes order, the Columbiformes order, or the Struthioniformes order. In some embodiments, when derived from the Galliformes order, the genetically modified avian PGC is derived from an avian of the Numidia family or the Phasianidae family comprising the Gallus genus or the Meleagris genus, respectively.

In other aspects, provided herein is a method for producing a sterile genetically modified avian or a population of sterile genetically modified avians from two fertile independently genetically modified avians, the method comprising: (a) obtaining a first primordial germ cell (PGC) from an avian; (b) integrating into a chromosome of interest in the first PGC a first agent, the first agent comprising a first exogenous polynucleotide, the first exogenous polynucleotide comprising: (i) a first promoter; and (ii) a first element of interest operably linked to the promoter, the first element of interest encoding a first protein moiety of interest, wherein the first genetically modified avian having a genome comprising the first agent retains fertility and viability while producing a primordial germ cell (PGC) having a genome comprising the first agent without impairing viability of the PGC; (c) producing first pure PGC colonies comprising the first agent; (d) transplanting a first pure PGC colony into a male chick embryo to produce a first chimera male chick embryo and transplanting a first pure PGC colony into a female chick embryo to produce a first chimera female chick embryo; (e) producing a population of first chimera founder adult avians by hatching and rearing the first chimera founder chicks to sexual maturity, by mating first chimera founder chicks at adulthood to produce first chimera offspring, or both; (f) screening the first population of chimera founder adult avians to verify homozygosity for the first agent; (g) obtaining a second primordial germ cell (PGC) from an avian; (h) integrating into a chromosome of interest in the second PGC a second agent, the first agent comprising a second exogenous polynucleotide, the second exogenous polynucleotide comprising: (i) a second promoter; and (ii) a second element of interest operably linked to the promoter, the second element of interest encoding a second protein moiety of interest, wherein the second genetically modified avian having a genome comprising the second agent retains fertility and viability while producing a primordial germ cell (PGC) having a genome comprising the second agent without impairing viability of the PGC; (h) producing second pure PGC colonies comprising the second agent; (i) transplanting a second pure PGC colony into a male chick embryo to produce a second chimera male chick embryo and transplanting a second pure PGC colony into a female chick embryo to produce a second chimera female chick embryo; (i) producing a population of second chimera founder adult avians by hatching and rearing the second chimera founder chicks to sexual maturity, by mating chimera founder chicks at adulthood to produce offspring, or both; (j) screening the second population of chimera founder adult avians to verify homozygosity for the second agent; (k) selecting a male homozygous for the first agent from the first population of adult avians; (l) selecting a female homozygous for the second agent from the second population of adult avians; and (m) breeding the male adult avian from the first population with the female adult avian from the second population to produce a population of one or more sterile genetically modified progeny avian embryos, the first agent and the second agent, when co-expressed in the one or more genetically modified progeny avian embryos, inducing sterility or inhibiting fertility in the one or more genetically modified progeny avian embryos without impairing viability.

In some embodiments, the first promoter, the second promoter, or both comprises: (a) a promoter specific to a primordial germ cell (PGC); (b) a tissue-specific promoter; or (c) a ubiquitous promoter.

In some embodiments, the method further comprises: (a) obtaining a first primordial germ cell (PGC) from an avian; (b) integrating into a chromosome of interest in the first PGC a first agent, the first agent comprising a first exogenous polynucleotide, the first exogenous polynucleotide comprising: (i) a first promoter; and (ii) a first element of interest operably linked to the promoter, the first element of interest encoding a first protein moiety of interest, wherein the first protein moiety of interest is a functionally inactive first protein moiety of interest, wherein the first genetically modified avian having a genome comprising the first agent retains fertility and viability while producing a primordial germ cell (PGC) having a genome comprising the first agent without impairing viability of the PGC; (c) integrating into a first targeted gene of interest (GOI) on a chromosome of interest in the first PGC a third agent, the third agent comprising a third exogenous polynucleotide, the third exogenous polynucleotide comprising a functionally active first GOI sequence or a functionally active fragment thereof operatively linked to an one or more target sites specific to a genomic modifier, the first GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active first gene of interest (GOI), the modified trait inducing sterility or inhibiting fertility in the functionally modified and genetically modified progeny avian embryo or progeny avian without impairing viability; (d) producing first pure PGC colonies comprising the first agent and the third agent; (e) transplanting a first pure PGC colony into a male chick embryo to produce a first chimera male chick embryo and transplanting a first pure PGC colony into a female chick embryo to produce a first chimera female chick embryo; (f) producing a population of first chimera founder adult avians by hatching and rearing the first chimera founder chicks to sexual maturity, by mating first chimera founder chicks at adulthood to produce first chimera offspring, or both; (g) screening the first population of chimera founder adult avians to verify homozygosity for the first agent and the third agent; (h) obtaining a second primordial germ cell (PGC) from an avian; (i) integrating into a chromosome of interest in the second PGC a second agent, the second agent comprising a second exogenous polynucleotide, the second exogenous polynucleotide comprising: (i) a second promoter; and (ii) a second element of interest operably linked to the promoter, the second element of interest encoding a second protein moiety of interest, wherein the second protein moiety of interest is a functionally inactive second protein moiety of interest, wherein the second genetically modified avian having a genome comprising the second agent retains fertility and viability while producing a primordial germ cell (PGC) having a genome comprising the second agent without impairing viability of the PGC; (j) integrating into a second targeted gene of interest (GOI) on a chromosome of interest in the second PGC a fourth agent, the fourth agent comprising a fourth exogenous polynucleotide, the fourth exogenous polynucleotide comprising a functionally active second GOI sequence or a functionally active fragment thereof operatively linked to one or more target sites specific to the genomic modifier, the second GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active second gene of interest (GOI), the modified trait inducing sterility or inhibiting fertility in the functionally modified and genetically modified progeny avian embryo or progeny avian without impairing viability; (k) producing second pure PGC colonies comprising the second agent and the fourth agent; (l) transplanting a second pure PGC colony into a male chick embryo to produce a second chimera male chick embryo and transplanting a second pure PGC colony into a female chick embryo to produce a second chimera female chick embryo; (m) producing a population of second chimera founder adult avians by hatching and rearing the second chimera founder chicks to sexual maturity, by mating chimera founder chicks at adulthood to produce offspring, or both; (n) screening the second population of chimera founder adult avians to verify homozygosity for the second agent; (o) selecting a male homozygous for the first agent and homozygous for the third agent from the first population of adult avians; (p) selecting a female homozygous for the second agent and homozygous for the fourth agent from the second population of adult avians; and (q) breeding the male adult avian from the first population with the female adult avian from the second population to produce a population of one or more sterile genetically modified progeny avian embryos, the first agent and the second agent, when co-expressed, comprise a functionally active protein of interest or fragment thereof comprising a genomic modifier, the genomic modifier targeting the one or more target sites in the first GOI and the second GOI in the one or more genetically modified progeny avian embryos, inducing sterility or inhibiting fertility in the one or more genetically modified progeny avian embryos without impairing viability.

In some embodiments, (a) the functionally active first GOI sequence or functionally active fragment thereof and the functionally active second GOI sequence or functionally active fragment thereof, or (b) the third agent and the fourth agent are identical.

In some embodiments, (a) the functionally active first GOI sequence or functionally active fragment thereof is distinct from the functionally active second GOI sequence or functionally active fragment thereof, or (b) the third agent is distinct from the fourth agent.

In some embodiments, the first gene of interest (GOI), the second GOI, or both have (a) an isolated function specific to a PGC, or (b) a function specific to gametogenesis, meiosis, gamete maturation, gamete function, or gamete fertilization, in the genetically modified progeny avian embryo or progeny avian, wherein deletion, disruption, or functional modification of the gene reduces or inhibits survival, maturation, or differentiation of a PGC or the specific gametogenesis, gamete maturation, or gamete functional modification reduces or inhibits gametogenesis, meiosis, gamete function, or gamete fertilization in an avian.

In some embodiments, the first gene of interest (GOI) sequence or fragment thereof, the second GOI sequence or fragment thereof, or both comprise a deleted in azoospermia-like (DAZL) gene, a deleted in azoospermia 1 (DAZ1) gene, a zona pellucida binding protein 1/2 (ZPBP1/2) gene, a cyclin-dependent kinases regulatory subunit 2 (CKS2; CDC28 Protein Kinase Regulatory Subunit 2) gene, a spermatogenesis associated 16 (SPATA16) gene, a DEAD-box helicase 4 (DDX4) gene, a serine/threonine-protein phosphatase PP1-gamma catalytic subunit (PPP1CC) gene, an Izumo sperm-egg fusion 1 (IZUMO1) gene, a synaptonemal complex central element protein 1 (SYCE1) gene, a YTH domain-containing 2 (YTHDC2) gene, a Meiosis Specific With Coiled-Coil Domain (MEIOC) gene, a septin-4 (SEPT4) gene, a stromal antigen 3 (STAG3) gene, a Nanos C2HC-type zinc finger 3 (NANOS3) gene, or a combination of any of these.

In some embodiments, the genomic modifier comprises a site-specific recombinase enzyme or functionally active fragment thereof.

In some embodiments, the first protein moiety of interest is a functionally inactive first protein moiety of interest and the second protein moiety of interest is a functionally inactive second protein moiety of interest, wherein the functionally inactive first protein moiety of interest and the functionally inactive second protein moiety of interest, dimerized or bound covalently in the genetically modified progeny avian embryo or progeny avian, comprise at least one functionally active protein of interest or fragment thereof, the functionally active protein of interest or fragment thereof comprising a toxin inducing cell death of a PGC, either without impairing viability of somatic cells or limited to benign somatic cell loss or both, in the functionally modified and genetically modified progeny avian embryo or progeny avian, and inducing sterility or inhibiting fertility in the functionally modified and genetically modified progeny avian embryo or progeny avian without impairing viability.

In some embodiments, the first protein moiety of interest is a functionally inactive first protein moiety of interest and the second protein moiety of interest is a functionally inactive second protein moiety of interest, wherein the functionally inactive first protein moiety of interest and the functionally inactive second protein moiety of interest, when co-expressed in the genetically modified progeny avian embryo or progeny avian, comprise at least one functionally active protein of interest or fragment thereof, the co-expression of which inducing cell death of a PGC without impairing viability of somatic cells in the functionally modified and genetically modified progeny avian embryo or progeny avian and inducing sterility or inhibiting fertility in the functionally modified and genetically modified avian without impairing viability.

In some embodiments, the method further comprises: (a) obtaining a first primordial germ cell (PGC) from an avian; (b) integrating into a chromosome of interest in the first PGC a first agent, the first agent comprising a first exogenous polynucleotide, the first exogenous polynucleotide comprising: (i) a first promoter; and (ii) a first element of interest operably linked to the promoter, the first element of interest encoding a first protein moiety of interest, the first protein moiety of interest comprising a functionally active first genomic modifier, wherein the first genetically modified avian having a genome comprising the first agent retains fertility and viability while producing a primordial germ cell (PGC) having a genome comprising the first agent without impairing viability of the PGC; (c) integrating into a first targeted gene of interest (GOI) on a chromosome of interest in the first PGC a third agent, the third agent comprising a third exogenous polynucleotide, the third exogenous polynucleotide comprising a functionally active first GOI sequence or a functionally active fragment thereof operatively linked to an one or more target sites specific to a second genomic modifier, the second genomic modifier not recognizing the target sites of the first genomic modifier, the first GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active first gene of interest (GOI), the modified trait inducing sterility or inhibiting fertility in the functionally modified and genetically modified progeny avian embryo or progeny avian without impairing viability; (d) producing first pure PGC colonies comprising the first agent and the third agent; (e) transplanting a first pure PGC colony into a male chick embryo to produce a first chimera male chick embryo and transplanting a first pure PGC colony into a female chick embryo to produce a first chimera female chick embryo; (f) producing a population of first chimera founder adult avians by hatching and rearing the first chimera founder chicks to sexual maturity, by mating first chimera founder chicks at adulthood to produce first chimera offspring, or both; (g) screening the first population of chimera founder adult avians to verify homozygosity for the first agent and the third agent; (h) obtaining a second primordial germ cell (PGC) from an avian; (i) integrating into a chromosome of interest in the second PGC a second agent, the second agent comprising a second exogenous polynucleotide, the second exogenous polynucleotide comprising: (i) a second promoter; and (ii) a second element of interest operably linked to the promoter, the second element of interest encoding a second protein moiety of interest, the second protein moiety of interest comprising a functionally active second genomic modifier, wherein the second genetically modified avian having a genome comprising the second agent retains fertility and viability while producing a primordial germ cell (PGC) having a genome comprising the second agent without impairing viability of the PGC; (j) integrating into a second targeted gene of interest (GOI) on a chromosome of interest in the second PGC a fourth agent, the fourth agent comprising a fourth exogenous polynucleotide, the fourth exogenous polynucleotide comprising a functionally active second GOI sequence or a functionally active fragment thereof operatively linked to an one or more target sites specific to the first genomic modifier, the first genomic modifier not recognizing the target sites of the second genomic modifier, the second GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active second gene of interest (GOI), the modified trait inducing sterility or inhibiting fertility in the functionally modified and genetically modified progeny avian embryo or progeny avian without impairing viability; (k) producing second pure PGC colonies comprising the second agent and the fourth agent; (l) transplanting a second pure PGC colony into a male chick embryo to produce a second chimera male chick embryo and transplanting a second pure PGC colony into a female chick embryo to produce a second chimera female chick embryo; (m) producing a population of second chimera founder adult avians by hatching and rearing the second chimera founder chicks to sexual maturity, by mating chimera founder chicks at adulthood to produce offspring, or both; (n) screening the second population of chimera founder adult avians to verify homozygosity for the second agent; (o) selecting a male homozygous for the first agent and homozygous for the third agent from the first population of adult avians; (p) selecting a female homozygous for the second agent and homozygous for the fourth agent from the second population of adult avians; and (q) breeding the male adult avian from the first population with the female adult avian from the second population to produce a population of one or more sterile genetically modified progeny avian embryos, the first genomic modifier targeting the one or more target sites in the second GOI and the second genomic modifier targeting one or more target sites in the first GOI in the one or more genetically modified progeny avian embryos, inducing sterility or inhibiting fertility in the one or more genetically modified progeny avian embryos without impairing viability.

In some embodiments, the gene of interest (GOI) sequence or fragment thereof has (a) an isolated function specific to a PGC; or (b) a function specific to gametogenesis, meiosis, gamete maturation, gamete function, or gamete fertilization in the genetically modified avian, wherein deletion, disruption, or functional modification of the gene reduces or inhibits survival, maturation, or differentiation of a PGC or the specific gametogenesis, gamete maturation, or gamete functional modification reduces or inhibits gametogenesis, meiosis, gamete function, or gamete fertilization in an avian.

In some embodiments, the first gene of interest (GOI) sequence or fragment thereof, the second gene of interest (GOI) sequence or fragment thereof, or both independently comprise a deleted in azoospermia-like (DAZL) gene, a deleted in azoospermia 1 (DAZ1) gene, a zona pellucida binding protein 1/2 (ZPBP1/2) gene, a cyclin-dependent kinases regulatory subunit 2 (CKS2; CDC28 Protein Kinase Regulatory Subunit 2) gene, a spermatogenesis associated 16 (SPATA16) gene, a DEAD-box helicase 4 (DDX4) gene, a serine/threonine-protein phosphatase PP1-gamma catalytic subunit (PPP1CC) gene, an Izumo sperm-egg fusion 1 (IZUMO1) gene, a synaptonemal complex central element protein 1 (SYCE1) gene, a YTH domain-containing 2 (YTHDC2) gene, a Meiosis Specific With Coiled-Coil Domain (MEIOC) gene, a septin-4 (SEPT4) gene, a stromal antigen 3 (STAG3) gene, a Nanos C2HC-type zinc finger 3 (NANOS3) gene, or a combination of any of these.

In some embodiments, the first PGC and the second PGC are both derived from an avian of the Galliformes order, the Anseriformes order, the Otidiformes order, the Columbiformes order, or the Struthioniformes order. In some embodiments, when derived from the Galliformes order, the first PGC and the second PGC are both derived from an avian of the Numidia family or the Phasianidae family comprising the Gallus genus or the Meleagris genus, respectively.

In some embodiments, a modification of the GOI comprises modification of a GOI having an isolated function specific to a PGC or modification of a gene having a function specific to gametogenesis, gamete maturation, or gamete function. In some embodiments, a modification of the GOI eliminates a function specific to PGCs. In some embodiments, a modification of the GOI reduces a function specific to PGCs. In some embodiments, a modification of the GOI eliminates a function specific to gametogenesis, gamete maturation, or gamete function. In some embodiments, a modification of the GOI reduces a function specific to gametogenesis, gamete maturation, or gamete function.

In some embodiments, a modification of the GOI having an isolated function specific to a PGC reduces or inhibits survival, maturation, or differentiation of a PGC derived from the genetically modified progeny avian. In some embodiments, a modification of the GOI having a function specific to gametogenesis, gamete maturation, or gamete function reduces or inhibits gametogenesis, meiosis, gamete function, or gamete fertilization in the genetically modified progeny avian.

In some embodiments, a modification of the GOI comprises modification of a gene encoding a protein, said protein comprising a Zona Pellucida Binding Protein 1/2 (ZPBP1/2) protein, Cyclin-Dependent Kinases Regulatory Subunit 2 (CKS2) protein, Spermatogenesis Associated 16 (SPATA16) protein, DEAD-Box Helicase 4 (DDX4) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis Specific With Coiled-Coil Domain (MEIOC) protein, Septin-4 (SEPT4) protein, Stromal Antigen 3 (STAG3) protein, Nanos C2HC-Type Zinc Finger 3 (NANOS3) protein, Deleted In Azoospermia 1 (DAZ1) protein, and Deleted In Azoospermia-Like (DAZL) protein. In some embodiments, the modification of the GOI comprises modification of a gene encoding a Deleted In Azoospermia-Like (DAZL) protein. In some embodiments, the modification of the GOI comprises modification of a gene encoding a Deleted In Azoospermia-Like (DAZL) protein. In some embodiments, the modification of the GOI comprises modification of a gene encoding a DEAD-Box Helicase 4 (DDX4) protein.

A skilled artisan would appreciate that there may be redundancy mechanisms or shared activities between proteins. Therefore, in some embodiments, the genetic modification comprises modification of a combination of genes, i.e., two or more GOI. In some embodiments, the modification of the GOI comprises modification of a combination of at least 2 genes, said genes comprising those encoding a protein comprising a Zona Pellucida Binding Protein 1/2 (ZPBP1/2) protein, Cyclin-Dependent Kinases Regulatory Subunit 2 (CKS2) protein, Spermatogenesis Associated 16 (SPATA16) protein, DEAD-Box Helicase 4 (DDX4) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis Specific With Coiled-Coil Domain (MEIOC) protein, Septin-4 (SEPT4) protein, Stromal Antigen 3 (STAG3) protein, Nanos C2HC-Type Zinc Finger 3 (NANOS3) protein, Deleted In Azoospermia 1 (DAZ1) protein, and Deleted In Azoospermia-Like (DAZL) protein. In some embodiments, a combination comprises mutations in two genes. In some embodiments, a combination comprises mutations in more than two genes. In some embodiments, a combination comprises mutations in at least 2, 3, 4, or 5 genes. In some embodiments, a combination comprises a combination of mutations such that the function specific to gametogenesis, gamete maturation, or gamete function is eliminated. In some embodiments, a combination comprises a combination of mutations such that the function specific to gametogenesis, gamete maturation, or gamete function is reduced.

In some embodiments, the genetically modified avian PGC is disclosed herein, wherein the chromosome is an autosomal chromosome.

In some embodiments, the marker is a fluorescent protein, a luminescent protein, or a chromoprotein. In some embodiments, the marker is (a) a fluorescent protein comprising Green Fluorescent Protein (GFP), Enhanced Green Fluorescent Protein (EGF), Emerald, Superfolder GFP, Azami Green, mWasabi, Tag-Green Fluorescent Protein (TagGFP), Turbo-Green Fluorescent Protein (TurboGFP), mNeonGreen, mUKG, acGFP, ZsGreen, Cloverm Sapphire, T-Sapphire, Enhanced Blue Fluorescent Protein (EBFP), Enhanced Blue Fluorescent Protein 2 (EBFP2), Azurite, Tag-Enhanced Blue Fluorescent Protein (TagBFP), mTagBFP, mKalama1, Cyan Fluorescent Protein (CFP), mCFP, Enhanced Cyan Fluorescent Protein (ECFP), mECFP, Cerulean, SCFP3A, mTurquoise, mTurquoise2, CyPet, AmCyan1, Midori-Ishi Cyan, Tag-Cyan Fluorescent Protein (TagCFP), mTFP1 (Teal), Yellow Fluorescent Protein (YFP), Enhanced Yellow Fluorescent Protein (EYFP), Super Yellow Fluorescent Protein (SYFP), Topaz, Venus, Citrine, mCitrine, YPet, Tag-Yellow Fluorescent Protein (TagYFP), Turbo-Yellow Fluorescent Protein (TurboYFP), Phi-Yellow Fluorescent Protein (PhiYFP), ZsYellow1, mBanana, Kusabira Orange, Kusabira Orange2, mOrange, mOrange2, dTomato, dTomato-Tandem, Red Fluorescent Protein (RFP), Turbo-Red Fluorescent Protein (TurboRFP), TurboFP602, TurboFP635, Tag-Red Fluorescent Protein (RFP), TagRFP-T, DsRed, DsRed2, DsRed-Express (T1), DsRed-Monomer, mTangerine, mKeima-Red, mRuby, mRuby2, mApple, mStrawberry, AsRed2, mRFP1, J-Red, mCherry, mKate (TagFP635), mKate2, HcRed1, mRaspberry, dKeima-Tandem, HcRed-Tandem, mPlum, mNeptune, NirFP, Sinus, TagFRP657, AQ143, Kaede, KikGR1, PX-CFP2, mEos2, IrisFP, meOS3.2, PSmOrange, PAGFP, Dronpa, Allowphycocyanin, GFPuv, R-Phycoerythrin (RPE), Peridinin Chlorophyll (PerCP), P3, Katusha, B-Phycoerythrin (BPE), and mKO; or (b) a chromoprotein comprising ShadowR, Stichodactyla gigantea (sgBP), Heteractis crispa (hcCP), Anemonia sulcata (asCP), Cnidopus japonicus (cjBlue), or Goniopora tenuidens (gtCP). In some embodiments, the marker is mCherry.

In some embodiments, the marker is a green fluorescent protein (GFP) encoded by SEQ ID NO: 15 or SEQ ID NO: 55. In some embodiments, the GFP has a sequence at least 95% identical to or SEQ ID NO: 16 or SEQ ID NO: 77. In some embodiments, the GFP has a sequence comprising SEQ ID NO: 16 or SEQ ID NO: 77.

In some embodiments, the genetically modified avian PGC is derived from an avian of the Galliformes order, the Anseriformes order, the Otidiformes order, the Columbiformes order, or the Struthiomformes order. In some embodiments, the genetically modified avian PGC derived from an avian of the Galliformes order comprises the Phasianidae family or the Numididae family. In some embodiments, the genetically modified avian PGC is derived from an avian of the Phasianidae family comprising the Gallus genus or the Meleagris genus. In some embodiments, the genetically modified avian PGC is derived from an avian of the Anseriformes order comprising the Anatidae family, the Anseranatidae family, or the Anhimidae family. In some embodiments, the genetically modified avian PGC is derived from an avian of the Otidiformes order comprising the Otididae family. In some embodiments, the genetically modified avian PGC is derived from an avian of the Columbiformes order comprising the Columbidae family. In some embodiments, the genetically modified avian PGC is derived from an avian of the Struthioniformes order comprising the Struthionidae family.

In some embodiments, the modification or disruption of a gene comprises modification or disruption of a gene encoding a protein selected from the group consisting of Zona Pellucida Binding Protein 1/2 (ZPBP1/2) protein, Cyclin-Dependent Kinases Regulatory Subunit 2 (CKS2) protein, Spermatogenesis Associated 16 (SPATA16) protein, DEAD-Box Helicase 4 (DDX4) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis-Specific With Coiled-Coil Domain-Containing Protein (MEIOC) protein, Septin-4 (SEPT4) protein, Stromal Antigen 3 (STAG3) protein, Nanos C2HC-Type Zinc Finger 3 (NANOS3) protein, Deleted In Azoospermia 1 (DAZ1) protein, and Deleted In Azoospermia-Like (DAZL) protein. In some embodiments, the modification or disruption of a gene comprises modification or disruption of a gene encoding a Deleted In Azoospermia-Like (DAZL) protein.

In some embodiments, the modification or disruption of a gene comprises modification or disruption of at least 2 genes, said genes comprising those encoding a protein selected from the group consisting of Zona Pellucida Binding Protein 1/2 (ZPBP1/2) protein, Cyclin-Dependent Kinases Regulatory Subunit 2 (CKS2) protein, Spermatogenesis Associated 16 (SPATA16) protein, DEAD-Box Helicase 4 (DDX4) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis-Specific With Coiled-Coil Domain-Containing Protein (MEIOC) protein, Septin-4 (SEPT4) protein, Stromal Antigen 3 (STAG3) protein, Nanos C2HC-Type Zinc Finger 3 (NANOS3) protein, Deleted In Azoospermia 1 (DAZ1) protein, and Deleted In Azoospermia-Like (DAZL) protein. In some embodiments, the modification or disruption of a gene comprises modification or disruption of at least 2, 3, 4, or 5 genes.

In some embodiments, the chromosome of interest is an autosomal chromosome.

In some embodiments, the sterile phenotype induces sterility in a male progeny avian or a female progeny avian.

### Avians

“Aves,” “avians,” or “birds” are a class of warm-blooded vertebrates generally having feathers, forelimbs modified as wing, toothless beaked jaws, the laying of hard-shelled eggs, a high metabolic rate and body temperature, a four-chambered heart, and a lightweight skeleton often characterized by large air-filled cavities (pneumatic cavities) connecting with the respiratory system. In flying birds and in most reluctant flyers, the sternum is keeled for the attachment of flight muscles. The avian immune system includes the bursa of Fabricius, which is unique to avians, and which is the site of hematopoiesis and B cell development.

Avian reproduction takes place via cloacal kiss, and many female avians have sperm storage mechanisms enabling sperm from one or more males to remain viable within the female for a period of time following copulation. Following fertilization, the shell is applied to the egg, and the egg is then laid and incubated externally by one or both parents, by a non-parent male partner, or by a non-parent (even of another species, as in brood parasitism). The degree of monogamy depends on the species.

As used herein, the terms “ave,” “avian,” or “bird” refer to any avian species, including, but are not limited to, chicken, turkey, duck, goose, quail, pheasant, guinea fowl, pigeon, and ostrich. In certain embodiments, the bird is a species of fowl. In certain embodiments, the bird is a species of poultry. In certain embodiments, the bird is a domestic bird. In certain embodiments, the bird is Gallus. In certain embodiments, the bird is a domestic Gallus. In certain embodiments, the bird is Gallus gallus domesticus.

In certain embodiments, the bird is a female. In certain embodiments, the bird is a male. In certain embodiments, the bird is a broiler. In certain embodiments, the bird is a hen. In certain embodiments, the bird is layer hen. In certain embodiments, the bird is a domestic chicken. In certain embodiments, the bird is Gallus gallus domesticus layer hen.

“Domestication” by humans is a sustained multi-generational relationship in which humans significantly influence the reproduction and care of another species to secure a more predictable supply of, e.g., resources from the other species. Domestication is characterized by conscious selective breeding in which humans directly select for desirable traits, in contrast to unconscious selection in which traits evolve as a by-product of natural selection or from selection on other traits. As a result, there are genetic differences between wild and domesticated populations, even of the same species.

“Fowl” are primitive avians belonging to one of two orders—Galliformes (gamefowl or landfowl) and Anseriformes (waterfowl), which together form the fowl clade Galloanserae. Galloanserae are noted for being very prolific, for having a high rate of polygamy compared with other avians, for hybridization and increased ability to interbreed (even where not closely related), and for precocious young.

“Poultry” are domesticated birds or birds that are captive-raised for meat, eggs, feathers, and the like. Most poultry belong to the Galloanserae clade, but there are a few exceptions (e.g., ostriches). Popular examples of poultry include, but are not limited to, chicken, turkey, duck, goose, quail, pheasant, guinea fowl, pigeon, and ostrich.

Galliformes (gamefowl or landfowl) is an avian order characterized by a heavy body and ground-feeding and includes, but is not limited to, chicken, turkey, grouse, quail (both New World and Old World), ptarmigan, partridge, pheasant, francolin, junglefowl, and the Cracidae. The order comprises five families: Phasianidae (e.g., chicken, quail, partridge, pheasant, turkey, peafowl, grouse); Odontophoridae (e.g., New World quail); Numididae (e.g., guineafowl); Cracidae (e.g., chachalacas, curassows); and Megapodiidae (e.g., malleefowl, brush-turkey). The Phasianidae family includes, but is not limited to, the Gallus genus (e.g., Gallus gallus [wild or domestic chicken]) and the Meleagris genus (e.g., Meleagris gallopavo [wild or domestic turkey] or Meleagris ocellate [ocellated turkey]).

The Numididae family of guineafowl includes the Agelastes, Numida, Guttera, and Acryllium genera.

Anseriformes (waterfowl) is an avian order characterized by aquatic lifestyle and swimming skills and includes, but is not limited to duck, goose, swan, magpie-goose, and screamer. The order comprises three families: Anhimidae (e.g., screamer); Anseranatidae (e.g., magpie-goose); and Anatidae (over 146 species in 43 genera, including e.g., duck, goose, swan). Unlike most birds, all except the screamers have phalli. The Anatidae family includes, but is not limited to, numerous species of ducks; three genera of gees (e.g., Answer, Branta, and Chen); and the Cygnus genus (e.g., Cygnus [swan]). The Anhimidae family includes, but is not limited to, the Anhima and Chauna genera.

Otidiformes (e.g., Otididae [bustards]) is an avian order characterized by large, terrestrial birds living mainly in dry grassland areas and on the steppes of the Old World.

They make up the family Otididae (formerly, Otidae or Gryzajidae). Bustards are omnivorous and opportunistic, eating leaves, buds, seeds, fruit, small vertebrates, and invertebrates. Twenty-six species currently recognized. Bustards include, but are not limited to, floricans and korhaans. The order comprises several families, including: Lissotis, Ardeotis, Neotis, Tetrax, Otis, Chlamydotis, Houbaropsis, Sypheotides, Lophotis, Eupodotis, and Afrotis. Chlamydotis (Houbara) is a genus of large birds in the bustard family.

Other avians used for meat, eggs, feathers, and the like include, but are not limited to members of the Columbiformes order (e.g., Columba livia [pigeon]), the Struthioniformes order (e.g., Struthionidae camelus [ostrich]).

As used herein, the term “egg” refers to an avian egg that contains a viable or a live embryonic bird. In one embodiment, the term “egg” is intended to refer to a fertilized avian egg. In one embodiment, an egg is an egg containing an avian embryo that is capable of undergoing normal embryogenesis.

### Primordial Germ Cells (PGCs)

An “anlage” or “primordium” is an organ or tissue in its earliest recognizable stage of development or the simplest set of cells capable of triggering growth of the would-be organ or tissue and the initial foundation from which an organ or tissue is able to grow.

A skilled artisan would appreciate that the terms “pluripotent cells” and “totipotent cells” of the primordium encompass “primordial cells.”

A “primordial germ cell” (PGC), also known as a “precursor germ cell” or a “gonocyte,” is a pluripotent diploid germ cell prior to its maturation as a gamete and is one of a small group of cells set aside during embryonic gastrulation to eventually form an oocyte or a spermatozoon. In birds and mammals, germ cells are formed during development in response to signals controlled by zygotic genes. In birds and reptiles, the PGC comes from the epiblast and migrates to the germinal crescent (an anterior extraembryonic structure). The PGC then enters into a blood vessel and uses the circulatory system for transport to a gonadal ridge (genital ridge), where it exits the blood vessel and enters the gonad. During its migration, it will divide repeatedly. Upon reaching the gonad, it becomes a “germ cell.” A germ cell gives rise to one or more “gametes” (ovum or sperm) and is the only type of cell capable of both meiosis and mitosis.

“Gametogenesis” refers to, e.g., the development of a diploid germ cell into a haploid ovum (oocyte, egg) or sperm (“oogenesis” or “spermatogenesis,” respectively). The process, especially oogenesis, may be arrested for a period of time prior to full “gamete maturation.” Once matured, a haploid male gamete and a haploid female gamete have the ability to unite to form a new, diploid cell (“zygote”).

PGCs, germ cells and gametes each have many unique properties. Two examples of features that make PGCs unique are their ability to maintain totipotency and differentiate into functional gametes, and their ability to migrate along the route from their initial location of formation to the gonads. With respect to migration, some genes involved in this process may also be involved in other functions in somatic cells. With respect to totipotency and gametogenesis, there are several genes active in these processes, but only a few of them are known to be expressed solely in PGCs without having a redundant alternative gene (e.g., deleted in AZoospermia-Like [DAZL]).

Modification of a gene having an isolated function specific to a PGC includes, but is not limited to, reduction or inhibition of PGC survival, maturation, or differentiation or a combination thereof of a PGC derived from a genetically modified avian.

Modification of a gene having a function specific to gametogenesis, gamete maturation, or gamete function includes, but is not limited to, reduction or inhibition or a combination thereof, of gametogenesis, meiosis, gamete function, or gamete fertilization in the genetically modified avian.

For example, at sexual maturity of the organism, the primary oocyte secretes proteins to form a coat called zona pellucida and also produces cortical granules containing enzymes and proteins needed for fertilization. In addition, large non-mammalian oocytes accumulate egg yolk, glycogen, lipids, ribosomes, and the mRNA needed for protein synthesis during embryonic growth. In another example, the sperm cell undergoes nuclear condensation, ejection of the cytoplasm, and formation of the acrosome and flagellum.

The injected donor PGCs may be genetically modified in a certain embodiment, e.g., to generate GM avians. In another embodiment, the donor injected PGCs may be unmodified, for example, for the use of cryopreservation and re-derivation of avian strains or to generate surrogate avians of one type to lay eggs of a different type (e.g., to generate layer type surrogate chickens to lay broiler-type eggs).

In some embodiments, a primordial germ cell is genetically modified and implanted into a sterile avian embryo, which upon maturity, produces genetically modified avian progeny.

In some embodiments, a primordial germ cell of a breed of interest is not genetically modified, but is implanted into a sterile avian embryo, which upon maturity, produces avian progeny of the breed of interest that are not genetically modified.

### Binary Elements

In some aspects, the binary elements—semi-inactivating elements (SIEs), each comprise an inactive unit, such that when expressed together, the combination results in an inactivation of FRGs. In other aspects, each of the SIEs consist of exogenous inactive element, and following breeding, when are co-expressed, they induce sterility. The induction of sterility can be done by interfering with FRG activity, e.g., by preventing PGCs to mature to functioning gametes, and/or by inducing PGCs death. Any combination of the above options can also be used. Examples of these options are described in detail below.

Provided herein is a solution based on a general formula in which two separate breeds are created, each breed having one semi-inactivating element (SIE) that, by itself, has no activity. By crossing between the two breeds, each of the resulting embryos will receive two SIEs, one from each parent. The two SIEs lead to a deleterious effect on the activity of fertility-required genes (FRGs). Having only inactivated FRGs, the embryo will develop into a sterile organism. Alternatively, two SIEs will activate the sterility-inducing factor (SIF) which will affect the survival of the PGCs or will interrupt their function. This is a binary-based activation process in which two separate breeds are created, each having one SIE, and by crossing between the two breeds, the resulting embryos receive a copy of each element, which, dimerized or covalently bound, become active and lead to deleterious mutation in fertility-required genes (FRG) or to active the SIF, which will affect the survival of the PGCs or will interrupt their function.

FIGS. 2A-2B depict an exemplary general method of making and using the binary elements for providing a population of sterile avians, while maintaining founder populations. However, this exemplary general method contemplates various types of binary elements and various methods of making and/or using them. In one exemplary method, the binary elements comprise two inactive units, which when expressed together, result in genomic modification, which render the FRG inactive. In another exemplary method, the two inactive units consist of two inactive parts of a toxin which are expressed specifically in PGCs, and when co-expressed, they induce the death of the PGCs, but not of somatic cells, or they induce the death of the PGCs while incurring only benign loss of somatic cells, or a combination of both. In still another exemplary alternative, the two inactive elements consist of two proteins which when co-expressed in PGCs, induce their death, or their inability to mature into functioning gametes. In still another exemplary method.

In some embodiments, the first protein moiety of interest is a functionally inactive first protein moiety of interest and the second protein moiety of interest is a functionally inactive second protein moiety of interest, wherein: the functionally inactive first protein moiety of interest and the functionally inactive second protein moiety of interest, dimerized or bound covalently, comprise at least one functionally active protein of interest or fragment thereof, wherein: the functionally active protein of interest or fragment thereof comprises a genomic modifier, the genomic modifier targeting a gene of interest (GOI) or fragment thereof on a chromosome, the GOI modified to introduce one or more target sites specific to the genomic modifier, and the GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active gene of interest (GOI), the modified trait inducing sterility or inhibiting fertility in the functionally modified and genetically modified progeny avian embryo or progeny avian without impairing viability; the functionally active protein of interest or fragment thereof comprises a toxin inducing cell death of a PGC, either without impairing viability of somatic cells or limited to benign somatic cell loss or both, in the functionally modified and genetically modified progeny avian embryo or progeny avian, and inducing sterility or inhibiting fertility in the functionally modified and genetically modified progeny avian embryo or progeny avian without impairing viability; or a combination thereof, or the functionally inactive first protein moiety of interest and the functionally inactive second protein moiety of interest, when co-expressed, comprise at least one functionally active protein of interest or fragment thereof, the co-expression of which inducing cell death of a PGC without impairing viability of somatic cells in the functionally modified and genetically modified progeny avian embryo or progeny avian and inducing sterility or inhibiting fertility in the functionally modified and genetically modified avian without impairing viability; or a combination of any of the above.

In some embodiments, the toxin comprises a Pseudomonas exotoxin, a diphtheria toxin, a ricin, or a recombinant toxin (e.g., those used to fight cancer cells), or a combination of any of these. In some embodiments, the toxin does not impair somatic cell viability. In some embodiments, the toxin has a limited effect on somatic cells, namely, benign somatic cell loss.

Apoptosis, or programmed cell death (PCD), is a mechanism in embryonic development that occurs naturally in organisms. As cells rapidly proliferate during development, many of them undergo apoptosis. For example, if a PGC expresses a toxin and dies, the toxin secreted from the dead cell potentially may locally affect neighboring somatic cells, but in the context of the very early embryo, the loss of these cells is often benign, as greater than 70% of somatic cells naturally die within the first 24 hours after fertilization, and the remaining somatic cells are often pluripotent or at least not fully differentiated, thereby enabling them to replace any neighboring somatic cells affected by the toxin. “Benign somatic cell loss” comprises somatic cell loss that is not harmful in general effect to the embryo overall, e.g., of a mild type or character that does not threaten the health or life of the embryo or one having no significant long-term effect on the embryo or on the resulting avian.

In some embodiments, the first protein moiety of interest comprising a functionally active first genomic modifier and the second protein moiety of interest comprising a functionally active second genomic modifier, wherein: the functionally active second genomic modifier targeting a first gene of interest (GOI) or fragment thereof on a chromosome, the first GOI or fragment thereof modified to introduce one or more target sites specific to the second genomic modifier, and the first GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active first GOI, the modified trait inducing sterility or inhibiting fertility in the functionally modified and genetically modified progeny avian embryo or progeny avian without impairing viability; and the functionally active first genomic modifier targeting a second gene of interest (GOI) or fragment thereof on a chromosome, the second GOI or fragment thereof modified to introduce one or more target sites specific to the second genomic modifier, and the second GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active second gene of interest, the modified trait inducing sterility or inhibiting fertility in the functionally modified and genetically modified progeny avian embryo or progeny avian without impairing viability.

Ordinarily, protein splicing is an intramolecular reaction of a particular protein in which an internal protein segment (called an intein) is removed from a precursor protein with a ligation of C-terminal and N-terminal external proteins (called exteins) on both sides. Normally, splicing is associated only with pre-mRNA splicing. This precursor protein contains three segments—an N-extein followed by the intein followed by a C-extein. After splicing has taken place, the resulting protein contains the N-extein linked to the C-extein; this splicing product is also termed an extein. Inteins are enzymes. Split into N-terminal and C-terminal fragments, they are able to form a functionally active enzyme that fuses two distinct exteins via a splicing reaction. Essentially, each separate part of the intein has no functional activity. When expressed together, however, they dimerize (see, e.g., Shah et al. (2014) Chem. Sci. 5(1):446-461.

In some embodiments, the first exogenous polynucleotide further encodes a first intein moiety operably linked to the first promoter and to the first element of interest encoding the functionally inactive first protein moiety of interest; and the second exogenous polynucleotide further encodes a second intein moiety distinct from the first intein moiety and operably linked to the second promoter and to the second element of interest encoding the functionally inactive second protein moiety of interest, wherein the first intein moiety and the second intein moiety, dimerized or covalently bound, comprise a functionally active intein or fragment thereof, the functionally active intein or fragment thereof splicing the functionally inactive first protein moiety of interest and the functionally inactive second protein moiety of interest to produce the functionally active protein of interest or fragment thereof. In some embodiments, the first intein moiety is a functionally inactive intein moiety and wherein the second intein moiety is a functionally inactive intein moiety.

In a non-limiting example, the intein reconstitution system is a technology for irreversible conjugation of recombinant proteins. The N-terminal intein protein moiety and the C-terminal intein protein moiety together form a functionally active intein enzyme. The DNA sequence encoding the desired N-terminal intein protein moiety is fused with the DNA sequence encoding a protein of interest in one breed, while the DNA sequence encoding the desired C-terminal intein moiety is fused with the DNA sequence encoding a protein of interest in another breed. These fusion proteins, when coexpressed in the progeny genetically modified PGC or avian, ligate the functionally inactive first protein moiety of interest and the functionally inactive second protein moiety of interest, via intein-mediated protein splicing, to form the functionally active protein of interest or fragment thereof.

In some embodiments, the first exogenous polynucleotide further encodes a first intein moiety operably linked to the first promoter and to the first element of interest encoding the functionally inactive first protein moiety of interest; and the second exogenous polynucleotide further encodes a second intein moiety distinct from the first intein moiety and operably linked to the second promoter and to the second element of interest encoding the functionally inactive second protein moiety of interest.

In certain embodiments, the first intein moiety is encoded by a nucleic acid sequence 50% to 100% identical in sequence to SEQ ID NO: 7 or SEQ ID NO: 50. In certain embodiments, the first intein moiety is encoded by a nucleic acid sequence 80% to 100% identical in sequence to SEQ ID NO: 7 or SEQ ID NO: 50. In certain embodiments, the first intein moiety is encoded by a nucleic acid sequence 85% to 100% identical in sequence to SEQ ID NO: 7 or SEQ ID NO: 50. In certain embodiments, the first intein moiety is encoded by a nucleic acid sequence 90% to 100% identical in sequence to SEQ ID NO: 7 or SEQ ID NO: 50. In certain embodiments, the first intein moiety is encoded by a nucleic acid sequence 95% to 100% identical in sequence to SEQ ID NO: 7 or SEQ ID NO: 50. In certain embodiments, the first intein moiety is encoded by a nucleic acid sequence 98% to 100% identical in sequence SEQ ID NO: 7 or SEQ ID NO: 50. In certain embodiments, the first intein moiety is encoded by a nucleic acid sequence 99% to 100% identical in sequence to SEQ ID NO: 7 or SEQ ID NO: 50. In certain embodiments, the first intein moiety is encoded by a nucleic acid sequence 100% identical in sequence to SEQ ID NO: 7 or SEQ ID NO: 50.

In certain embodiments, the second intein moiety is encoded by a nucleic acid sequence 50% to 100% identical in sequence to SEQ ID NO: 9 or SEQ ID NO: 52. In certain embodiments, the second intein moiety is encoded by a nucleic acid sequence 80% to 100% identical in sequence to SEQ ID NO: 9 or SEQ ID NO: 52. In certain embodiments, the second intein moiety is encoded by a nucleic acid sequence 85% to 100% identical in sequence to SEQ ID NO: 9 or SEQ ID NO: 52. In certain embodiments, the second intein is encoded by a nucleic acid sequence 90% to 100% identical in sequence to SEQ ID NO: 9 or SEQ ID NO: 52. In certain embodiments, second intein is encoded by a nucleic acid sequence 95% to 100% identical in sequence to SEQ ID NO: 9 or SEQ ID NO: 52. In certain embodiments, the second intein is encoded by a nucleic acid sequence 98% to 100% identical in sequence SEQ ID NO: 9 or SEQ ID NO: 52. In certain embodiments, the second intein is encoded by a nucleic acid sequence 99% to 100% identical in sequence to SEQ ID NO: 9 or SEQ ID NO: 52. In certain embodiments, the second intein is encoded by a nucleic acid sequence 100% identical in sequence to SEQ ID NO: 9 or SEQ ID NO: 52.

In certain embodiments, the first intein is 50% to 100% identical in sequence to SEQ ID NO: 8 or SEQ ID NO: 72. In certain embodiments, the first intein is 80% to 100% identical in sequence to SEQ ID NO: 8 or SEQ ID NO: 72. In certain embodiments, the first intein is 85% to 100% identical in sequence to SEQ ID NO: 8 or SEQ ID NO: 72. In certain embodiments, the first intein is 90% to 100% identical in sequence to SEQ ID NO: 8 or SEQ ID NO: 72. In certain embodiments, the first intein is 95% to 100% identical in sequence to SEQ ID NO: 8 or SEQ ID NO: 72. In certain embodiments, the first intein is 98% to 100% identical in sequence SEQ ID NO: 8 or SEQ ID NO: 72. In certain embodiments, the first intein is 99% to 100% identical in sequence to SEQ ID NO: 8 or SEQ ID NO: 72. In certain embodiments, the first intein is 100% identical in sequence to SEQ ID NO: 8 or SEQ ID NO: 72.

In certain embodiments, the second intein is 50% to 100% identical in sequence to SEQ ID NO: 10 or SEQ ID NO: 74. In certain embodiments, the second intein is 80% to 100% identical in sequence to SEQ ID NO: 10 or SEQ ID NO: 74. In certain embodiments, the second intein is 85% to 100% identical in sequence to SEQ ID NO: 10 or SEQ ID NO: 74. In certain embodiments, the second intein is 90% to 100% identical in sequence to SEQ ID NO: 10 or SEQ ID NO: 74. In certain embodiments, the second intein is 95% to 100% identical in sequence to SEQ ID NO: 10 or SEQ ID NO: 74. In certain embodiments, the second intein is 98% to 100% identical in sequence SEQ ID NO: 10 or SEQ ID NO: 74. In certain embodiments, the second intein is 99% to 100% identical in sequence to SEQ ID NO: 10 or SEQ ID NO: 74. In certain embodiments, the second intein is 100% identical in sequence to SEQ ID NO: 10 or SEQ ID NO: 74.

In some embodiments, the first intein has a sequence at least 95% identical to SEQ ID NO: 8 or SEQ ID NO: 72 and is encoded by a nucleic acid sequence at least 95% identical to SEQ ID NO: 7 or SEQ ID NO: 50; the second intein has a sequence at least 95% identical to SEQ ID NO: 10 or SEQ ID NO: 74 and is encoded by a nucleic acid sequence at least 95% identical to SEQ ID NO: 9 or SEQ ID NO: 52; or both. In some embodiments, the first intein is encoded by a nucleic acid sequence corresponding to SEQ ID NO: 7 or SEQ ID NO: 50; the second intein is encoded by a nucleic acid sequence corresponding to SEQ ID NO: 9 or SEQ ID NO: 52; or both.

In some embodiments, the first intein has a sequence at least 95% identical to SEQ ID NO: 8 or SEQ ID NO: 72 or SEQ ID NO: 72; the second intein has a sequence at least 95% identical to SEQ ID NO: 10 or SEQ ID NO: 74; or both. In some embodiments, the first intein has a sequence corresponding to SEQ ID NO: 8 or SEQ ID NO: 72 or SEQ ID NO: 72; the second intein has a sequence corresponding to SEQ ID NO: 10 or SEQ ID NO: 74; or both.

It will be appreciated that more than one nucleic acid coding strand is encompassed. The degenerate genetic code, also known as the redundancy of the genetic code, refers to the fact that multiple codons, or sets of three nucleotides, can code for the same amino acid during protein synthesis, namely, that different codons can encode for the same amino acid, but no codon can encode for more than one amino acid. Therefore, in some instances, multiple codons code for the same amino acid, and some amino acids are coded for by as many as six different codons, and a nucleic acid substitution in an underlying nucleic acid coding sequence will not necessarily result in a change in the amino acid encoded by the codon corresponding to the nucleic acid substitution.

It will also be appreciated that not all amino acid replacements have the same effect on function or structure of protein. A conservative amino acid replacement (conservative substitution, conservative mutation) comprises a replacement in a protein that changes the amino acid to a different amino acid with similar biochemical properties (e.g., charge, hydrophobicity, and/or size). Examples of conservative amino acid replacement include, but are not limited to, changes within amino acid groups that are aliphatic (Gly, Ala, Val, Leu, and/or Ile); hydroxyl or sulfur/selenium-containing (Ser, Cys, seleno-Cys, Thr, and/or Met); cyclic (Pro); aromatic (Phe, Tyr, Trp); basic (His, Lys, Arg); and acidic and their amides (Asp, Glu, Asn, Gln).

It will be appreciated that the term “modification” can encompass an amino acid modification such as an amino acid substitution, insertion, and/or deletion in a polypeptide sequence. The term “homolog” as used herein refers to a polypeptide having a sequence homology of a certain amount, namely of at least 70%, e.g., at least 80%, 90%, 95%, 96%, 97%, 98%, 99% of the amino acid sequence it is referred to. Homology refers to the magnitude of identity between two sequences. Homolog sequences have the same or similar characteristics, in particular, have the same or similar property of the sequence as identified.

The term “variant” as used herein refers to a polypeptide wherein the amino acid sequence exhibits substantially 70, 80, 95, or 99% homology with the amino acid sequence as set forth in the sequence listing. It should be appreciated that the variant may result from a modification of the native amino acid sequences, or by modifications including insertion, substitution or deletion of one or more amino acids. The term “isoform” as used herein refers to variants of a polypeptide that are encoded by the same gene, but that differ in their isoelectric point (pI) or molecular weight (MW), or both. Such isoforms can differ in their amino acid composition (e.g., as a result of alternative splicing or limited proteolysis) and in addition, or in the alternative, may arise from differential post-translational modification (e.g., glycosylation, acylation, phosphorylation deamidation, or sulphation). As used herein, the term “isoform” also refers to a protein that exists in only a single form, i.e., it is not expressed as several variants. The term “fragment” as used herein refers to any portion of the full-length amino acid sequence of protein of a polypeptide disclosed herein associated with the sterile avian embryos, products and uses thereof, which has less amino acids than the full-length amino acid sequence of a polypeptide disclosed herein associated with the sterile avian embryos, products and uses thereof. The fragment may or may not possess a functional activity of such polypeptides.

In another embodiment, the amino acid sequences of nIntein are at least 80% (e.g., at least 85%, 90%, 95%, 96%, 97%, 98%, or 99%) identical to the amino acid sequences set forth above and below. In another embodiment, the amino acid sequences of cIntein are at least 80% (e.g., at least 85%, 90%, 95%, 96%, 97%, 98%, or 99%) identical to the amino acid sequences set forth above and below. In another embodiment, the amino acid sequences of Intein are at least 80% (e.g., at least 85%, 90%, 95%, 96%, 97%, 98%, or 99%) identical to the amino acid sequences set forth above below. One skilled in the art would appreciate that percent sequence identity may be determined using any of a number of publicly available software application, for example but not limited to BlastP software of the National Center of Biotechnology Information (NCBI) using default parameters.

A skilled artisan would appreciate that percent identity (% identity) provides a number that describes how similar the query sequence is to the target sequence (i.e., how many amino acids in each sequence are identical). The higher the percent identity is, the more significant the match.

When used in relation to polypeptide (or protein) sequences, the term “identity” refers to the degree of identity between two or more polypeptide (or protein) sequences or fragments thereof. Typically, the degree of similarity between two or more polypeptide (or protein) sequences refers to the degree of similarity of the composition, order, or arrangement of two or more amino acids of the two or more polypeptides (or proteins).

A skilled artisan would appreciate that the term “homology”, and grammatical forms thereof, encompasses the degree of similarity between two or more structures. The term “homologous sequences” refers to regions in macromolecules that have a similar order of monomers. When used in relation to nucleic acid sequences, the term “homology” refers to the degree of similarity between two or more nucleic acid sequences (e.g., genes) or fragments thereof. Typically, the degree of similarity between two or more nucleic acid sequences refers to the degree of similarity of the composition, order, or arrangement of two or more nucleotide bases (or other genotypic feature) of the two or more nucleic acid sequences. The term “homologous nucleic acids” generally refers to nucleic acids comprising nucleotide sequences having a degree of similarity in nucleotide base composition, arrangement, or order. The two or more nucleic acids may be of the same or different species or group. The term “percent homology” when used in relation to nucleic acid sequences, refers generally to a percent degree of similarity between the nucleotide sequences of two or more nucleic acids.

When used in relation to polypeptide (or protein) sequences, the term “homology” refers to the degree of similarity between two or more polypeptide (or protein) sequences (e.g., genes) or fragments thereof. Typically, the degree of similarity between two or more polypeptide (or protein) sequences refers to the degree of similarity of the composition, order, or arrangement of two or more amino acids of the two or more polypeptides (or proteins). The two or more polypeptides (or proteins) may be of the same or different species or group. The term “percent homology” when used in relation to polypeptide (or protein) sequences, refers generally to a percent degree of similarity between the amino acid sequences of two or more polypeptide (or protein) sequences. The term “homologous polypeptides” or “homologous proteins” generally refers to polypeptides or proteins, respectively, that have amino acid sequences and functions that are similar. Such homologous polypeptides or proteins may be related by having amino acid sequences and functions that are similar but are derived or evolved from different or the same species using the techniques described herein.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

In certain embodiments, substantially homologous is at least 50% identical. In certain embodiments, substantially homologous is at least 60% identical. In certain embodiments, substantially homologous is at least 70% identical. In certain embodiments, substantially homologous is at least 80% identical. In certain embodiments, substantially homologous is at least 90% identical. In certain embodiments, substantially homologous is at least 95% identical. In certain embodiments, substantially homologous is at least 98% identical. In certain embodiments, substantially homologous is at least 99% identical.

In some embodiments, the first exogenous polynucleotide further encodes a first conjugating element operably linked to the first promoter and to the first element of interest encoding the functionally inactive first protein moiety of interest; and the second exogenous polynucleotide further encodes a second conjugating element operably linked to the second promoter and to the second element of interest encoding the functionally inactive second protein moiety of interest, the first conjugating element conjugating to the second conjugating element to produce the functionally active protein of interest or fragment thereof.

In some embodiments, the first exogenous polynucleotide further encodes a first functionally inactive marker moiety operably linked to the first promoter, the first conjugating element, and the first protein moiety of interest and the second exogenous polynucleotide further encodes a second functionally inactive marker moiety operably linked to the second promoter, the second conjugating element, and the second protein moiety of interest, the first conjugating element conjugating to the second conjugating element to produce a functionally active marker or fragment thereof.

Examples of binary conjugating elements include, but are not limited to, a SpyTag/SpyCatcher binary system, which conjugates via a covalent bond (see, e.g., Wei et al., J. Biol. Chem. (2021) 297(4):101119; https://doi.org/10.1016/j.jbc.2021.101119); the Split-recombinase system (e.g., the Split-Cre system, which utilizes a dimerization mechanism [see, e.g., Hirrlinger et al., PLoS ONE (2009) 4(1): e4286; https:/journals.plos.org/plosone/article?id=10.1371/journal.pone.0004286]); or two inactive moieties of CRISPR.

In a non-limiting example, the SpyTag/SpyCatcher system is a technology for irreversible conjugation of recombinant proteins. The peptide SpyTag spontaneously reacts with the protein SpyCatcher to form an intermolecular isopeptide bond between the pair. DNA sequence encoding SpyTag is fused with the DNA sequence encoding a protein of interest in one breed, while SpyCatcher is fused with the DNA sequence encoding a protein of interest in another breed. These fusion proteins become covalently linked when coexpressed in the progeny genetically modified PGC or avian, thereby bringing together the functionally inactive first protein moiety of interest and the functionally inactive second protein moiety of interest to form the functionally active protein of interest or fragment thereof.

In another non-limiting example, the genomic modifier comprises a recombinase (e.g., a Split-Cre recombinase system). Recombinases are genetic recombination enzymes involved in homologous recombination. In some embodiments, the Split-recombinase system is a Split-Cre recombinase system. Cre recombinase (Cre) is able to recombine specific sequences of DNA without the need for cofactors, and it catalyzes the site-specific recombination event between two DNA recognition target sites (LoxP sites; locus of crossover in phage P1). In the Split-Cre recombinase system, DNA sequence encoding the N-terminal portion of Cre (NCre) is fused with the DNA sequence encoding a protein of interest in one breed, while DNA sequence encoding the C-terminal portion of Cre (CCre) is fused with the DNA sequence encoding a protein of interest in another breed. In some embodiments, when the fusion proteins are co-expressed in the progeny, the interaction domain comprises the GCN4-coiled coil domain to dimerize the Ncre and CCre moieties.

In another non-limiting example, two inactive moieties of CRISPR are used, which when both expressed in the progeny, provide a functional CRISPR system to target the GOI.

In some embodiments, the first conjugating element comprises the first element of interest and the second conjugating element comprises the second element of interest. In some embodiments, (a) the first conjugating element comprises a functionally inactive first moiety of a recombinase and the second conjugating element comprises a functionally inactive second moiety of the recombinase, the second moiety of the recombinase distinct from the first moiety of the recombinase, the first conjugating element conjugating to the second conjugating element to produce the functionally active recombinase or fragment thereof, (b) the first conjugating element comprises a functionally inactive first moiety of a CRISPR protein and the second conjugating element comprises a functionally inactive second moiety of the CRISPR protein, the second moiety of the CRISPR protein distinct from the first moiety of the CRISPR protein, the first conjugating element conjugating to the second conjugating element to produce the functionally active CRISPR protein or fragment thereof, or (c) a combination thereof.

Using the Tag/Catcher pair, bioconjugation can be achieved between two recombinant proteins that would otherwise be restrictive or impossible with traditional direct genetic fusion between the two proteins. For example, issues regarding protein folding, suboptimal expression host, and specialized post-translational modifications can be alleviated by separating the production of the proteins with the modularity of the Tag/Catcher system.

To direct the first moiety of interest, the second protein moiety of interest, or both to the GOI in the cell nucleus, a nuclear localization signal (NLS) may be used. In some embodiments, the first exogenous polynucleotide encodes a nuclear localization signal (NLS) operably linked to the first protein moiety of interest; the second exogenous polynucleotide encodes a nuclear localization signal (NLS) operably linked to the second protein moiety of interest; or a combination thereof.

In some embodiments, the first exogenous polynucleotide, the second exogenous polynucleotide, or both further comprises at least one self-cleaving peptide. Examples of self-cleaving peptides include, but are not limited to, 2A self-cleaving peptides. 2A self-cleaving peptides, or 2A peptides, form a class of 18-22 aa-long peptides, which can induce ribosomal skipping during translation of a protein in a cell. These peptides share a core sequence motif of DxExNPGP, and are found in a wide range of viral families. They help generating polyproteins by causing the ribosome to fail at making a peptide bond. Examples of 2A self-cleaving peptides include, but are not limited to, P2A, E2A, F2A, and T2A.

In some embodiments, the at least one self-cleaving peptide comprises a P2A peptide, a T2A peptide, or both.

In some embodiments, the P2A peptide is encoded by SEQ ID NO: 13 or SEQ ID NO: 53. In some embodiments, the P2A peptide has a sequence at least 80% identical to SEQ ID NO: 14 or SEQ ID NO: 75. In some embodiments, the P2A peptide has a sequence at least 85% identical to SEQ ID NO: 14 or SEQ ID NO: 75. In some embodiments, the P2A peptide has a sequence at least 90% identical to SEQ ID NO: 14 or SEQ ID NO: 75.

In some embodiments, the P2A peptide has a sequence at least 95% identical to SEQ ID NO: 14 or SEQ ID NO: 75. In some embodiments, the P2A peptide has a sequence at least 96% identical to SEQ ID NO: 14 or SEQ ID NO: 75. In some embodiments, the P2A peptide has a sequence at least 97% identical to SEQ ID NO: 14 or SEQ ID NO: 75. In some embodiments, the P2A peptide has a sequence at least 98% identical to SEQ ID NO: 14 or SEQ ID NO: 75. In some embodiments, the P2A peptide has a sequence at least 99% identical to SEQ ID NO: 14 or SEQ ID NO: 75. In some embodiments, the P2A peptide has a sequence at least 100% identical to SEQ ID NO: 14 or SEQ ID NO: 75. In some embodiments, the P2A peptide has a sequence comprising SEQ ID NO: 14 or SEQ ID NO: 75.

In some embodiments, the T2A peptide is encoded by SEQ ID NO: 17 or SEQ ID NO: 54. In some embodiments, the T2A peptide has a sequence at least 80% identical to SEQ ID NO: 18 or SEQ ID NO: 76. In some embodiments, the T2A peptide has a sequence at least 85% identical to SEQ ID NO: 18 or SEQ ID NO: 76. In some embodiments, the T2A peptide has a sequence at least 90% identical to SEQ ID NO: 18 or SEQ ID NO: 76.

In some embodiments, the T2A peptide has a sequence at least 95% identical to SEQ ID NO: 18 or SEQ ID NO: 76. In some embodiments, the T2A peptide has a sequence at least 96% identical to SEQ ID NO: 18 or SEQ ID NO: 76. In some embodiments, the T2A peptide has a sequence at least 97% identical to SEQ ID NO: 18 or SEQ ID NO: 76. In some embodiments, the T2A peptide has a sequence at least 98% identical to SEQ ID NO: 18 or SEQ ID NO: 76. In some embodiments, the T2A peptide has a sequence at least 99% identical to SEQ ID NO: 18 or SEQ ID NO: 76. In some embodiments, the T2A peptide has a sequence at least 100% identical to SEQ ID NO: 18 or SEQ ID NO: 76. In some embodiments, the T2A peptide has a sequence comprising SEQ ID NO: 18 or SEQ ID NO: 76.

One or more self-cleaving peptides may also be utilized. In some embodiments, the first exogenous polynucleotide further comprises one or more self-cleaving peptides operably linked to the first protein moiety of interest and wherein the second exogenous polynucleotide further comprises one or more self-cleaving peptides operably linked to the second protein moiety of interest.

In still another exemplary method, the binary elements comprise two genomic modifiers. In a non-limiting example, the first protein moiety of interest comprising a functionally active first genomic modifier and the second protein moiety of interest comprising a functionally active second genomic modifier, wherein: the functionally active second genomic modifier targeting a first gene of interest (GOI) or fragment thereof on a chromosome, the first GOI or fragment thereof modified to introduce one or more target sites specific to the second genomic modifier, and the first GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active first gene of interest (GOI), the modified trait inducing sterility or inhibiting fertility in the functionally modified and genetically modified progeny avian embryo or progeny avian without impairing viability; and the functionally active first genomic modifier targeting a second gene of interest (GOI) or fragment thereof on a chromosome, the second GOI or fragment thereof modified to introduce one or more target sites specific to the second genomic modifier, and the second GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active second gene of interest, the modified trait inducing sterility or inhibiting fertility in the functionally modified and genetically modified progeny avian embryo or progeny avian without impairing viability. In some embodiments, the first GOI and the second GOI comprise the same GOI. In some embodiments, the fragment of the first GOI and the fragment of the second GOI are distinct fragments of the same GOI. In some embodiments, the first GOI and the second GOI comprise distinct GOI.

Typically, the first GOI and the second GOI are the same GOI. Otherwise, by crossing the first genetically modified avian and the second genetically modified avian, the resulting genetically modified progeny avian will have one wild-type allele of either the first GOI or the second GOI. However, the fragments (e.g., of the identical first GOI and second GOI) may be different so that they are directed to different areas of the same GOI (e.g., different exons; different fragments of the same exon; a fragment of an exon and a fragment of promoter region).

Any combination of the above options can also be used. Examples of representations of these options are described in detail below.

### Target Genes for Sterility

Several genes are associated with sterility (fertility-required genes [FRGs]) in basic research studies, as well as in clinical medicine. These can be broadly divided into two groups: the first, genes which are associated with sterility and also function in somatic cells, and the second, are genes with isolated function in PGCs, gametes maturation or gametes function. The latter group are targeted, in some cases favorably, to induce embryonic sterility while leaving the organism otherwise healthy. These include, but are not limited to, zona pellucida binding protein 1/2 (ZPBP1/2) gene, Cyclin-dependent kinases regulatory subunit 2 (CKS2; CDC28 Protein Kinase Regulatory Subunit 2) gene, spermatogenesis associated 16 (SPATA16) gene, DEAD-box helicase 4 (DDX4) gene, serine/threonine-protein phosphatase PP1-gamma catalytic subunit (PPP1CC) gene, Izumo sperm-egg fusion 1 (IZUMO1) gene, synaptonemal complex central element protein 1 (SYCE1) gene, YTH domain-containing 2 (YTHDC2) gene, Meiosis Specific With Coiled-Coil Domain (MEIOC) gene, septin-4 (SEPT4) gene, Stromal antigen 3 (STAG3) gene, Nanos C2HC-type zinc finger 3 (NANOS3) gene, deleted in azoospermia 1 (DAZ1) gene, deleted in azoospermia-like (DAZL) gene and many more. Collectively, these genes each have an isolated role in one or more aspects of PGC survival, maturation or differentiation in one or more aspects of gametogenesis, meiosis, gametes function or fertilization. In certain cases, the proteins encoded by these genes may have a redundancy of function or mechanism.

Thus, in some instances, it may be advantageous to mutate at least two genes in order to reduce or eliminate a specific function, e.g., functions associated with sterility, gametes maturation, or gametes function or a combination thereof. From these genes, the ones associated with both sexes' sterility, located on autosomal chromosomes and having well annotated evolutionary-conserved orthologous sequences in chickens or other avians, are advantageous.

In some embodiments, the first nucleic acid sequence comprises a mutated or null GOI sequence or a fragment thereof or encodes an endonuclease enzyme that can carry out genome editing; or wherein insertion of the first nucleic acid sequence in the chromosome of interest modifies or disrupts the targeted GOI, the targeted GOI has: an isolated function specific to a PGC; or a function specific to gametogenesis, gamete maturation, or gamete function in the genetically modified avian. In some embodiments, the modification or disruption of the gene has an isolated function specific to a PGC reduces or inhibits survival, maturation, or differentiation of a PGC derived from the genetically modified avian. In some embodiments, the modification or disruption of a gene has a function specific to gametogenesis, game maturation, or gamete function reduces or inhibits gametogenesis, meiosis, gamete function, or gamete fertilization in the genetically modified avian.

In some embodiments, the modification or disruption of the targeted GOI has an isolated function specific to a PGC reduces or inhibits survival, maturation, or differentiation of a PGC derived from the genetically modified avian.

A skilled artisan would appreciate that the term “isolated function” may encompass a situation wherein if a GOI were knocked-out (KO), no other systems will be affected. This is a restriction that that is adopted as a measure of caution, i.e., one would not KO a gene that causes sterility if it causes blindness as well. Combining two GOI to maximize the effect of sterility, in case one is insufficient, means that the two genes have isolated function in the gametes. In certain embodiments, the term “isolated function” encompasses the function of a GOI that is only active in gametes.

In some embodiments, the modification or disruption of the targeted GOI has a function specific to gametogenesis, game maturation, or gamete function reduces or inhibits gametogenesis, meiosis, gamete function, or gamete fertilization in the genetically modified avian.

Notably, in some embodiments, the RNA binding protein deleted in AZoospermia-Like (DAZL) is a key determinant of germ cell maturation and entry into meiosis in many species. Highly conserved in evolution the chicken DAZL (Smorag et al. 2014. Wiley Interdiscip. Rev.: RNA 5: 527-535), located on chromosome 2 in a well annotated region (Chr2: 34429592.34442834; GRCg6a). In accordance with its roles, DAZL is expressed in PGCs in both male and female embryos, thus it serves as marker for PGCs population. DAZL is a member of the Daz family genes, which share conserved evolutionary role in numerous model-organisms, including Caenorhabditis elegans, Drosophila melanogaster fly, fish, frog, mice and human patients (Fu et al. [2015] Intl. J. Biol. Sci. 11: 1226-1235), that result in sterility due to PGCs failure to form or to mature to functional adult gametes.

Examples of genes having an isolated function specific to a PGC or genes having a function specific to gametogenesis, gamete maturation, or gamete function include, but are not limited to, zona pellucida binding protein 1/2 (ZPBP1/2) gene, cyclin-dependent kinases regulatory subunit 2 (CKS2; CDC28 Protein Kinase Regulatory Subunit 2) gene, spermatogenesis associated 16 (SPATA16) gene, DEAD-box helicase 4 (DDX4) gene, serine/threonine-protein phosphatase PP1-gamma catalytic subunit (PPP1CC) gene, Izumo sperm-egg fusion 1 (IZUMO1) gene, synaptonemal complex central element protein 1 (SYCE1) gene, YTH domain-containing 2 (YTHDC2) gene, meiosis specific with coiled-coil domain (MEIOC) gene, septin-4 (SEPT4) gene, stromal antigen 3 (STAG3) gene, Nanos C2HC-type zinc finger 3 (NANOS3) gene, deleted in azoospermia 1 (DAZ1) gene, deleted in azoospermia-like (DAZL) gene, respectively encoding Zona Pellucida Binding Protein 1/2 (ZPBP1/2) protein, Cytokine-Dependent Kinases Regulatory Subunit 2 (CKS2; CDC28 Protein Kinase Regulatory Subunit 2) protein, Spermatogenesis Associated 16 (SPATA16) protein, DEAD-Box Helicase 4 (DDX4) protein, Serine/Threonine-Protein Phosphatase PP1-Gamma Catalytic Subunit (PPP1CC) protein, Izumo Sperm-Egg Fusion 1 (IZUMO1) protein, Synaptonemal Complex Central Element Protein 1 (SYCE1) protein, YTH Domain-Containing 2 (YTHDC2) protein, Meiosis Specific With Coiled-Coil Domain (MEIOC) protein, Septin-4 (SEPT4) protein, Stromal Antigen 3 (STAG3) protein, Nanos C2HC-Type Zinc Finger 3 (NANOS3) protein, Deleted In Azoospermia 1 (DAZ1) protein, and Deleted In Azoospermia-Like (DAZL) protein. While these genes may encode proteins comprising isolated functions, for example those specific to a PGC or specific to gametogenesis, gamete maturation, or gamete function, a skilled artisan would appreciate that in some embodiments, proteins may have redundant mechanisms or a shared function.

In some embodiments, the chromosome of interest is an autosomal chromosome. In some embodiments, the sterile phenotype induces sterility in a male progeny avian or a female progeny avian.

### Genome Editing

Genome editing using engineered endonucleases refers to a genetic method using nucleases to cut and create specific double-stranded breaks at a desired location(s) in the genome (e.g., on the chromosome of interest of a bird), which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize only a few base pairs on the DNA as their target and the probability is very high that the recognized base pair combination will be found in many locations across the genome, resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and the CRISPR/Cas system.

Meganucleases—They are commonly grouped into four families: the LAGLIDADG family, the GIY-YIG family, the His-Cys box family and the HNH family. These families are characterized by structural motifs that affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp), thus making them naturally very specific for cutting at a desired location. This can be exploited to make site-specific double-stranded breaks in genome editing. One of skilled in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514.

ZFNs and TALENs—Two distinct classes of engineered nucleases, zinc-finger nucleases (ZFNs) and transcription activator-like effector nucleases (TALENs), have both been proven to be effective at producing targeted double-stranded breaks. Basically, ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separated from each other is selected. The cleaving portion is separated and then linked to a DNA binding domain, thereby yielding an endonuclease with very high specificity for a desired sequence. An exemplary restriction enzyme with such properties is Fokl. Additionally, Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example, ZFNs and TALENs can be constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the non-homologous end-joining (NHEJ) pathway most often results in INDELs which are small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (see, e.g., Carlson et al., 2012, Proc Natl Acad Sci USA.; 109(43):17382-7; Lee et al., 2010, Trends Biotechnol.; 28(9):445-6). In addition, when a fragment of DNA with homology to the targeted region is introduced in conjunction with the nuclease pair, the double-stranded break can be repaired via homology directed repair to generate specific modifications (see, e.g., Li et al., 2011, Nucleic Acids Res. 39(1):359-72; Miller et al., 2010, Nat. Struct. Mol. Biol. 17(9):1144-51; Urnov et al., 2005, Nature 435(7042):646-51).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others.

CRISPR-Cas system—Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (e.g. Cho et al., 2013, Nat. Biotechnol. 31(3):230-2; Cong et al., 2013, Science 339(6121):819-23; DiCarlo et al., 2013, Nucleic Acids Res. 41(7):4336-43; Hwang et al., 2013, Nat. Biotechnol. 31(3):227-9; Jinek et al., 2013, Elife. 2013 Jan. 29; 2:e00471; Mali et al., 2013, Nat. Methods 10(10):957-63).

As used herein, the terms “gRNA” and “sgRNA” may be used interchangeably, having all the same meanings and qualities.

It is known that the CRIPSR/Cas system for genome editing contains two distinct components: a guide RNA (gRNA) and an endonuclease e.g., Cas9. The gRNA is typically a 20-nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded breaks produced by CRISPR/Cas can undergo homologous recombination or NHEJ. In certain embodiments, the CRISPR/Cas system comprises single guide RNA (sgRNA) and a Cas protein. In certain embodiments, the CRISPR/Cas system comprises a complex of single guide RNA (sgRNA) and a Cas protein. In certain embodiments, the Cas of the CRISPR/Cas system comprises a single polypeptide. In certain embodiments, the Cas of the CRISPR/Cas system is an endonuclease.

In certain embodiments, the CRISPR/Cas is CRISPR/Cas9. In certain embodiments, the sgRNA sequence includes a protospacer adjacent motif (PAM) sequence, which is a short nucleotide sequence (usually 2-6 bp) enabling the Cas nuclease in a CRISPR system to cut. In certain embodiments, a PAM sequence is added to the 3′-end of the sgRNA. In certain embodiments the PAM sequence comprises an additional three nucleotide., “AGG” (“PAM sequence”) at the 3′-end of the sgRNA.

sgRNA may comprise a ribonucleic acid, or it may comprise a deoxyribonucleic acid. sgRNA may encode a ribonucleic acid, or it may encode a deoxyribonucleic acid. In one embodiment, sgRNA-5′ssODN (SEQ ID NO: 21) codes for a RNA sequence. In one embodiment, SEQ ID NO: 21 codes for a DNA sequence, which may be used as a template for preparing sgRNA. In some embodiments, the complement of SEQ ID NO: 21 may be used as a template for preparing sgRNA. In one embodiment, sgRNA-3′ssODN (SEQ ID NO: 22) codes for a RNA sequence. In one embodiment, SEQ ID NO: 22 codes for a DNA sequence, which may be used as a template for preparing sgRNA. In some embodiments, the complement of SEQ ID NO: 22 may be used as a template for preparing sgRNA. In one embodiment, TV-sgRNA (SEQ ID NO: 25) codes for a RNA sequence. In one embodiment, SEQ ID NO: 25 codes for a DNA sequence, which may be used as a template for preparing sgRNA. In some embodiments, the complement of SEQ ID NO: 25 may be used as a template for preparing sgRNA.

The Cas9 nuclease has two functional domains: RuvC and HNH, each cutting a different DNA strand. When both domains are active, the Cas9 causes double strand breaks in the genomic DNA. A significant advantage of CRISPR/Cas is that the high efficiency of this system coupled with the ability to easily create synthetic gRNAs enables multiple genes to be targeted simultaneously. Apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription. In certain embodiments, the CRISPR/Cas is CRISPR/dCas9.

In order to use the CRISPR system, both gRNA and Cas9 should be expressed in a target cell. The insertion vector can contain both cassettes on a single plasmid or the cassettes are expressed from two separate plasmids. CRISPR plasmids are publicly available such as the px330 plasmid from Addgene®. Additionally, mRNA encoding Cas9 and the gRNA can be introduced to the target cells as well as recombinant Cas9 protein in complex with the gRNA (i.e. insert the RNP complex into the cell).

In certain embodiments, the CRISPR/Cas system is a Class 1 CRISPR/Cas system. In certain embodiments, a Class 1 CRISPR/Cas system comprises a multi-subunit crRNA-effector complex. In certain embodiments, the CRISPR/Cas system is a type I CRISPR-Cas system. In certain embodiments, the CRISPR/Cas system is a type III CRISPR/Cas system. In certain embodiments, the CRISPR/Cas system is a type IV CRISPR-Cas system.

In certain embodiments, the CRISPR/Cas system is a Class 2 CRISPR/Cas system. In certain embodiments, a Class 2 CRISPR/Cas system comprises a single subunit crRNA-effector module. In certain embodiments, the CRISPR/Cas system is a type II CRISPR-Cas system. In certain embodiments, the CRISPR/Cas system is a type V CRISPR/Cas system.

In certain embodiments, the Cas in the Class 2 CRISPR/Cas system can be Cas9, Cpf1, C2c1, C2c2 or C2c3. A person of ordinary skill in the art would understand the classification of CRISPR/Cas systems as it is well-known in the art (e.g., Nat. Rev Microbiol. 2017 March, 15(3): 169-182; Nat. Rev. Microbiol. 2015 November, 13(11): 722-736), and that this classification is evolving with time (Mol. Cell. 2015 November 5, 60(3): 385-397). In some embodiments, the CRISPR/Cas is any CRISPR associated protein (CAS) endonuclease known in the art.

Genome editing using recombinant adeno-associated viruses (rAAVs) is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross or subtle endogenous gene alterations in a cell or a combination thereof rAAV genome editing has the advantage in that it targets a single allele and does not result in any off-target genomic alterations.

### DNA Editing Agent

The technology described herein provides, in certain aspects and embodiments, a DNA-editing system. The DNA editing system may be constructed using recombinant DNA technology well known to persons skilled in the art.

In one embodiment, the DNA editing system disclosed herein may be comprised in a single nucleic acid construct or comprised in a combination of nucleic acid constructs. In one embodiment, the DNA editing system comprises at least two key elements as described below:

A first agent comprising a first exogenous polynucleotide, the first exogenous polynucleotide comprising: a first promoter; and a first element of interest operably linked to the promoter, the first element of interest encoding a first protein moiety of interest, wherein a first genetically modified avian having a genome comprising the first agent retains fertility and viability while producing a primordial germ cell (PGC) having a genome comprising the first agent without impairing viability of the PGC; and a second agent comprising a second exogenous polynucleotide, the second exogenous polynucleotide comprising: a second promoter; and a second element of interest operably linked to the promoter, the second element of interest encoding a second protein moiety of interest, wherein a second genetically modified avian having a genome comprising the second agent retains fertility and viability while producing a primordial germ cell (PGC) having a genome comprising the second agent without impairing viability of the PGC, the first agent and the second agent, when co-expressed, in a genetically modified progeny avian embryo or progeny avian, wherein the genetically modified progeny avian embryo or progeny avian is a progeny of a first genetically modified avian arising from the first genetically modified PGC comprising the first agent and an opposite-gendered second genetically modified avian arising from the second genetically modified PGC comprising the second agent, inducing sterility or inhibiting fertility in the genetically modified progeny avian embryo or progeny avian without impairing viability.

In some embodiments, the first nucleic acid sequence encodes a sterility-inducing protein or an endonuclease enzyme that can carry out genome editing. In some embodiments, insertion of the first nucleic acid sequence in the chromosome of interest modifies or disrupts the GOI, e.g., where the GOI has an isolated function specific to a PGC (e.g., reducing or inhibiting survival, maturation, or differentiation of a PGC derived from a genetically modified avian derived from the DNA edited PGC), or where the GOI has a function specific to gametogenesis, gamete maturation, or gamete function in the genetically modified avian (e.g., reducing or inhibiting gametogenesis, meiosis, gamete function, or gamete fertilization in the genetically modified avian) or a combination thereof. Examples of target genes for sterility are described elsewhere herein. In some embodiments, the chromosome of interest is an autosomal chromosome. In some embodiments, the sterile phenotype induces sterility in a male genetically modified progeny avian or a female genetically modified progeny avian.

“Recombinases” are genetic recombination enzymes involved in homologous recombination (HR). Cre recombinase (Cre) is able to recombine specific sequences of DNA without the need for cofactors, and it catalyzes the site specific recombination event between two DNA recognition target sites (LoxP sites; locus of crossover in phage P1). Each 34 base pair (bp) loxP recognition target site consists of two 13 bp palindromic sequences which flank an 8 bp spacer region. The Flp-FRT recombination system is also a site-directed recombination technology, analogous to Cre-lox recombination but involving the recombination of sequences between short flippase recognition target (FRT) sites by the recombinase flippase (Flp). Other tyrosine recombinase systems include, but are not limited to, the Dre recombinase and the Rox recognition target site, as well as the Vika recombinase and the Vox recognition target site. “Floxing” refers to the sandwiching of a DNA sequence (which is then said to be floxed) between two lox P sites. The terms are constructed upon th “phrase “flanking/flanked” by LoxP”. Recombination between LoxP sites is catalyzed by Cre recombinase. Floxing a gene allows it to be deleted (knocked out), translocated or inverted, e.g., in a process called “Cre-Lox recombination.”

In some embodiments, the genomic modifier comprises a site-specific recombinase enzyme (e.g., a tyrosine recombinase or a serine recombinase) or a functionally active fragment thereof. In some embodiments, the tyrosine recombinase comprises Cre, Dre, Flp, or Vika, and the target site comprises a recombinases recognition site, the recombinase recognition site comprising, respectively, Lox, Rox, FRT, or Fox. For example, the tyrosine recombinase comprises Cre and the recombinase recognition site comprises LoxP; or the tyrosine recombinase comprises Flp and the recombinase recognition site comprises FRT. In some embodiments, the recombinase is a Split-recombinase, as described herein. In some embodiments, the Split-recombinase is a Split-Cre recombinase.

In some embodiments, the first protein moiety of interest is a functionally inactive first protein moiety of interest and the second protein moiety of interest is a functionally inactive second protein moiety of interest.

In certain embodiments, the functionally inactive first Cre protein moiety of interest is encoded by a nucleic acid sequence 50% to 100% identical in sequence to SEQ ID NO: 5 or SEQ ID NO: 49. In certain embodiments, the functionally inactive first Cre protein moiety of interest is encoded by a nucleic acid sequence 80% to 100% identical in sequence to SEQ ID NO: 5 or SEQ ID NO: 49. In certain embodiments, the functionally inactive first Cre protein moiety of interest is encoded by a nucleic acid sequence 85% to 100% identical in sequence to SEQ ID NO: 5 or SEQ ID NO: 49. In certain embodiments, the functionally inactive first Cre protein moiety of interest is encoded by a nucleic acid sequence 90% to 100% identical in sequence to SEQ ID NO: 5 or SEQ ID NO: 49. In certain embodiments, the functionally inactive first Cre protein moiety of interest is encoded by a nucleic acid sequence 95% to 100% identical in sequence to SEQ ID NO: 5 or SEQ ID NO: 49. In certain embodiments, the functionally inactive first Cre protein moiety of interest is encoded by a nucleic acid sequence 98% to 100% identical in sequence SEQ ID NO: 5 or SEQ ID NO: 49. In certain embodiments, the functionally inactive first Cre protein moiety of interest is encoded by a nucleic acid sequence 99% to 100% identical in sequence to SEQ ID NO: 5 or SEQ ID NO: 49. In certain embodiments, the functionally inactive first Cre protein moiety of interest is encoded by a nucleic acid sequence 100% identical in sequence to SEQ ID NO: 5 or SEQ ID NO: 49.

In certain embodiments, the functionally inactive second Cre protein moiety of interest is encoded by a nucleic acid sequence 50% to 100% identical in sequence to SEQ ID NO: 11 or SEQ ID NO: 51. In certain embodiments, the functionally inactive second Cre protein moiety of interest is encoded by a nucleic acid sequence 80% to 100% identical in sequence to SEQ ID NO: 11 or SEQ ID NO: 51. In certain embodiments, the functionally inactive second Cre protein moiety of interest is encoded by a nucleic acid sequence 85% to 100% identical in sequence to SEQ ID NO: 11 or SEQ ID NO: 51. In certain embodiments, the functionally inactive second Cre protein moiety of interest is encoded by a nucleic acid sequence 90% to 100% identical in sequence to SEQ ID NO: 11 or SEQ ID NO: 51. In certain embodiments, the functionally inactive second Cre protein moiety of interest is encoded by a nucleic acid sequence 95% to 100% identical in sequence to SEQ ID NO: 11 or SEQ ID NO: 51. In certain embodiments, the functionally inactive second Cre protein moiety of interest is encoded by a nucleic acid sequence 98% to 100% identical in sequence SEQ ID NO: 11 or SEQ ID NO: 51. In certain embodiments, the functionally inactive second Cre protein moiety of interest is encoded by a nucleic acid sequence 99% to 100% identical in sequence to SEQ ID NO: 11 or SEQ ID NO: 51. In certain embodiments, the functionally inactive second Cre protein moiety of interest is encoded by a nucleic acid sequence 100% identical in sequence to SEQ ID NO: 11 or SEQ ID NO: 51.

In certain embodiments, the functionally inactive first Cre protein moiety of interest is 50% to 100% identical in sequence to SEQ ID NO: 6 or SEQ ID NO: 71. In certain embodiments, the functionally inactive first Cre protein moiety of interest is 80% to 100% identical in sequence to SEQ ID NO: 6 or SEQ ID NO: 71. In certain embodiments, the functionally inactive first Cre protein moiety of interest is 85% to 100% identical in sequence to SEQ ID NO: 6 or SEQ ID NO: 71. In certain embodiments, the functionally inactive first Cre protein moiety of interest is 90% to 100% identical in sequence to SEQ ID NO: 6 or SEQ ID NO: 71. In certain embodiments, the functionally inactive first Cre protein moiety of interest is 95% to 100% identical in sequence to SEQ ID NO: 6 or SEQ ID NO: 71. In certain embodiments, the functionally inactive first Cre protein moiety of interest is 98% to 100% identical in sequence SEQ ID NO: 6 or SEQ ID NO: 71. In certain embodiments, the functionally inactive first Cre protein moiety of interest is 99% to 100% identical in sequence to SEQ ID NO: 6 or SEQ ID NO: 71. In certain embodiments, the functionally inactive first Cre protein moiety of interest is 100% identical in sequence to SEQ ID NO: 6 or SEQ ID NO: 71.

In certain embodiments, the functionally inactive second Cre protein moiety of interest is 50% to 100% identical in sequence to SEQ ID NO: 12 or SEQ ID NO: 73. In certain embodiments, the functionally inactive second Cre protein moiety of interest is 80% to 100% identical in sequence to SEQ ID NO: 12 or SEQ ID NO: 73. In certain embodiments, the functionally inactive second Cre protein moiety of interest is 85% to 100% identical in sequence to SEQ ID NO: 12 or SEQ ID NO: 73. In certain embodiments, the functionally inactive second Cre protein moiety of interest is 90% to 100% identical in sequence to SEQ ID NO: 12 or SEQ ID NO: 73. In certain embodiments, the functionally inactive second Cre protein moiety of interest is 95% to 100% identical in sequence to SEQ ID NO: 12 or SEQ ID NO: 73. In certain embodiments, the functionally inactive second Cre protein moiety of interest is 98% to 100% identical in sequence SEQ ID NO: 12 or SEQ ID NO: 73. In certain embodiments, the functionally inactive second Cre protein moiety of interest is 99% to 100% identical in sequence to SEQ ID NO: 12 or SEQ ID NO: 73. In certain embodiments, the functionally inactive second Cre protein moiety of interest is 100% identical in sequence to SEQ ID NO: 12 or SEQ ID NO: 73.

In some embodiments, the functionally inactive first Cre protein moiety of interest has a sequence at least 95% identical to SEQ ID NO: 6 or SEQ ID NO: 71 and is encoded by a nucleic acid sequence at least 95% identical to SEQ ID NO: 5 or SEQ ID NO: 49; the functionally inactive second Cre protein moiety of interest has a sequence at least 95% identical to SEQ ID NO: 12 or SEQ ID NO: 73 and is encoded by a nucleic acid sequence at least 95% identical to SEQ ID NO: 11 or SEQ ID NO: 51; or both. In some embodiments, the functionally inactive first Cre protein moiety of interest is encoded by a nucleic acid sequence corresponding to SEQ ID NO: 5 or SEQ ID NO: 49; the functionally inactive second Cre protein moiety of interest is encoded by a nucleic acid sequence corresponding to SEQ ID NO: 11 or SEQ ID NO: 51; or both.

In some embodiments, the functionally inactive first Cre protein moiety of interest has a sequence at least 95% identical to SEQ ID NO: 6 or SEQ ID NO: 71; the functionally inactive second Cre protein moiety of interest has a sequence at least 95% identical to SEQ ID NO: 12 or SEQ ID NO: 73 or SEQ ID NO: 73; or both. In some embodiments, the functionally inactive first Cre protein moiety of interest has a sequence corresponding to SEQ ID NO: 6 or SEQ ID NO: 71; the functionally inactive second Cre protein moiety of interest has a sequence corresponding to SEQ ID NO: 12 or SEQ ID NO: 73; or both.

It will be appreciated that more than one nucleic acid coding strand is encompassed. The degenerate genetic code, also known as the redundancy of the genetic code, refers to the fact that multiple codons, or sets of three nucleotides, can code for the same amino acid during protein synthesis, namely, that different codons can encode for the same amino acid, but no codon can encode for more than one amino acid. Therefore, in some instances, multiple codons code for the same amino acid, and some amino acids are coded for by as many as six different codons, and a nucleic acid substitution in an underlying nucleic acid coding sequence will not necessarily result in a change in the amino acid encoded by the codon corresponding to the nucleic acid substitution.

It will also be appreciated that not all amino acid replacements have the same effect on function or structure of protein. A conservative amino acid replacement (conservative substitution, conservative mutation) comprises a replacement in a protein that changes the amino acid to a different amino acid with similar biochemical properties (e.g., charge, hydrophobicity, and/or size). Examples of conservative amino acid replacement include, but are not limited to, changes within amino acid groups that are aliphatic (Gly, Ala, Val, Leu, and/or Ile); hydroxyl or sulfur/selenium-containing (Ser, Cys, seleno-Cys, Thr, and/or Met); cyclic (Pro); aromatic (Phe, Tyr, Trp); basic (His, Lys, Arg); and acidic and their amides (Asp, Glu, Asn, Gln).

It will be appreciated that the term “modification” can encompass an amino acid modification such as an amino acid substitution, insertion, and/or deletion in a polypeptide sequence. The term “homolog” as used herein refers to a polypeptide having a sequence homology of a certain amount, namely of at least 70%, e.g., at least 80%, 90%, 95%, 96%, 97%, 98%, 99% of the amino acid sequence it is referred to. Homology refers to the magnitude of identity between two sequences. Homolog sequences have the same or similar characteristics, in particular, have the same or similar property of the sequence as identified.

The term ‘variant’ as used herein refers to a polypeptide wherein the amino acid sequence exhibits substantially 70, 80, 95, or 99% homology with the amino acid sequence as set forth in the sequence listing. It should be appreciated that the variant may result from a modification of the native amino acid sequences, or by modifications including insertion, substitution or deletion of one or more amino acids. The term “isoform” as used herein refers to variants of a polypeptide that are encoded by the same gene, but that differ in their isoelectric point (pI) or molecular weight (MW), or both. Such isoforms can differ in their amino acid composition (e.g., as a result of alternative splicing or limited proteolysis) and in addition, or in the alternative, may arise from differential post-translational modification (e.g., glycosylation, acylation, phosphorylation deamidation, or sulphation). As used herein, the term “isoform” also refers to a protein that exists in only a single form, i.e., it is not expressed as several variants. The term “fragment” as used herein refers to any portion of the full-length amino acid sequence of protein of a polypeptide disclosed herein associated with the sterile avian embryos, products and uses thereof, which has less amino acids than the full-length amino acid sequence of a polypeptide disclosed herein associated with the sterile avian embryos, products and uses thereof. The fragment may or may not possess a functional activity of such polypeptides.

In some embodiments, the NLS is encoded by SEQ ID NO: 3 or SEQ ID NO: 48. In some embodiments, the NLS has a sequence at least 80% identical to SEQ ID NO: 4 or SEQ ID NO: 70. In some embodiments, the NLS has a sequence at least 85% identical to SEQ ID NO: 4 or SEQ ID NO: 70. In some embodiments, the NLS has a sequence at least 90% identical to SEQ ID NO: 4 or SEQ ID NO: 70. In some embodiments, the NLS has a sequence at least 95% identical to SEQ ID NO: 4 or SEQ ID NO: 70. In some embodiments, the NLS has a sequence at least 96% identical to SEQ ID NO: 4 or SEQ ID NO: 70. In some embodiments, the NLS has a sequence at least 97% identical to SEQ ID NO: 4 or SEQ ID NO: 70. In some embodiments, the NLS has a sequence at least 98% identical to SEQ ID NO: 4 or SEQ ID NO: 70. In some embodiments, the NLS has a sequence at least 99% identical to SEQ ID NO: 4 or SEQ ID NO: 70. In some embodiments, the NLS has a sequence 100% identical to SEQ ID NO: 4 or SEQ ID NO: 70. In some embodiments, the NLS has a sequence comprising SEQ ID NO: 4 or SEQ ID NO: 70.

In another embodiment, the amino acid sequences of nCre are at least 80% (e.g., at least 85%, 90%, 95%, 96%, 97%, 98%, or 99%) identical to the amino acid sequences set forth above and below. In another embodiment, the amino acid sequences of cCre are at least 80% (e.g., at least 85%, 90%, 95%, 96%, 97%, 98%, or 99%) identical to the amino acid sequences set forth above and below. In another embodiment, the amino acid sequences of Cre with NLS are at least 80% (e.g., at least 85%, 90%, 95%, 96%, 97%, 98%, or 99%) identical to the amino acid sequences set forth above and below. In another embodiment, the amino acid sequences of Flp with NLS are at least 80% (e.g., at least 85%, 90%, 95%, 96%, 97%, 98%, or 99%) identical to the amino acid sequences set forth above and below.

One skilled in the art would appreciate that percent sequence identity may be determined using any of a number of publicly available software application, for example but not limited to BlastP software of the National Center of Biotechnology Information (NCBI) using default parameters.

A skilled artisan would appreciate that percent identity (% identity) provides a number that describes how similar the query sequence is to the target sequence (i.e., how many amino acids in each sequence are identical). The higher the percentage identity is, the more significant the match.

When used in relation to polypeptide (or protein) sequences, the term “identity” refers to the degree of identity between two or more polypeptide (or protein) sequences or fragments thereof. Typically, the degree of similarity between two or more polypeptide (or protein) sequences refers to the degree of similarity of the composition, order, or arrangement of two or more amino acids of the two or more polypeptides (or proteins).

A skilled artisan would appreciate that the term “homology”, and grammatical forms thereof, encompasses the degree of similarity between two or more structures. The term “homologous sequences” refers to regions in macromolecules that have a similar order of monomers. When used in relation to nucleic acid sequences, the term “homology” refers to the degree of similarity between two or more nucleic acid sequences (e.g., genes) or fragments thereof. Typically, the degree of similarity between two or more nucleic acid sequences refers to the degree of similarity of the composition, order, or arrangement of two or more nucleotide bases (or other genotypic feature) of the two or more nucleic acid sequences. The term “homologous nucleic acids” generally refers to nucleic acids comprising nucleotide sequences having a degree of similarity in nucleotide base composition, arrangement, or order. The two or more nucleic acids may be of the same or different species or group. The term “percent homology” when used in relation to nucleic acid sequences, refers generally to a percent degree of similarity between the nucleotide sequences of two or more nucleic acids.

When used in relation to polypeptide (or protein) sequences, the term “homology” refers to the degree of similarity between two or more polypeptide (or protein) sequences (e.g., genes) or fragments thereof. Typically, the degree of similarity between two or more polypeptide (or protein) sequences refers to the degree of similarity of the composition, order, or arrangement of two or more amino acid of the two or more polypeptides (or proteins). The two or more polypeptides (or proteins) may be of the same or different species or group. The term “percent homology” when used in relation to polypeptide (or protein) sequences, refers generally to a percent degree of similarity between the amino acid sequences of two or more polypeptide (or protein) sequences. The term “homologous polypeptides” or “homologous proteins” generally refers to polypeptides or proteins, respectively, that have amino acid sequences and functions that are similar. Such homologous polypeptides or proteins may be related by having amino acid sequences and functions that are similar but are derived or evolved from different or the same species using the techniques described herein.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

In certain embodiments, substantially homologous is at least 50% identical. In certain embodiments, substantially homologous is at least 60% identical. In certain embodiments, substantially homologous is at least 70% identical. In certain embodiments, substantially homologous is at least 80% identical. In certain embodiments, substantially homologous is at least 90% identical. In certain embodiments, substantially homologous is at least 95% identical. In certain embodiments, substantially homologous is at least 98% identical. In certain embodiments, substantially homologous is at least 99% identical.

In other embodiments, the first protein moiety of interest comprises a functionally active first genomic modifier, the functionally active first genomic modifier targeting a second gene of interest (GOI) or fragment thereof, and the second protein moiety of interest comprises a functionally active second genomic modifier, the functionally active second genomic modifier targeting a first gene of interest (GOI) or fragment thereof. In some embodiments the first GOI or fragment thereof and the second GOI or fragment thereof, respectively, comprise the same GOI or fragment thereof, respectively.

In some embodiments, the first genomic modifier comprises a functionally active tyrosine recombinase comprising a Flippase recombinase (Flp) moiety, operably linked to a nuclear localizing signal (NLS), and the first GOI comprises a recombinase recognition site, the recombinase recognition site comprising LoxP; and the second genomic modifier comprises a functionally active tyrosine recombinase comprising Cre recombinase moiety, operably linked to a nuclear localizing signal (NLS), and the second GOI comprises a recombinase recognition site, the recombinase recognition site comprising FRT.

In certain embodiments, the functionally active Cre moiety with NLS is encoded by a nucleic acid sequence 50% to 100% identical in sequence to SEQ ID NO: 26. In certain embodiments, the functionally active Cre moiety with NLS is encoded by a nucleic acid sequence 80% to 100% identical in sequence to SEQ ID NO: 26. In certain embodiments, the functionally active Cre moiety with NLS is encoded by a nucleic acid sequence 85% to 100% identical in sequence to SEQ ID NO: 26. In certain embodiments, the functionally active Cre moiety with NLS is encoded by a nucleic acid sequence 90% to 100% identical in sequence to SEQ ID NO: 26. In certain embodiments, the functionally active Cre moiety with NLS is encoded by a nucleic acid sequence 95% to 100% identical in sequence to SEQ ID NO: 26. In certain embodiments, the functionally active Cre moiety with NLS is encoded by a nucleic acid sequence 96% to 100% identical in sequence SEQ ID NO: 26. In certain embodiments, the functionally active Cre moiety with NLS is encoded by a nucleic acid sequence 97% to 100% identical in sequence SEQ ID NO: 26. In certain embodiments, the functionally active Cre moiety with NLS is encoded by a nucleic acid sequence 98% to 100% identical in sequence SEQ ID NO: 26. In certain embodiments, the functionally active Cre moiety with NLS is encoded by a nucleic acid sequence 99% to 100% identical in sequence to SEQ ID NO: 26. In certain embodiments, the functionally active Cre moiety with NLS is encoded by a nucleic acid sequence 100% identical in sequence to SEQ ID NO: 26.

In certain embodiments, the functionally active Cre moiety with NLS is 50% to 100% identical in sequence to SEQ ID NO: 27. In certain embodiments, the functionally active Cre moiety with NLS is 80% to 100% identical in sequence to SEQ ID NO: 27. In certain embodiments, the functionally active Cre moiety with NLS is 85% to 100% identical in sequence to SEQ ID NO: 27. In certain embodiments, the functionally active Cre moiety with NLS is 90% to 100% identical in sequence to SEQ ID NO: 27. In certain embodiments, the functionally active Cre moiety with NLS is 95% to 100% identical in sequence to SEQ ID NO: 27. In certain embodiments, the functionally active Cre moiety with NLS is 96% to 100% identical in sequence SEQ ID NO: 27. In certain embodiments, the functionally active Cre moiety with NLS is 97% to 100% identical in sequence SEQ ID NO: 27. In certain embodiments, the functionally active Cre moiety with NLS is 98% to 100% identical in sequence SEQ ID NO: 27. In certain embodiments, the functionally active Cre moiety with NLS is 99% to 100% identical in sequence to SEQ ID NO: 27. In certain embodiments, the functionally active Cre moiety with NLS is 100% identical in sequence to SEQ ID NO: 27.

In certain embodiments, the functionally active Flp moiety with NLS is encoded by a nucleic acid sequence 50% to 100% identical in sequence to SEQ ID NO: 28. In certain embodiments, the functionally active Flp moiety with NLS is encoded by a nucleic acid sequence 80% to 100% identical in sequence to SEQ ID NO: 28. In certain embodiments, the functionally active Flp moiety with NLS is encoded by a nucleic acid sequence 85% to 100% identical in sequence to SEQ ID NO: 28. In certain embodiments, the functionally active Flp moiety with NLS is encoded by a nucleic acid sequence 90% to 100% identical in sequence to SEQ ID NO: 28. In certain embodiments, the functionally active Flp moiety with NLS is encoded by a nucleic acid sequence 95% to 100% identical in sequence to SEQ ID NO: 28. In certain embodiments, the functionally active Flp moiety with NLS is encoded by a nucleic acid sequence 96% to 100% identical in sequence SEQ ID NO: 28. In certain embodiments, the functionally active Flp moiety with NLS is encoded by a nucleic acid sequence 97% to 100% identical in sequence SEQ ID NO: 28. In certain embodiments, the functionally active Flp moiety with NLS is encoded by a nucleic acid sequence 98% to 100% identical in sequence SEQ ID NO: 28. In certain embodiments, the functionally active Flp moiety with NLS is encoded by a nucleic acid sequence 99% to 100% identical in sequence to SEQ ID NO: 28. In certain embodiments, the functionally active Flp moiety with NLS is encoded by a nucleic acid sequence 100% identical in sequence to SEQ ID NO: 28.

In certain embodiments, the functionally active Flp moiety with NLS is 50% to 100% identical in sequence to SEQ ID NO: 29. In certain embodiments, the functionally active Flp moiety with NLS is 80% to 100% identical in sequence to SEQ ID NO: 29. In certain embodiments, the functionally active Flp moiety with NLS is 85% to 100% identical in sequence to SEQ ID NO: 29. In certain embodiments, the functionally active Flp moiety with NLS is 90% to 100% identical in sequence to SEQ ID NO: 29. In certain embodiments, the functionally active Flp moiety with NLS is 95% to 100% identical in sequence to SEQ ID NO: 29. In certain embodiments, the functionally active Flp moiety with NLS is 96% to 100% identical in sequence to SEQ ID NO: 29. In certain embodiments, the functionally active Flp moiety with NLS is 97% to 100% identical in sequence to SEQ ID NO: 29. In certain embodiments, the functionally active Flp moiety with NLS is 98% to 100% identical in sequence SEQ ID NO: 29. In certain embodiments, the functionally active Flp moiety with NLS is 99% to 100% identical in sequence to SEQ ID NO: 29. In certain embodiments, the functionally active Flp moiety with NLS is 100% identical in sequence to SEQ ID NO: 29.

In some embodiments, the functionally active Cre moiety has a sequence at least 95% identical to SEQ ID NO: 27 and is encoded by a nucleic acid sequence at least 95% identical to SEQ ID NO: 26; the functionally Flp protein moiety has a sequence at least 95% identical to SEQ ID NO: 29 and is encoded by a nucleic acid sequence at least 95% identical to SEQ ID NO: 28; or both. In some embodiments, the functionally active Cre moiety is encoded by a nucleic acid sequence corresponding to SEQ ID NO: 26; the functionally active Flp moiety is encoded by a nucleic acid sequence corresponding to SEQ ID NO: 28; or both.

In some embodiments, the functionally active Cre moiety has a sequence at least 95% identical to SEQ ID NO: 27; the functionally active Flp moiety has a sequence at least 95% identical to SEQ ID NO: 29; or both. In some embodiments, the functionally active Cre moiety has a sequence corresponding to SEQ ID NO: 27; the functionally active Flp moiety has a sequence corresponding to SEQ ID NO: 29; or both.

In certain embodiments, the first agent has a sequence at least 80% identical to SEQ ID NO: 78 and the second agent has a sequence at least 80% identical to SEQ ID NO: 79 In certain embodiments, the first agent has a sequence at least 85% identical to SEQ ID NO: 78 and the second agent has a sequence at least 85% identical to SEQ ID NO: 79. In certain embodiments, the first agent has a sequence at least 90% identical to SEQ ID NO: 78 and the second agent has a sequence at least 90% identical to SEQ ID NO: 79. In certain embodiments, the first agent has a sequence at least 95% identical to SEQ ID NO: 78 and the second agent has a sequence at least 95% identical to SEQ ID NO: 79. In certain embodiments, the first agent has a sequence at least 96% identical to SEQ ID NO: 78 and the second agent has a sequence at least 96% identical to SEQ ID NO: 79. In certain embodiments, the first agent has a sequence at least 97% identical to SEQ ID NO: 78 and the second agent has a sequence at least 97% identical to SEQ ID NO: 79. In certain embodiments, the first agent has a sequence at least 98% identical to SEQ ID NO: 78 and the second agent has a sequence at least 98% identical to SEQ ID NO: 79. In certain embodiments, the first agent has a sequence at least 99% identical to SEQ ID NO: 78 and the second agent has a sequence at least 99% identical to SEQ ID NO: 79. In certain embodiments, the first agent has a sequence 100% identical to SEQ ID NO: 78 and the second agent has a sequence 100% identical to SEQ ID NO: 79. In certain embodiments, the first agent has a sequence comprising SEQ ID NO: 78 and the second agent has a sequence comprising SEQ ID NO: 79.

In some embodiments, the LoxP site is at least 80% identical to SEQ ID NO: 1. In some embodiments, the LoxP site is at least 85% identical to SEQ ID NO: 1. In some embodiments, the LoxP site is at least 90% identical to SEQ ID NO: 1. In some embodiments, the LoxP site is at least 95% identical to SEQ ID NO: 1. In some embodiments, the LoxP site is at least 96% identical to SEQ ID NO: 1. In some embodiments, the LoxP site is at least 97% identical to SEQ ID NO: 1. In some embodiments, the LoxP site is at least 98% identical to SEQ ID NO: 1. In some embodiments, the LoxP site is at least 99% identical to SEQ ID NO: 1. In some embodiments, the LoxP site is at least 100% identical to SEQ ID NO: 1. In some embodiments, the LoxP site has a sequence comprising SEQ ID NO: 1.

In some embodiments, the FRT site is at least 80% identical to SEQ ID NO: 30. In some embodiments, the FRT site is at least 85% identical to SEQ ID NO: 30. In some embodiments, the FRT site is at least 90% identical to SEQ ID NO: 30. In some embodiments, the FRT site is at least 95% identical to SEQ ID NO: 30. In some embodiments, the FRT site is at least 96% identical to SEQ ID NO: 30. In some embodiments, the FRT site is at least 97% identical to SEQ ID NO: 30. In some embodiments, the FRT site is at least 98% identical to SEQ ID NO: 30. In some embodiments, the FRT site is at least 99% identical to SEQ ID NO: 30. In some embodiments, the FRT site is at least 100% identical to SEQ ID NO: 30. In some embodiments, the FRT site has a sequence comprising SEQ ID NO: 30.

In some embodiments, the sequence for directing the first nucleic acid sequence, to the chromosome of interest of the first PGC comprises a first 5′ HA nucleotide sequence that is substantially homologous to the 5′ region flanking a target gene locus in the chromosome of interest of the first PGC; and a first 3′ HA nucleotide sequence that is substantially homologous to the 3′ region flanking the target gene locus in the chromosome of interest of the first PGC (i.e., Breed A).

In some embodiments, the sequence for directing the second nucleic acid sequence, to the chromosome of interest of the second PGC comprises a second 5′ HA nucleotide sequence that is substantially homologous to the 5′ region flanking a target gene locus in the chromosome of interest of the second PGC; and a second 3′ HA nucleotide sequence that is substantially homologous to the 3′ region flanking the target gene locus in the chromosome of interest of the second PGC (i.e., Breed B).

In another aspect, provided herein is a genomic modifying vector pair comprising: (a) a first vector comprising a first promoter operably linked to a first exogenous polynucleotide encoding a first nuclear localization signal (NLS), a functionally inactive first protein moiety of interest, and a first intein moiety; and (b) a second vector comprising a second promoter operably linked to a second exogenous polynucleotide encoding a second nuclear localization signal (NLS), a functionally inactive second protein moiety of interest, and a second intein moiety, wherein: (i) the functionally inactive first protein moiety of interest and the functionally inactive second protein moiety of interest, dimerized or bound covalently, comprise at least one functionally active protein of interest or fragment thereof, wherein: (a) the functionally active protein of interest or fragment thereof comprises a genomic modifier, the genomic modifier targeting a gene of interest (GOI) or fragment thereof on a chromosome, the GOI modified to introduce one or more target sites specific to the genomic modifier, and the GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active gene of interest (GOI), the modified trait inducing sterility or inhibiting fertility in the functionally modified and genetically modified progeny avian embryo or progeny avian without impairing viability; (b) the functionally active protein of interest or fragment thereof comprises a toxin inducing cell death of a PGC, either without impairing viability of somatic cells or limited to benign somatic cell loss or both, in the functionally modified and genetically modified progeny avian embryo or progeny avian, and inducing sterility or inhibiting fertility in the functionally modified and genetically modified progeny avian embryo or progeny avian without impairing viability; or (c) a combination thereof, (ii) the functionally inactive first protein moiety of interest and the functionally inactive second protein moiety of interest, when co-expressed, comprise at least one functionally active protein of interest or fragment thereof, the co-expression of which inducing cell death of a PGC without impairing viability of somatic cells in the functionally modified and genetically modified progeny avian embryo or progeny avian and inducing sterility or inhibiting fertility in the functionally modified and genetically modified avian without impairing viability; or (c) a combination of any of the above.

In some embodiments, the genomic modifier comprises a recombinase. In some embodiments, (a) the target site comprises a Lox site; and (b) the first exogenous polynucleotide encodes a protein comprising SEQ ID NO: 4, SEQ ID NO: 6, and SEQ ID NO: 8 and the second exogenous polynucleotide encodes a protein comprising SEQ ID NO: 4, SEQ ID NO: 12, and SEQ ID NO: 10; the first exogenous polynucleotide encodes a protein comprising SEQ ID NO: 70, SEQ ID NO: 71, and SEQ ID NO: 72, and the second exogenous polynucleotide encodes a protein comprising SEQ ID NO: 70, SEQ ID NO: 73, and SEQ ID NO: 74; or the first exogenous polynucleotide encodes a protein comprising SEQ ID NO: 78 and the second exogenous polynucleotide encodes a protein comprising SEQ ID NO: 79.

A person of skill in the art would understand that the term “DNA editing agent” generally refers to any molecule, such as a nucleotide sequence or an enzyme, which promotes a change in a genome of an organism, such as a bird. The change may be an addition to the DNA, e.g., by the agent being integrated to the DNA, a replacement of a sequence of the DNA, e.g., by homological recombination, or a deletion of the DNA.

In one embodiment, the DNA editing agent may be constructed in a viral vector (e.g., using a single vector or multiple vectors). Such vectors are commonly used in gene transfer and gene therapy applications. Different viral vector systems have their own unique advantages and disadvantages. Viral vectors that may be used to integrate the first nucleotide sequence of certain embodiments into the chromosome of interest of a bird include, but are not limited to, adenoviral vectors, adeno-associated viral vectors, alphavirus vectors, herpes simplex viral vectors, retroviral vectors, or lentiviral vectors.

A viral construct such as a retroviral construct includes at least one transcriptional promoter/enhancer or locus-defining element(s), or other elements that control gene expression by means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. In certain embodiments, the signal sequence can be a mammalian signal sequence. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, or dendrimers.

The DNA editing agent may encode a reporter protein that is readily detectable either by its presence or activity, including, but not limited to, luciferase, fluorescent protein (e.g., green fluorescent protein), chloramphenicol acetyl transferase, beta-galactosidase, secreted placental alkaline phosphatase, beta-lactamase, human growth hormone, and other secreted enzyme reporters. Generally, a reporter gene encodes a polypeptide not otherwise produced by the host cell, which is detectable by analysis of the cell(s), e.g., by the direct fluorometric, radioisotopic or spectrophotometric analysis of the cell(s) and typically without the need to kill the cells for signal analysis. In certain embodiments, a reporter gene encodes an enzyme, which produces a change in fluorometric properties of the host cell, which is detectable by qualitative, quantitative, or semi-quantitative function or transcriptional activation. Exemplary enzymes include esterases, β-lactamase, phosphatases, peroxidases, proteases (tissue plasminogen activator or urokinase) and other enzymes whose function can be detected by appropriate chromogenic or fluorogenic substrates known to those skilled in the art or developed in the future. The reporter gene may report on successful integration of the construct into the chromosome of interest.

In certain embodiments, the DNA editing agent may comprise a nucleotide sequence that encodes a detectable marker (e.g., a reporter polypeptide). In some embodiments, the marker comprises a fluorescent protein, a luminescent protein, or a chromoprotein.

In certain embodiments, the DNA editing agent further comprises a positive or a negative selection marker, or a combination thereof, for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations or elimination of a marker sequence (e.g. positive marker), or combinations thereof. Non-limiting examples of such negative markers include the herpes simplex-thymidine kinase (HSV-TK) which converts ganciclovir (GCV) into a cytotoxic nucleoside analog, hypoxanthine phosphoribosyltransferase (HIPRT), Diphtheria toxin (DT) and adenine phosphoribosyltransferase (ARPT).

In certain embodiments, the codons encoding the proteins of the DNA editing agent are “optimized” codons, i.e., the codons are those that appear frequently in, e.g., highly expressed genes in the bird species, instead of those codons that are frequently used by, for example, an influenza virus. Such codon usage provides for efficient expression of the protein in avian cells. Codon usage patterns are known in the literature for highly expressed genes of many species (e.g., Nakamura et al., 1996, Nucleic Acids Res. 24(1):214-5; McEwan et al., 1998, Biotechniques 24(1):131-6, 138).

In certain embodiments, the DNA editing agent may further include self-cleaving peptides such as the 2A, including but not limited to P2A, T2A, E2A (Wang et al., Scientific Report 5, Article 16273 (2015)), or internal ribosome entry site (IRES) sequences.

In some embodiments, the at least one self-cleaving peptide comprises a P2A peptide, a T2A peptide, or both.

In some embodiments, the P2A peptide is encoded by SEQ ID NO: 13 or SEQ ID NO: 53. In some embodiments, the P2A peptide has a sequence at least 80% identical to SEQ ID NO: 14 or SEQ ID NO: 75. In some embodiments, the P2A peptide has a sequence at least 85% identical to SEQ ID NO: 14 or SEQ ID NO: 75. In some embodiments, the P2A peptide has a sequence at least 90% identical to SEQ ID NO: 14 or SEQ ID NO: 75.

In some embodiments, the P2A peptide has a sequence at least 95% identical to SEQ ID NO: 14 or SEQ ID NO: 75. In some embodiments, the P2A peptide has a sequence at least 96% identical to SEQ ID NO: 14 or SEQ ID NO: 75. In some embodiments, the P2A peptide has a sequence at least 97% identical to SEQ ID NO: 14 or SEQ ID NO: 75. In some embodiments, the P2A peptide has a sequence at least 98% identical to SEQ ID NO: 14 or SEQ ID NO: 75. In some embodiments, the P2A peptide has a sequence at least 99% identical to SEQ ID NO: 14 or SEQ ID NO: 75. In some embodiments, the P2A peptide has a sequence at least 100% identical to SEQ ID NO: 14 or SEQ ID NO: 75. In some embodiments, the P2A peptide has a sequence comprising SEQ ID NO: 14 or SEQ ID NO: 75.

In some embodiments, the T2A peptide is encoded by SEQ ID NO: 17 or SEQ ID NO: 54. In some embodiments, the T2A peptide has a sequence at least 80% identical to SEQ ID NO: 18 or SEQ ID NO: 76. In some embodiments, the T2A peptide has a sequence at least 85% identical to SEQ ID NO: 18 or SEQ ID NO: 76. In some embodiments, the T2A peptide has a sequence at least 90% identical to SEQ ID NO: 18 or SEQ ID NO: 76.

In some embodiments, the T2A peptide has a sequence at least 95% identical to SEQ ID NO: 18 or SEQ ID NO: 76. In some embodiments, the T2A peptide has a sequence at least 96% identical to SEQ ID NO: 18 or SEQ ID NO: 76. In some embodiments, the T2A peptide has a sequence at least 97% identical to SEQ ID NO: 18 or SEQ ID NO: 76. In some embodiments, the T2A peptide has a sequence at least 98% identical to SEQ ID NO: 18 or SEQ ID NO: 76. In some embodiments, the T2A peptide has a sequence at least 99% identical to SEQ ID NO: 18 or SEQ ID NO: 76. In some embodiments, the T2A peptide has a sequence at least 100% identical to SEQ ID NO: 18 or SEQ ID NO: 76. In some embodiments, the T2A peptide has a sequence comprising SEQ ID NO: 18 or SEQ ID NO: 76.

### 5′ and 3′ Homology Arms

In some embodiments, the first exogenous polynucleotide further comprises a first 5′ homology arm (HA) (first left homology arm [LHA]) and a first 3′ homology arm (HA) (first right homology arm [RHA]), said first 5′ HA and said first 3′ HA specific for a first insertion site of interest on the avian genome and wherein the second exogenous polynucleotide further comprises a second 5′ homology arm (HA) (second left homology arm [LHA]) and a second 3′ homology arm (HA) (second right homology arm [RHA]), said second 5′ HA and said second 3′ HA specific for a second insertion site of interest on the avian genome, wherein the first 5′ HA has a nucleotide sequence that is substantially homologous to the 5′ region flanking a first gene of interest (GOI) in a first chromosome of interest and the first 3′ HA has a nucleotide sequence that is substantially homologous to the 3′ region flanking the first GOI in the first chromosome of interest; the second 5′ HA has a nucleotide sequence that is substantially homologous to the 5′ region flanking a second GOI in a second chromosome of interest and the second 3′ HA, or both has a nucleotide sequence that is substantially homologous to the 3′ region flanking the second GOI in the second chromosome of interest; or both. In some embodiments, the first GOI and the second GOI are the same GOI and the first chromosome of interest and the second chromosome of interest are the same chromosome of interest.

In some embodiments, the gene of interest (GOI) or a fragment thereof comprises the Deleted in Azoospermia-Like (DAZL) gene or a fragment thereof and the first 5′ HA, the second 5′ HA, or both has a sequence at least 95% identical to SEQ ID NO: 23 or SEQ ID NO: 46, and the first 3′ HA, the second 3′ HA, or both has a sequence at least 95% identical to SEQ ID NO: 24 or SEQ ID NO: 47. In some embodiments, the first 5′ HA, the second 5′ HA, or both has a sequence at least 96% identical to SEQ ID NO: 23 or SEQ ID NO: 46, and the first 3′ HA, the second 3′ HA, or both has a sequence at least 96% identical to SEQ ID NO: 24 or SEQ ID NO: 47. In some embodiments, the first 5′ HA, the second 5′ HA, or both has a sequence at least 97% identical to SEQ ID NO: 23 or SEQ ID NO: 46, and the first 3′ HA, the second 3′ HA, or both has a sequence at least 97% identical to SEQ ID NO: 24 or SEQ ID NO: 47. In some embodiments, the first 5′ HA, the second 5′ HA, or both has a sequence at least 98% identical to SEQ ID NO: 23 or SEQ ID NO: 46, and the first 3′ HA, the second 3′ HA, or both has a sequence at least 98% identical to SEQ ID NO: 24 or SEQ ID NO: 47. In some embodiments, the first 5′ HA, the second 5′ HA, or both has a sequence at least 99% identical to SEQ ID NO: 23 or SEQ ID NO: 46, and the first 3′ HA, the second 3′ HA, or both has a sequence at least 99% identical to SEQ ID NO: 24 or SEQ ID NO: 47. In some embodiments, the first 5′ HA, the second 5′ HA, or both has a sequence 100% identical to SEQ ID NO: 23 or SEQ ID NO: 46, and the first 3′ HA, the second 3′ HA, or both has a sequence 100% identical to SEQ ID NO: 24 or SEQ ID NO: 47. In some embodiments, the gene of interest (GOI) or a fragment thereof comprises the Deleted in Azoospermia-Like (DAZL) gene or a fragment thereof and the first 5′ HA, the second 5′ HA, or both comprises the sequence of SEQ ID NO: 23 or SEQ ID NO: 46, and the first 3′ HA, the second 3′ HA, or both comprises the sequence of SEQ ID NO: 24 or SEQ ID NO: 47.

In certain embodiments, (i) the length of the first 5′ HA is about 0.5 to about 5 kilobases (kb); (ii) the length of the first 3′ HA is about 0.5 to about 5 kb; (iii) the length of the second 5′ HA is about 0.5 to about 5 kb; (iv) the length of the second 3′ HA is about 0.5 to about 5 kb; or (v) any combination of (i), (ii), (iii), and/or (iv). In certain embodiments, (i) the length of the first 5′ HA is about 1.5 kb; (ii) the length of the first 3′ HA is about 1.5 kb; (iii) the length of the second 5′ HA is about 1.5 kb; (iv) the length of the second 3′ HA is about 1.5 kb; or (v) any combination of (i), (ii), (iii), and/or (iv).

In certain embodiments, the length of the first 5′ HA and/or the second 5′ HA is about 0.5 to about 5 kilobases (kb). In certain embodiments, the length of the first 5′ HA and/or the second 5′ HA is about 0.5 kb. In certain embodiments, the length of the first 5′ HA and/or the second 5′ HA is about 1 kb. In certain embodiments, the length of the first 5′ HA and/or the second 5′ HA is about 1.5 kb. In certain embodiments, the length of the first 5′ HA and/or the second 5′ HA is about 2 kb. In certain embodiments, the length of the first 5′ HA and/or the second 5′ HA is about 2.5 kb. In certain embodiments, the length of the first 5′ HA and/or the second 5′ HA is about 3 kb. In certain embodiments, the length of the first 5′ HA and/or the second 5′ HA is about 3.5 kb. In certain embodiments, the length of the first 5′ HA and/or the second 5′ HA is about 4 kb. In certain embodiments, the length of the first 5′ HA and/or the second 5′ HA is about 4.5 kb. In certain embodiments, the length of the first 5′ HA and/or the second 5′ HA is about 5 kb.

In certain embodiments, the length of the first 3′ HA and/or the second 3′ HA is about 0.5 to about 5 kilobases (kb). In certain embodiments, the length of the first 3′ HA and/or the second 3′ HA is about 0.5 kb. In certain embodiments, the length of the first 3′ HA and/or the second 3′ HA is about 1 kb. In certain embodiments, the length of the first 3′ HA and/or the second 3′ HA is about 1.5 kb. In certain embodiments, the length of the first 3′ HA and/or the second 3′ HA is about 2 kb. In certain embodiments, the length of the first 3′ HA and/or the second 3′ HA is about 2.5 kb. In certain embodiments, the length of the first 3′ HA and/or the second 3′ HA is about 3 kb. In certain embodiments, the length of the first 3′ HA and/or the second 3′ HA is about 3.5 kb. In certain embodiments, the length of the first 3′ HA and/or the second 3′ HA is about 4 kb. In certain embodiments, the length of the first 3′ HA and/or the second 3′ HA is about 4.5 kb. In certain embodiments, the length of the first 3′ HA and/or the second 3′ HA is about 5 kb.

In certain embodiments, the length of each of the first 5′ HA and first 3′ HA is about 0.5 kb. In certain embodiments, the length of each of the first 5′ HA and first 3′ HA is about 1 kb. In certain embodiments, the length of each of the first 5′ HA and first 3′ HA is about 1.5 kb. In certain embodiments, the length of each of the first 5′ HA and first 3′ HA is about 2 kb. In certain embodiments, the length of each of the first 5′ HA and first 3′ HA is about 2.5 kb. In certain embodiments, the length of each of the first 5′ HA and first 3′ HA is about 3 kb. In certain embodiments, the length of each of the first 5′ HA and first 3′ HA is about 3.5 kb. In certain embodiments, the length of each of the first 5′ HA and first 3′ HA is about 4 kb. In certain embodiments, the length of each of the first 5′ HA and first 3′ HA is about 4.5 kb. In certain embodiments, the length of each of the first 5′ HA and first 3′ HA is about 5 kb.

In certain embodiments, the length of each of the second 5′ HA and second 3′ HA is about 0.5 kb. In certain embodiments, the length of each of the second 5′ HA and second 3′ HA is about 1 kb. In certain embodiments, the length of each of the second 5′ HA and second 3′ HA is about 1.5 kb. In certain embodiments, the length of each of the second 5′ HA and second 3′ HA is about 2 kb. In certain embodiments, the length of each of the second 5′ HA and second 3′ HA is about 2.5 kb. In certain embodiments, the length of each of the second 5′ HA and second 3′ HA is about 3 kb. In certain embodiments, the length of each of the second 5′ HA and second 3′ HA is about 3.5 kb. In certain embodiments, the length of each of the second 5′ HA and second 3′ HA is about 4 kb. In certain embodiments, the length of each of the second 5′ HA and second 3′ HA is about 4.5 kb. In certain embodiments, the length of each of the second 5′ HA and second 3′ HA is about 5 kb.

In certain embodiments, the length of each of the left and the right homology arms is sufficient to allow specific recombination into chromosomal DNA of a bird. In one embodiment, the first 5′ HA, the first 3′ HA, the second 5′ HA or the second 3′ HA or any combination of one or more thereof, are at least 500 nucleotides long, for example, between 500-3000 nucleotides long. Typically, the required size of the first 5′ HA and/or the first 3′ HA, or the second 5′ HA and/or the second 3′ HA or any combination of any or all of these relies on the length of the cassettes which are flanked by these arms. Smaller cassettes require shorter arms and vice versa.

In certain embodiments, (i) the first 5′ HA is substantially homologous to a corresponding first nucleotide sequence located in an openly transcribed region on the chromosome of interest of a bird; (ii) the first 3′ HA is substantially homologous to a corresponding second nucleotide sequence located in an openly transcribed region on the chromosome of interest of a bird; or (iii) both (i) and (ii). In certain embodiments, (i) the second 5′ HA is substantially homologous to a corresponding first nucleotide sequence located in an openly transcribed region on the chromosome of interest of a bird; (ii) the second 3′ HA is substantially homologous to a corresponding second nucleotide sequence located in an openly transcribed region on the chromosome of interest of a bird; or (iii) both (i) and (ii).

As it would be apparent to those skilled in the art, a first sequence is “substantially homologous” to a second sequence if the first sequence and the second sequence are similar or identical in sequence, as long as the first sequence and the second sequence can replace one another by homologous recombination. Method to test and identify homologous recombination are well-known in the art.

In certain embodiments, substantially homologous is at least 50% identical. In certain embodiments, substantially homologous is at least 60% identical. In certain embodiments, substantially homologous is at least 70% identical. In certain embodiments, substantially homologous is at least 80% identical. In certain embodiments, substantially homologous is at least 90% identical. In certain embodiments, substantially homologous is at least 95% identical. In certain embodiments, substantially homologous is at least 96% identical. In certain embodiments, substantially homologous is at least 97% identical. In certain embodiments, substantially homologous is at least 98% identical. In certain embodiments, substantially homologous is at least 99% identical.

In certain embodiments, the first 5′ HA nucleotide sequence is 50% to 100% identical in sequence to a first corresponding nucleotide sequence on the chromosome of interest. In certain embodiments, the first 5′ HA nucleotide sequence is 80% to 100% identical in sequence to a first corresponding nucleotide sequence on the chromosome of interest. In certain embodiments, the first 5′ HA nucleotide sequence is 85% to 100% identical in sequence to a first corresponding nucleotide sequence on the chromosome of interest. In certain embodiments, the first 5′ HA nucleotide sequence is 90% to 100% identical in sequence to a first corresponding nucleotide sequence on the chromosome of interest. In certain embodiments, the first 5′ HA nucleotide sequence is 95% to 100% identical in sequence to a first corresponding nucleotide sequence on the chromosome of interest. In certain embodiments, the first 5′ HA nucleotide sequence is 96% to 100% identical in sequence to a first corresponding nucleotide sequence on the chromosome of interest. In certain embodiments, the first 5′ HA nucleotide sequence is 97% to 100% identical in sequence to a first corresponding nucleotide sequence on the chromosome of interest. In certain embodiments, the first 5′ HA nucleotide sequence is 98% to 100% identical in sequence to a first corresponding nucleotide sequence on the chromosome of interest. In certain embodiments, the first 5′ HA nucleotide sequence is 99% to 100% identical in sequence to a first corresponding nucleotide sequence on the chromosome of interest. In certain embodiments, the first 5′ HA nucleotide sequence is 100% identical in sequence to a first corresponding nucleotide sequence on the chromosome of interest.

In certain embodiments, the first 3′ HA nucleotide sequence is 50% to 100% identical in sequence to a second corresponding nucleotide sequence on the chromosome of interest. In certain embodiments, the first 3′ HA nucleotide sequence is 80% to 100% identical in sequence to a second corresponding nucleotide sequence on the chromosome of interest. In certain embodiments, the first 3′ HA nucleotide sequence is 85% to 100% identical in sequence to a second corresponding nucleotide sequence on the chromosome of interest. In certain embodiments, the first 3′ HA nucleotide sequence is 90% to 100% identical in sequence to a second corresponding nucleotide sequence on the chromosome of interest. In certain embodiments, the first 3′ HA nucleotide sequence is 95% to 100% identical in sequence to a second corresponding nucleotide sequence on the chromosome of interest. In certain embodiments, the first 3′ HA nucleotide sequence is 96% to 100% identical in sequence to a second corresponding nucleotide sequence on the chromosome of interest. In certain embodiments, the first 3′ HA nucleotide sequence is 97% to 100% identical in sequence to a second corresponding nucleotide sequence on the chromosome of interest. In certain embodiments, the first 3′ HA nucleotide sequence is 98% to 100% identical in sequence to a second corresponding nucleotide sequence on the chromosome of interest. In certain embodiments, the first 3′ HA nucleotide sequence is 99% to 100% identical in sequence to a second corresponding nucleotide sequence on the chromosome of interest. In certain embodiments, the first 3′ HA nucleotide sequence is 100% identical in sequence to a second corresponding nucleotide sequence on the chromosome of interest.

In certain embodiments, the second 5′ HA nucleotide sequence is 50% to 100% identical in sequence to a third corresponding nucleotide sequence on the chromosome of interest. In certain embodiments, the second 5′ HA nucleotide sequence is 80% to 100% identical in sequence to a third corresponding nucleotide sequence on the chromosome of interest. In certain embodiments, the second 5′ HA nucleotide sequence is 85% to 100% identical in sequence to a third corresponding nucleotide sequence on the chromosome of interest. In certain embodiments, the second 5′ HA nucleotide sequence is 90% to 100% identical in sequence to a third corresponding nucleotide sequence on the chromosome of interest. In certain embodiments, the second 5′ HA nucleotide sequence is 95% to 100% identical in sequence to a third corresponding nucleotide sequence on the chromosome of interest. In certain embodiments, the second 5′ HA nucleotide sequence is 96% to 100% identical in sequence to a third corresponding nucleotide sequence on the chromosome of interest. In certain embodiments, the second 5′ HA nucleotide sequence is 97% to 100% identical in sequence to a third corresponding nucleotide sequence on the chromosome of interest. In certain embodiments, the second 5′ HA nucleotide sequence is 98% to 100% identical in sequence to a third corresponding nucleotide sequence on the chromosome of interest. In certain embodiments, the second 5′ HA nucleotide sequence is 99% to 100% identical in sequence to a third corresponding nucleotide sequence on the chromosome of interest. In certain embodiments, the second 5′ HA nucleotide sequence is 100% identical in sequence to a third corresponding nucleotide sequence on the chromosome of interest.

In certain embodiments, the second 3′ HA nucleotide sequence is 50% to 100% identical in sequence to a fourth corresponding nucleotide sequence on the chromosome of interest. In certain embodiments, the second 3′ HA nucleotide sequence is 80% to 100% identical in sequence to a fourth corresponding nucleotide sequence on the chromosome of interest. In certain embodiments, the second 3′ HA nucleotide sequence is 85% to 100% identical in sequence to a fourth corresponding nucleotide sequence on the chromosome of interest. In certain embodiments, the second 3′ HA nucleotide sequence is 90% to 100% identical in sequence to a fourth corresponding nucleotide sequence on the chromosome of interest. In certain embodiments, the second 3′ HA nucleotide sequence is 95% to 100% identical in sequence to a fourth corresponding nucleotide sequence on the chromosome of interest. In certain embodiments, the second 3′ HA nucleotide sequence is 96% to 100% identical in sequence to a fourth corresponding nucleotide sequence on the chromosome of interest. In certain embodiments, the second 3′ HA nucleotide sequence is 97% to 100% identical in sequence to a fourth corresponding nucleotide sequence on the chromosome of interest. In certain embodiments, the second 3′ HA nucleotide sequence is 98% to 100% identical in sequence to a fourth corresponding nucleotide sequence on the chromosome of interest. In certain embodiments, the second 3′ HA nucleotide sequence is 99% to 100% identical in sequence to a fourth corresponding nucleotide sequence on the chromosome of interest. In certain embodiments, the second 3′ HA nucleotide sequence is 100% identical in sequence to a fourth corresponding nucleotide sequence on the chromosome of interest.

In certain embodiments, the first 5′ HA nucleotide sequence is 50% to 100% complementary in sequence to a first corresponding nucleotide sequence on the chromosome of interest. In certain embodiments, the first 5′ HA nucleotide sequence is 80% to 100% complementary in sequence to a first corresponding nucleotide sequence on the chromosome of interest. In certain embodiments, the first 5′ HA nucleotide sequence is 85% to 100% complementary in sequence to a first corresponding nucleotide sequence on the chromosome of interest. In certain embodiments, the first 5′ HA nucleotide sequence is 90% to 100% complementary in sequence to a first corresponding nucleotide sequence on the chromosome of interest. In certain embodiments, the first 5′ HA nucleotide sequence is 95% to 100% complementary in sequence to a first corresponding nucleotide sequence on the chromosome of interest. In certain embodiments, the first 5′ HA nucleotide sequence is 96% to 100% complementary in sequence to a first corresponding nucleotide sequence on the chromosome of interest. In certain embodiments, the first 5′ HA nucleotide sequence is 97% to 100% complementary in sequence to a first corresponding nucleotide sequence on the chromosome of interest. In certain embodiments, the first 5′ HA nucleotide sequence is 98% to 100% complementary in sequence to a first corresponding nucleotide sequence on the chromosome of interest. In certain embodiments, the first 5′ HA nucleotide sequence is 99% to 100% complementary in sequence to a first corresponding nucleotide sequence on the chromosome of interest. In certain embodiments, the first 5′ HA nucleotide sequence is 100% complementary in sequence to a first corresponding nucleotide sequence on the chromosome of interest.

In certain embodiments, the first 3′ HA nucleotide sequence is 50% to 100% complementary in sequence to a second corresponding nucleotide sequence on the chromosome of interest. In certain embodiments, the first 3′ HA nucleotide sequence is 80% to 100% complementary in sequence to a second corresponding nucleotide sequence on the chromosome of interest. In certain embodiments, the first 3′ HA nucleotide sequence is 85% to 100% complementary in sequence to a second corresponding nucleotide sequence on the chromosome of interest. In certain embodiments, the first 3′ HA nucleotide sequence is 90% to 100% complementary in sequence to a second corresponding nucleotide sequence on the chromosome of interest. In certain embodiments, the first 3′ HA nucleotide sequence is 95% to 100% complementary in sequence to a second corresponding nucleotide sequence on the chromosome of interest. In certain embodiments, the first 3′ HA nucleotide sequence is 96% to 100% complementary in sequence to a second corresponding nucleotide sequence on the chromosome of interest. In certain embodiments, the first 3′ HA nucleotide sequence is 97% to 100% complementary in sequence to a second corresponding nucleotide sequence on the chromosome of interest. In certain embodiments, the first 3′ HA nucleotide sequence is 98% to 100% complementary in sequence to a second corresponding nucleotide sequence on the chromosome of interest. In certain embodiments, the first 3′ HA nucleotide sequence is 99% to 100% complementary in sequence to a second corresponding nucleotide sequence on the chromosome of interest. In certain embodiments, the first 3′ HA nucleotide sequence is 100% complementary in sequence to a second corresponding nucleotide sequence on the chromosome of interest.

In certain embodiments, the second 5′ HA nucleotide sequence is 50% to 100% complementary in sequence to a third corresponding nucleotide sequence on the chromosome of interest. In certain embodiments, the second 5′ HA nucleotide sequence is 80% to 100% complementary in sequence to a third corresponding nucleotide sequence on the chromosome of interest. In certain embodiments, the second 5′ HA nucleotide sequence is 85% to 100% complementary in sequence to a third corresponding nucleotide sequence on the chromosome of interest. In certain embodiments, the second 5′ HA nucleotide sequence is 90% to 100% complementary in sequence to a third corresponding nucleotide sequence on the chromosome of interest. In certain embodiments, the second 5′ HA nucleotide sequence is 95% to 100% complementary in sequence to a third corresponding nucleotide sequence on the chromosome of interest. In certain embodiments, the second 5′ HA nucleotide sequence is 96% to 100% complementary in sequence to a third corresponding nucleotide sequence on the chromosome of interest. In certain embodiments, the second 5′ HA nucleotide sequence is 97% to 100% complementary in sequence to a third corresponding nucleotide sequence on the chromosome of interest. In certain embodiments, the second 5′ HA nucleotide sequence is 98% to 100% complementary in sequence to a third corresponding nucleotide sequence on the chromosome of interest. In certain embodiments, the second 5′ HA nucleotide sequence is 99% to 100% complementary in sequence to a third corresponding nucleotide sequence on the chromosome of interest. In certain embodiments, the second 5′ HA nucleotide sequence is 100% complementary in sequence to a third corresponding nucleotide sequence on the chromosome of interest.

In certain embodiments, the second 3′ HA nucleotide sequence is 50% to 100% complementary in sequence to a fourth corresponding nucleotide sequence on the chromosome of interest. In certain embodiments, the second 3′ HA nucleotide sequence is 80% to 100% complementary in sequence to a fourth corresponding nucleotide sequence on the chromosome of interest. In certain embodiments, the second 3′ HA nucleotide sequence is 85% to 100% complementary in sequence to a fourth corresponding nucleotide sequence on the chromosome of interest. In certain embodiments, the second 3′ HA nucleotide sequence is 90% to 100% complementary in sequence to a fourth corresponding nucleotide sequence on the chromosome of interest. In certain embodiments, the second 3′ HA nucleotide sequence is 95% to 100% complementary in sequence to a fourth corresponding nucleotide sequence on the chromosome of interest. In certain embodiments, the second 3′ HA nucleotide sequence is 96% to 100% complementary in sequence to a fourth corresponding nucleotide sequence on the chromosome of interest. In certain embodiments, the second 3′ HA nucleotide sequence is 97% to 100% complementary in sequence to a fourth corresponding nucleotide sequence on the chromosome of interest. In certain embodiments, the second 3′ HA nucleotide sequence is 98% to 100% complementary in sequence to a fourth corresponding nucleotide sequence on the chromosome of interest. In certain embodiments, the second 3′ HA nucleotide sequence is 99% to 100% complementary in sequence to a fourth corresponding nucleotide sequence on the chromosome of interest. In certain embodiments, the second 3′ HA nucleotide sequence is 100% complementary in sequence to a fourth corresponding nucleotide sequence on the chromosome of interest.

In certain embodiments, the first 5′ HA or the first 3′ HA or both, are homologous or show homology or identity of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to at least one nucleotide sequence within a target loci within the chromosome of interest of a bird that serves as the integration site.

In certain embodiments, the second 5′ HA or the second 3′ HA or both, are homologous or show homology or identity of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% to at least one nucleotide sequence within a target loci within the chromosome of interest of a bird that serves as the integration site.

A person of skill in the art would understand that the term “openly transcribed region in a chromosome” generally refers to regions of the chromosome which include genes that are transcribed in a level sufficient to allow other genes to be easily transcribed as well.

Non-limiting examples of openly transcribed regions are regions in proximity to house-keeping genes which are highly transcribed during the life of the cell or the organism. Other non-limiting examples of openly transcribed regions are regions in-between loci (e.g., chromatin regulatory elements, non-coding DNA, “junk DNA”, etc.). Non-limiting examples of poorly transcribed regions are regions at the ends of each chromosomes, called telomers, which are not transcribed during the life of the cell or the organism.

In certain embodiments, the openly transcribed region is located on chromosome 2 of a bird. In certain embodiments, the openly transcribed region is located on chromosome 2 of Gallus gallus.

In one embodiment, the first 5′ HA or the first 3′ HA or both correspond to a genomic sequence which is present on chromosome 2 in birds. In one embodiment, the second 5′ HA or the second 3′ HA or both correspond to a genomic sequence which is present on chromosome 2 in birds.

The first 5′ HA, the first 3′ HA, the second 5′ HA, or the second 3′ HA targeting sequences or both, may be selected such that the first 5′ HA, the first 3′ HA, the second 5′ HA, or the second 3′ HA targeting sequence or both integrate specifically into the chromosome of interest and not any other chromosome of the cell, e.g. by spontaneous homologous recombination or by homology directed repair (HDR). Homologous recombination can occur spontaneously. Furthermore, the first 5′ HA, the first 3′ HA, the second 5′ HA, or the second 3′ HA targeting sequence or both may be selected depending on what method is being relied upon to integrate the first targeting sequence into the chromosome. Methods of integrating nucleotide sequences into chromosomes are well known in the art, including targeted homologous recombination, site specific recombinases and genome editing by engineered nucleases (see e.g. Menke D. Genesis (2013) 51:-618; Capecchi, Science (1989) 244:1288-1292; Santiago et al., Proc Natl Acad Sci USA (2008) 105:5809-5814; International Patent Application Nos. WO 2014/085593, WO 2009/071334 and WO 2011/146121; U.S. Pat. Nos. 8,771,945, 8,586,526, 6,774,279 and US Patent Application Publication Nos. 2003/0232410, 2005/0026157, 2006/0014264). PB transposases are also contemplated. Agents for introducing nucleic acid alterations to a gene of interest can be designed by publicly available resources.

In certain embodiments, the first 5′ nucleotide of 5′ HA (LHA) corresponds to chromosome 2, position 34439663. In certain embodiments, the first corresponding nucleotide sequence of 5′ HA (LHA) is located at chromosome 2, position 34439663 to position 34438203.

In certain embodiments, the first 5′ nucleotide of 5′ HA (LHA) corresponds to Gallus gallus chromosome 2, Assembly GRCg6a, NC_006089.5https://www.ncbi.nlm.nih.gov/nucleotide/NC_006089.5?report=genbank&lo g$=nuclalign&blast_rank=1&RID=HDGPY1G3014, position 34439663. In certain embodiments, the first corresponding nucleotide sequence of 5′ HA (LHA) is located at Gallus gallus chromosome 2, Assembly GRCg6a, NC_006089.5, position 34439663 to position 34438203.

In certain embodiments, the first 5′ nucleotide of 3′ HA (RHA) corresponds to chromosome 2, position 34437739. In certain embodiments, the second corresponding nucleotide sequence of 3′ HA (RHA) is located at chromosome 2, position 34437739 to position 34436209.

In certain embodiments, the first 5′ nucleotide of 3′ HA (RHA) corresponds to Gallus gallus chromosome 2, Assembly GRCg6a, NC_006089.5, position 34437739. In certain embodiments, the second corresponding nucleotide sequence of 3′ HA (RHA) is located at Gallus gallus chromosome 2, Assembly GRCg6a, NC_006089.5, position 34437739 to position 34436209.

### Detectable Markers

Genetic modification of the avian PGC chromosome may include a detectable marker that is detectable, e.g., in the PGC, in a genetically modified avian produced by the PGC, or in a PGC produced by the genetically modified avian produced by the PGC.

For example, in some embodiments, the first exogenous polynucleotide further encodes a first functionally inactive marker moiety operably linked to the first promoter, the first conjugating element, and the first protein moiety of interest and the second exogenous polynucleotide further encodes a second functionally inactive marker moiety operably linked to the second promoter, the second conjugating element, and the second protein moiety of interest, the first conjugating element conjugating to the second conjugating element to produce a functionally active marker or fragment thereof. In some embodiments, the first exogenous polynucleotide encodes a functionally active marker operably linked to the first protein moiety of interest; the second exogenous polynucleotide encodes a functionally active marker operably linked to the second protein moiety of interest; or a combination thereof. In some embodiments, the marker encoded by the first exogenous polynucleotide is distinct from the marker encoded by the second exogenous polynucleotide. In some embodiments, the marker is a fluorescent protein, a luminescent protein, or a chromoprotein.

In some embodiments, the detectable marker comprises a reporter polypeptide. Examples of a “detectable marker” include, but are not limited to, a fluorescent protein, a luminescent protein, a chromoprotein, an audible (vibrating) protein, a sonic protein, a metabolic marker, or a selective chelating protein. Examples of a fluorescent protein include, but are not limited to, green fluorescent protein (GFP), enhanced green fluorescent protein (EGF), Emerald, Superfolder GFP, Azami Green, mWasabi, TagGFP, TurboGFP, mNeonGreen, mUKG, acGFP, ZsGreen, Cloverm Sapphire, T-Sapphire, Enhanced blue fluorescent protein (EBFP), EBFP2, Azurite, TagBFP, mTagBFP, mKalama1, Cyan fluorescent protein (CFP), mCFP, Enhanced cyan fluorescent protein (ECFP), mECFP, Cerulean, SCFP3A, mTurquoise, mTurquoise2, CyPet, AmCyan1, Midori-Ishi Cyan, TagCFP, mTFP1 (Teal), Yellow fluorescent protein (YFP), Enhanced yellow fluorescent protein (EYFP), Super yellow fluorescent protein (SYFP), Topaz, Venus, Citrine, mCitrine, YPet, TagYFP, TurboYFP, PhiYFP, ZsYellow1, mBanana, Kusabira Orange, Kusabira Orange2, mOrange, mOrange2, dTomato, dTomato-Tandem, Red fluorescent protein (RFP), TurboRFP, TurboFP602, TurboFP635, Tag ref fluorescent protein (RFP), TagRFP-T, DsRed, DsRed2, DsRed-Express (T1), DsRed-Monomer, mTangerine, mKeima-Red, mRuby, mRuby2, mApple, mStrawberry, AsRed2, mRFP1, J-Red, mCherry, mKate (TagFP635), mKate2, HcRed1, mRaspberry, dKeima-Tandem, HcRed-Tandem, mPlum, mNeptune, NirFP, Sinus, TagFRP657, AQ143, Kaede, KikGR1, PX-CFP2, mEos2, IrisFP, meOS3.2, PSmOrange, PAGFP, Dronpa, Allowphycocyanin, GFPuv, R-phycoerythrin (RPE), Peridinin Chlorophyll (PerCP), P3, Katusha, B-phycoerythrin (BPE), and mKO, as well as derivatives and combinations thereof. In some embodiments, the marker is mCherry.

In some embodiments, the marker is a green fluorescent protein (GFP) encoded by SEQ ID NO: 15 or SEQ ID NO: 55. In some embodiments, the GFP has a sequence at least 95% identical to or SEQ ID NO: 16 or SEQ ID NO: 77. In some embodiments, the GFP has a sequence comprising SEQ ID NO: 16 or SEQ ID NO: 77.

Examples of chromoproteins include, but are not limited to, a chromoprotein comprising ShadowR, Stichodactyla gigantea (sgBP), Heteractis crispa (hcCP), Anemonia sulcata (asCP), Cnidopus japonicus (cjBlue), or Goniopora tenuidens (gtCP), as well as derivatives and combinations thereof.

In certain embodiments, the detectable marker can be a green fluorescence protein (GFP) or mCherry/RFP.

### Chimeric PGCs and Chimeric Avians

In another aspect, provided herein is a primordial germ cell (PGC) system comprising a first genetically modified avian primordial germ cell (PGC) and a second genetically modified avian primordial germ cell (PGC): (a) the first genetically modified avian PGC comprising a first agent, the first agent comprising a first exogenous polynucleotide, the first exogenous polynucleotide comprising: (i) a first promoter; and (ii) a first element of interest operably linked to the promoter, the first element of interest encoding a first protein moiety of interest, wherein a first genetically modified avian having a genome comprising the first agent retains fertility and viability while producing a primordial germ cell (PGC) having a genome comprising the first agent without impairing viability of the PGC; and (b) the second genetically modified avian PGC comprising a second agent, the second agent comprising a second exogenous polynucleotide, the second exogenous polynucleotide comprising: (i) a second promoter; and (ii) a second element of interest operably linked to the promoter, the second element of interest encoding a second protein moiety of interest, wherein a second genetically modified avian having a genome comprising the second agent retains fertility and viability while producing a primordial germ cell (PGC) having a genome comprising the second agent without impairing viability of the PGC, the first agent and the second agent, when co-expressed in a genetically modified progeny avian embryo or progeny avian, wherein the genetically modified progeny avian embryo or progeny avian is a progeny of a first genetically modified avian arising from the first genetically modified PGC comprising the first agent and an opposite-gendered second genetically modified avian arising from the second genetically modified PGC comprising the second agent, inducing sterility or inhibiting fertility in the genetically modified progeny avian embryo or progeny avian without impairing viability.

As used herein, the term “chimeric PGC” refers to a gene-edited or genetically modified avian PGC that contains the DNA editing agent disclosed herein, either a gene-edited or genetically modified PGC transformed by genetic modification or a PGC from a genetically modified avian (chimeric avian). In some embodiments, provided herein is a cell colony comprising gene-edited or genetically modified avian PGCs derived from a single individual parent PGC. In some embodiments, provided herein is a cell population comprising gene-edited or genetically modified avian cells, the cell population derived from a single parent PGC.

As used herein, the term “chimera”, “chimeric chick,” “chimeric adult avian,” or “chimeric avian” refers to a bird cell that contains the DNA editing agent disclosed herein, or a bird that has cells containing the DNA editing agent disclosed herein. Representative examples of chimeric bird cells include, but are not limited to, bird primordial germ cells (PGCs) such as gonadal PGCs, blood PGCs, germinal crescent PGCs, or gametes that contain the DNA editing agent disclosed herein. Representative examples of chimeric bird include, but are not limited to, chicken, turkey, duck, geese, quail, pheasant, or ostrich that has cells containing the DNA editing agent disclosed herein.

In certain embodiments, the cells of the bird comprising the exogenous polynucleotide cassette comprise bird primordial germ cells (PGCs). In certain embodiments, the bird PGCs can be gonadal PGCs, blood PGCs, or germinal crescent PGCs.

In certain embodiments, the cells of the bird comprising the exogenous polynucleotide cassette comprise bird primordial germ cells (PGCs). In certain embodiments, the bird PGCs can be gonadal PGCs, blood PGCs, or germinal crescent PGCs.

As used herein, the terms “primordial germ cell” and “PGC” refer to a diploid cell that is present in the early embryo and that can differentiate/develop into haploid gametes (i.e. spermatozoa and ova) in an adult bird.

In certain aspects, provided herein are methods for producing a sterile genetically modified avian from two fertile independently genetically modified avians, the method comprising: (i) a first promoter; and (ii) a first element of interest operably linked to the promoter, the first element of interest encoding a first protein moiety of interest, wherein the first genetically modified avian having a genome comprising the first agent retains fertility and viability while producing a primordial germ cell (PGC) having a genome comprising the first agent without impairing viability of the PGC; (c) producing first pure PGC colonies comprising the first agent; (d) transplanting a first pure PGC colony into a male chick embryo to produce a first chimera male chick embryo and transplanting a first pure PGC colony into a female chick embryo to produce a first chimera female chick embryo (e.g., first surrogate host); (e) producing a population of first chimera founder adult avians by hatching and rearing the first chimera founder chicks to sexual maturity, by mating first chimera founder chicks at adulthood to produce first chimera offspring, or both; (f) screening the first population of chimera founder adult avians to verify homozygosity for the first agent; (g) obtaining a second primordial germ cell (PGC) from an avian; (h) integrating into a chromosome of interest in the second PGC a second agent, the first agent comprising a second exogenous polynucleotide, the second exogenous polynucleotide comprising: (i) a second promoter; and (ii) a second element of interest operably linked to the promoter, the second element of interest encoding a second protein moiety of interest, wherein the second genetically modified avian having a genome comprising the second agent retains fertility and viability while producing a primordial germ cell (PGC) having a genome comprising the second agent without impairing viability of the PGC; (h) producing second pure PGC colonies comprising the second agent; (i) transplanting a second pure PGC colony into a male chick embryo to produce a second chimera male chick embryo and transplanting a second pure PGC colony into a female chick embryo to produce a second chimera female chick embryo (e.g., second surrogate host); (i) producing a population of second chimera founder adult avians by hatching and rearing the second chimera founder chicks to sexual maturity, by mating chimera founder chicks at adulthood to produce offspring, or both; (j) screening the second population of chimera founder adult avians to verify homozygosity for the second agent; (k) selecting a male homozygous for the first agent from the first population of adult avians; (l) selecting a female homozygous for the second agent from the second population of adult avians; and (m) breeding the male adult avian from the first population with the female adult avian from the second population to produce a population of one or more sterile genetically modified progeny avian embryos, the first agent and the second agent, when co-expressed in the one or more genetically modified progeny avian embryos, inducing sterility or inhibiting fertility in the one or more genetically modified progeny avian embryos without impairing viability.

In some embodiments, the method further comprises: (a) obtaining a first primordial germ cell (PGC) from an avian; (b) integrating into a chromosome of interest in the first PGC a first agent, the first agent comprising a first exogenous polynucleotide, the first exogenous polynucleotide comprising: (i) a first promoter; and (ii) a first element of interest operably linked to the promoter, the first element of interest encoding a first protein moiety of interest, wherein the first protein moiety of interest is a functionally inactive first protein moiety of interest, wherein the first genetically modified avian having a genome comprising the first agent retains fertility and viability while producing a primordial germ cell (PGC) having a genome comprising the first agent without impairing viability of the PGC; (c) integrating into a first targeted gene of interest (GOI) on a chromosome of interest in the first PGC a third agent, the third agent comprising a third exogenous polynucleotide, the third exogenous polynucleotide comprising a functionally active first GOI sequence or a functionally active fragment thereof operatively linked to an one or more target sites specific to a genomic modifier, the first GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active first gene of interest (GOI), the modified trait inducing sterility or inhibiting fertility in the functionally modified and genetically modified progeny avian embryo or progeny avian without impairing viability; (d) producing first pure PGC colonies comprising the first agent and the third agent; (e) transplanting a first pure PGC colony into a male chick embryo to produce a first chimera male chick embryo and transplanting a first pure PGC colony into a female chick embryo to produce a first chimera female chick embryo; (f) producing a population of first chimera founder adult avians by hatching and rearing the first chimera founder chicks to sexual maturity, by mating first chimera founder chicks at adulthood to produce first chimera offspring, or both; (g) screening the first population of chimera founder adult avians to verify homozygosity for the first agent and the third agent; (h) obtaining a second primordial germ cell (PGC) from an avian; (i) integrating into a chromosome of interest in the second PGC a second agent, the second agent comprising a second exogenous polynucleotide, the second exogenous polynucleotide comprising: (i) a second promoter; and (ii) a second element of interest operably linked to the promoter, the second element of interest encoding a second protein moiety of interest, wherein the second protein moiety of interest is a functionally inactive second protein moiety of interest, wherein the second genetically modified avian having a genome comprising the second agent retains fertility and viability while producing a primordial germ cell (PGC) having a genome comprising the second agent without impairing viability of the PGC; (j) integrating into a second targeted gene of interest (GOI) on a chromosome of interest in the second PGC a fourth agent, the fourth agent comprising a fourth exogenous polynucleotide, the fourth exogenous polynucleotide comprising a functionally active second GOI sequence or a functionally active fragment thereof operatively linked to one or more target sites specific to the genomic modifier, the second GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active second gene of interest (GOI), the modified trait inducing sterility or inhibiting fertility in the functionally modified and genetically modified progeny avian embryo or progeny avian without impairing viability; (k) producing second pure PGC colonies comprising the second agent and the fourth agent; (l) transplanting a second pure PGC colony into a male chick embryo to produce a second chimera male chick embryo and transplanting a second pure PGC colony into a female chick embryo to produce a second chimera female chick embryo; (m) producing a population of second chimera founder adult avians by hatching and rearing the second chimera founder chicks to sexual maturity, by mating chimera founder chicks at adulthood to produce offspring, or both; (n) screening the second population of chimera founder adult avians to verify homozygosity for the second agent; (o) selecting a male homozygous for the first agent and homozygous for the third agent from the first population of adult avians; (p) selecting a female homozygous for the second agent and homozygous for the fourth agent from the second population of adult avians; and (q) breeding the male adult avian from the first population with the female adult avian from the second population to produce a population of one or more sterile genetically modified progeny avian embryos, the first agent and the second agent, when co-expressed, comprise a functionally active protein of interest or fragment thereof comprising a genomic modifier, the genomic modifier targeting the one or more target sites in the first GOI and the second GOI in the one or more genetically modified progeny avian embryos, inducing sterility or inhibiting fertility in the one or more genetically modified progeny avian embryos without impairing viability.

In some embodiments, the method further comprises: (a) obtaining a first primordial germ cell (PGC) from an avian; (b) integrating into a chromosome of interest in the first PGC a first agent, the first agent comprising a first exogenous polynucleotide, the first exogenous polynucleotide comprising: (i) a first promoter; and (ii) a first element of interest operably linked to the promoter, the first element of interest encoding a first protein moiety of interest, the first protein moiety of interest comprising a functionally active first genomic modifier, wherein the first genetically modified avian having a genome comprising the first agent retains fertility and viability while producing a primordial germ cell (PGC) having a genome comprising the first agent without impairing viability of the PGC; (c) integrating into a first targeted gene of interest (GOI) on a chromosome of interest in the first PGC a third agent, the third agent comprising a third exogenous polynucleotide, the third exogenous polynucleotide comprising a functionally active first GOI sequence or a functionally active fragment thereof operatively linked to an one or more target sites specific to a second genomic modifier, the second genomic modifier not recognizing the target sites of the first genomic modifier, the first GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active first gene of interest (GOI), the modified trait inducing sterility or inhibiting fertility in the functionally modified and genetically modified progeny avian embryo or progeny avian without impairing viability; (d) producing first pure PGC colonies comprising the first agent and the third agent; (e) transplanting a first pure PGC colony into a male chick embryo to produce a first chimera male chick embryo and transplanting a first pure PGC colony into a female chick embryo to produce a first chimera female chick embryo; (f) producing a population of first chimera founder adult avians by hatching and rearing the first chimera founder chicks to sexual maturity, by mating first chimera founder chicks at adulthood to produce first chimera offspring, or both; (g) screening the first population of chimera founder adult avians to verify homozygosity for the first agent and the third agent; (h) obtaining a second primordial germ cell (PGC) from an avian; (i) integrating into a chromosome of interest in the second PGC a second agent, the second agent comprising a second exogenous polynucleotide, the second exogenous polynucleotide comprising: (i) a second promoter; and (ii) a second element of interest operably linked to the promoter, the second element of interest encoding a second protein moiety of interest, the second protein moiety of interest comprising a functionally active second genomic modifier, wherein the second genetically modified avian having a genome comprising the second agent retains fertility and viability while producing a primordial germ cell (PGC) having a genome comprising the second agent without impairing viability of the PGC; (j) integrating into a second targeted gene of interest (GOI) on a chromosome of interest in the second PGC a fourth agent, the fourth agent comprising a fourth exogenous polynucleotide, the fourth exogenous polynucleotide comprising a functionally active second GOI sequence or a functionally active fragment thereof operatively linked to an one or more target sites specific to the first genomic modifier, the first genomic modifier not recognizing the target sites of the second genomic modifier, the second GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active second gene of interest (GOI), the modified trait inducing sterility or inhibiting fertility in the functionally modified and genetically modified progeny avian embryo or progeny avian without impairing viability; (k) producing second pure PGC colonies comprising the second agent and the fourth agent; (l) transplanting a second pure PGC colony into a male chick embryo to produce a second chimera male chick embryo and transplanting a second pure PGC colony into a female chick embryo to produce a second chimera female chick embryo; (m) producing a population of second chimera founder adult avians by hatching and rearing the second chimera founder chicks to sexual maturity, by mating chimera founder chicks at adulthood to produce offspring, or both; (n) screening the second population of chimera founder adult avians to verify homozygosity for the second agent; (o) selecting a male homozygous for the first agent and homozygous for the third agent from the first population of adult avians; (p) selecting a female homozygous for the second agent and homozygous for the fourth agent from the second population of adult avians; and (q) breeding the male adult avian from the first population with the female adult avian from the second population to produce a population of one or more sterile genetically modified progeny avian embryos, the first genomic modifier targeting the one or more target sites in the second GOI and the second genomic modifier targeting one or more target sites in the first GOI in the one or more genetically modified progeny avian embryos, inducing sterility or inhibiting fertility in the one or more genetically modified progeny avian embryos without impairing viability.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As is known to those of skill in the art, primordial germ cells can be isolated from different developmental stages and from various sites in a developing avian embryo such as, but not limited to, genital ridge, developing gonad, blood, and germinal crescent (Chang et al., Cell Biol. Int. 21:495-9, 1997; Chang et al., Cell Biol. Int. 19:143-9, 1995; Allioli et al., Dev. Biol. 165:30-7, 1994; Swift, Am. J. Physiol. 15:483-516; International Patent Application Publication No. WO 99/06533). The genital ridge is a section of a developing embryo that is known to a person of ordinary skill in the art (Strelchenko, Theriogenology 45: 130-141, 1996; Lavoir, J. Reprod Dev. 37: 413-424, 1994). Typically, PGCs can be stained positively by the periodic acid-Schiff (PAS) technique. In several species, PGCs can be identified using an anti-SSEA antibody (one notable exception being turkeys, the PGCs from which do not display the SSEA antigen). Various techniques for isolation and purification of PGCs are known in the art, including the concentration of PGCs from blood using Ficoll density gradient centrifugation (Yasuda et al., J. Reprod Fertil. 96:521-528, 1992).

The in-vitro culture of PGCs is possible using a medium containing chicken and bovine serum, conditioned media, feeder cells and growth factors such as FGF2 (van de Lavoir et al. 2006, Nature 441:766-769. doi:10.1038/nature04831; Choi et al. 2010, PLoS ONE 5:e12968. doi:10.1371/journal.pone.0012968; MacDonald et al., 2010. PLoS ONE 5:e15518. doi:10.1371/journal.pone.0015518). It has been shown that a feeder replacement medium containing growth factors to activate the FGF, insulin and TGF-β signaling pathways could be used to propagate PGCs (Whyte et al. 2015, Stem Cell Rep. 5:1171-1182. doi:10.1016/j.stemcr.2015.10.008).

Primordial germ cells (PGCs) can be provided and formulated for carrying out the presently disclosed subject matter by any suitable technique, and stored, frozen, cultured, or the like prior to use as desired. For example, primordial germ cells can be collected from donor embryos at an appropriate embryonic stage. Stages of avian development are referred to herein by one of two art-recognized staging systems: the Eyal-Giladi & Kochav system (EG&K; Eyal-Giladi & Kochav, Dev. Biol. 49:321-327, 1976), which uses Roman numerals to refer the pre-primitive streak stages of development, and the Hamburger & Hamilton staging system (H&H; Hamburger & Hamilton, J. Morphol. 88:49-92, 1951), which uses Arabic numerals to reference the post-laying stages. Unless otherwise indicated, the stages referred to herein are stages as per the H&H staging system. In certain embodiments, PGCs are derived from blood isolated from stage 14 (H&H) embryos. In certain embodiments, PGCs are derived from blood isolated from stage 15 (H&H) embryos. In certain embodiments, PGCs are derived from blood isolated from stage 16 (H&H) embryos.

In one embodiment, PGCs can be isolated at stage 4, or the germinal crescent stage, through stage 30, with cells being collected from blood, genital ridge, or gonad in the later stages. The primordial germ cells are, in general, twice the size of somatic cells and can easily be distinguished and separated on the basis of size. Male (or homogametic) primordial germ cells (ZZ) can be distinguished from heterogametic primordial germ cells (ZW by any suitable technique, such as collecting germ cells from a particular donor and typing other cells from that donor, the collected cells being of the same chromosome type as the typed cells.

An alternative to the use of PGCs is the direct transfection of spermatozoa using a DNA editing agent disclosed herein (Cooper et al., 2016 Transgenic Res. 26:331-347, doi:10.1007/s 11248-016-0003-0).

In one embodiment, to produce chimeric birds from PGCs edited in-vitro, the exogenous edited cells are injected intravenously into surrogate host embryos at a stage when their endogenous PGCs are migrating to the genital ridge. The “donor” PGCs may be of the same breed or species as the surrogate host embryo or of a different breed or species. The edited “donor” PGCs must remain viable and in one embodiment, out-compete the endogenous PGCs if they are to colonize the forming gonad and transmit the edited chromosome(s) through the germline. To provide donor PGCs with an advantage, the number of endogenous PGCs can be reduced by chemical or genetic ablation (Smith et al., 2015, Andrology 3:1035-1049. doi:10.1111/andr.12107). Exposing the blastoderm of surrogate embryos to emulsified Busulfan has been shown to increase germline transmission of donor PGCs to over 90%, though this rate drops significantly if PGCs have been cultured or cryopreserved (Nakamura et al., 2008, Reprod. Fertil. Dev. 20:900-907. doi:10.1071/RD08138; Naito et al., 2015, Anim. Reprod Sci. 153:50-61. doi:10.1016/j.anireprosci.2014.12.003). Other methods of skewing the ratio of edited PGCs to native PGCs are described in US Patent Application No. 2006/0095980.

In certain embodiments, genetically modified PGCs can be transplanted into adult gonads as known in the art (Trefil et al., 2017 Sci. Rep. Oct 27; 7(1):14246 doi: 10.1038/s41598-017-14475-w).

The genetically modified cells (e.g. PGCs) can be formulated for administration to other birds by dissociating the cells (e.g., by mechanical dissociation) and intimately admixing the cells with a pharmaceutically acceptable carrier (e.g., phosphate buffered saline solution). In one embodiment, the primordial germ cells are gonadal primordial germ cells, or blood primordial germ cells (“gonad” or “blood” referring to the tissue of origin of the original embryonic donor). In one embodiment, the PGCs can be administered in physiologically acceptable carrier at a pH of from about 6 to about 8 or 8.5, in a suitable amount to achieve the desired effect (e.g., 100 to 30,000 PGCs per embryo). The PGCs can be administered free of other ingredients or cells, or other cells and ingredients can be administered along with the PGCs.

Administration of the primordial germ cells to the recipient animal in-ovo can be carried out at any suitable time at which the PGCs can still migrate to the developing gonads. In one embodiment, the administration is carried out from about stage IX according to the Eyal-Giladi & Kochav (EG&K) staging system to about stage 30 according to the Hamburger & Hamilton staging system of embryonic development, or in another embodiment, at stage 15. For chickens, the time of administration is thus during days 1, 2, 3, or 4 of embryonic development, for example, day 2 to day 2.5. Administration is typically done by injection into any suitable target site, such as the region defined by the amnion (including the embryo), the yolk sac, etc. In one embodiment, the cells are injected into the embryo itself (including the embryo body wall). In alternative embodiments, intravascular or intracoelomic injection into the embryo can be employed. In other embodiments, the injection is performed into the heart. The methods of the presently disclosed subject matter can be carried out with prior sterilization of the recipient bird in-ovo (e.g. by chemical treatment using Busulfan of by gamma or X-ray irradiation). As used herein, the term “sterilization” refers to render partially or completely incapable of producing gametes derived from endogenous PGCs. When donor gametes are collected from such a recipient, they can be collected as a mixture with gametes of the donor and the recipient. This mixture can be used directly, or the mixture can be further processed to enrich the proportion of donor gametes therein. Thus, a skilled artisan would appreciate that a sterile avian disclosed herein comprises an avian with a reduced capacity for producing gametes derived from endogenous PGCs, compared to an isogenic avian lacking the first genetic modification.

The in-ovo administration of the primordial germ cells can be carried out by any suitable technique, either manually or in an automated manner. In one embodiment, in-ovo administration is performed by injection. The mechanism of in-ovo administration is not critical, but the mechanism should not unduly damage the tissues and organs of the embryo or the extraembryonic membranes surrounding it so that the treatment will not unduly decrease hatch rate. A hypodermic syringe fitted with a needle of about 18 to 26 gauge is suitable for the purpose. A sharpened pulled glass pipette with an opening of about 20-50 microns diameter may be used. Depending on the precise stage of development and position of the embryo, a one-inch needle will terminate either in the fluid above the chick or in the chick itself. A pilot hole can be punched or drilled through the shell prior to insertion of the needle to prevent damaging or dulling of the needle. If desired, the egg can be sealed with a substantially bacteria-impermeable sealing material such as wax or the like to prevent subsequent entry of undesirable bacteria. It is envisioned that a high-speed injection system for avian embryos would be suitable for practicing the presently disclosed subject matter.

All such devices, as adapted for practicing the methods disclosed herein, comprise an injector containing a formulation of the primordial germ cells as described herein, with the injector positioned to inject an egg carried by the apparatus. In addition, a sealing apparatus operatively connected to the injection apparatus can be provided for sealing the hole in the egg after injection. In another embodiment, a pulled glass micropipette can be used to introduce the PGCs into the appropriate location within the egg, for example directly into the blood stream, either to a vein or an artery or directly into the heart.

Once the eggs have been injected with the modified or unmodified PGCs, the chimeric embryo is incubated until hatch. In one embodiment, the chick is raised to sexual maturity, wherein the chimeric bird produces gametes derived from the donor PGCs.

In certain embodiments, the cells of the bird comprise bird gametes. The gametes, (either eggs or sperm) from the chimeras (or from material that has been directly genetically manipulated, as described herein above) are then used to raise founder chickens (F1).

Molecular biology techniques known in the art (e.g. PCR or Southern blot or both) may be used to confirm germline transmission. F1 chickens may be back-crossed to generate homozygous carrier males and carrier females (F2). Gametes from founder chickens F2 can then be used to expand the breeding colonies. The colonies are typically grown until sexual maturity.

In certain embodiments, the method further comprises incubating the chimeric bird embryo, in-ovo, until hatching. In certain embodiments, the method further comprises raising the chimeric bird to sexual maturity, wherein the chimeric bird produces gametes derived from the administered cells.

In certain embodiments, the genome-edited cells are administered by in-ovo injection. In certain embodiments, the administrated cell population is derived from the same avian species as the recipient bird embryo. In certain embodiments, the administrated cell population is derived from a different avian species as the recipient bird embryo.

In certain embodiments, the genome-edited bird cell population is administered when the recipient embryo is about stage IX according to the Eyal-Giladi & Kochav staging system. In certain embodiments, the bird cell population is administered when the recipient embryo is about stage 30 according to the Hamburger & Hamilton staging system. In certain embodiments, the bird cell population is administered when the recipient embryo is about stage IX according to the Eyal-Giladi & Kochav staging system; and about stage 30 according to the Hamburger & Hamilton staging system. In certain embodiments, the bird cell population is administered when the recipient embryo is after stage 14 according to the Hamburger & Hamilton staging system.

In certain embodiments, the genome-edited bird cell population is administered after irradiation of the embryo. In certain embodiments, the irradiation comprises γ-irradiation or X-ray irradiation. In certain embodiments, the irradiation comprises 600-800 rad of γ irradiation. In certain embodiments, the irradiation comprises 600-800 rad of irradiation. In certain embodiments, the irradiation comprises 400-1000 rad of irradiation. In certain embodiments, the irradiation comprises 200-1200 rad of irradiation.

Further provided, in another aspect, is a chimeric bird obtainable from the methods described above.

### Genome Editing and Transforming PGCs in Chickens

Transcription Activator-like Effector Nuclease (TALEN), Zinc finger nucleases, and Clustered Regularly Interspaced Short Palindromic Repeats (CRISPR)-Cas9 (Taylor et al. [2017] Devel. (Cambridge, England) 144: 928-934) are among the variety of useful tools for genome editing in chickens. For example, CRISPR-Cas9 benefits from easy construction, rapid application and high efficiency.

In some embodiments, the CRISPR-Cas9 system is introduced to PGCs as transient episomal plasmids or as a recombinant sgRNA-Cas9 protein complex (RNP). Apart from the genome editing per se, the strategies leave no alien DNA traces of the CRISPR system. Generating DNA breaks invokes the DNA repair mechanism that could result in INDELs (i.e., insertion or deletion of 1-10,000 bases in the genome of the organism) in the break site. These INDELs may render the targeted gene inactive. This inactivation can be achieved by the non-homologous-end-joining repair mechanism. On the other hand, the homologous recombination repair mechanism facilitates targeted integration of a foreign DNA sequence, which can replace the genomic DNA sequence flanking the break point. Using targeted integration to the genome, a gene of interest (GOI), such as a reporter gene, could substitute for an endogenous gene to be under the regulation of the endogenous promoter.

In other embodiments, to knockout the expression of a sterility-inducing gene, a conditional-activated mechanism can also be used, such as the Cre-LoxP or Flp-FRT systems. To this end, using homologous recombination, two LoxP sites (for example) can be introduced in intronic regions, flanking an essential exon. This method allows for normal expression of the gene. However, by crossing with Cre expressing strain, the LoxP flanked exon is removed, and the gene becomes inactive.

sgRNA may comprise a ribonucleic acid. sgRNA may encode a ribonucleic acid.

In one embodiment, sgRNA-5′ssODN (SEQ ID NO: 21) codes for a RNA sequence. In one embodiment, SEQ ID NO: 21 codes for a DNA sequence, which may be used as a template for preparing sgRNA. In some embodiments, the complement of SEQ ID NO: 21 may be used as a template for preparing sgRNA. In one embodiment, sgRNA-3′ssODN (SEQ ID NO: 22) codes for a RNA sequence. In one embodiment, SEQ ID NO: 22 codes for a DNA sequence, which may be used as a template for preparing sgRNA. In some embodiments, the complement of SEQ ID NO: 22 may be used as a template for preparing sgRNA. In one embodiment, TV-sgRNA (SEQ ID NO: 25) codes for a RNA sequence. In one embodiment, SEQ ID NO: 25 codes for a DNA sequence, which may be used as a template for preparing sgRNA. In some embodiments, the complement of SEQ ID NO: 25 may be used as a template for preparing sgRNA.

Importantly, the rate of intrinsic homologous recombination events in PGCs, without DNA break, is high. Thus, transfection of targeting vector alone can result in a specific homologous recombination-mediated genomic integration, without risking damaging the genomic DNA by off-target modifications known to be generated by genome editing agents.

### Gene Knockout

In some embodiments, the gene-editing method for producing sterile chicks comprises a gene knockout method. In some embodiments, the genome editing techniques above (e.g., specific gene knockout chickens via TALEN-mediated, CRISPR/Cas9, or other methods known in the art) are adapted to be used to knock out a gene (e.g., to silence its expression), including, but not limited to removal, replacement, inactivation, mutation, and the like for knocking out or otherwise silencing or inactivating the gene. Other methods include, but are not limited to, the use of viral vectors (e.g., avian leukosis virus, lentivirus, retrovirus, and other vectors), e.g., via microinjection, electroporation, or other techniques.

### Genetically Modified Chicks

In some embodiments, the method for producing sterile chicks comprises genetic modification to alter the genome of the chick. In some embodiments, the altered genome comprises a DNA sequence or gene from a different species to produce a transgenic chick.

Genetic modification methods include, but are not limited to, the methods described herein, including the use of endonucleases and genome modifiers, such as CRISPR, TALEN, etc., with or without the combination of homologous recombination repair mechanisms. Examples of DNA sequences or genes from a different species include, but are not limited to, the examples described herein, including green fluorescent protein (e.g., jellyfish Aequorea victoria), 3-phosphoglycerate kinase (Pgk) promoter (e.g., mouse), cytomegalovirus (CMV) promoter (cytomegalovirus), internal ribosome entry site (IRES) (poliovirus [PV]), and encephalomyocarditis virus (EMCV).

Unless otherwise indicated, all numbers expressing quantities, ratios, and numerical properties of ingredients, reaction conditions, and so forth used in the specification and claims are to be understood as being modified in all instances by the term “about.” All parts, percentages, ratios, etc., herein are by weight unless indicated otherwise.

As used herein, the terms “inactivate” and “deactivate” are used interchangeably and fall within each meaning to denote “to stop action or activity,” “to suppress action or activity,” or “to render incapable of action or activity.”

As used herein, the singular forms “a” or “an” or “the” are used interchangeably and intended to include the plural forms as well and fall within each meaning, unless expressly stated otherwise or unless the context clearly dictates otherwise. For example, the term “a compound” or “at least one compound” may include a plurality of compounds, including mixtures thereof.

Also as used herein, “at least one” is intended to mean “one or more” of the listed elements. Singular word forms are intended to include plural word forms and are likewise used herein interchangeably where appropriate and fall within each meaning, unless expressly stated otherwise. Except where noted otherwise, capitalized and non-capitalized forms of all terms fall within each meaning.

“Consisting of” shall thus mean excluding more than traces of other elements.

The skilled artisan would appreciate that while, in some embodiments the term “comprising” is used, such a term may be replaced by the term “consisting of”, wherein such a replacement would narrow the scope of inclusion of elements not specifically recited. The terms “comprises”, “comprising”, “includes”, “including”, “having” and their conjugates encompass “including but not limited to”.

The term “about” or “approximately” means within an acceptable error range for the particular value as determined by one of ordinary skill in the art, which will depend in part on how the value is measured or determined. In some embodiments, the term “about” refers to a deviance of between 0.0001-5% from the indicated number or range of numbers.

In some embodiments, the term “about” refers to a deviance of between 1-10% from the indicated number or range of numbers. In some embodiments, the term “about” refers to a deviance of up to 25% from the indicated number or range of numbers. In some embodiments, the term “about” refers to +10%.

Throughout this application, various embodiments may be presented in a range format. It should be understood that the description in range format is merely for convenience and brevity and should not be construed as an inflexible limitation on the scope of certain embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Described herein is a system to produce totally sterile populations of embryos and birds, for example but not limited to chickens. A skilled artisan would appreciate that the term “totally” refers to the population of embryos of avians so “totally sterile” encompasses a population of embryos that are all sterile. i.e., 100% of the population is sterile. This is in contrast to a mendelian system for producing sterility by crossing between two heterozygote individuals carrying deleteriously-modified FRGs located on the Z sex chromosome produces sterility in only 25% of the population. Similarly, a method producing totally sterile population of embryos, as described and exemplified herein, would be understood by a skilled artisan to encompass a method that produces a population of embryos and birds, for example but not limited to chickens, that is completely 100% sterile.

Any patent, patent application publication, or scientific publication, cited herein, is incorporated by reference herein in its entirety.

The following examples are presented in order to more fully illustrate some embodiments of the sterile avian embryos, products and uses thereof. They should, in no way be construed, however, as limiting the broad scope of the sterile avian embryos, products and uses thereof. One skilled in the art can readily devise many variations and modifications of the principles disclosed herein without departing from the scope of the sterile avian embryos, products and uses thereof.

### EXAMPLES

### Example 1: Production of Sterile Avian Embryos Devoid of Functional Primordial Germ Cells (PGCs) While Maintaining Founder Populations

Objective: To produce a population of sterile avian embryos devoid of functional PGCs while maintaining founder populations.

PGCs of a desired avian species or breed are isolated and cultured.

Via targeted genome-editing of primordial germ cells (PGCs), two breeds are independently created, Breed A and Breed B, and enriched or optionally purified PGC colonies of each breed are obtained and analyzed or verified to confirm the desired genetic modifications, followed by transplantation of Breed A PGC colonies into a first population of surrogate avian embryos (chimera avian embryos) and transplantation of Breed B PGC colonies into a second population of surrogate avian embryos (chimera avian embryos). The chimera avians are reared to sexual maturity and screened for Breed A and Breed B founder avians. Male and female Breed A founder avians are bred to form a population of Breed A birds, while male and female Breed B avians are independently bred to form a population of Breed B birds.

In each breed, there is a distinct semi-inactivating element (SIE). Breed A comprises a first agent having a first exogenous polynucleotide comprising a first promoter and a first element of interest operably linked to the first promoter and encoding a first protein moiety of interest. Breed B comprises a second agent having a second exogenous polynucleotide comprising a second promoter and a second element of interest operably linked to the second promoter and encoding a second protein moiety of interest.

The SIEs are a binary mechanism that directly inactivates a gene of interest (GOI), such as a fertility-required gene (FRG) and/or a binary mechanism that controls wild-type or exogenous sterility-inducing factors (SIFs) and/or a binary mechanism that produces or activates a toxin inducing cell death of a PGC, either without impairing viability of somatic cells or limited to benign somatic cell loss or both, and/or a combination of any of these. For example, a FRG, the product of which has a function related to fertility (e.g., having an isolated function in PGCs, gamete maturation, or gamete function), is identified, such that deletion or mutation of the gene product will lead to sterility, preferably without having an impact on viability. In each of the breeds, there is a copy of an active FRG, thus, within itself, each breed is fertile. A SIF is identified that inactivates the FRG or induces sterility by another means. In each of the breeds, the SIF is not active, thus, within itself, each breed is fertile. A toxin is identified that induces cell death of PGCs, either without impairing viability of somatic cells or limited to benign somatic cell loss or both. In each of the breeds, the toxin is not active, thus, within itself, each breed is fertile.

By crossing Breed A with Breed B, the offspring Breed AB arises. Breed AB contains the two SIE copies, one from each parent. When co-expressed, the first agent and the second agent in a progeny avian embryo or a progeny avian of Breed AB will either deactivate the FRG, activate the SIF, produce or activate the toxin, or a combination thereof. The inactivated FRG(s), activated SIF, activated toxin, or any combination thereof renders the organisms of Breed AB sterile.

### Example 2: Production of Sterile Avian Embryos Devoid of Functional Primordial Germ Cells (PGCs) While Maintaining Founder Populations

Objective: To produce a population of sterile avian embryos devoid of functional PGCs while maintaining founder populations.

PGCs of a desired avian species or breed are isolated and cultured. A gene of interest (GOI) (e.g., a fertility-related gene (FRG)), the product of which has a function related to fertility (e.g., having an isolated function in PGCs, gamete maturation, or gamete function), is identified, such that deletion or mutation of the gene product will lead to sterility, preferably without having an impact on viability.

Two breeds are created, Breed A and Breed B, as described above, and the genetic background of them is represented by an illustration of a chromosome in blue and red (FIGS. 2A-2B). In each breed, there is a semi-inactivating element (SIE), depicted in Breed A (top left) and Breed B (top right) in yellow and green ovals, respectively. In each of the breeds, there is a copy of an active fertility-required gene (FRG), depicted in white, thus, within itself, each breed is fertile. The SIEs are a binary mechanism that directly inactivates the FRGs, and/or a binary mechanism that controls wild-type or exogenous sterility-inducing factors (SIFs) (e.g., indirectly inactivating the FRGs or by inducing sterility by another mechanism) and/or a toxin inducing cell death of a PGC, either without impairing viability of somatic cells or limited to benign somatic cell loss or both, and/or a combination thereof. By crossing Breed A with Breed B, the offspring Breed AB (bottom) arises. Breed AB contains the two SIE copies, one from each parent. Having these two elements together, will either deactivate the FRG or activate the SIF or produce or activate the toxin (yellow arrow). The inactivated FRG, depicted in the black oval, renders the organisms of Breed AB sterile.

### Example 3: Production of Sterile Avian Embryos Devoid of Functional Primordial Germ Cells (PGCs) While Maintaining Founder Populations by Directly Inactivating a Fertility Required Gene (FRG)

Objective: To produce a population of sterile avian embryos devoid of functional PGCs while maintaining founder populations by directly inactivating an FRG.

PGCs of a desired avian species or breed are isolated and cultured. A gene of interest (GOI) (e.g., a fertility-related gene (FRG)), the product of which has a function related to fertility (e.g., having an isolated function in PGCs, gamete maturation, or gamete function), is identified, such that deletion or mutation of the gene product will lead to sterility, preferably without having an impact on viability.

Two breeds are created, Breed A and Breed B, as described above. In each breed, there is a semi-inactivating element (SIE), in Breed A and Breed B. In each of the breeds, there is a copy of an active fertility-required gene (FRG), thus, within itself, each breed is fertile. The SIEs are a binary mechanism that directly inactivates the FRGs.

By crossing Breed A with Breed B, the offspring Breed AB arises. Breed AB contains the two SIE copies, one from each parent. Having these two elements together, will deactivate the FRG directly. The inactivated FRG renders the organisms of Breed AB sterile.

### Example 4: Production of Sterile Avian Embryos Devoid of Functional Primordial Germ Cells (PGCs) While Maintaining Founder Populations by Activating a Sterility-Inducing Factor (SIF)

Objective: To produce a population of sterile avian embryos devoid of functional PGCs while maintaining founder populations by indirectly inactivating a FRG.

PGCs of a desired avian species or breed are isolated and cultured. A gene of interest (GOI) (e.g., a fertility-related gene (FRG)), the product of which has a function related to fertility (e.g., having an isolated function in PGCs, gamete maturation, or gamete function), is identified, such that deletion or mutation of the gene product will lead to sterility, preferably without having an impact on viability.

Two breeds are created, Breed A and Breed B, as described above. In each breed, there is a semi-inactivating element (SIE). In each of the breeds, there is a copy of an inactive SIF, thus, within itself, each breed is fertile. The SIEs are a binary mechanism that controls wild-type or exogenous sterility-inducing factors (SIFs) (e.g., indirectly inactivating the FRGs or inducing sterility by another mechanism).

By crossing Breed A with Breed B, the offspring Breed AB arises. Breed AB contains the two SIE copies, one from each parent. When co-expressed, the first agent and the second agent in a progeny avian embryo or a progeny avian of Breed AB will produce or activate the SIF, or a combination thereof. Having these two elements together, will produce or activate the SIF, rendering the organisms of Breed AB sterile.

### Example 5: Production of Sterile Avian Embryos Devoid of Functional Primordial Germ Cells (PGCs) While Maintaining Founder Populations by a Toxin Inducing Cell Death of PGCs

Objective: To produce a population of sterile avian embryos devoid of functional PGCs while maintaining founder populations by a toxin inducing cell death of PGCs or limited to benign somatic cell loss or both.

PGCs of a desired avian species or breed are isolated and cultured. A toxin is identified that induces cell death of PGCs without impairing viability of somatic cells.

Two breeds are created, Breed A and Breed B, as described above. In each breed, there is a semi-inactivating element (SIE). The SIEs are a binary mechanism that controls toxin formation or activation. In each of the breeds, the toxin (e.g., a Pseudomonas exotoxin, a diphtheria toxin, a ricin, or a recombinant toxin such as, e.g., those used to fight cancer cells) is not active, thus, within itself, each breed is fertile.

By crossing Breed A with Breed B, the offspring Breed AB arises. Breed AB contains the two SIE copies, one from each parent. When co-expressed, the first agent and the second agent in a progeny avian embryo or a progeny avian of Breed AB will form or activate the toxin, or a combination thereof. The presence or activation of the toxin renders the organisms of Breed AB sterile.

### Example 6: Production of Sterile Avian Embryos Devoid of Functional Primordial Germ Cells (PGCs) while Maintaining Founder Populations Via Genomic Modifier Moieties

Objective: To produce a population of sterile avian embryos devoid of functional PGCs while maintaining founder populations using genomic modifier (e.g., recombinase) moieties.

PGCs of a desired avian species or breed are isolated and cultured. A gene of interest (GOI), the product of which has a function related to fertility (e.g., having an isolated function in PGCs, gamete maturation, or gamete function), is identified, such that deletion or mutation of the gene product will lead to sterility, preferably without having an impact on viability. The PGCs are subjected to targeted genome editing of the GOI and cultured.

Two breeds are created, Breed A and Breed B, as described above. In each breed, there is a semi-inactivating element (SIE), as described above. In Breed A, the SIE is a first functionally inactive first protein moiety of a genomic modifier. In Breed B, the STE is a second functionally inactive protein moiety of the genomic modifier. In addition, one or more target sites capable of recognition by a corresponding activated form of the genomic modifier are introduced into the GOI sequence(s) in Breed A and Breed B. In each of the breeds there is a copy of an active GOI (e.g., an active FRG or inactive SIF) into which one or more target sites have been introduced, thus, within itself, each breed is fertile, while each breed contains an inactive protein moiety of the genomic modifier and one or more corresponding target sites. The SIEs are a binary mechanism that directly inactivates the FRGs, and/or a binary mechanism that controls wild-type or exogenous sterility-inducing factors (SIFs), and/or a binary mechanism that produces or activates a toxin inducing cell death of a PGC, either without impairing viability of somatic cells or limited to benign somatic cell loss or both, and/or a combination of any of these, as described above.

By crossing Breed A with Breed B, the offspring Breed AB arises. Breed AB contains the two SIE copies (i.e., the first protein moiety of the genomic modifier and the second protein moiety of the genomic modifier), one from each parent. Having these two elements together (e.g., dimerized or covalently bound), will produce a functionally active protein of interest or fragment thereof comprising a genomic modifier, which targets the target sites in the GOI or a fragment thereof in Breed AB, such that the GOI is, e.g., deleted, disrupted, or functionally modified, modifying a trait, the modified trait inducing sterility or inhibiting fertility in the Breed AB progeny avian embryo or progeny avian without impairing viability, either by deactivating the FRG, activating the SIF, producing or activating the toxin, or a combination thereof.

### Example 7: Production of Sterile Avian Embryos Devoid of Functional Primordial Germ Cells (PGCs) While Maintaining Founder Populations Via Cre Recombinase Moieties

Objective: To produce a population of sterile avian embryos devoid of functional PGCs while maintaining founder populations using Cre recombinase moieties.

PGCs of a desired avian species or breed are isolated and cultured. A gene of interest (GOI), the product of which has a function related to fertility (e.g., having an isolated function in PGCs, gamete maturation, or gamete function), is identified, such that deletion or mutation of the gene product will lead to sterility, preferably without having an impact on viability. The PGCs are subjected to targeted genome editing of the GOI and cultured.

Two breeds are created, Breed A and Breed B, as described above. In each breed, there is a semi-inactivating element (SIE), as described above. In Breed A, the SIE is a first functionally inactive first protein moiety of a Cre recombinase. In Breed B, the SIE is a second functionally inactive protein moiety of the Cre recombinase. In addition, Lox target sites (e.g., LoxP) capable of recognition by a corresponding activated form of the Cre recombinase are introduced into the GOI sequence(s) in Breed A and Breed B. In each of the breeds there is a copy of an active GOI (e.g., an active FRG or inactive SIF) into which one or more target sites have been introduced, thus, within itself, each breed is fertile, while each breed contains a distinct inactive protein moiety of the Cre recombinase and corresponding Lox (e.g., LoxP) target sites. The SIEs are a binary mechanism that directly inactivates the FRGs, and/or a binary mechanism that controls wild-type or exogenous sterility-inducing factors (SIFs), and/or a binary mechanism that produces or activates a toxin inducing cell death of a PGC, either without impairing viability of somatic cells or limited to benign somatic cell loss or both, and/or a combination of any of these, as described above.

By crossing Breed A with Breed B, the offspring Breed AB arises. Breed AB contains the two SIE copies (i.e., the first protein moiety of the Cre recombinase and the second protein moiety of the Cre recombinase), one from each parent. Having these two elements together (e.g., dimerized or covalently bound), will produce a functionally active protein of interest or fragment thereof comprising a Cre recombinase, which targets the Lox (e.g., LoxP) target sites in the GOI or a fragment thereof in Breed AB, such that the GOI is, e.g., deleted, disrupted, or functionally modified, modifying a trait, the modified trait inducing sterility or inhibiting fertility in the Breed AB progeny avian embryo or progeny avian without impairing viability, either by deactivating the FRG, activating the SIF, producing or activating the toxin, or a combination thereof.

### Example 8: Production of Sterile Avian Embryos Devoid of Functional Primordial Germ Cells (PGCs) while Maintaining Founder Populations Via Dre Recombinase Moieties

Objective: To produce a population of sterile avian embryos devoid of functional PGCs while maintaining founder populations using Dre recombinase moieties.

PGCs of a desired avian species or breed are isolated and cultured. A gene of interest (GOI), the product of which has a function related to fertility (e.g., having an isolated function in PGCs, gamete maturation, or gamete function), is identified, such that deletion or mutation of the gene product will lead to sterility, preferably without having an impact on viability. The PGCs are subjected to targeted genome editing of the GOI and cultured.

Two breeds are created, Breed A and Breed B, as described above. In each breed, there is a semi-inactivating element (SIE), as described above. In Breed A, the SIE is a first functionally inactive first protein moiety of a Dre recombinase. In Breed B, the SIE is a second functionally inactive protein moiety of the Dre recombinase. In addition, Rox target sites capable of recognition by a corresponding activated form of the Dre recombinase are introduced into the GOI sequence(s) in Breed A and Breed B. In each of the breeds there is a copy of an active GOI (e.g., an active FRG or inactive SIF) into which Rox target sites have been introduced, thus, within itself, each breed is fertile, while each breed contains an inactive protein moiety of the Dre recombinase and one or more corresponding Rox target sites. The SIEs are a binary mechanism that directly inactivates the FRGs, and/or a binary mechanism that controls wild-type or exogenous sterility-inducing factors (SIFs), and/or a binary mechanism that produces or activates a toxin inducing cell death of a PGC, either without impairing viability of somatic cells or limited to benign somatic cell loss or both, and/or a combination of any of these, as described above.

By crossing Breed A with Breed B, the offspring Breed AB arises. Breed AB contains the two SIE copies (i.e., the first protein moiety of the Dre recombinase and the distinct second protein moiety of the Dre recombinase), one from each parent. Having these two elements together (e.g., dimerized or covalently bound), will produce a functionally active protein of interest or fragment thereof comprising a Dre recombinase, which targets the Rox target sites in the GOI or a fragment thereof in Breed AB, such that the GOI is, e.g., deleted, disrupted, or functionally modified, modifying a trait, the modified trait inducing sterility or inhibiting fertility in the Breed AB progeny avian embryo or progeny avian without impairing viability, either by deactivating the FRG, activating the SIF, producing or activating the toxin, or a combination thereof.

### Example 9: Production of Sterile Avian Embryos Devoid of Functional Primordial Germ Cells (PGCs) while Maintaining Founder Populations Via Flp Recombinase Moieties

Objective: To produce a population of sterile avian embryos devoid of functional PGCs while maintaining founder populations using Flp recombinase moieties.

PGCs of a desired avian species or breed are isolated and cultured. A gene of interest (GOI), the product of which has a function related to fertility (e.g., having an isolated function in PGCs, gamete maturation, or gamete function), is identified, such that deletion or mutation of the gene product will lead to sterility, preferably without having an impact on viability. The PGCs are subjected to targeted genome editing of the GOI and cultured.

Two breeds are created, Breed A and Breed B, as described above. In each breed, there is a semi-inactivating element (SIE), as described above. In Breed A, the SIE is a first functionally inactive first protein moiety of a Flp recombinase. In Breed B, the SIE is a second functionally inactive protein moiety of the Flp recombinase. In addition, FRT target sites capable of recognition by a corresponding activated form of the Flp recombinase are introduced into the GOI sequence(s) in Breed A and Breed B. In each of the breeds there is a copy of an active GOI (e.g., an active FRG or inactive SIF) into which FRT target sites have been introduced, thus, within itself, each breed is fertile, while each breed contains an inactive protein moiety of the Flp recombinase and one or more corresponding FRT target sites. The SIEs are a binary mechanism that directly inactivates the FRGs, and/or a binary mechanism that controls wild-type or exogenous sterility-inducing factors (SIFs), and/or a binary mechanism that produces or activates a toxin inducing cell death of a PGC, either without impairing viability of somatic cells or limited to benign somatic cell loss or both, and/or a combination of any of these, as described above.

By crossing Breed A with Breed B, the offspring Breed AB arises. Breed AB contains the two SIE copies (i.e., the first protein moiety of the Flp recombinase and the distinct second protein moiety of the Flp recombinase), one from each parent. Having these two elements together (e.g., dimerized or covalently bound), will produce a functionally active protein of interest or fragment thereof comprising a Flp recombinase, which targets the FRT target sites in the GOI or a fragment thereof in Breed AB, such that the GOI is, e.g., deleted, disrupted, or functionally modified, modifying a trait, the modified trait inducing sterility or inhibiting fertility in the Breed AB progeny avian embryo or progeny avian without impairing viability, either by deactivating the FRG, activating the SIF, producing or activating the toxin, or a combination thereof.

### Example 10: Production of Sterile Avian Embryos Devoid of Functional Primordial Germ Cells (PGCs) While Maintaining Founder Populations Via Vika Recombinase Moieties

Objective: To produce a population of sterile avian embryos devoid of functional PGCs while maintaining founder populations using Vika recombinase moieties.

PGCs of a desired avian species or breed are isolated and cultured. A gene of interest (GOI), the product of which has a function related to fertility (e.g., having an isolated function in PGCs, gamete maturation, or gamete function), is identified, such that deletion or mutation of the gene product will lead to sterility, preferably without having an impact on viability. The PGCs are subjected to targeted genome editing of the GOI and cultured.

Two breeds are created, Breed A and Breed B, as described above. In each breed, there is a semi-inactivating element (SIE), as described above. In Breed A, the SIE is a first functionally inactive first protein moiety of a Vika recombinase. In Breed B, the SIE is a second functionally inactive protein moiety of the Vika recombinase. In addition, one or more Vox target sites capable of recognition by a corresponding activated form of the Vika recombinase are introduced into the GOI sequence(s) in Breed A and Breed B. In each of the breeds there is a copy of an active GOI (e.g., an active FRG or inactive SIF) into which Vox target sites have been introduced, thus, within itself, each breed is fertile, while each breed contains an inactive protein moiety of the Vika recombinase and corresponding Vox target sites. The SIEs are a binary mechanism that directly inactivates the FRGs, and/or a binary mechanism that controls wild-type or exogenous sterility-inducing factors (SIFs), and/or a binary mechanism that produces or activates a toxin inducing cell death of a PGC, either without impairing viability of somatic cells or limited to benign somatic cell loss or both, and/or a combination of any of these, as described above.

By crossing Breed A with Breed B, the offspring Breed AB arises. Breed AB contains the two SIE copies (i.e., the first protein moiety of the Vika recombinase and the second protein moiety of the Vika recombinase), one from each parent. Having these two elements together (e.g., dimerized or covalently bound), will produce a functionally active protein of interest or fragment thereof comprising a Vika recombinase, which targets the Vox target sites in the GOI or a fragment thereof in Breed AB, such that the GOI is, e.g., deleted, disrupted, or functionally modified, modifying a trait, the modified trait inducing sterility or inhibiting fertility in the Breed AB progeny avian embryo or progeny avian without impairing viability, either by deactivating the FRG, activating the SIF, producing or activating the toxin, or a combination thereof.

### Example 11: Production of Sterile Avian Embryos Devoid of Functional Primordial Germ Cells (PGCs) while Maintaining Founder Populations Via Multiple Genomic Modifier Moieties

Objective: To produce a population of sterile avian embryos devoid of functional PGCs while maintaining founder populations using multiple genomic modifier moieties (e.g., distinct recombinases).

PGCs of a desired avian species or breed are isolated and cultured. One or more gene(s) of interest (GOI), the product of which has a function related to fertility (e.g., having an isolated function in PGCs, gamete maturation, or gamete function), is/are identified, such that deletion or mutation of the one or more gene product(s) will lead to sterility, preferably without having an impact on viability. The PGCs are subjected to targeted genome editing of the GOI and cultured.

Two breeds are created, Breed A and Breed B, as described above. In each breed, there is a semi-inactivating element (SIE), as described above. In Breed A, the SIE is a first functionally active genomic modifier. In Breed B, the SIE is a second functionally active genomic modifier. In addition, one or more target sites capable of recognition by a corresponding active form of the genomic modifier are introduced into the GOI sequence(s) in Breed A and Breed B, with the one or more target sites capable of recognition by the first genomic modifier introduced into the GOI sequence(s) of Breed B, while the one or more target sites capable of recognition by the second genomic modifier introduced into the GOI sequence(s) of Breed A. The GOI may be the same GOI in both breeds, or there may be a different GOI in each breed. In each of the breeds there is a copy of one or more active GOI (e.g., an active FRG and/or inactive SIF) into which one or more target sites have been introduced, thus, within itself, each breed is fertile, while each breed contains an inactive protein moiety of the genomic modifier and one or more corresponding target sites. In Breed A, there is a copy of one or more active GOI (at least one of which comprises one or more target sites for the second functionally active genomic modifier) and the first functionally active genomic modifier. In Breed B, there is a copy of one or more active GOI (at least one of which comprises one or more target sites for the first functionally active genomic modifier) and the second functionally active genomic modifier. The double SIEs are a binary mechanism that directly inactivates the FRGs, and/or a binary mechanism that controls wild-type or exogenous sterility-inducing factors (SIFs), and/or a binary mechanism that produces or activates a toxin inducing cell death of a PGC, either without impairing viability of somatic cells or limited to benign somatic cell loss or both, and/or a combination of any of these, as described above.

By crossing Breed A with Breed B, the offspring Breed AB arises. Breed AB contains the two SIE copies (i.e., the first functionally active genomic modifier and the second functionally active genomic modifier), one from each parent, as well as the one or more GOI comprising both the one or more target sites for the first functionally active genomic modifier and the one or more target sites for the second functionally active genomic modifier. Having these elements together (i.e., two genomic modifiers, together with their respective target sites), will bring together two genomic modifiers, along with their respective target sites in the one or more GOI or a fragment thereof in Breed AB, such that the GOI is, e.g., deleted, disrupted, or functionally modified, modifying a trait, the modified trait inducing sterility or inhibiting fertility in the Breed AB progeny avian embryo or progeny avian without impairing viability, either by deactivating the FRG, activating the SIF, producing or activating the toxin, or a combination thereof.

### Example 12: Production of Sterile Avian Embryos Devoid of Functional Primordial Germ Cells (PGCs) while Maintaining Founder Populations Via an Intein Reconstitution System

Objective: To produce a population of sterile avian embryos devoid of functional PGCs while maintaining founder populations using an intein reconstitution system.

PGCs of a desired avian species or breed are isolated and cultured. A gene of interest (GOI) or fertility-related gene (FRG), the product of which has a function related to fertility (e.g., having an isolated function in PGCs, gamete maturation, or gamete function), is identified, such that deletion or mutation of the gene product will lead to sterility, preferably without having an impact on viability.

Two breeds are created, Breed A and Breed B, as described above. In each breed, there is a semi-inactivating element (SIE). In Breed A, the SIE is a first functionally inactive first protein moiety of an intein (e.g., N-terminal intein fragment) operably linked to a promoter and to the first element of interest. In Breed B, the SIE is a second functionally inactive protein moiety of the intein (e.g., C-terminal intein fragment) operably linked to a promoter and to the second element of interest. In each of the breeds there is a copy of an active fertility-required gene (FRG), thus, within itself, each breed is fertile, while each breed contains an inactive protein moiety of the intein. A SIF is identified that inactivates the FRG or induces sterility by another means. In each of the breeds, the SIF is not active, thus, within itself, each breed is fertile. A toxin is identified that induces cell death of PGCs without impairing viability of somatic cells. In each of the breeds, the toxin is not active, thus, within itself, each breed is fertile.

By crossing Breed A with Breed B, the offspring Breed AB arises. Breed AB contains the two SIE copies (i.e., the first protein moiety of an intein [e.g., the N-terminal intein] and the second protein moiety of the intein [e.g., the C-terminal intein]), one from each parent. Having these two elements together, they will form a functionally active intein enzyme (e.g., via splicing) that will fuse the first element of interest and the second element of interest, which in turn will either deactivate the FRG, activate the SIF, produce or active the toxin, or a combination thereof. The inactivated FRG, activated SIF, activated toxin, or any combination thereof renders the organisms of Breed AB sterile.

### Example 13: Production of Sterile Avian Embryos Devoid of Functional Primordial Germ Cells (PGCs) while Maintaining Founder Populations Via a Conjugating Element System

Objective: To produce a population of sterile avian embryos devoid of functional PGCs while maintaining founder populations using a conjugating element system.

PGCs of a desired avian species or breed are isolated and cultured. A gene of interest (GOI) or fertility-related gene (FRG), the product of which has a function related to fertility (e.g., having an isolated function in PGCs, gamete maturation, or gamete function), is identified, such that deletion or mutation of the gene product will lead to sterility, preferably without having an impact on viability.

Two breeds are created, Breed A and Breed B, as described above. In each breed, there is a semi-inactivating element (SIE). In Breed A, the SIE is a functionally inactive first conjugating element operably linked to a promoter and to the first element of interest.

In Breed B, the SIE is a functionally inactive second conjugating element operably linked to a promoter and to the second element of interest. In each of the breeds there is a copy of an active fertility-required gene (FRG), thus, within itself, each breed is fertile, while each breed contains an inactive conjugating element. A SIF is identified that inactivates the FRG or induces sterility by another means. In each of the breeds, the SIF is not active, thus, within itself, each breed is fertile. A toxin is identified that induces cell death of PGCs, either without impairing viability of somatic cells or limited to benign somatic cell loss or both. In each of the breeds, the toxin is not active, thus, within itself, each breed is fertile.

By crossing Breed A with Breed B, the offspring Breed AB arises. Breed AB contains the two SIE copies (i.e., the first conjugating element and the second conjugating element), one from each parent. Having these two elements together, they will form a functionally active conjugating element (e.g., via dimerization, via covalent binding, etc.) that will fuse the first element of interest and the second element of interest, which in turn will either deactivate the FRG, activate the SIF, produce or active the toxin, or a combination thereof. The inactivated FRG, activated SIF, activated toxin, or any combination thereof renders the organisms of Breed AB sterile.

### Example 14: Production of Sterile Avian Embryos Devoid of Functional Primordial Germ Cells (PGCs) while Maintaining Founder Populations Via a SpyTag-SpyCatcher Conjugating Element System

Objective: To produce a population of sterile avian embryos devoid of functional PGCs while maintaining founder populations using a SpyTag-SpyCatcher conjugating element system (see, e.g., Wei et al., J. Biol. Chem. (2021297(4): 101119; https://doi.org/10.1016/j.jbc.2021.101119).

PGCs of a desired avian species or breed are isolated and cultured. A gene of interest (GOI) or fertility-related gene (FRG), the product of which has a function related to fertility (e.g., having an isolated function in PGCs, gamete maturation, or gamete function), is identified, such that deletion or mutation of the gene product will lead to sterility, preferably without having an impact on viability.

Two breeds are created, Breed A and Breed B, as described above. In each breed, there is a semi-inactivating element (SIE). In Breed A, the SIE is a functionally inactive SpyTag conjugating element operably linked to a promoter and to the first element of interest. In Breed B, the SIE is a functionally inactive SpyCatcher conjugating element operably linked to a promoter and to the second element of interest. In each of the breeds there is a copy of an active fertility-required gene (FRG), thus, within itself, each breed is fertile, while each breed contains an inactive conjugating element—inactive due to the absence of its corresponding conjugating element. A SIF is identified that inactivates the FRG or induces sterility by another means. In each of the breeds, the SIF is not active, thus, within itself, each breed is fertile. A toxin is identified that induces cell death of PGCs, either without impairing viability of somatic cells or limited to benign somatic cell loss or both. In each of the breeds, the toxin is not active, thus, within itself, each breed is fertile.

By crossing Breed A with Breed B, the offspring Breed AB arises. Breed AB contains the two SIE copies (i.e., the SpyTag conjugating element and the SpyCatcher conjugating element), one from each parent. Having these two elements together, they will form a functionally active SpyTag-SpyCatcher conjugating element (e.g., via dimerization, via covalent binding, etc.) that will fuse the first element of interest and the second element of interest, which in turn will either deactivate the FRG, activate the SIF, produce or active the toxin, or a combination thereof. The inactivated FRG, activated SIF, activated toxin, or any combination thereof renders the organisms of Breed AB sterile.

### Example 15: Production of Sterile Avian Embryos Devoid of Functional Primordial Germ Cells (PGCs) while Maintaining Founder Populations Via a Split-Cre Dimerization System

Objective: To produce a population of sterile avian embryos devoid of functional PGCs while maintaining founder populations using a Split-Cre dimerization system the Split-Cre system (see, e.g., Hirrlinger et al., PLoS ONE (2009) 4(1): 286; https://journals.plos.org/plosone/article?id=10.1371/journal.pone.0004286).

PGCs of a desired avian species or breed are isolated and cultured. A gene of interest (GOI) or fertility-related gene (FRG), the product of which has a function related to fertility (e.g., having an isolated function in PGCs, gamete maturation, or gamete function), is identified, such that deletion or mutation of the gene product will lead to sterility, preferably without having an impact on viability.

Two breeds are created, Breed A and Breed B, as described above. In each breed, there is a semi-inactivating element (SIE). In Breed A, the SIE is a functionally inactive N-terminal Cre recombinase moiety (NCre) operably linked to a promoter and to the first element of interest. In Breed B, the SIE is a functionally inactive C-terminal Cre recombinase moiety (CCre) operably linked to a promoter and to the second element of interest. Together the two non-functional Cre moieties dimerize to form a functional Cre.

In each of the breeds there is a copy of an active fertility-required gene (FRG), thus, within itself, each breed is fertile, while each breed contains an inactive conjugating element—inactive due to the absence of its corresponding conjugating element. A SIF is identified that inactivates the FRG or induces sterility by another means. In each of the breeds, the SIF is not active, thus, within itself, each breed is fertile. A toxin is identified that induces cell death of PGCs, either without impairing viability of somatic cells or limited to benign somatic cell loss. In each of the breeds, the toxin is not active, thus, within itself, each breed is fertile.

By crossing Breed A with Breed B, the offspring Breed AB arises. Breed aB contains the two SIe copies (i.e., the NCre and the CCre), one from each parent. Having these two elements together, they will form a functionally active Cre recombinase (e.g., via dimerization) that will fuse the first element of interest and the second element of interest, which in turn will either deactivate the FRG, activate the SIF, produce or active the toxin, or a combination thereof. The inactivated FRG, activated SIF, activated toxin, or any combination thereof renders the organisms of Breed AB sterile.

### Example 16: Production of Sterile Avian Embryos Devoid of Functional Primordial Germ Cells (PGCs) while Maintaining Founder Populations Via a CRISPR Moieties System

Objective: To produce a population of sterile avian embryos devoid of functional PGCs while maintaining founder populations using a CRISPR moieties system.

PGCs of a desired avian species or breed are isolated and cultured. A gene of interest (GOI) or fertility-related gene (FRG), the product of which has a function related to fertility (e.g., having an isolated function in PGCs, gamete maturation, or gamete function), is identified, such that deletion or mutation of the gene product will lead to sterility, preferably without having an impact on viability.

Two breeds are created, Breed A and Breed B, as described above. In each breed, there is a semi-inactivating element (SIE). In Breed A, the SIE is a functionally inactive CRISPR moiety operably linked to a promoter and to the first element of interest. In Breed B, the SIE is a functionally inactive CRISPR moiety operably linked to a promoter and to the second element of interest. Together the two non-functional CRISPR moieties dimerize to form a functional CRISPR. In each of the breeds there is a copy of an active fertility-required gene (FRG), thus, within itself, each breed is fertile, while each breed contains an inactive conjugating element—inactive due to the absence of its corresponding conjugating element. A SIF is identified that inactivates the FRG or induces sterility by another means.

In each of the breeds, the SIF is not active, thus, within itself, each breed is fertile. A toxin is identified that induces cell death of PGCs, either without impairing viability of somatic cells or limited to benign somatic cell loss. In each of the breeds, the toxin is not active, thus, within itself, each breed is fertile.

By crossing Breed A with Breed B, the offspring Breed AB arises. Breed AB contains the two SIE copies (i.e., the two separately non-functional CRISPR moieties), one from each parent. Having these two elements together, they will form a functionally active CRISPR (e.g., via dimerization, etc.) that will fuse the first element of interest and the second element of interest, which in turn will either deactivate the FRG, activate the SIF, produce or active the toxin, or a combination thereof. The inactivated FRG, activated SIF, activated toxin, or any combination thereof renders the organisms of Breed AB sterile.

### Sequences for Examples 17-21

To produce a population of sterile avian embryos devoid of functional PGCs while maintaining founder populations via different methods, various constructs are prepared. Table 1 provides a listing of sequences relevant to the Examples that follow.

TABLE 1
Sequences Pertaining to DNA Editing Systems.

| Name | |
|---|---|
| (Genomic location | |
| with GRCg7w, if | Sequence |
| applicable) | (SEQ ID NO:) |
| LoxP site | ATAACTTCGTATAATGTATGCTATACGAAGTTAT |
| | (SEQ ID NO: 1) |
| pCAGG promoter | ACTAGTTATTAATAGTAATCAATTACGGGGTCATTAGTTCATAGC |
| | CCATATATGGAGTTCCGCGTTACATAACTTACGGTAAATGGCCCG |
| | CCTGGCTGACCGCCCAACGACCCCCGCCCATTGACGTCAATAATG |
| | ACGTATGTTCCCATAGTAACGCCAATAGGGACTTTCCATTGACGT |
| | CAATGGGTGGAGTATTTACGGTAAACTGCCCACTTGGCAGTACAT |
| | CAAGTGTATCATATGCCAAGTACGCCCCCTATTGACGTCAATGAC |
| | GGTAAATGGCCCGCCTGGCATTATGCCCAGTACATGACCTTATGG |
| | GACTTTCCTACTTGGCAGTACATCTACGTATTAGTCATCGCTATT |
| | ACCATGGTCGAGGTGAGCCCCACGTTCTGCTTCACTCTCCCCATC |
| | TCCCCCCCCTCCCCACCCCCAATTTTGTATTTATTTATTTTTTAA |
| | TTATTITGTGCAGCGATGGGGGCGGGGGGGGGGGGGGGGCGCGCG |
| | CCAGGCGGGGCGGGGGGGGCGAGGGGCGGGGCGGGGCGAGGCGGA |
| | GAGGTGCGGCGGCAGCCAATCAGAGCGGCGCGCTCCGAAAGTTTC |
| | CTTTTATGGCGAGGCGGCGGCGGCGGCGGCCCTATAAAAAGCGAA |
| | GCGCGCGGCGGGCGGGGAGTCGCTGCGACGCTGCCTTCGCCCCGT |
| | GCCCCGCTCCGCCGCCGCCTCGCGCCGCCCGCCCCGGCTCTGACT |
| | GACCGCGTTACTCCCACAGGTGAGCGGGCGGGACGGCCCTTCTCC |
| | TCCGGGCTGTAATTAGCGCTTGGTTTAATGACGGCTTGTTTCTTT |
| | TCTGTGGCTGCGTGAAAGCCTTGAGGGGCTCCGGGAGGGCCCTTT |
| | GTGCGGGGGGAGCGGCTCGGGGGGTGCGTGCGTGTGTGTGTGCGT |
| | GGGGAGCGCCGCGTGCGGCTCCGCGCTGCCCGGCGGCTGTGAGCG |
| | CTGCGGGCGCGGCGCGGGGCTTTGTGCGCTCCGCAGTGTGCGCGA |
| | GGGGAGCGCGGCCGGGGGCGGTGCCCCGCGGTGCGGGGGGGGCTG |
| | CGAGGGGAACAAAGGCTGCGTGCGGGGTGTGTGCGTGGGGGGGTG |
| | AGCAGGGGGTGTGGGCGCGTCGGTCGGGCTGCAACCCCCCCTGCA |
| | CCCCCCTCCCCGAGTTGCTGAGCACGGCCCGGCTTCGGGTGCGGG |
| | GCTCCGTACGGGGCGTGGCGCGGGGCTCGCCGTGCCGGGCGGGGG |
| | GTGGCGGCAGGTGGGGGTGCCGGGCGGGGCGGGGCCGCCTCGGGC |
| | CGGGGAGGGCTCGGGGGAGGGGCGCGGCGGCCCCCGGAGCGCCGG |
| | CGGCTGTCGAGGCGCGGCGAGCCGCAGCCATTGCCTTTTATGGTA |
| | ATCGTGCGAGAGGGCGCAGGGACTTCCTTTGTCCCAAATCTGTGC |
| | GGAGCCGAAATCTGGGAGGCGCCGCCGCACCCCCTCTAGCGGGCG |
| | CGGGGCGAAGCGGTGCGGCGCCGGCAGGAAGGAAATGGGCGGGGA |
| | GGGCCTTCGTGCGTCGCCGCGCCGCCGTCCCCTTCTCCCTCTCCA |
| | GCCTCGGGGCTGTCCGCGGGGGGACGGCTGCCTTCGGGGGGGACG |
| | GGGCAGGGCGGGGTTCGGCTTCTGGCGTGTGACCGGCGGCTCTAG |
| | AGCCTCTGCTAACCATGTTCATGCCTTCTTCTTTTTCCTACAGCT |
| | CCTGGGCAACGTGCTGGTTA |
| | (SEQ ID NO: 2) |
| NLS | CCCAAGAAGAAGAGGAAGGTG |
| | (SEQ ID NO: 3) |
| NLS | PKKKRKV |
| | (SEQ ID NO: 4) |
| nCre 19-59 | ACGAGTGATGAGGTTCGCAAGAACCTGATGGACATGTTCAGGGAT |
| | CGCCAGGCGTTTTCTGAGCATACCTGGAAAATGCTTCTGTCCGTT |
| | TGCCGGTCGTGGGCGGCATGGTGCAAGTTGAAT |
| | (SEQ ID NO: 5) |
| nCre 19-59 | TSDEVRKNLMDMFRDRQAFSEHTWKMLLSVCRSWAAWCKLN |
| | (SEQ ID NO: 6) |
| nIntein | AAGATGGTTCAGTAATCCGAGCTACCTCTGACCACCGCTTTTTAA |
| | CCACCGATTATCAACTGTTGGCGATCGAAGAAATTTTTGCTAGGC |
| | AACTGGACTTGTTGACTTTAGAAAATATTAAGCAAACTGAAGAAG |
| | CTCTTGACAACCATCGTCTTCCCTTTCCATTACTTGACGCTGGGA |
| | CAATTAAATAA |
| | (SEQ ID NO: 7) |
| nIntein | KFAEYCLSFGTEILTVEYGPLPIGKIVSEEINCSVYSVDPEGRVY |
| | TQAIAQWHDRGEQEVLEYELEDGSVIRATSDHRFLTTDYQLLAIE |
| | EIFARQLDLLTLENIKQTEEALDNHRLPFPLLDAGTIK |
| | (SEQ ID NO: 8) |
| cIntein | ATGGTTAAAGTTATCGGTCGTCGTTCCCTCGGAGTGCAAAGAATA |
| | TTTGATATTGGTCTTCCCCAAGACCATAATTTTCTGCTAGCCAAT |
| | GGGGCGATCGCCGCCAATTGTTTTAACAAATCC |
| | (SEQ ID NO: 9) |
| cIntein | MVKVIGRRSLGVQRIFDIGLPQDHNFLLANGAIAANCFNKS |
| | (SEQ ID NO: 10) |
| cCre 60-343 | AACCGGAAATGGTTTCCCGCAGAACCTGAAGATGTTCGCGATTAT |
| | CTTCTATATCTTCAGGCGCGCGGTCTGGCAGTAAAAACTATCCAG |
| | CAACATTTGGGCCAGCTAAACATGCTTCATCGTCGGTCCGGGCTG |
| | CCACGACCAAGTGACAGCAATGCTGTTTCACTGGTTATGCGGCGG |
| | ATCCGAAAAGAAAACGTTGATGCCGGTGAACGTGCAAAACAGGCT |
| | CTAGCGTTCGAACGCACTGATTTCGACCAGGTTCGTTCACTCATG |
| | GAAAATAGCGATCGCTGCCAGGATATACGTAATCTGGCATTTCTG |
| | GGGATTGCTTATAACACCCTGTTACGTATAGCCGAAATTGCCAGG |
| | ATCAGGGTTAAAGATATCTCACGTACTGACGGTGGGAGAATGTTA |
| | ATCCATATTGGCAGAACGAAAACGCTGGTTAGCACCGCAGGTGTA |
| | GAGAAGGCACTTAGCCTGGGGGTAACTAAACTGGTCGAGCGATGG |
| | ATTTCCGTCTCTGGTGTAGCTGATGATCCGAATAACTACCTGTTT |
| | TGCCGGGTCAGAAAAAATGGTGTTGCCGCGCCATCTGCCACCAGC |
| | CAGCTATCAACTCGCGCCCTGGAAGGGATTTTTGAAGCAACTCAT |
| | CGATTGATTTACGGCGCTAAGGATGACTCTGGTCAGAGATACCTG |
| | GCCTGGTCTGGACACAGTGCCCGTGTCGGAGCCGCGCGAGATATG |
| | GCCCGCGCTGGAGTTTCAATACCGGAGATCATGCAAGCTGGTGGC |
| | TGGACCAATGTAAATATTGTCATGAACTATATCCGTAACCTGGAT |
| | AGTGAAACAGGGGCAATGGTGCGCCTGCTGGAAGATGGCGATTAG |
| | (SEQ ID NO: 11) |
| cCre 60-343 | NRKWFPAEPEDVRDYLLYLQARGLAVKTIQQHLGQLNMLHRRSGL |
| | PRPSDSNAVSLVMRRIRKENVDAGERAKQALAFERTDFDQVRSLM |
| | ENSDRCQDIRNLAFLGIAYNTLLRIAEIARIRVKDISRTDGGRML |
| | IHIGRTKTLVSTAGVEKALSLGVTKLVERWISVSGVADDPNNYLF |
| | CRVRKNGVAAPSATSQLSTRALEGIFEATHRLIYGAKDDSGQRYL |
| | AWSGHSARVGAARDMARAGVSIPEIMQAGGWTNVNIVMNYIRNLD |
| | SETGAMVRLLEDGD |
| | (SEQ ID NO: 12) |
| P2A | GGCAGCGGCGCCACCAACTTCAGCCTGCTGAAGCAGGCCGGCGAC |
| | GTGGAGGAGAACCCCGGCCCC |
| | (SEQ ID NO: 13) |
| P2A | GSGATNFSLLKQAGDVEENPGP |
| | (SEQ ID NO: 14) |
| GFP | GTGAGCAAGGGCGAGGAGCTGTTCACCGGGGTGGTGCCCATCCTG |
| | GTCGAGCTGGACGGCGACGTAAACGGCCACAAGTTCAGCGTGTCC |
| | GGCGAGGGCGAGGGCGATGCCACCTACGGCAAGCTGACCCTGAAG |
| | TTCATCTGCACCACCGGCAAGCTGCCCGTGCCCTGGCCCACCCTC |
| | GTGACCACCCTGACCTACGGCGTGCAGTGCTTCAGCCGCTACCCC |
| | GACCACATGAAGCAGCACGACTTCTTCAAGTCCGCCATGCCCGAA |
| | GGCTACGTCCAGGAGCGCACCATCTTCTTCAAGGACGACGGCAAC |
| | TACAAGACCCGCGCCGAGGTGAAGTTCGAGGGCGACACCCTGGTG |
| | AACCGCATCGAGCTGAAGGGCATCGACTTCAAGGAGGACGGCAAC |
| | ATCCTGGGGCACAAGCTGGAGTACAACTACAACAGCCACAACGTC |
| | TATATCATGGCCGACAAGCAGAAGAACGGCATCAAGGTGAACTTC |
| | AAGATCCGCCACAACATCGAGGACGGCAGCGTGCAGCTCGCCGAC |
| | CACTACCAGCAGAACACCCCCATCGGCGACGGCCCCGTGCTGCTG |
| | CCCGACAACCACTACCTGAGCACCCAGTCCGCCCTGAGCAAAGAC |
| | CCCAACGAGAAGCGCGATCACATGGTCCTGCTGGAGTTCGTGACC |
| | GCCGCCGGGATCACTCTCGGCATGGACGAGCTGTACAAG |
| | (SEQ ID NO: 15) |
| GFP | VSKGEELFTGVVPILVELDGDVNGHKFSVSGEGEGDATYGKLTLK |
| | FICTTGKLPVPWPTLVTTLTYGVQCFSRYPDHMKQHDFFKSAMPE |
| | GYVQERTIFFKDDGNYKTRAEVKFEGDTLVNRIELKGIDFKEDGN |
| | ILGHKLEYNYNSHNVYIMADKQKNGIKVNFKIRHNIEDGSVQLAD |
| | HYQQNTPIGDGPVLLPDNHYLSTQSALSKDPNEKRDHMVLLEFVT |
| | AAGITLGMDELYK |
| | (SEQ ID NO: 16) |
| T2A | GGCAGTGGAGAGGGCAGAGGAAGTCTGCTAACATGCGGTGACGTC |
| | GAGGAGAATCCTGGCCCA |
| | (SEQ ID NO: 17) |
| T2A | GSGEGRGSLLTCGDVEENPGP |
| | (SEQ ID NO: 18) |
| 5′ssODN (with LoxP) | TATGCAGATGCCTATGTTTAGATAGTATGGTAGCCCCAATAGCTT |
| | TACCAAAATTCCTCCTATAACTTCGTATAATGTATGCTATACGAA |
| | GTTATATAGTGAACATAAATATAACATTTTTTTGTAAGAGCTTCT |
| | TTATTGCTTCTATCTAACCCA |
| | (SEQ ID NO: 19) |
| 3′ssODN (with LoxP) | TCCACTTTCCTCTGACCTGAACTGAGTGAGAAACTTTGAGGCTGT |
| | GAGTTACGTTCAAGTCATAACTTCGTATAATGTATGC |
| | TATACGAAGTTATTAAAGGGTGCACGTGGAATGTGGGTGTGCGAA |
| | GCACATCACCGCTGTAGTTATTCCATTAC |
| | (SEQ ID NO: 20) |
| sgRNA-5′ssODN | ATATTTATGTTCACTATAGG |
| (ch2: | (SEQ ID NO: 21) |
| 34,397,674 . . . | |
| 34,397,693 | |
| white leghorn | |
| layer GRCg7w) | |
| sgRNA-3′ssODN | TGAGTTACGTTCAAGTCTAA |
| (ch2: | (SEQ ID NO: 22) |
| 34,392,740 . . . | |
| 34,392,759 | |
| white leghorn | |
| layer GRCg7w) | |
| 5′ HA of TV | GAATTGTGGAAGAATTGCAGCTACTAAAAAGGCAGACTGGGTAGC |
| to the last | ACTTCCCACCTTACTCAGATTTAGGAGTCTCAAGACATGTAGATG |
| exon of DAZL | GGTGAAATTTATTATTCTTTTCTCTGCTTGCTTTTCTGGAAAGCT |
| (ch2: | ACCTCTATTTGGCCAGCAGTTGGCTCTGAATTCCTTTCTTTAAAG |
| 34,384,285 . . . | GAGACCTGACTGCTCTCCTGTCTGCCTCTTACACAGCTGTACTGG |
| 34,385,615 | TGTGTCACTGATAGAAACGTTTACCTTGAAAGTGACAAGTGGGGG |
| white leghorn | CGTTGATACCCATTAACAACATTAAAGGGAAAATTTCAGGAAACA |
| layer GRCg7w) | AGAAAACTGAGAGGAGTTCAGAATGAAATGTAAGCCCTGTGACAG |
| | ACTCACACTGATGGTAGTAGGGTAACTGATGGAGATGCATCTATA |
| | ACAATTCTAACTTTTATTTCAGAAGTCTGTGGACAGGAGCATACA |
| | AACAGTAGTATCTTGTCTGTTTAACCCTGAAAACCGTCTGAGGAA |
| | CACCTTTGTATCACAAGAAGACTACTTCAGGGTATGTAGTGAAAG |
| | CAAAACTAAATCTCTAAAACCAGCTGATCTTAACTTTGTTCATAT |
| | ACAGGATGCCATGAAGACAAAGGTTTATCTGTACTTGCTTGAGAT |
| | TTTACTTTTTGTATGTGCATTGAGATGAATGACTGGTTAGCTCTC |
| | AGTTGGTTAATACTCAATGAAAATTGCAGAAATTGAGTTCAGTGG |
| | ATTTAAGTGCATTGATACAAGGCTGCATAAGAACTGCTGAGCAGT |
| | CAGCTATTGGATATTTAGAGCTAATCATCCCTCAGGAGCAATCTG |
| | CTGAAAGGTAAGCAGATTGCATCTTGTAAACGAGAGAGCAGAGTT |
| | AAACGTTTGTGGTCTTTGCTCCACTTAAGTACCCACAGTGGATGT |
| | CTATCTGTACTTTTGGTTAAAAAATAGATAATTTCTACAACAGAC |
| | TGTAGTTAGGTCAGTACCTGGTCATCAGGTATTAATGGAAAGCTT |
| | TTTGCTGTGCATTCAATCTTCATAGAGGTCTGCTTTCCAATTACA |
| | TTCTCTTTGTTTTAAGTATAACTTTGAAACTCAAATATAAAAGCC |
| | TGCTACTTTTTTTTGTTCTAAAAAGACAGATGTGGACTAGGCACC |
| | TGTACTGTGAAACCAGAATAGAGAAGCTGTTGGGCAGATITGAAG |
| | AGGATTGAGTAATATAGAGAATGTCTTGAGATATTTGTGAAGCTT |
| | TTATTGCTTTGTTGCAATTGTTGTCTAGAAACATGGGCTTTTTCT |
| | GTTTTTTCTCCTATTCCAGGAGAGGAGGGCGCATCACTTTAGGAA |
| | AGGAAGAGCAGTGCTCAAAAGTGTT |
| | (SEQ ID NO: 23) |
| 3′ HA of TV | TGATGAACAAAGACTTTGAAGTACATAAATGTATTACTTTGATGT |
| to the last | TCCTACAGTTCAGTTTAGTAAGATGTGTAGTAAAAAGTGTAACCT |
| exon of DAZL | TGTTCAAAAAGTTGCTTCAAGTTGATGTTTGTGTTCTGTTTTACC |
| (ch2: | TGTTCCAGAATAGCTATTTTTGCTTGAGAAGTTTGAAGTTGTAAG |
| 34,382,718 . . . | AGTTGAAATATTTCCAGGTTTTATTACTAGCTTGCATGCTTTTCC |
| 34,384,284 | TGCTAACTAACTGAAATGCTAATCTTAAGGAATTTATATGGGGAA |
| white leghorn | GGGGAAAAAAGAAAAACACTTTGTTTGGTACGTGTGGATTTTCTT |
| layer GRCg7w) | CTGAGCTTTAAGGTACAGTTTGTTGCATGTTAAAATTTAGTTCTT |
| | ATTAAACCACAACTTTAAGTTACTAACGTCAACCAGTTACCTCTT |
| | GCAGTTCAAAAGTTGAAGCAGTTCCTTGTCCAAGATGGAGTATTT |
| | TAAAACTGAGCTCTTAATCAGTGGAACAGAAGACGTCACGGTGTA |
| | ACTCAACTGAAGCCCTTTAAGTCCCGGTTCTCTTTAGACTACCTA |
| | ATCAATGTCTTTGTTTGCTAACGACAGTTTATCTATGTGAATCCT |
| | AAAATTCCTATATGTAACTTAAGATGCAAGAATGTAATTAGTTAC |
| | ATTGGCTGCTCAGTGGAGTATGACTTTTTTTTTTACTGGATTAAT |
| | TTTAGCAATACCTGTATCTTAAAATTGTGAGAAAATACTGCATTT |
| | AAAATATGCCTAACTTTGTGATGCAATATGTTAATCAAAGAATAC |
| | ATGTAAGCATATTTTAATAATAATTATGTAGATTITAGTCATGTA |
| | TTTTGAAACAATTAAAATTTTTAATTTTGACTTACCTTCCCAGTG |
| | TGAGTGACATCCTAATATAATACTTCTAAATCTTAAGCTGCTTTG |
| | AGAAAGGCATGCAGCGTATTTATTGAAGGAATTGAAGATTTCTTA |
| | CCCTACATAAGAATTCCAGTTAGGACAAGTTTATAGCAACAAACT |
| | ITCACATTTGCTGTTAGTTCTACCTGCGATTTTGAAGGAGTACAA |
| | CTGAGAACAGCACTCAGTCTTGTATGTGTGTTGGGTCCTAGTCTG |
| | ATTCATTTTTCTTATTACTACCCTTATACCTCAGTCTCCAAGTAA |
| | AAAAGGAAATAACTCCTCCTTTGTAGACGTGTATATGTGAATGAA |
| | TAGAATGGCATGTCCCACTTCAAATGTCTAGAAGTAGATGTTGGT |
| | GAAACATGCAATAAGAGCTGAGTTGCTCTGTACCTGGATAGTGGG |
| | CTGTAAGATGCAGCACAGGAGTGTCCCAGGACTGTTGTTCAGGAG |
| | TAGGAGTCAGGGGAGGGAGGCAGGGCTTGGGGGATAGCAATAATA |
| | GTTGGTGCCGTTCTCCATAAAATTACTCAGAAGCAATGTTCTGGC |
| | AGCATAAATCGACCTAAAGTTGTTAACTTTTTCTCTGCTCAGACC |
| | GTTCCCTAGCACTAAAGATGCTGTGTCCTTAAGTGTGTTTCCATA |
| | CTATCCATCACCATGTTTTTCCCAACCTATTGCTTTAATAGTATT |
| | GTTAGTTTGATATACTTAACTTCTTCCGCGTTTCC |
| | (SEQ ID NO: 24) |
| TV-sgRNA | AGGGCGCATCACTTCAGAAA |
| | (SEQ ID NO: 25) |
| Cre recombinase | ATGGCACCCAAGAAGAAGAGGAAGGTGTCCAATTTACTGACCGTA |
| including NLS | CACCAAAATTTGCCTGCATTACCGGTCGATGCAACGAGTGATGAG |
| | GTTCGCAAGAACCTGATGGACATGTTCAGGGATCGCCAGGCGTTT |
| | TCTGAGCATACCTGGAAAATGCTTCTGTCCGTTTGCCGGTCGTGG |
| | GCGGCATGGTGCAAGTTGAATAACCGGAAATGGTTTCCCGCAGAA |
| | CCTGAAGATGTTCGCGATTATCTTCTATATCTTCAGGCGCGCGGT |
| | CTGGCAGTAAAAACTATCCAGCAACATTTGGGCCAGCTAAACATG |
| | CTTCATCGTCGGTCCGGGCTGCCACGACCAAGTGACAGCAATGCT |
| | GTTTCACTGGTTGTGCGGCGGATCCGAAAAGAAAACGTTGATGCC |
| | GGTGAACGTGCAAAACAGGCTCTAGCGTTCGAACGCACTGATTTC |
| | GACCAGGTTCGTTCACTCATGGAAAATAGCGATCGCTGCCAGGAT |
| | ATACGTAATCTGGCATTTCTGGGGATTGCTTATAACACCCTGTTA |
| | CGTATAGCCGAAATTGCCAGGATCAGGGTTAAAGATATCTCACGT |
| | ACTGACGGTGGGAGAATGTTAATCCATATTGGCAGAACGAAAACG |
| | CTGGTTAGCACCGCAGGTGTAGAGAAGGCACTTAGCCTGGGGGTA |
| | ACTAAACTGGTCGAGCGATGGATTTCCGTCTCTGGTGTAGCTGAT |
| | GATCCGAATAACTACCTGTTTTGCCGGGTCAGAAAAAATGGTGTT |
| | GCCGCGCCATCTGCCACCAGCCAGCTATCAACTCGCGCCCTGGAA |
| | GGGATTTTTGAAGCAACTCATCGATTGATTTACGGCGCTAAGGAT |
| | GACTCTGGTCAGAGATACCTGGCCTGGTCTGGACACAGTGCCCGT |
| | GTCGGAGCCGCGCGAGATATGGCCCGCGCTGGAGTTTCAATACCG |
| | GAGATCATGCAAGCTGGTGGCTGGACCAATGTAAATATTGTCATG |
| | AACTATATCCGTAACCTGGATAGTGAAACAGGGGCAATGGTGCGC |
| | CTGCTGCAAGATGGCGAT |
| | (SEQ ID NO: 26) |
| Cre recombinase | MAPKKKRKVSNLLTVHQNLPALPVDATSDEVRKNLMDMFRDRQAF |
| including NLS | SEHTWKMLLSVCRSWAAWCKLNNRKWFPAEPEDVRDYLLYLQARG |
| | LAVKTIQQHLGQLNMLHRRSGLPRPSDSNAVSLVVRRIRKENVDA |
| | GERAKQALAFERTDFDQVRSLMENSDRCQDIRNLAFLGIAYNTLL |
| | RIAEIARIRVKDISRTDGGRMLIHIGRTKTLVSTAGVEKALSLGV |
| | TKLVERWISVSGVADDPNNYLFCRVRKNGVAAPSATSQLSTRALE |
| | GIFEATHRLIYGAKDDSGQRYLAWSGHSARVGAARDMARAGVSIP |
| | EIMQAGGWTNVNIVMNYIRNLDSETGAMVRLLQDGD |
| | (SEQ ID NO: 27) |
| Flippase recombinase | ATGGCTCCTAAGAAGAAGAGGAAGGTGATGAGCCAGTTCGACATC |
| including NLS | CTGTGCAAGACCCCCCCCAAGGTGCTGGTGCGGCAGTTCGTGGAG |
| | AGATTCGAGAGGCCCAGCGGCGAGAAGATCGCCAGCTGTGCCGCC |
| | GAGCTGACCTACCTGTGCTGGATGATCACCCACAACGGCACCGCC |
| | ATCAAGAGGGCCACCTTCATGAGCTACAACACCATCATCAGCAAC |
| | AGCCTGAGCTTCGACATCGTGAACAAGAGCCTGCAGTTCAAGTAC |
| | AAGACCCAGAAGGCCACCATCCTGGAGGCCAGCCTGAAGAAGCTG |
| | ATCCCCGCCTGGGAGTTCACCATCATCCCTTACAACGGCCAGAAG |
| | CACCAGAGCGACATCACCGACATCGTGTCCAGCCTGCAGCTGCAG |
| | TTCGAGAGCAGCGAGGAGGCCGACAAGGGCAACAGCCACAGCAAG |
| | AAGATGCTGAAGGCCCTGCTGTCCGAGGGCGAGAGCATCTGGGAG |
| | ATCACCGAGAAGATCCTGAACAGCTTCGAGTACACCAGCAGGTTC |
| | ACCAAGACCAAGACCCTGTACCAGTTCCTGTTCCTGGCCACATTC |
| | ATCAACTGCGGCAGGTTCAGCGACATCAAGAACGTGGACCCCAAG |
| | AGCTTCAAGCTGGTGCAGAACAAGTACCTGGGCGTGATCATTCAG |
| | TGCCTGGTGACCGAGACCAAGACAAGCGTGTCCAGGCACATCTAC |
| | TTTTTCAGCGCCAGAGGCAGGATCGACCCCCTGGTGTACCTGGAC |
| | GAGTTCCTGAGGAACAGCGAGCCCGTGCTGAAGAGAGTGAACAGG |
| | ACCGGCAACAGCAGCAGCAACAAGCAGGAGTACCAGCTGCTGAAG |
| | GACAACCTGGTGCGCAGCTACAACAAGGCCCTGAAGAAGAACGCC |
| | CCCTACCCCATCTTCGCTATCAAGAACGGCCCTAAGAGCCACATC |
| | GGCAGGCACCTGATGACCAGCTTTCTGAGCATGAAGGGCCTGACC |
| | GAGCTGACAAACGTGGTGGGCAACTGGAGCGACAAGAGGGCCTCC |
| | GCCGTGGCCAGGACCACCTACACCCACCAGATCACCGCCATCCCC |
| | GACCACTACTTCGCCCTGGTGTCCAGGTACTACGCCTACGACCCC |
| | ATCAGCAAGGAGATGATCGCCCTGAAGGACGAGACCAACCCCATC |
| | GAGGAGTGGCAGCACATCGAGCAGCTGAAGGGCAGCGCCGAGGGC |
| | AGCATCAGATACCCCGCCTGGAACGGCATCATCAGCCAGGAGGTG |
| | CTGGACTACCTGAGCAGCTACATCAACAGGCGGATC |
| | (SEQ ID NO: 28) |
| Flippase recombinase | MAPKKKRKVMSQFDILCKTPPKVLVRQFVERFERPSGEKIASCAA |
| including NLS | ELTYLCWMITHNGTAIKRATFMSYNTIISNSLSFDIVNKSLQFKY |
| | KTQKATILEASLKKLIPAWEFTIIPYNGQKHQSDITDIVSSLQLQ |
| | FESSEEADKGNSHSKKMLKALLSEGESIWEITEKILNSFEYTSRF |
| | TKTKTLYQFLFLATFINCGRFSDIKNVDPKSFKLVQNKYLGVIIQ |
| | CLVTETKTSVSRHIYFFSARGRIDPLVYLDEFLRNSEPVLKRVNR |
| | TGNSSSNKQEYQLLKDNLVRSYNKALKKNAPYPIFAIKNGPKSHI |
| | GRHLMTSFLSMKGLTELTNVVGNWSDKRASAVARTTYTHQITAIP |
| | DHYFALVSRYYAYDPISKEMIALKDETNPIEEWQHIEQLKGSAEG |
| | SIRYPAWNGIISQEVLDYLSSYINRRI |
| | (SEQ ID NO: 29) |
| FRT | GAAGTTCCTATTCCGAAGTTCCTATTCTCTAGAAAGTATAGGAAC |
| | TTC |
| | (SEQ ID NO: 30) |
| FRT-5′ssODN (with | TATGCAGATGCCTATGTTTAGATAGTATGGTAGCCCCAATAGCTT |
| FRT) | TACCAAAATTCCTCCTGAAGTTCCTATTCCGAAGTTCCTATTCTC |
| | TAGAAAGTATAGGAACTTCATAGTGAACATAAATATAACATTTTT |
| | TTGTAAGAGCTTCTTTATTGCTTCTATCTAACCCA |
| | (SEQ ID NO: 31) |
| FRT-3′ssODN (with | TCCACTTTCCTCTGACCTGAACTGAGTGAGAAACTTTGAGGCTGT |
| FRT) | GAGTTACGTTCAAGTCGAAGTTCCTATTCCGAAGTTCCTATTCTC |
| | TAGAAAGTATAGGAACTTCTAAAGGGTGCACGTGGAATGTGGGTG |
| | TGCGAAGCACATCACCGCTGTAGTTATTCCATTAC |
| | (SEQ ID NO: 32) |
| Dazl CRISPR1 | ATATTTATGTTCACTATAGGAGG |
| (artificial) | (SEQ ID NO: 33) |
| Dazl CRISPR2 | TGAGTTACGTTCAAGTCTAAAGG |
| (artificial) | (SEQ ID NO: 34) |
| DAZL ssODN1 | TATGCAGATGCCTATGTTTAGATAGTATGGTAGCCCCAATAGCTT |
| (artificial) | TACCAAAATTCCTCCTATAACTTCGTATAATGTATGCTATACGAA |
| | GTTATATAGTGAACATAAATATAACATTTTTTTGTAAGAGCTTCT |
| | TTATTGCTTCTATCTAACCCA |
| | (SEQ ID NO: 35) |
| DAZL ssODN2 | TCCACTTTCCTCTGACCTGAACTGAGTGAGAAACTTTGAGGCTGT |
| (artificial) | GAGTTACGTTCAAGTCATAACTTCGTATAATGTATGCTATACGAA |
| | GTTATTAAAGGGTGCACGTGGAATGTGGGTGTGCGAAGCACATCA |
| | CCGCTGTAGTTATTCCATTAC |
| | (SEQ ID NO: 36) |
| F-Pre-D-ssODN1 | TGCTTTAGTGAAAGATGTCCC |
| (artificial) | (SEQ ID NO: 37) |
| R-Pre-D-ssODN1 | GTTACTTTATTTGGCCTAATACC |
| (artificial) | (SEQ ID NO: 38) |
| F-Pre-D-ssODN2 | TGTAATGTTTGAAGTGTGTTTTAG |
| (artificial) | (SEQ ID NO: 39) |
| R-Pre-D-ssODN2 | GCACCTCAGAAAAAAAGTTTTAG |
| (artificial) | (SEQ ID NO: 40) |
| Whole Genomic | AGTGGGAGACTGAGTGAGATGACCTCCTGAAGTTCCCTCCTATCT |
| Sequence (genomic | CAGCTATTCTATCTACAGAAATACTGAACTCTTTTCAAGAGGATG |
| sequence of | CCTCAAAACTATCTTACTATCTAAAATATATATATATATITTAAT |
| unmodified PGCs) | AGTATTTGTAGCACCTCTTCTAAGACAGGTATGTTCCAATTAACA |
| | GCAAACAGGAGATAACTGTTCCTTAAGAATGCTTCTAGTTAGGGC |
| | CATAATAGAAGTGAAAAGAGGGCAACTGTTTCCCTCCTCCCCCCC |
| | CTCCCCCCCCCAAAAAAAGAAAAACAAACAAACAAAAAACCAACC |
| | CAACCTGGTGTCTGCTCTATTTTGGCACAATGAAACAAGCTTAGA |
| | TTCAGCAGCTATCACAAGCAGATTTACTCTTTTTGTGCTCTAGAA |
| | ATACTGGTACTGATGTGTTGCCACTCTTGAAATCAAATATGAATC |
| | TATTTAATTGAAAAAGATAAGTATTTTTCCTTCACTGTTGGATTT |
| | TATTTTCAAGGTTCCAGACATTTGAAACAAAAATGGAAAAGAGCT |
| | GATACAGTATTGTAGAGCCTCCCTTGTTTTTGTTTGTTTTTCCCC |
| | CAAGATGAGGGTGGTGAGACACTGGGACAGGTTGCCTAGAGGCTT |
| | TGTGTGCCCCATCATTAGAAGAATTCATAGATGGGTTGGTCAATG |
| | CTTTGAGCAACATGCTTTAGTGAAAGATGTCCCTGCCCATGGCAA |
| | GGAGGATGGACTAGAATATCTCTAAGGGCCCTTTCCAACCCATAT |
| | CATTCTTGATTCTATTTAAGGGACATTTGTTGCATGAAAACCTAT |
| | GCAGATGCCTATGTTTAGATAGTATGGTAGCCCCAATAGCTTTAC |
| | CAAAATTCCTCCTATAGTGAACATAAATATAACATTTTTTTGTAA |
| | GAGCTTCTTTATTGCTTCTATCTAACCCAACTCTGAGTCCAAATA |
| | CATCTAGGGCAGCAAGCTATTCTGGATTATTAGTGTCAGCATTTA |
| | AAAAGTTGCACATTTGGTATGCTTTTAAAAACGATAGGGGTATTA |
| | GGCCAAATAAAGTAACATGTGAGAAAACAGGCTGCCTGTTATATG |
| | CCCTCCAGTTGATCAGTTTAACTTTGTACTTTTTTTTTTCCCCAG |
| | ATTAATCAGTCTTATACCTTTGAAATTTATAGAAAGTTGCTAAGC |
| | TAGCACAGTCAAGAGTTTGTCACAGCATCCATAATGTTCCCTTCC |
| | CCAGAAGGGTCTGAAAATCTAGATATATTCTCAAGAGAAGAAAAC |
| | AAAAAGTATTTAAAACCAGCTTCCAAAGGCAGTGGAAGCACCTTA |
| | TTAACATCGAAAAAGAAAAGCTTCCAGAGATATGTCCCTTCTAGA |
| | AAGATCTTAACAGTCTAGTGAGAAGGTGTTAGAAAAGGCATTATG |
| | GTGTAATGGAACCAGCCTGCTCCAGAGTATCCATATAACATCGGT |
| | GTGAGAAAACATTGCCAAAACGTTCAGCTTTGCCTCAAAGTGGGC |
| | AACTCTAACCAAAGCCTCCTCAAGCAGTACAACCTCACTGGGCAT |
| | ATAACGCGGCTCTTCTGACAGGAAGGTGCTACCACTACGCTCTCT |
| | GAACAGCAGGGCCTCAAACAATGGCCCTACCTAATCTCCTAACAA |
| | AGGAAAAGGTTACGGCACACATCGCCCTTCGTCTTCAGCCCTCTC |
| | CTGAAACACACACCGAGGGCTGAACTCTCCCACACCGCGGCGGTG |
| | CGGTGCGGTGCGGTGCGGGCCGGACCAGGCCAGGCCAGGCCAGGC |
| | CAGCGGGGCGGGTGGGGCTTTCCCCGCCCCTTCCCCCGGGGTCTA |
| | CTTAAGAAGAGCGGCCGTCGAAAAGAACTGCCTCTTTCGCACCCC |
| | TCTTAAAAAAGAAAAGAAAGAAAAAAAAAAAAAAGACAAAAAAAA |
| | TACAAACACAAAAAAGTGGGGTTCTTTAGTGTCTGTTTTCCCAAC |
| | ACTCCAATTGTTTTTGTCTTGAAGGCCTCGTTTGTTTTTAAGTGT |
| | GCGGGCGCTGTCACAGCTCCGGGGAACGATGGTAAGTCGGGACCG |
| | GGGACAACGGGGGAGCGGAGCGGTGGTCGGGCGGGTGGAGAGCAG |
| | GGTGTGAGGTGCGCCGGGCCGGCGGGCAGTTGGCGGGGCCTCCTG |
| | AAGCTCGCGTCGTGCCGTGGTGGCAGGGCGGGGGACGGGGGAGTC |
| | CCGCAGCTGCGGCGCTGCCGCCTCACAGCGCCTTGCGGAGACTCT |
| | GAGAATTACTCCCTTTTACAATAGGTTCTAGAATCTTACGTGTAC |
| | GGTATGAATTTCAGTGATACTGCGAGAAAATGATAAGAAAAGAAG |
| | GCTGAGACATGAGTTGGTGTTGTGGTCAGAGGCGTGGTGTCAGGA |
| | GGACTTCGAGGCAGGAGACGCGTCGTGCCGCGAGCGGCCATTTTG |
| | TGACTCCGTGCACAGTCACGGCCCTTCTACTCCGCCGCCTCCGGT |
| | CGGTGCCCCGAGGTGGCAGTTGCACCCGCTCGGCGCTGGAGCCCG |
| | GCGTGACGCCTTGCCCTGCGACCGCGGGCTCTCCATCAGCTCCGT |
| | CTATTCTCAGCCTTTTGGCACTCCCTCCCCTTTCCCTTTTCCTAA |
| | GCCCGTCCAGGCCCGTGACCCGTTTCCCGGCGCCGAGCTGTTGAG |
| | CCGCCCGAGCGGACCGTTATCACCTCGGGAGCTCCGGCCCCACCA |
| | GGCGGTTTCCGGCAGCGCTCCGCGGGCCCCCAGCTGTGGTCAGCG |
| | GGGATGCTGTCCGGCCCCGCGCGTGCCCCTTCCATGCAAAGAGAC |
| | CTTCGTCTTCGAGAGTTTTCTGCGTAATGTGGTCCCCGTTGGGGC |
| | GAAGCCTCGCGTCGCTGGGTGCGGGTACAGTTCTGGTTGGTGCTA |
| | AACGTGTGCAGGGAAGGGAGCGGTAAAGCGTTCCTGTCGAGGTGA |
| | CTGTGCTCTGGTACACGAGCCGGCAGCTGAAACTAAACTGGTTGC |
| | ACACAGTACTGTTTCTCAAGAAATTCTTTATTTTCGGGTTTTTTT |
| | TACGCACGGTATCTGCTATTCTAACGCTGACACCTCAGCAACTTC |
| | CAGGCAGGACCGGGTAGTGGAGTCGAGCAGCGCTGCTGTATGGCT |
| | CCCCCTGCCGCCTCCGGGCGGGCGCAACGCCGCGGAGCCGCCGCC |
| | ATCTTCTCCTTCCGCAGCGGCTGCCATGGCAACCGAGTGCTCGTC |
| | GGGAAGGCGGGTAGCGGGCGTGAATACGGCGCAGAATGCCCCTTA |
| | TTTTAAATGAACCCCGTGTGAGTTCACGGAGGAAGCAGTCTGAGA |
| | CATCAGGAAGCAGTGCTTGTGTGGCCGAAGGAAAAAGTGAAGGTT |
| | TTCCTCCCCTAGACAGAGTTGCAGACATCACTCAGGCAGTTCCAC |
| | CTCTTTTGTTTACCACCCCCTGTTGTAATTAAGGGGTTATTCTAT |
| | TTTCCTGCCTTGAAGTCGATGCTTTGTGAAACAATGCAGAGAGTA |
| | AATTACTTGATTTACCTTGTGATAAAGGAAAAAGGGCTAGGGGGA |
| | AAGAAATTCAGCAGGTTGAAAAACTGAATAAGCTGTGAGTCTCTT |
| | AACACCTAGAAATTGGTTTTCAAAGTTACTTTGTGAGCTGCAGAG |
| | ACAGACAGAGAAGCTTTGCTTAAGGCAGTTTTGAATGGTCTGGTC |
| | CCTTTAGTTGGACTCAGTGATCCTTATGGGTCCTCTCCAGCTCGG |
| | AATGTTCTATGATTCAGCTGATGCTTGAGCTTCATGGTACGTAAT |
| | TCATCCATTTAAGTGTTGTTTACAGTTTTGGGAATTGGAAATGGA |
| | GCAGGCTTTGGCTACTGATTCAGCAGCAGTGCAGTTTGCCCGTAG |
| | TATGCACAGAAGTAAGGCTGCATTGGGTAGTAAAATAACAATGAC |
| | ATTTTGTGCTGATAAAATAAGGGTGATGCGTTGCTTGAATTGACA |
| | TGTTTTCTGTTGTAGTACTGAAGGCTTCAAAAATAACTTCGAAAG |
| | GAGAAAGACATTGTTTCATGTCTTCATACTGTAATACTTTTTCAT |
| | CATGTTCACCAGTAGCTCCTTTTTATGTTCTGAGAAATAAGCATG |
| | TTAATGCATCTAAAAGCTTGTTTTTGATGTTGAAAAGTTTGTCAC |
| | ACTGCTTAAAGAACTTACTTGCCCCTTTAGATATTTAACATGCAG |
| | AAGGGTTAAAGAACTGTGTAAGAACTGTATTTTCTTTGAAAGTTT |
| | TATTCTGCATCATTATTGCGAGAAACTAATGATGTGCTTCTATGG |
| | CAGCCAAGGCCGTTTCTTACCTGCATGTCAGCAGTATCTGTCTGC |
| | CGACCCACTTTCTTGTGCTGATTCTGGCTCTCCTGGGTGCATTTG |
| | CTGTGAGCTGCTTCCTGTTGAAAGTTGCCAGAGGATTTGCACAGC |
| | CAAGTGAATGCTTTCTTTGAGCTTCGGTCACTGAAACAGGTCCAA |
| | AAAGAGGGAACAGTAGATGCTAACAGGTCAGCTCAAGGTTGCTGT |
| | GGAGTTGGTCTCATATTTGAGTTAAAATGTTCTTTTTCTGGGGAT |
| | ATGGTCAAATAAGTGGTCTCCAGCTCTCCTCTTGTTTCCTTCTTG |
| | CTGTGATGTGTTCTCATGCTAGAAGTGTTTACACAACTGTGGAAT |
| | GAGCTTGGATCAGAGGCAGTTGTGTTGGAAAACCACAACCCATTT |
| | TCTCCTGTAAAATGTTGGGCCTTATTCCCTTTGCAGTGGTGTGAA |
| | GAGCTGTCCTGATACAATAGGAAGGAAGGGAGAAGAGTATTCTCT |
| | ATCTGCGTACAACAGAAGTAATCTGTGATACAATTTGCAGTTGGT |
| | AGCATATAATTGATGAAGTAAACAGAATTATATACAAGAACATGG |
| | TACTGGATGTGATGGAGCCAGGTACTTCAAGGTCTAGCTTCATTA |
| | GATGCTGTTGAACTGTGCTTCTCAGACTAGGTCTTTTCCATCTCC |
| | ATTTCTGATGGAGATGAAACTTAATCTGAGGTGTATCTGATGCTT |
| | ATCTGATAGCACAGTAAGCTGATTCTATTGTCTAAAATAACAGAA |
| | TACTAATCTGGTCAAAACTTGCTCTCTTGATGCTGTTCTTCCTCA |
| | AAAACTAAATGTGAACTGACAGCTGTTGGATCTTCTGCAGAGGAG |
| | GTGTAAGAGAAGAGATGGAGCAAACTGTCTCTGCTTTTTAGGGGT |
| | AGTCTGGTGTTAATTTGGCTCACTCATGTTGAAATCACACACGTG |
| | GAAAGCTGAGTTCTAGTTCTGGCGTTACTTAATTGCTGGTTGAAT |
| | CAGGCCACATGGACAGTTTGGTTCTTGTAATTTTCTTAGTCCCTT |
| | ACCGGATGTCCAATTTGAATGAACATTGATCACTTGCAGGTGCAG |
| | TTTAAATAACTCTGTGTTAAACGTCTAATTTGTTACAATCACGAG |
| | AGCCCATTTTTCAATGTAAATTATTCTTAGGTTTCAAAGTATCAG |
| | TAACCTCAACTAAATCAAAAGATCTGCCTATCTGAAATAGGGATA |
| | ATGCTACACCAGGTGAGCTGCAAGGAAAAGGTTATTAATGTTTTG |
| | AGATGTCTTAATACAGACAAATGAGCACAAATAAGGTGGTCAAAG |
| | TAGTTGTTTTTTTTACAAGCCAGATAAAGAATGACATGTACATAG |
| | AACCATTCATTCAGTTGAGAAGATGTGGAACCAAAGTTTCATCCA |
| | TCTGAGGTGTATTTCAGTTCTTGCAAATATCTTTGTGTAATGTTT |
| | GAAGTGTGTTTTAGAGTATGGAACACGTCTTGGTGTCATCAGCAA |
| | CAAGAAATGAAATTGTGTGGTCTCTGTGAACAAATGATTCCCTAA |
| | ATAAACAGTAATCCAGAATCCACTTTCCTCTGACCTGAACTGAGT |
| | GAGAAACTTTGAGGCTGTGAGTTACGTTCAAGTCTAAAGGGGGCG |
| | CGTGGAATGTGGGTGTGCGAAGCACATCACCGCCGTAGTTATTCC |
| | ATTACACCATGTAGATATGTGCAGCGCACTCTTAAGATCCTGCTT |
| | CGGTGTGTGCCACTCAGTGACAAGATCAGTCGTTCATATTTCTCT |
| | TGTAGTTAAATTGACTAAAACTTTTTTTCTGAGGTGCACATAAAG |
| | TGAATATTCTACCAAGACGGTGTCATCTACCTCACAGCTAAATTG |
| | TAGTATAACTGTACCATCGTTCCAAGGAATTTCATAAGCTTTACT |
| | CACTTCTTGAGCATCACAGGCTTTTGATCAGGAAAATGGAGTTCA |
| | TTCATCGGATAATCAATTCACAGCGTAGAACTTAAGAATTTCTTG |
| | CTTGCATCTAAAAGGAATTGTGTAAAAATTTGCGATGAATAATTT |
| | CGGGGTTCTCATTGTAAGTTGGGTAGTAGCAACAATGGTTGTGAA |
| | GCTTCCAGTCAGGCAAGGCTGCTTAGTGTAGCCTAGCTTGAGTCT |
| | TGATCTCAAAGAAGAGTAGGTTCAAACTTGTCCATTTGAGTGTTC |
| | CATTTGCAGTAAGTACTTCCTTTAAGTAATTGGAAAGATGCTATT |
| | GATACTTATGGTCTCAAACTGTATCAATAGGAAGATGGAGGCCTA |
| | CCTAGTAGTGTATTACAATGTGACTGAAATATTTCTGTTTAACCT |
| | TTTCAGTCTGCAAATGCGGAAGCCCAGTGTGGAACTATCTCAGAG |
| | GATAATACCCATTCGTCAACAACCTGCCAAGGATATGTTTTACCA |
| | GAAGGAAAAATCATGCCAAATACAGTCTTTGTTGGTGGAATTGAT |
| | ATAAGGGTATTTATGTACTTTCAATGGTITTAAACTACATATGAC |
| | ACGCTGTAGTGGGAAAGAAATAAGAATTTTAACTTCTGGAGGGCT |
| | TTTTTTTAATTGGGTCTTTACTGATCTTGAAATAATGCATTATGG |
| | TAAGAGAACTTTGAAACAAACAAAAGAGATTTTCCTGGAATATTA |
| | GGAGATGTGTTTAAAAATGGTACTTGTTGCTTTAAAACAATTGTA |
| | ACCGTTTACTGTGTCTGTGAAGTAGTTCAAGACTTGGTTTCTTTT |
| | AGATGAATGAAGCAGAAATTCGGAGTTACTTTGAACAATATGGTA |
| | CTGTGAAGGAGGTGAAAATAATCACTGACAGAACTGGTGTTTCCA |
| | AAGGGTGAGCAGAATGTCATTAGTTACTGCTTTTGTAGATGTAAT |
| | TCTAACATAAATGATGTCTGTTGTTAAGTAGTTGGTCACTTACCA |
| | TGCTTAAGCCTTTCAAATTGAGATGAATTAAAGTGAAACATGTAA |
| | AACTCGACTGATCTTAAATCTGTATAATCTTACAATTGAGAACTG |
| | GACATATTTTATGGTGTACCTGTTCAGGGAAAAT |
| | (SEQ ID NO: 41) |
| Verified sequence of | TGCTTTAGTGAAAGATGTCCCTGCCCATGGCAAGGAGGATGGACT |
| PCR product ssODN1 | AGAATATCTCTAAGGGCCCTTTCCAACCCATATCATTCTTGATTC |
| (artificial) | TATTTAAGGGACATTTGTTGCATGAAAACCTATGCAGATGCCTAT |
| | GTTTAGATAGTATGGTAGCCCCAATAGCTTTACCAAAATTCCTCC |
| | TATAACTTCGTATAATGTATGCTATACGAAGTTATATAGTGAACA |
| | TAAATATAACATTTTTTTGTAAGAGCTTCTTTATTGCTTCTATCT |
| | AACCCAACTCTGAGTCCAAATACATCTAGGGCAGCAAGCTATTCT |
| | GGATTATTAGTGTCAGCATTTAAAAAGTTGCACATTTGGTATGCT |
| | TTTAAAAACGATAGGGGTATTAGGCCAAATAAAGTAAC |
| | (SEQ ID NO: 42) |
| Verified sequence of | TGTAATGTTTGAAGTGTGTTTTAGAGTACGGAACACGTCTTGGTG |
| PCR product ssODN2 | TCATCAGCAACAAGAAATGAAATTGTGTGTGATCTCTGTGAACAA |
| (artificial) | ATGATTCCCTAAATAAACAGTAATCCAGAATCCACTTTCCTCTGA |
| | CCTGAACTGAGTGAGAAACTTTGAGGCTGTGAGTTACGTTCAAGT |
| | CATAACTTCGTATAATGTATGCTATACGAAGTTATTAAAGGGTGC |
| | ACGTGGAATGTGGGTGTGCGAAGCACATCACCGCTGTAGTTATTC |
| | CATTACACCATGTAGATATGTGCAGTGCACTCTTAAGATCCTGCT |
| | TCGGTGTGTGCCACTCAGTGACAAGATCAGTCGTTCATATTTCTC |
| | TTGTAGTTAAATTGACTAAAACTTTTTTTCTGAGGTGC |
| | (SEQ ID NO: 43) |
| Whole Genomic | AGTGGGAGACTGAGTGAGATGACCTCCTGAAGTTCCCTCCTATCT |
| Sequence with LoxP | CAGCTATTCTATCTACAGAAATACTGAACTCTTTTCAAGAGGATG |
| Insertions | CCTCAAAACTATCTTACTATCTAAAATATATATATATATITTAAT |
| (artificial) | AGTATTTGTAGCACCTCTTCTAAGACAGGTATGTTCCAATTAACA |
| | GCAAACAGGAGATAACTGTTCCTTAAGAATGCTTCTAGTTAGGGC |
| | CATAATAGAAGTGAAAAGAGGGCAACTGTTTCCCTCCTCCCCCCC |
| | CTCCCCCCCCCAAAAAAAGAAAAACAAACAAACAAAAAACCAACC |
| | CAACCTGGTGTCTGCTCTATTTTGGCACAATGAAACAAGCTTAGA |
| | TTCAGCAGCTATCACAAGCAGATTTACTCTTTTTGTGCTCTAGAA |
| | ATACTGGTACTGATGTGTTGCCACTCTTGAAATCAAATATGAATC |
| | TATTTAATTGAAAAAGATAAGTATTTTTCCTTCACTGTTGGATTT |
| | TATTTTCAAGGTTCCAGACATTTGAAACAAAAATGGAAAAGAGCT |
| | GATACAGTATTGTAGAGCCTCCCTTGTTTTTGTTTGTTTTTCCCC |
| | CAAGATGAGGGTGGTGAGACACTGGGACAGGTTGCCTAGAGGCTT |
| | TGTGTGCCCCATCATTAGAAGAATTCATAGATGGGTTGGTCAATG |
| | CTTTGAGCAACATGCTTTAGTGAAAGATGTCCCTGCCCATGGCAA |
| | GGAGGATGGACTAGAATATCTCTAAGGGCCCTTTCCAACCCATAT |
| | CATTCTTGATTCTATTTAAGGGACATTTGTTGCATGAAAACCGAT |
| | AGTATGGTAGCCCCAATAGCTTTACCAAAATTCCTCCTATAACTT |
| | CGTATAATGTATGCTATACGAAGTTATATAGTGAACATAAATATA |
| | ACATTTTTTTGTAAGAGCTTCTTTATTGCTTCTATCTAACCCAAC |
| | TCTGAGTCCAAATACATCTAGGGCAGCAAGCTATTCTGGATTATT |
| | AGTGTCAGCATITAAAAAGTTGCACATTTGGTATGCTTTTAAAAA |
| | CGATAGGGGTATTAGGCCAAATAAAGTAACATGTGAGAAAACAGG |
| | CTGCCTGTTATATGCCCTCCAGTTGATCAGTTTAACTTTGTACTT |
| | TTTTTTTTCCCCAGATTAATCAGTCTTATACCTTTGAAATTTATA |
| | GAAAGTTGCTAAGCTAGCACAGTCAAGAGTTTGTCACAGCATCCA |
| | TAATGTTCCCTTCCCCAGAAGGGTCTGAAAATCTAGATATATTCT |
| | CAAGAGAAGAAAACAAAAAGTATTTAAAACCAGCTTCCAAAGGCA |
| | GTGGAAGCACCTTATTAACATCGAAAAAGAAAAGCTTCCAGAGAT |
| | ATGTCCCTTCTAGAAAGATCTTAACAGTCTAGTGAGAAGGTGTTA |
| | GAAAAGGCATTATGGTGTAATGGAACCAGCCTGCTCCAGAGTATC |
| | CATATAACATCGGTGTGAGAAAACATTGCCAAAACGTTCAGCTTT |
| | GCCTCAAAGTGGGCAACTCTAACCAAAGCCTCCTCAAGCAGTACA |
| | ACCTCACTGGGCATATAACGCGGCTCTTCTGACAGGAAGGTGCTA |
| | CCACTACGCTCTCTGAACAGCAGGGCCTCAAACAATGGCCCTACC |
| | TAATCTCCTAACAAAGGAAAAGGTTACGGCACACATCGCCCTTCG |
| | TCTTCAGCCCTCTCCTGAAACACACACCGAGGGCTGAACTCTCCC |
| | ACACCGCGGCGGTGCGGTGCGGTGCGGTGCGGGCCGGACCAGGCC |
| | AGGCCAGGCCAGGCCAGCGGGGCGGGTGGGGCTTTCCCCGCCCCT |
| | TCCCCCGGGGTCTACTTAAGAAGAGCGGCCGTCGAAAAGAACTGC |
| | CTCTTTCGCACCCCTCTTAAAAAAGAAAAGAAAGAAAAAAAAAAA |
| | AAAGACAAAAAAAATACAAACACAAAAAAGTGGGGTTCTTTAGTG |
| | TCTGTTTTCCCAACACTCCAATTGTTTTTGTCTTGAAGGCCTCGT |
| | TTGTTTTTAAGTGTGCGGGCGCTGTCACAGCTCCGGGGAACGATG |
| | GTAAGTCGGGACCGGGGACAACGGGGGAGCGGAGCGGTGGTCGGG |
| | CGGGTGGAGAGCAGGGTGTGAGGTGCGCCGGGCCGGCGGGCAGTT |
| | GGCGGGGCCTCCTGAAGCTCGCGTCGTGCCGTGGTGGCAGGGCGG |
| | GGGACGGGGGAGTCCCGCAGCTGCGGCGCTGCCGCCTCACAGCGC |
| | CTTGCGGAGACTCTGAGAATTACTCCCTITTACAATAGGTTCTAG |
| | AATCTTACGTGTACGGTATGAATTTCAGTGATACTGCGAGAAAAT |
| | GATAAGAAAAGAAGGCTGAGACATGAGTTGGTGTTGTGGTCAGAG |
| | GCGTGGTGTCAGGAGGACTTCGAGGCAGGAGACGCGTCGTGCCGC |
| | GAGCGGCCATTTTGTGACTCCGTGCACAGTCACGGCCCTTCTACT |
| | CCGCCGCCTCCGGTCGGTGCCCCGAGGTGGCAGTTGCACCCGCTC |
| | GGCGCTGGAGCCCGGCGTGACGCCTTGCCCTGCGACCGCGGGCTC |
| | TCCATCAGCTCCGTCTATTCTCAGCCTTTTGGCACTCCCTCCCCT |
| | TTCCCTTTTCCTAAGCCCGTCCAGGCCCGTGACCCGTTTCCCGGC |
| | GCCGAGCTGTTGAGCCGCCCGAGCGGACCGTTATCACCTCGGGAG |
| | CTCCGGCCCCACCAGGCGGTTTCCGGCAGCGCTCCGCGGGCCCCC |
| | AGCTGTGGTCAGCGGGGATGCTGTCCGGCCCCGCGCGTGCCCCTT |
| | CCATGCAAAGAGACCTTCGTCTTCGAGAGTTTTCTGCGTAATGTG |
| | GTCCCCGTTGGGGCGAAGCCTCGCGTCGCTGGGTGCGGGTACAGT |
| | TCTGGTTGGTGCTAAACGTGTGCAGGGAAGGGAGCGGTAAAGCGT |
| | TCCTGTCGAGGTGACTGTGCTCTGGTACACGAGCCGGCAGCTGAA |
| | ACTAAACTGGTTGCACACAGTACTGTTTCTCAAGAAATTCTTTAT |
| | TTTCGGGTTTTTTTTACGCACGGTATCTGCTATTCTAACGCTGAC |
| | ACCTCAGCAACTTCCAGGCAGGACCGGGTAGTGGAGTCGAGCAGC |
| | GCTGCTGTATGGCTCCCCCTGCCGCCTCCGGGCGGGCGCAACGCC |
| | GCGGAGCCGCCGCCATCTTCTCCTTCCGCAGCGGCTGCCATGGCA |
| | ACCGAGTGCTCGTCGGGAAGGCGGGTAGCGGGCGTGAATACGGCG |
| | CAGAATGCCCCTTATTTTAAATGAACCCCGTGTGAGTTCACGGAG |
| | GAAGCAGTCTGAGACATCAGGAAGCAGTGCTTGTGTGGCCGAAGG |
| | AAAAAGTGAAGGTTTTCCTCCCCTAGACAGAGTTGCAGACATCAC |
| | TCAGGCAGTTCCACCTCTTTTGTTTACCACCCCCTGTTGTAATTA |
| | AGGGGTTATTCTATTTTCCTGCCTTGAAGTCGATGCTTTGTGAAA |
| | CAATGCAGAGAGTAAATTACTTGATTTACCTTGTGATAAAGGAAA |
| | AAGGGCTAGGGGGAAAGAAATTCAGCAGGTTGAAAAACTGAATAA |
| | GCTGTGAGTCTCTTAACACCTAGAAATTGGTTTTCAAAGTTACTT |
| | TGTGAGCTGCAGAGACAGACAGAGAAGCTTTGCTTAAGGCAGTTT |
| | TGAATGGTCTGGTCCCTTTAGTTGGACTCAGTGATCCTTATGGGT |
| | CCTCTCCAGCTCGGAATGTTCTATGATTCAGCTGATGCTTGAGCT |
| | TCATGGTACGTAATTCATCCATTTAAGTGTTGTTTACAGTTTTGG |
| | GAATTGGAAATGGAGCAGGCTTTGGCTACTGATTCAGCAGCAGTG |
| | CAGTTTGCCCGTAGTATGCACAGAAGTAAGGCTGCATTGGGTAGT |
| | AAAATAACAATGACATTITGTGCTGATAAAATAAGGGTGATGCGT |
| | TGCTTGAATTGACATGTTTTCTGTTGTAGTACTGAAGGCTTCAAA |
| | AATAACTTCGAAAGGAGAAAGACATTGTTTCATGTCTTCATACTG |
| | TAATACTTTTTCATCATGTTCACCAGTAGCTCCTTTTTATGTTCT |
| | GAGAAATAAGCATGTTAATGCATCTAAAAGCTTGTTTTTGATGTT |
| | GAAAAGTTTGTCACACTGCTTAAAGAACTTACTTGCCCCTTTAGA |
| | TATTTAACATGCAGAAGGGTTAAAGAACTGTGTAAGAACTGTATT |
| | TTCTTTGAAAGTTTTATTCTGCATCATTATTGCGAGAAACTAATG |
| | ATGTGCTTCTATGGCAGCCAAGGCCGTTTCTTACCTGCATGTCAG |
| | CAGTATCTGTCTGCCGACCCACTTTCTTGTGCTGATTCTGGCTCT |
| | CCTGGGTGCATTTGCTGTGAGCTGCTTCCTGTTGAAAGTTGCCAG |
| | AGGATTTGCACAGCCAAGTGAATGCTTTCTTTGAGCTTCGGTCAC |
| | TGAAACAGGTCCAAAAAGAGGGAACAGTAGATGCTAACAGGTCAG |
| | CTCAAGGTTGCTGTGGAGTTGGTCTCATATTTGAGTTAAAATGTT |
| | CTTTTTCTGGGGATATGGTCAAATAAGTGGTCTCCAGCTCTCCTC |
| | TTGTTTCCTTCTTGCTGTGATGTGTTCTCATGCTAGAAGTGTTTA |
| | CACAACTGTGGAATGAGCTTGGATCAGAGGCAGTTGTGTTGGAAA |
| | ACCACAACCCATTTTCTCCTGTAAAATGTTGGGCCTTATTCCCTT |
| | TGCAGTGGTGTGAAGAGCTGTCCTGATACAATAGGAAGGAAGGGA |
| | GAAGAGTATTCTCTATCTGCGTACAACAGAAGTAATCTGTGATAC |
| | AATTTGCAGTTGGTAGCATATAATTGATGAAGTAAACAGAATTAT |
| | ATACAAGAACATGGTACTGGATGTGATGGAGCCAGGTACTTCAAG |
| | GTCTAGCTTCATTAGATGCTGTTGAACTGTGCTTCTCAGACTAGG |
| | TCTTTTCCATCTCCATTTCTGATGGAGATGAAACTTAATCTGAGG |
| | TGTATCTGATGCTTATCTGATAGCACAGTAAGCTGATTCTATTGT |
| | CTAAAATAACAGAATACTAATCTGGTCAAAACTTGCTCTCTTGAT |
| | GCTGTTCTTCCTCAAAAACTAAATGTGAACTGACAGCTGTTGGAT |
| | CTTCTGCAGAGGAGGTGTAAGAGAAGAGATGGAGCAAACTGTCTC |
| | TGCTTTTTAGGGGTAGTCTGGTGTTAATTTGGCTCACTCATGTTG |
| | AAATCACACACGTGGAAAGCTGAGTTCTAGTTCTGGCGTTACTTA |
| | ATTGCTGGTTGAATCAGGCCACATGGACAGTTTGGTTCTTGTAAT |
| | TTTCTTAGTCCCTTACCGGATGTCCAATTTGAATGAACATTGATC |
| | ACTTGCAGGTGCAGTTTAAATAACTCTGTGTTAAACGTCTAATTT |
| | GTTACAATCACGAGAGCCCATTTTTCAATGTAAATTATTCTTAGG |
| | TTTCAAAGTATCAGTAACCTCAACTAAATCAAAAGATCTGCCTAT |
| | CTGAAATAGGGATAATGCTACACCAGGTGAGCTGCAAGGAAAAGG |
| | TTATTAATGTTTTGAGATGTCTTAATACAGACAAATGAGCACAAA |
| | TAAGGTGGTCAAAGTAGTTGTTTTTTTTACAAGCCAGATAAAGAA |
| | TGACATGTACATAGAACCATTCATTCAGTTGAGAAGATGTGGAAC |
| | CAAAGTTTCATCCATCTGAGGTGTATTTCAGTTCTTGCAAATATC |
| | TTTGTGTAATGTTTGAAGTGTGTTTTAGAGTATGGAACACGTCTT |
| | GGTGTCATCAGCAACAAGAAATGAAATTGTGTGGTCTCTGTGAAC |
| | AAATGATTCCCTAAATAAACAGTAATCCAGAATCCACTTTCCTCT |
| | GACCTGAACTGAGTGAGAAACTTTGAGGCTGTGAGTTACGTTCAA |
| | GTCATAACTTCGTATAATGTATGCTATACGAAGTTATTAAAGGGT |
| | GCACGTGGAATGTGGGTGTGCGAAGCACATCACCGCTGTAGTTAT |
| | TCCATTACACCATGTAGATATGTGCAGCGCACTCTTAAGATCCTG |
| | CTTCGGTGTGTGCCACTCAGTGACAAGATCAGTCGTTCATATTTC |
| | TCTTGTAGTTAAATTGACTAAAACTTTTTTTCTGAGGTGCACATA |
| | AAGTGAATATTCTACCAAGACGGTGTCATCTACCTCACAGCTAAA |
| | TTGTAGTATAACTGTACCATCGTTCCAAGGAATTTCATAAGCTTT |
| | ACTCACTTCTTGAGCATCACAGGCTTTTGATCAGGAAAATGGAGT |
| | TCATTCATCGGATAATCAATTCACAGCGTAGAACTTAAGAATTTC |
| | TTGCTTGCATCTAAAAGGAATTGTGTAAAAATTTGCGATGAATAA |
| | TTTCGGGGTTCTCATTGTAAGTTGGGTAGTAGCAACAATGGTTGT |
| | GAAGCTTCCAGTCAGGCAAGGCTGCTTAGTGTAGCCTAGCTTGAG |
| | TCTTGATCTCAAAGAAGAGTAGGTTCAAACTTGTCCATITGAGTG |
| | TTCCATTTGCAGTAAGTACTTCCTTTAAGTAATTGGAAAGATGCT |
| | ATTGATACTTATGGTCTCAAACTGTATCAATAGGAAGATGGAGGC |
| | CTACCTAGTAGTGTATTACAATGTGACTGAAATATTTCTGTTTAA |
| | CCTTTTCAGTCTGCAAATGCGGAAGCCCAGTGTGGAACTATCTCA |
| | GAGGATAATACCCATTCGTCAACAACCTGCCAAGGATATGTTTTA |
| | CCAGAAGGAAAAATCATGCCAAATACAGTCTTTGTTGGTGGAATT |
| | GATATAAGGGTATTTATGTACTTTCAATGGTITTAAACTACATAT |
| | GACACGCTGTAGTGGGAAAGAAATAAGAATTTTAACTTCTGGAGG |
| | GCTTTTTTTTAATTGGGTCTTTACTGATCTTGAAATAATGCATTA |
| | TGGTAAGAGAACTTTGAAACAAACAAAAGAGATTTTCCTGGAATA |
| | TTAGGAGATGTGTTTAAAAATGGTACTTGTTGCTTTAAAACAATT |
| | GTAACCGTTTACTGTGTCTGTGAAGTAGTTCAAGACTTGGTTTCT |
| | TTTAGATGAATGAAGCAGAAATTCGGAGTTACTTTGAACAATATG |
| | GTACTGTGAAGGAGGTGAAAATAATCACTGACAGAACTGGTGTTT |
| | CCAAAGGGTGAGCAGAATGTCATTAGTTACTGCTTTTGTAGATGT |
| | AATTCTAACATAAATGATGTCTGTTGTTAAGTAGTTGGTCACTTA |
| | CCATGCTTAAGCCTTTCAAATTGAGATGAATTAAAGTGAAACATG |
| | TAAAACTCGACTGATCTTAAATCTGTATAATCTTACAATTGAGAA |
| | CTGGACATATTTTATGGTGTACCTGTTCAGGGAAAATAGTGTTAA |
| | TGTCCCTCAACCAGTGGGAGCAAAGCATAAAACTTAGTGGATGCT |
| | TGTGGGGACTGTTTCACAGGCTGAAATTTTGACTTTATGATGGCC |
| | ATAGCAATTAAGCAGCCATCAGTGTAGTACCACTAATGTAATTGA |
| | GACAGGGAGTAGACTTTCATTGGGGCAGTTGGACTGCAGTCTTTT |
| | GTTGCTCAGGGGTAACTTAGAGGCAATCAAACTGTTTCAGGTGGT |
| | GAGTGAAACTTAAAGGATGGTAGAAAGCTAGAGACATTCCCATTG |
| | GATATGTAGAAAGTACTCTGATCTGTAGTGAAGAACTTAAGTGAA |
| | GATGCCTAGGACTCTGCCCAGTTGAGTTCAGAGGAAGCTCTCCCA |
| | GCTTTGAAATTAGACTTGCTTTGCGAGGAAGACTTCACCTCTAAA |
| | GATGCACCAATTGTTTTCTCTGAGCAGGTTCCAAAAAGTAGCATT |
| | TTTTTTTTCATAGACACATATAGTAACGAGCTGAAAATACTGAGC |
| | TTAATGTCTCTTGCCTGGTCTTTGTGGTGAATTCTAATGTGTGAT |
| | TAGCAAGCATATGTTCTGATTATTGATAAATTGCTGTATGTCAAT |
| | CAATGGAATACTCTACTGCGGTTCTGAGAATTGTCTCCAATATTA |
| | AGGCTTAAATAAACAAGAGGTAGTGAGATAAATTGAAAACCTCTT |
| | TTGGGATCGCTTCCTCCAATAGTGTAATTATTCCTGTAGTTCCTC |
| | CTTTCATTCAAACCTCTGCAGGAAGTACAGAATTTAGTACATACT |
| | AATTGAAGGAGCTTTTGGCTTTCTGATGCTACTAATATTAACAGT |
| | AGTACTCACTTGAGTAATTTAAATGAGAGAATATTGAATCTGGCA |
| | TTTAATTCCTTTCATTTGGCCCAGTGTGCTGTCAGTCAGCAGCAA |
| | ATGTACTTTCATGCTGAATTATATATTAATGTCCTGTTAATATCA |
| | GTTAATGTTCTTTTTACTGTTTTAGTTTTTTTAAAAAGAACTAAC |
| | AGCGGTCAAAAAATGAAAATGTAGTATTTGAATAATATTTTTTTT |
| | CTTTTCAGGTATGGATTTGTTTCATTCCTGGACAATGTGGATGTT |
| | CAAAAGATAGTAGAAGTAAGCTCTTTATGTCTTAAGTTGTCAGAA |
| | GAACCTTCTGTATGAAGGTTGTAGGTGTGGTTAGGGGATACCAGT |
| | CCCAACTGAGAAAATAAAAAAGACTAGAAGTGCCCCAAAGTAAAC |
| | TTGCTTAAATATTGTTGTGATTTACCCCAGCAGATTGGGAAGTAC |
| | CATGTACTATTTTCCTCACTGCACTCCTTCCCAGTGGTATGGGAA |
| | GAGAAAAAACAAAAACCCCAAAAAAACACCACAAAGTAGAGCTTG |
| | TAAGTTGAGATGAAAACTATTTACCAAGAGAATAGGAAGAGGAAA |
| | AATGGGTAACGGGGT |
| | (SEQ ID NO: 44) |
| DAZL crl End | AGGGCGCATCACTTCAGAAA |
| (artificial) | (SEQ ID NO: 45) |
| DAZL Cre-Intein | GAATTGTGGAAGAATTGCAGCTACTAAAAAGGCAGACTGGGTAGC |
| Targeting Vectors | ACTTCCCACCTTACTCAGATTTAGGAGTCTCAAGACATGTAGATG |
| (5′ homology arm) | GGTGAAATTTATTATTCTTTTCTCTGCTTGCTTTTCTGGAAAGCT |
| (artificial) | ACCTCTATTTGGCCAGCAGTTGGCTCTGAATTCCTTTCTTTAAAG |
| | GAGACCTGACTGCTCTCCTGTCTGCCTCTTACACAGCTGTACTGG |
| | TGTGTCACTGATAGAAACGTTTACCTTGAAAGTGACAAGTGGGGG |
| | CGTTGATACCCATTAACAACATTAAAGGGAAAATTTCAGGAAACA |
| | AGAAAACTGAGAGGAGTTCAGAATGAAATGTAAGCCCTGTGACAG |
| | ACTCACACTGATGGTAGTAGGGTAACTGATGGAGATGCATCTATA |
| | ACAATTCTAACTTTTATTTCAGAAGTCTGTGGACAGGAGCATACA |
| | AACAGTAGTATCTTGTCTGTITAACCCTGAAAACCGTCTGAGGAA |
| | CACCTTTGTATCACAAGAAGACTACTTCAGGGTATGTAGTGAAAG |
| | CAAAACTAAATCTCTAAAACCAGCTGATCTTAACTTTGTTCATAT |
| | ACAGGATGCCATGAAGACAAAGGTTTATCTGTACTTGCTTGAGAT |
| | TTTACTTTTTGTATGTGCATTGAGATGAATGACTGGTTAGCTCTC |
| | AGTTGGTTAATACTCAATGAAAATTGCAGAAATTGAGTTCAGTGG |
| | ATTTAAGTGCATTGATACAAGGCTGCATAAGAACTGCTGAGCAGT |
| | CAGCTATTGGATATTTAGAGCTAATCATCCCTCAGGAGCAATCTG |
| | CTGAAAGGTAAGCAGATTGCATCTTGTAAACGAGAGAGCAGAGTT |
| | AAACGTTTGTGGTCTTTGCTCCACTTAAGTACCCACAGTGGATGT |
| | CTATCTGTACTTTTGGTTAAAAAATAGATAATTTCTACAACAGAC |
| | TGTAGTTAGGTCAGTACCTGGTCATCAGGTATTAATGGAAAGCTT |
| | TTTGCTGTGCATTCAATCTTCATAGAGGTCTGCTTTCCAATTACA |
| | TTCTCTTTGTTTTAAGTATAACTTTGAAACTCAAATATAAAAGCC |
| | TGCTACTTTTTTTTGTTCTAAAAAGACAGATGTGGACTAGGCACC |
| | TGTACTGTGAAACCAGAATAGAGAAGCTGTTGGGCAGATTTGAAG |
| | AGGATTGAGTAATATAGAGAATGTCTTGAGATATTTGTGAAGCTT |
| | TTATTGCTTTGTTGCAATTGTTGTCTAGAAACATGGGCTTTTTCT |
| | GTTTTTTCTCCTATTCCAGGAGAGGAGGGCGCATCACTTCAGAAA |
| | AGGAAGAGCAGTGCTCAAAAGTGTT |
| | (SEQ ID NO: 46) |
| DAZL Cre-Intein | TGATGAACAAAGACTTTGAAGTACATAAATGTATTACTTTGATGT |
| Targeting Vectors | TCCTACAGTTCAGTTTAGTAAGATGTGTAGTAAAAAGTGTAACCT |
| (3′ homology arm) | TGTTCAAAAAGTTGCTTCAAGTTGATGTTTGTGTTCTGTTTTACC |
| (artificial) | TGTTCCAGAATAGCTATTTTTGCTTGAGAAGTTTGAAGTTGTAAG |
| | AGTTGAAATATTTCCAGGTTTTATTACTAGCTTGCATGCTTTTCC |
| | TGCTAACTAACTGAAATGCTAATCTTAAGGAATTTATATGGGGAA |
| | GGGGAAAAAAGAAAAACACTTTGTTTGGTATGTGTGGATTTTCTT |
| | CTGAGCTTTAAGGTACAGTTTGTTGCATGTTAAAATTTAGTTCTT |
| | ATTAAACCACAACTTTAAGTTACTAACGTCAACCAGTTACCTCTT |
| | GCAGTTCAAAAGTTGAAGCAGTTCCTTGTCCAAGATGGAGTATTT |
| | TAAAACTGAGCTCTTAATCAGTGGAACAGAAGACGTCACGGTGTA |
| | ACTCAACTGAAGCCCTTTAAGTCCCGGTTCTCTTTAGACTACCTA |
| | ATCAATGTCTTTGTTTGCTAACGACAGTTCATCTATGTGAATCCT |
| | AAAATTCCTATATGTAACTTAAGATGCAAGAATGTAATTAGTTAC |
| | ATTGGCTGCTCAGTGGAGTATGACTTTTTTTTTTACTGGATTAAT |
| | TTTAGCAATACCTGTATCTTAAAATTGTGAGAAAATACTGCATTT |
| | AAAATATGCCTAACTTTGTGATGCAATATGTTAATCAAAGAATAC |
| | ATGTAAGCATATTTTAATAATAATTATGTAGATTTTAGTCATGTA |
| | TTTTGAAACAATTAAAATTTTTAATTTTGACTTACCTTCCCAGTG |
| | TGAGTGACATCCTAATATAATACTTCTAAATCTTAAGCTGCTTTG |
| | AGAAGGGCATGCAGCGTATTTATTGAAGGAATTGAAGATTTCTTA |
| | CCCTACATAAGAATTCCAGTTAGGACAAGTTTATAGCAACAAACT |
| | TTCACATTTGCTGTTAGTTCTACCTGCGATTTTGAAGGAGTACAA |
| | CTGAGAACAGCACTCAGTCTTGTATGTGTGTTGGGTCCTAGTCTG |
| | ATTCATTTTTCTTATTACTACCCTTATACCTCAGTCTCCAAGTAA |
| | AAAAGGAAATAACTCCTCCTTTGTAGACGTGTATATGTGAATGAA |
| | TAGAATGGCATGTCCCACTTCAAATGTCTAGAAGTAGATGTTGGT |
| | GAAACATGCAATAAGAGCTGAGTTGCTCTGGACCTGGATAGTGGG |
| | CTGTAAGATGCAGCACAGGAGTGTCCCAGGACTGTTGTTCAGGAG |
| | TAGGAGTCAGGGGAGGGAGGCAGGGCTTGGGGGATAGCAATAATA |
| | GTTGGTGCCGTTCTCCATAAAATTACTCAGAAGCAATGTTCTGGC |
| | AGCATAAATCGACCTAAAGTTGTTAACTTTTTCTCTGCTCAGACC |
| | GTTCCCTATCACTAAAGATGCTGTGTCCTTAAGTGTGTTTCCATA |
| | CTATCTATCACCATGTTTTTCCCAACCTATTGCTTTAATAGTATT |
| | GTTAGTTTGATATACTTAACTTCTTCCGCGTTTCCAGCTTCTGTG |
| | TAAATGGTTTTCAATGGTACACAATTAAACTTCACATACAAGTAA |
| | TCTGGAGTTTTCTGAGTACAAGGACCCTTTCCTTCGCAACTTTAA |
| | GTACCTGAGCAGCAGCCGACTTGGATTGTTACATCTGTGATGTCA |
| | GTTTATTGATAAAGGTAATTGTTTCTTCGCAAATGCAGAATTCAT |
| | TTTGGCTTAATGTTCTAGTTGGCTT |
| | (SEQ ID NO: 47) |
| DAZL NLS-nCre- | CCCAAGAAGAAGAGGAAGGTG |
| nIntein Targeting | (SEQ ID NO: 48) |

Vector (NLS)

| | |
|---|---|
| (NLS) | |
| (artificial) | |
| DAZL NLS-nCre- | PKKKRKV |
| nIntein Targeting | (SEQ ID NO: 70) |
| Vector (NLS) | |
| (NLS AA) | |
| (artificial) | |
| DAZL NLS-nCre- | ACGAGTGATGAGGTTCGCAAGAACCTGATGGACATGTTCAGGGAT |
| nIntein Targeting | CGCCAGGCGTTTTCTGAGCATACCTGGAAAATGCTTCTGTCCGTT |
| Vector | TGCCGGTCGTGGGCGGCATGGTGCAAGTTGAATAA |
| (nCre recombinase) | (SEQ ID NO: 49) |
| (artificial) | |
| DAZL NLS-nCre- | TSDEVRKNLMDMFRDRQAFSEHTWKMLLSVCRSWAAWCKLN |
| nIntein Targeting | (SEQ ID NO: 71) |
| Vector | |
| (nCre recombinase | |
| AA) | |
| (artificial) | |
| DAZL NLS-nCre- | ATTTGCGGAATATTGCCTCAGTTTTGGCACCGAAATTTTAACCGT |
| nIntein Targeting | TGAGTACGGCCCATTGCCCATTGGCAAAATTGTGAGTGAAGAAAT |
| Vector (nIntein) | TAATTGTTCTGTGTACAGTGTTGATCCAGAAGGGAGAGTTTACAC |
| (artificial) | CCAGGCGATCGCCCAATGGCATGACCGGGGAGAGCAGGAAGTATT |
| | GGAATATGAATTGGAAGATGGTTCAGTAATCCGAGCTACCTCTGA |
| | CCACCGCTTTTTAACCACCGATTATCAACTGTTGGCGATCGAAGA |
| | AATTTTTGCTAGGCAACTGGACTTGTTGACTTTAGAAAATATTAA |
| | GCAAACTGAAGAAGCTCTTGACAACCATCGTCTTCCCTTTCCATT |
| | ACTTGACGCTGGGACAATTAAA |
| | (SEQ ID NO: 50) |
| DAZL NLS-nCre- | FAEYCLSFGTEILTVEYGPLPIGKIVSEEINCSVYSVDPEGRVYT |
| nIntein Targeting | QAIAQWHDRGEQEVLEYELEDGSVIRATSDHRFLTTDYQLLAIEE |
| Vector (nIntein AA) | IFARQLDLLTLENIKQTEEALDNHRLPFPLLDAGTIK |
| (artificial) | (SEQ ID NO: 72) |
| DAZL cCre-cIntein | AACCGGAAATGGTTTCCCGCAGAACCTGAAGATGTTCGCGATTAT |
| Targeting Vector | CTTCTATATCTTCAGGCGCGCGGTCTGGCAGTAAAAACTATCCAG |
| (c-Cre recombinase) | CAACATTTGGGCCAGCTAAACATGCTTCATCGTCGGTCCGGGCTG |
| (artificial) | CCACGACCAAGTGACAGCAATGCTGTTTCACTGGTTATGCGGCGG |
| | ATCCGAAAAGAAAACGTTGATGCCGGTGAACGTGCAAAACAGGCT |
| | CTAGCGTTCGAACGCACTGATTTCGACCAGGTTCGTTCACTCATG |
| | GAAAATAGCGATCGCTGCCAGGATATACGTAATCTGGCATTTCTG |
| | GGGATTGCTTATAACACCCTGTTACGTATAGCCGAAATTGCCAGG |
| | ATCAGGGTTAAAGATATCTCACGTACTGACGGTGGGAGAATGTTA |
| | ATCCATATTGGCAGAACGAAAACGCTGGTTAGCACCGCAGGTGTA |
| | GAGAAGGCACTTAGCCTGGGGGTAACTAAACTGGTCGAGCGATGG |
| | ATTTCCGTCTCTGGTGTAGCTGATGATCCGAATAACTACCTGTTT |
| | TGCCGGGTCAGAAAAAATGGTGTTGCCGCGCCATCTGCCACCAGC |
| | CAGCTATCAACTCGCGCCCTGGAAGGGATTTTTGAAGCAACTCAT |
| | CGATTGATTTACGGCGCTAAGGATGACTCTGGTCAGAGATACCTG |
| | GCCTGGTCTGGACACAGTGCCCGTGTCGGAGCCGCGCGAGATATG |
| | GCCCGCGCTGGAGTTTCAATACCGGAGATCATGCAAGCTGGTGGC |
| | TGGACCAATGTAAATATTGTCATGAACTATATCCGTAACCTGGAT |
| | AGTGAAACAGGGGCAATGGTGCGCCTGCTGCAAGATGGCGAT |
| | (SEQ ID NO: 51) |
| DAZL cCre-cIntein | NRKWFPAEPEDVRDYLLYLQARGLAVKTIQQHLGQLNMLHRRSGL |
| Targeting Vector | PRPSDSNAVSLVMRRIRKENVDAGERAKQALAFERTDFDQVRSLM |
| (c-Cre recombinase | ENSDRCQDIRNLAFLGIAYNTLLRIAEIARIRVKDISRTDGGRML |
| AA) | IHIGRTKTLVSTAGVEKALSLGVTKLVERWISVSGVADDPNNYLF |
| (artificial) | CRVRKNGVAAPSATSQLSTRALEGIFEATHRLIYGAKDDSGQRYL |
| | AWSGHSARVGAARDMARAGVSIPEIMQAGGWTNVNIVMNYIRNLD |
| | SETGAMVRLLQDGD |
| | (SEQ ID NO: 73) |
| DAZL cCre-cIntein | GTTAAAGTTATCGGTCGTCGTTCCCTCGGAGTGCAAAGAATATTT |
| Targeting Vector | GATATTGGTCTTCCCCAAGACCATAATTTTCTGCTAGCCAATGGG |
| (c-Intein) | GCGATCGCCGCCAATTGTTTTAACAAATCC |
| (artificial) | (SEQ ID NO: 52) |
| DAZL cCre-cIntein | VKVIGRRSLGVQRIFDIGLPQDHNFLLANGAIAANCFNKS |
| Targeting Vector | (SEQ ID NO: 74) |
| (c-Intein AA) | |
| (artificial) | |
| DAZL Cre-Intein | GGCAGCGGCGCCACCAACTTCAGCCTGCTGAAGCAGGCCGGCGAC |
| Targeting Vectors | GTGGAGGAGAACCCCGGCCCC |
| (P2A) | (SEQ ID NO: 53) |
| (artificial) | |
| DAZL Cre-Intein | GSGATNFSLLKQAGDVEENPGP |
| Targeting Vectors | |
| (P2A AA) | (SEQ ID NO: 75) |
| (artificial) | |
| DAZL Cre-Intein | GGCAGTGGAGAGGGCAGAGGAAGTCTGCTAACATGCGGTGACGTC |
| Targeting Vectors | GAGGAGAATCCTGGCCCA |
| (T2A) | (SEQ ID NO: 54) |
| (artificial) | |
| DAZL Cre-Intein | GSGEGRGSLLTCGDVEENPGP |
| Targeting Vectors | (SEQ ID NO: 76) |
| (T2A AA) | |
| (artificial) | |
| DAZL Cre-Intein | GTGAGCAAGGGCGAGGAGCTGTTCACCGGGGTGGTGCCCATCCTG |
| Targeting Vectors | GTCGAGCTGGACGGCGACGTAAACGGCCACAAGTTCAGCGTGTCC |
| (GFP) | GGCGAGGGCGAGGGCGATGCCACCTACGGCAAGCTGACCCTGAAG |
| (artificial) | TTCATCTGCACCACCGGCAAGCTGCCCGTGCCCTGGCCCACCCTC |
| | GTGACCACCCTGACCTACGGCGTGCAGTGCTTCAGCCGCTACCCC |
| | GACCACATGAAGCAGCACGACTTCTTCAAGTCCGCCATGCCCGAA |
| | GGCTACGTCCAGGAGCGCACCATCTTCTTCAAGGACGACGGCAAC |
| | TACAAGACCCGCGCCGAGGTGAAGTTCGAGGGCGACACCCTGGTG |
| | AACCGCATCGAGCTGAAGGGCATCGACTTCAAGGAGGACGGCAAC |
| | ATCCTGGGGCACAAGCTGGAGTACAACTACAACAGCCACAACGTC |
| | TATATCATGGCCGACAAGCAGAAGAACGGCATCAAGGTGAACTTC |
| | AAGATCCGCCACAACATCGAGGACGGCAGCGTGCAGCTCGCCGAC |
| | CACTACCAGCAGAACACCCCCATCGGCGACGGCCCCGTGCTGCTG |
| | CCCGACAACCACTACCTGAGCACCCAGTCCGCCCTGAGCAAAGAC |
| | CCCAACGAGAAGCGCGATCACATGGTCCTGCTGGAGTTCGTGACC |
| | GCCGCCGGGATCACTCTCGGCATGGACGAGCTGTACAAG |
| | (SEQ ID NO: 55) |
| DAZL Cre-Intein | VSKGEELFTGVVPILVELDGDVNGHKFSVSGEGEGDATYGKLTLK |
| Targeting Vectors | FICTTGKLPVPWPTLVTTLTYGVQCFSRYPDHMKQHDFFKSAMPE |
| (GFP AA) | GYVQERTIFFKDDGNYKTRAEVKFEGDTLVNRIELKGIDFKEDGN |
| (artificial) | ILGHKLEYNYNSHNVYIMADKQKNGIKVNFKIRHNIEDGSVQLAD |
| | HYQQNTPIGDGPVLLPDNHYLSTQSALSKDPNEKRDHMVLLEFVT |
| | AAGITLGMDELYK |
| | (SEQ ID NO: 77) |
| Primer A | GAATTGTGGAAGAATTGCAGC |
| (F-DAZL- | (SEQ ID NO: 56) |
| ATGCodonNest) | |
| (artificial) | |
| Primer B | GGATGCCATGAAGACAAAGG |
| (F_DAZL_EndSeq1) | (SEQ ID NO: 57) |
| (artificial) | |
| Primer C | TGCAGCCTTGTATCAATGCAC |
| (R_DAZL_EndSeq1) | (SEQ ID NO: 58) |
| (artificial) | |
| Primer D | AGGAAGAGCAGTGCTCAAAAG |
| (F_DAZL_EndSeq2) | (SEQ ID NO: 59) |
| (artificial) | |
| Primer E | GGGGCCGGGGTTCTCCTC |
| (R-magP2A) | (SEQ ID NO: 60) |
| (artificial) | |
| Primer F | AGATCCGCCACAACATCGAG |
| (Fw-PBRAGE-EGFP) | (SEQ ID NO: 61) |
| (artificial) | |
| Primer G | TGCTCAGGTAGTGGTTGTCG |
| (Rv-PBRAGE-EGFP) | (SEQ ID NO: 62) |
| (artificial) | |
| Primer H | TGGCTGCTCAGTGGAGTATG |
| (F_DAZL_EndSeq3) | (SEQ ID NO: 63) |
| (artificial) | |
| Primer I | CCTTGTACTCAGAAAACTCCAG |
| (R-Pre-D3HA-Nest) | (SEQ ID NO: 64) |
| (artificial) | |
| Primer J | AAGCCAACTAGAACATTAAGCC |
| (R-Pre-D3HA) | (SEQ ID NO: 65) |
| (artificial) | |
| Genomic Sequence | GAAGGCTATCTTGCTTCCTTGAAACTAATAATACCTAGTTTGTAG |
| (This is genomic | TGTTTAGGGATGTGTAAAATGTGTTGATGTCAAGATTCTAGTTAG |
| DNA sequence from | TCTGATAAGAATTGTGGAAGAATTGCAGCTACTAAAAAGGCAGAC |
| unmodified PGCs- | TGGGTAGCACTTCCCACCTTACTCAGATTTAGGAGTCTCAAGACA |
| WT) | TGTAGATGGGTGAAATTTATTATTCTTTTCTCTGCTTGCTTTTGT |
| | GGAAAGCTACCTCTATTTGGCCAGCAGTTGGCTCTGAATTCCTTT |
| | CTTTAAAGGAGACCTGACTGCTCTCCTGTCTGCCTCTTACACAGT |
| | TGTACTGGTGTGTCACTGATAGAAACGTTTACCTTGAAAGTGACA |
| | AGTGGGGGCGTTGATACCCATTAACAACATTAAAGGGAAAATTTC |
| | AGGAAGCAAGAAAACTGAGAGGAGTTCAGAATGAAATGTAAGCCC |
| | TGTGACAGACTCACACTGATGGTAGTAGGGTAACTGATGGAGATG |
| | CATCTATAACAATTCTAACTTTTATTTCAGAAGTCTGTGGACAGG |
| | AGCATACAAACAGTAGTATCCTGTCTGTTTAACCCTGAAAACCGT |
| | CTGAGGAACACCTTTGTATCACAAGAAGACTACTTCAGGGTATGT |
| | AGTGAAAGCAAAACTAAATCTCTAAAACCAGCTGATCTTAACTTT |
| | GTTCATATACAGGATGCCATGAAGACAAAGGTTTATCTGTACTTG |
| | CTTGAGATTTTAATTTTTGTATGTGCATTGAGATGAATGACTGGT |
| | TAGCTCTCAGTTGGTTAATACTCAATGAAAATTGCAGAAATTGAG |
| | TTCAGTGGATTTAAGTGCATTGATACAAGGCTGCATAAGAACTGC |
| | TGAGCAGTCAGCTATTGGATATTTAGAGCTAATCATCCCTCAGGA |
| | GCAATCTGCTGAAAGGTAAGCAGATTGCATCTTGTAAACGAGAGA |
| | GCAGAGTTAAACGTTTGTGGTCTTTGCTCCACTTAAGTACCCACT |
| | GTGGATGTCTATCTGTACTTTTGGTTAAAAAATAGATAATTTCTA |
| | CAACAGACTGTAGTTAGGTCAGTACCTGGTCATCAGGTATTAATG |
| | GAAAGCTTTTTGCTGTGCATTCAATCTTCATAGAGGTCTGCTTTC |
| | CAATTACATTCTCTTTGTTTTAAGTATAACATTGAAACTCAAATA |
| | TAAAAGCCTGCTACTTTTTTTTGTTCTAAAAAGACAGATGTGGAC |
| | TAGGCACCTGTACTGTGAAACCAGAATAGAGAAGCTGTTGGGCAG |
| | ATTTGAAGAGGATTGAGTAATATAGAGAATGTCTTGAGATATTTG |
| | TGAAGCTTTTATTGCTTTGTTGCGATTGTTGTCTAGAAACATGGG |
| | CTTTTTCTGTTTTTTCTCCTATTCCAGGAGAGGAGGGCGCATCAC |
| | TTCAGAAAAGGAAGAGCAGTGCTCAAAAGTGTTTGATGAACAAAG |
| | ACTTTGAAGTACATAAATGTATTACTTTGATGTTCCTACAGTTCA |
| | CGTTTAGTAAGATGTGTAGTAAAAAGTGTAACCTTGTTCAAAAAG |
| | TTGCTTCAAGTTGATGTTTGTGTTCTGTTTTACCTGTTCCAGAAT |
| | AGCTATTTTTGCTTGAGAAGTTTGAAGTTGTAAGAGTTGAAATAT |
| | TTCCAGGTTTTATTACTAGCTTGCATGCTTTTCCTGCTAACTAAC |
| | TGAAATGCTAATCTTAAGGAATTTATATGGGGAAGGGGAAAAAAG |
| | AAAAACACTTTGTTTGGTATGTGTGGATTTTCTTCTGAGCTTTAA |
| | GGTACAGTTTGTTGCATGTTAAAATTTAGTTCTTATTAAACCACA |
| | ACTTTAAGTTACTAACGTCAACCAGTTACCTCTTGCAGTTCAAAA |
| | GTTGAAGCAGTTCCTTGTCCAAGATGGAGTATTTTAAAACTGAGC |
| | TCTTAATCAGTGGAACAGAAGACGTCACGGTGTAACTCAACTGAA |
| | GCCCTTTAAGTCCCGGTTCTCTTTAGACTACCTAATCAATGTCTT |
| | TGTTTGCTAACGACAGTTCATCTATGTGAATCCTAAAATTCCTAT |
| | ATGTAACTTAAGATGCAAGAATGTAATTAGTTACATTGGCTGCTC |
| | AGTGGAGTATGACTTTTTTTTTTACTGGATTAATTTTAGCAATAC |
| | CTGTATCTTAAAATTGTGAGAAAATACTGCATTTAAAATATGCCT |
| | AACTTTGTGATGCAATATGTTAATCAAAGAATACATGTAAGCATA |
| | TTTTAATAATAATTATGTAGATTTTAGTCATGTATTTTGAAACAA |
| | TTAAAATTTTTAATTTTGACTTACCTTCCCAGTGTGAGTGACATC |
| | CTAATATAATACTTCTAAATCTTAAGCTGCTTTGAGAAGGGCATG |
| | CAGCGTATTTATTGAAGGAATTGAAGATTTCTTACCCTACATAAG |
| | AATTCCAGTTAGGACAAGTTTATAGCAACAAACTTTCACATTTGC |
| | TGTTAGTTCTACCTGCGATTTTGAAGGAGTACAACTGAGAACAGC |
| | ACTCAGTCTTGTATGTGTGTTGGGTCCTAGTCTGATTCATTTTTC |
| | TTATTACTACCCTTATACCTCAGTCTCCAAGTAAAAAAGGAAATA |
| | ACTCCTCCTTTGTAGACGTGTATATGTGAATGAATAGAATGGCAT |
| | GTCCCACTTCAAATGTCTAGAAGTAGATGTTGGTGAAACATGCAA |
| | TAAGAGCTGAGTTGCTCTGGACCTGGATAGTGGGCTGTAAGATGC |
| | AGCACAGGAGTGTCCCAGGACTGTTGTTCAGGAGTAGGAGTCAGG |
| | GGAGGGAGGCAGGGCTTGGGGGATAGCAATAATAGTTGGTGCCGT |
| | TCTCCATAAAATTACTCAGAAGCAATGTTCTGGCAGCATAAATCG |
| | ACCTAAAGTTGTTAACTTTTTCTCTGCTCAGACCGTTCCCTATCA |
| | CTAAAGATGCTGTGTCCTTAAGTGTGTTTCCATACTATCTATCAC |
| | CATGTTTTTCCCAACCTATTGCTTTAATAGTATTGTTAGTTTGAT |
| | ATACTTAACTTCTTCCGCGTTTCCAGCTTCTGTGTAAATGGTTTT |
| | CAATGGTACACAATTAAACTTCACATACAAGTAATCTGGAGTTTT |
| | CTGAGTACAAGGACCCTTTCCTTCGCAACTTTAAGTACCTGAGCA |
| | GCAGCCGACTTGGATTGTTACATCTGTGATGTCAGTTTATTGATA |
| | AAGGTAATTGTTTCTTCGCAAATGCAGAATTCATTTTGGCTTAAT |
| | GTTCTAGTTGGCTT |
| | (SEQ ID NO: 66) |
| Genomic Sequence | GAAGGCTATCTTGCTTCCTTGAAACTAATAATACCTAGTTTGTAG |
| (NLS-nCre-nIntein | TGTTTAGGGATGTGTAAAATGTGTTTATGTCAAGATTTTAGTTAG |
| knockin to DAZL) | TCTGATAAGAATTGTGGAAGAATTGCAGCTACTAAAAAGGCAGAC |
| (artificial) | TGGGTAGCACTTCCCACCTTACTCAGATTTAGGAGTCTCAAGACA |
| | TGTAGATGGGTGAAATTTATTATTCTTTTCTCTGCTTGCTTTTGT |
| | GGAAAGCTACCTCTATTTGGCCAGCAGTTGGCTCTGAATTCCTTT |
| | CTTTAAAGGAGACCTGACTGCTCTCCTGTCTGCCTCTTACACAGC |
| | TGTACTGGTGTGTCACTGATAGAAACGTTTACCTTGAAAGTGACA |
| | AGTGGGGGCGTTGATACCCATTAACAACATTAAAGGGAAAATTTC |
| | AGGAAACAAGAAAACTGAGAGGAGTTCAGAATGAAATGTAAGCCC |
| | TGTGACAGACTCACACTGATGGTAGTAGGGTAACTGATGGAGATG |
| | CATCTATAACAATTCTAACTTTTATTTCAGAAGTCTGTGGACAGG |
| | AGCATACAAACAGTAGTATCTTGTCTGTTTAACCCTGAAAACCGT |
| | CTGAGGAACACCTTTGTATCACAAGAAGACTACTTCAGGGTATGT |
| | AGTGAAAGCAAAACTAAATCTCTAAAACCAGCTGATCTTAACTTT |
| | GTTCATATACAGGATGCCATGAAGACAAAGGTTTATCTGTACTTG |
| | CTTGAGATTTTACTTTTTGTATGTGCATTGAGATGAATGACTGGT |
| | TAGCTCTCAGTTGGTTAATACTCAATGAAAATTGCAGAAATTGAG |
| | TTCAGTGGATTTAAGTGCATTGATACAAGGCTGCATAAGAACTGC |
| | TGAGCAGTCAGCTATTGGATATTTAGAGCTAATCATCCCTCAGGA |
| | GCAATCTGCTGAAAGGTAAGCAGATTGCATCTTGTAAACGAGAGA |
| | GCAGAGTTAAACGTTTGTGGTCTTTGCTCCACTTAAGTACCCACT |
| | GTGGATGTCTATCTGTACTTTTGGTTAAAAAATAGATAATTTCTA |
| | CAACAGACTGTAGTTAGGTCAGTACCTGGTCATCAGGTATTAATG |
| | GAAAGCTTTTTGCTGTGCATTCAATCTTCATAGAGGTCTGCTTTC |
| | CAATTACATTCTCTTTGTTTTAAGTATAACTTTGAAACTCAAATA |
| | TAAAAGCCTGCTACTTTTTTTTGTTCTAAAAAGACAGATGTGGAC |
| | TAGGCACCTGTACTGTGAAACCAGAATAGAGAAGCTGTTGGGCAG |
| | ATTTGAAGAGGATTGAGTAATATAGAGAATGTCTTGAGATATTTG |
| | TGAAGCTTTTATTGCTTTGTTGCAATTGTTGTCTAGAAACATGGG |
| | CTTTTTCTGTTTTTTCTCCTATTCCAGGAGAGGAGGGCGCATCAC |
| | TTTAGGAAAGGAAGAGCAGTGCTCAAAAGTGTTGGCAGCGGCGCC |
| | ACCAACTTCAGCCTGCTGAAGCAGGCCGGCGACGTGGAGGAGAAC |
| | CCCGGCCCCCCCAAGAAGAAGAGGAAGGTGACGAGTGATGAGGIT |
| | CGCAAGAACCTGATGGACATGTTCAGGGATCGCCAGGCGTTTTCT |
| | GAGCATACCTGGAAAATGCTTCTGTCCGTTTGCCGGTCGTGGGCG |
| | GCATGGTGCAAGTTGAATAAATTTGCGGAATATTGCCTCAGTTTT |
| | GGCACCGAAATTTTAACCGTTGAGTACGGCCCATTGCCCATTGGC |
| | AAAATTGTGAGTGAAGAAATTAATTGTTCTGTGTACAGTGTTGAT |
| | CCAGAAGGGAGAGTTTACACCCAGGCGATCGCCCAATGGCATGAC |
| | CGGGGAGAGCAGGAAGTATTGGAATATGAATTGGAAGATGGTTCA |
| | GTAATCCGAGCTACCTCTGACCACCGCTTTTTAACCACCGATTAT |
| | CAACTGTTGGCGATCGAAGAAATTTTTGCTAGGCAACTGGACTTG |
| | TTGACTTTAGAAAATATTAAGCAAACTGAAGAAGCTCTTGACAAC |
| | CATCGTCTTCCCTTTCCATTACTTGACGCTGGGACAATTAAAGGC |
| | AGTGGAGAGGGCAGAGGAAGTCTGCTAACATGCGGTGACGTCGAG |
| | GAGAATCCTGGCCCAGTGAGCAAGGGCGAGGAGCTGTTCACCGGG |
| | GTGTTGCCCATCCTGGTCGAGCTGGACGGCGACGTAAACGGCCAC |
| | AAGTTCAGCGTGTCCGGCGAGGGCGAGGGCGATGCCACCTACGGC |
| | AAGCTGACCCTGAAGTTCATCTGCACCACCGGCAAGCTGCCCGTG |
| | CCCTGGCCCACCCTCGTGACCACCCTGACCTACGGCGTGCAGTGC |
| | TTCAGCCGCTACCCCGACCACATGAAGCAGCACGACTTCTTCAAG |
| | TCCGCCATGCCCGAAGGCTACGTCCAGGAGCGCACCATCTTCTTC |
| | AAGGACGACGGCAACTACAAGACCCGCGCCGAGGTGAAGTTCGAG |
| | GGCGACACCCTGGTGAACCGCATCGAGCTGAAGGGCATCGACTTC |
| | AAGGAGGACGGCAACATCCTGGGGCACAAGCTGGAGTACAACTAC |
| | AACAGCCACAACGTCTATATCATGGCCGACAAGCAGAAGAACGGC |
| | ATCAAGGTGAACTTCAAGATCCGCCACAACATCGAGGACGGCAGC |
| | GTGCAGCTCGCCGACCACTACCAGCAGAACACCCCCATCGGCGAC |
| | GGCCCCGTGCTGCTGCCCGACAACCACTACCTGAGCACCCAGTCC |
| | GCCCTGAGCAAAGACCCCAACGAGAAGCGCGATCACATGGTCCTG |
| | CTGGAGTTCGTGACCGCCGCCGGGATCACTCTCGGCATGGACGAG |
| | CTGTACAAGTGATGAACAAAGACTTTGAAGTACATAAATGTATTA |
| | CTTTGATGTTCCTACAGTTCAGTTTAGTAAGATGTGTAGTAAAAA |
| | GTGTAACCTTGTTCAAAAAGTTGCTTCAAGTTGATGTTTGTGTTC |
| | TGTTTTACCTGTTCCAGAATAGCTATTTTTGCTTGAGAAGTTTGA |
| | AGTTGTAAGAGTTGAAATATTTCCAGGTTTTATTACTAGCTTGCA |
| | TGCTTTTCCTGCTAACTAACTGAAATGCTAATCTTAAGGAATTTA |
| | TATGGGGAAGGGGAAAAAAGAAAAACACTTTGTTTGGTATGTGTG |
| | GATTTTCTTCTGAGCTTTAAGGTACAGTTTGTTGCATGTTAAAAT |
| | TTAGTTCTTATTAAACCACAACTTTAAGTTACTAACGTCAACCAG |
| | TTACCTCTTGCAGTTCAAAAGTTGAAGCAGTTCCTTGTCCAAGAT |
| | GGAGTATTTTAAAACTGAGCTCTTAATCAGTGGAACAGAAGACGT |
| | CACGGTGTAACTCAACTGAAGCCCTTTAAGTCCCGGTTCTCTTTA |
| | GACTACCTAATCAATGTCTTTGTTTGCTAACGACAGTTCATCTAT |
| | GTGAATCCTAAAATTCCTATATGTAACTTAAGATGCAAGAATGTA |
| | ATTAGTTACATTGGCTGCTCAGTGGAGTATGACTTTTTTTTTTAC |
| | TGGATTAATTTTAGCAATACCTGTATCTTAAAATTGTGAGAAAAT |
| | ACTGCATTTAAAATATGCCTAACTTTGTGATGCAATATGTTAATC |
| | AAAGAATACATGTAAGCATATTTTAATAATAATTATGTAGATITT |
| | AGTCATGTATTTTGAAACAATTAAAATTTTTAATTTTGACTTACC |
| | TTCCCAGTGTGAGTGACATCCTAATATAATACTTCTAAATCTTAA |
| | GCTGCTTTGAGAAGGGCATGCAGCATATTTATTGAAGGAATTGAA |
| | GATTTCTTACCCTACATAAGAATTCCAGTTAGGACAAGTTTATAG |
| | CAACAAACTTTTATGTTTGCTGTTAGTTCCTACCTGCAATTTTGA |
| | AGGAGTACAACTGAGAACAGCACTCAGTCTTGTATGTGTGTTGGG |
| | TCCTAGTCTGATTCATTTTTCTTATTACTACCCTTATACCTCAGT |
| | CTCCAAGTAAAAAAGGAAATAACTCCTCCTTTGTAGACGTGTATA |
| | TGTGAATGAATAGAATGGCATGTCCCACTTCAAATGTCTAGAAGT |
| | AGATGTTGGTGAAACATGCAATAAGAGCTGAGTTGCTCTGGACCT |
| | GGATAGTGGGCTGTAAGATGCAGCACAGGAGTGTCCCAGGACTGT |
| | TGTTCAGGAGTAGGAGTCAGGGGAGGGAGGCAGGGCTTGGGGGAT |
| | AGCAATAATAGTTGGTGCCGTTCTCCATAAAATTACTCAGAAGCA |
| | ATGTTCTGGCAGCATAAATCGACCTAAAGTTGTTAACTTTTTTCT |
| | CTGCTCAGACTGTTCCCTATCACTAAAGATGCTGTGTCCTTAAGT |
| | GTGTTTCCATACTATCTATCACCATGTTTTTCCCAACCTATTGCT |
| | TTAATAGTATTGTTAGTTTGATATACTTAACTTCTTCCGCGTTTC |
| | CAGCTTCTGTGTAAATGGTTTTCAATGGTACACAATTAAACTTCA |
| | CATACAAGTAATCTGGAGTTTTCTGAGTACAAGGACCCTTTCCTT |
| | CGCAACTTTAAGTACCTGAGCAGCAGCCGACTTGGATTGTTACAT |
| | CTGTGATGTCAGTTTATTGATAAAGGTAATTGTTTCTTCGCAAAT |
| | GCAGAATTCATTTTGGCTTAATGTTCTAGTTGGCTT |
| | (SEQ ID NO: 67) |
| Genomic Sequence | GAAGGCTATCTTGCTTCCTTGAAACTAATAATACCTAGTTTGTAG |
| (cCre-cIntein knockin | TGTTTAGGGATGTGTAAAATGTGTTTATGTCAAGATTTTAGTTAG |
| to DAZL) | TCTGATAAGAATTGTGGAAGAATTGCAGCTACTAAAAAGGCAGAC |
| (artificial) | TGGGTAGCACTTCCCACCTTACTCAGATTTAGGAGTCTCAAGACA |
| | TGTAGATGGGTGAAATTTATTATTCTTTTCTCTGCTTGCTTTTCT |
| | GGAAAGCTACCTCTATTTGGCCAGCAGTTGGCTCTGAATTCCTTT |
| | CTTTAAAGGAGACCTGACTGCTCTCCTGTCTGCCTCTTACACAGC |
| | TGTACTGGTGTGTCACTGATAGAAACGTTTACCTTGAAAGTGACA |
| | AGTGGGGGCGTTGATACCCATTAACAACATTAAAGGGAAAATTTC |
| | AGGAAACAAGAAAACTGAGAGGAGTTCAGAATGAAATGTAAGCCC |
| | TGTGACAGACTCACACTGATGGTAGTAGGGTAACTGATGGAGATG |
| | CATCTATAACAATTCTAACTTTTATTTCAGAAGTCTGTGGACAGG |
| | AGCATACAAACAGTAGTATCTTGTCTGTTTAACCCTGAAAACCGT |
| | CTGAGGAACACCTTTGTATCACAAGAAGACTACTTCAGGGTATGT |
| | AGTGAAAGCAAAACTAAATCTCTAAAACCAGCTGATCTTAACTTT |
| | GTTCATATACAGGATGCCATGAAGACAAAGGTTTATCTGTACTTG |
| | CTTGAGATTTTACTTTTTGTATGTGCATTGAGATGAATGACTGGT |
| | TAGCTCTCAGTTGGTTAATACTCAATGAAAATTGCAGAAATTGAG |
| | TTCAGTGGATTTAAGTGCATTGATACAAGGCTGCATAAGAACTGC |
| | TGAGCAGTCAGCTATTGGATATTTAGAGCTAATCATCCCTCAGGA |
| | GCAATCTGCTGAAAGGTAAGCAGATTGCATCTTGTAAACGAGAGA |
| | GCAGAGTTAAACGTTTGTGGTCTTTGCGTTAAAAAATAGATAATT |
| | TCTACAACAGACTGTAGTTAGGTCAGTACCTGGTCATCAGGTATT |
| | AATGGAAAGCTTTTTGCTGTGCATTCAATCTTCATAGAGGTCTGC |
| | TTTCCAATTACATTTCCACTTAAGTACCCACAGTGGATGTCTATC |
| | TGTACTTTTGCTCTTTGTTTTAAGTATAACTTTGAAACTCAAATA |
| | TAAAAGCCTGCTACTTTTTTTTGTTCTAAAAAGACAGATGTGGAC |
| | TAGGCACCTGTACTGTGAAACCAGAATAGAGAAGCTGTTGGGCAG |
| | ATTTGAAGAGGATTGAGTAATATAGAGAATGTCTTGAGATATTTG |
| | TGAAGCTTTTATTGCTTTGTTGCAATTGTTGTCTAGAAACATGGG |
| | CTTTTTCTGTTTTTTCTCCTATTCCAGGAGAGGAGGGCGCATCAC |
| | TTCAGAAAAGGAAGAGCAGTGCTCAAAAGTGTTGGCAGCGGCGCC |
| | ACCAACTTCAGCCTGCTGAAGCAGGCCGGCGACGTGGAGGAGAAC |
| | CCCGGCCCCGTTAAAGTTATCGGTCGTCGTTCCCTCGGAGTGCAA |
| | AGAATATTTGATATTGGTCTTCCCCAAGACCATAATTTTCTGCTA |
| | GCCAATGGGGCGATCGCCGCCAATTGTTTTAACAAATCCAACCGG |
| | AAATGGTTTCCCGCAGAACCTGAAGATGTTCGCGATTATCTTCTA |
| | TATCTTCAGGCGCGCGGTCTGGCAGTAAAAACTATCCAGCAACAT |
| | TTGGGCCAGCTAAACATGCTTCATCGTCGGTCCGGGCTGCCACGA |
| | CCAAGTGACAGCAATGCTGTTTCACTGGTTATGCGGCGGATCCGA |
| | AAAGAAAACGTTGATGCCGGTGAACGTGCAAAACAGGCTCTAGCG |
| | TTCGAACGCACTGATTTCGACCAGGTTCGTTCACTCATGGAAAAT |
| | AGCGATCGCTGCCAGGATATACGTAATCTGGCATTTCTGGGGATT |
| | GCTTATAACACCCTGTTACGTATAGCCGAAATTGCCAGGATCAGG |
| | GTTAAAGATATCTCACGTACTGACGGTGGGAGAATGTTAATCCAT |
| | ATTGGCAGAACGAAAACGCTGGTTAGCACCGCAGGTGTAGAGAAG |
| | GCACTTAGCCTGGGGGTAACTAAACTGGTCGAGCGATGGATTTCC |
| | GTCTCTGGTGTAGCTGATGATCCGAATAACTACCTGTTTTGCCGG |
| | GTCAGAAAAAATGGTGTTGCCGCGCCATCTGCCACCAGCCAGCTA |
| | TCAACTCGCGCCCTGGAAGGGATTTTTGAAGCAACTCATCGATTG |
| | ATTTACGGCGCTAAGGATGACTCTGGTCAGAGATACCTGGCCTGG |
| | TCTGGACACAGTGCCCGTGTCGGAGCCGCGCGAGATATGGCCCGC |
| | GCTGGAGTTTCAATACCGGAGATCATGCAAGCTGGTGGCTGGACC |
| | AATGTAAATATTGTCATGAACTATATCCGTAACCTGGATAGTGAA |
| | ACAGGGGCAATGGTGCGCCTGCTGCAAGATGGCGATGGCAGTGGA |
| | GAGGGCAGAGGAAGTCTGCTAACATGCGGTGACGTCGAGGAGAAT |
| | CCTGGCCCAGTGAGCAAGGGCGAGGAGCTGTTCACCGGGGTGGTG |
| | CCCATCCTGGTCGAGCTGGACGGCGACGTAAACGGCCACAAGTTC |
| | AGCGTGTCCGGCGAGGGCGAGGGCGATGCCACCTACGGCAAGCTG |
| | ACCCTGAAGTTCATCTGCACCACCGGCAAGCTGCCCGTGCCCTGG |
| | CCCACCCTCGTGACCACCCTGACCTACGGCGTGCAGTGCTTCAGC |
| | CGCTACCCCGACCACATGAAGCAGCACGACTTCTTCAAGTCCGCC |
| | ATGCCCGAAGGCTACGTCCAGGAGCGCACCATCTTCTTCAAGGAC |
| | GACGGCAACTACAAGACCCGCGCCGAGGTGAAGTTCGAGGGCGAC |
| | ACCCTGGTGAACCGCATCGAGCTGAAGGGCATCGACTTCAAGGAG |
| | GACGGCAACATCCTGGGGCACAAGCTGGAGTACAACTACAACAGC |
| | CACAACGTCTATATCATGGCCGACAAGCAGAAGAACGGCATCAAG |
| | GTGAACTTCAAGATCCGCCACAACATCGAGGACGGCAGCGTGCAG |
| | CTCGCCGACCACTACCAGCAGAACACCCCCATCGGCGACGGCCCC |
| | GTGCTGCTGCCCGACAACCACTACCTGAGCACCCAGTCCGCCCTG |
| | AGCAAAGACCCCAACGAGAAGCGCGATCACATGGTCCTGCTGGAG |
| | TTCGTGACCGCCGCCGGGATCACTCTCGGCATGGACGAGCTGTAC |
| | AAGTGATGAACAAAGACTTTGAAGTACATAAATGTATTACTTTGA |
| | TGTTCCTACAGTTCAGTTTAGTAAGATGTGTAGTAAAAAGTGTAA |
| | CCTTGTTCAAAAAGTTGCTTCAAGTTGATGTTTGTGTTCTGTTTT |
| | ACCTGTTCCAGAATAGCTATTTTTGCTTGAGAAGTTTGAAGTTGT |
| | AAGAGTTGAAATATTTCCAGGTTTTATTACTAGCTTGCATGCTTT |
| | TCCTGCTAACTAACTGAAATGCTAATCTTAAGGAATTTATATGGG |
| | GAAGGGGAAAAAAGAAAAACACTTTGTTTGGTACGTGTGGATTTT |
| | CTTCTGAGCTTTAAGGTACAGTTTGTTGCATGTTAAAATTTAGTT |
| | CTTATTAAACCACAACTTTAAGTTACTAACGTCAACCAGTTACCT |
| | CTTGCAGTTCAAAAGTTGAAGCAGTTCCTTGTCCAAGATGGAGTA |
| | TTTTAAAACTGAGCTCTTAATCAGTGGAACAGAAGACGTCACGGT |
| | GTAACTCAACTGAAGCCCTTTAAGTCCCGGTTCTCTTTAGACTAC |
| | CTAATCAATGTCTTTGTTTGCTAACGACAGTTTATCTATGTGAAT |
| | CCTAAAATTCCTATATGTAACTTAAGATGCAAGAATGTAATTAGT |
| | TACATTGGCTGCTCAGTGGAGTATGACTTTTTTTTTTACTGGATT |
| | AATTTTAGCAATACCTGTATCTTAAAATTGTGAGAAAATACTGCA |
| | TTTAAAATATGCCTAACTTTGTGATGCAATATGTTAATCAAAGAA |
| | TACATGTAAGCATATTTTAATAATAATTATGTAGATTTTAGTCAT |
| | GTATTTTGAAACAATTAAAATTTTTAATTTTGACTTACCTTCCCA |
| | GTGTGAGTGACATCCTAATATAATACTTCTAAATCTTAAGCTGCT |
| | TTGAGAAAGGCATGCAGCGTATTTATTGAAGGAATTGAAGATTTC |
| | TTACCCTACATAAGAATTCCAGTTAGGACAAGTITATAGCAACAA |
| | ACTITCACATTTGCTGTTAGTTCTACCTGCGATTTTGAAGGAGTA |
| | CAACTGAGAACAGCACTCAGTCTTGTATGTGTGTTGGGTCCTAGT |
| | CTGATTCATTTTTCTTATTACTACCCTTATACCTCAGTCTCCAAG |
| | TAAAAAAGGAAATAACTCCTCCTTTGTAGACGTGTATATGTGAAT |
| | GAATAGAATGGCATGTCCCACTTCAAATGTCTAGAAGTAGATGTT |
| | GGTGAAACATGCAATAAGAGCTGAGTTGCTCTGTACCTGGATAGT |
| | GGGCTGTAAGATGCAGCACAGGAGTGTCCCAGGACTGTTGTTCAG |
| | GAGTAGGAGTCAGGGGAGGGAGGCAGGGCTTGGGGGATAGCAATA |
| | ATAGTTGGTGCCGTTCTCCATAAAATTACTCAGAAGCAATGTTCT |
| | GGCAGCATAAATCGACCTAAAGTTGTTAACTTTTTCTCTGCTCAG |
| | ACCGTTCCCTAGCACTAAAGATGCTGTGTCCTTAAGTGTGTTTCC |
| | ATACTATCCATCACCATGTTTTTCCCAACCTATTGCTTTAATAGT |
| | ATTGTTAGTTTGATATACTTAACTTCTTCCGCGTTTCCAGCTTCT |
| | GTGTAAATGGTTTTCAATGGTACACAATTAAACTTCACATACAAG |
| | TAATCTGGAGTTTTCTGAGTACAAGGACCCTTTCCTTCGCAACTT |
| | TAAGTACCTGAGCAGCAGCCGACTTGGATTGTTACATCTGTGATG |
| | TCAGTTTATTGATAAAGGTAATTGTTTCTTCGCAAATGCAGAATT |
| | CATTTTGGCTTAATGTTCTAGTTGGCTT |
| | (SEQ ID NO: 68) |
| cre recombinase | ATGGCACCCAAGAAGAAGAGGAAGGTGTCCAATTTACTGACCGTA |
| (Cre Recombinase | CACCAAAATTTGCCTGCATTACCGGTCGATGCAACGAGTGATGAG |
| Expression Plasmid; | GTTCGCAAGAACCTGATGGACATGTTCAGGGATCGCCAGGCGTTT |
| this is the CDS of the | TCTGAGCATACCTGGAAAATGCTTCTGTCCGTTTGCCGGTCGTGG |
| Cre enzyme, which is | GCGGCATGGTGCAAGTTGAATAACCGGAAATGGTTTCCCGCAGAA |
| part of the full pCAG- | CCTGAAGATGTTCGCGATTATCTTCTATATCTTCAGGCGCGCGGT |
| Cre plasmid) | CTGGCAGTAAAAACTATCCAGCAACATTTGGGCCAGCTAAACATG |
| | CTTCATCGTCGGTCCGGGCTGCCACGACCAAGTGACAGCAATGCT |
| | GTTTCACTGGTTGTGCGGCGGATCCGAAAAGAAAACGTTGATGCC |
| | GGTGAACGTGCAAAACAGGCTCTAGCGTTCGAACGCACTGATTTC |
| | GACCAGGTTCGTTCACTCATGGAAAATAGCGATCGCTGCCAGGAT |
| | ATACGTAATCTGGCATTTCTGGGGATTGCTTATAACACCCTGTTA |
| | CGTATAGCCGAAATTGCCAGGATCAGGGTTAAAGATATCTCACGT |
| | ACTGACGGTGGGAGAATGTTAATCCATATTGGCAGAACGAAAACG |
| | CTGGTTAGCACCGCAGGTGTAGAGAAGGCACTTAGCCTGGGGGTA |
| | ACTAAACTGGTCGAGCGATGGATTTCCGTCTCTGGTGTAGCTGAT |
| | GATCCGAATAACTACCTGTTTTGCCGGGTCAGAAAAAATGGTGTT |
| | GCCGCGCCATCTGCCACCAGCCAGCTATCAACTCGCGCCCTGGAA |
| | GGGATTTTTGAAGCAACTCATCGATTGATTTACGGCGCTAAGGAT |
| | GACTCTGGTCAGAGATACCTGGCCTGGTCTGGACACAGTGCCCGT |
| | GTCGGAGCCGCGCGAGATATGGCCCGCGCTGGAGTTTCAATACCG |
| | GAGATCATGCAAGCTGGTGGCTGGACCAATGTAAATATTGTCATG |
| | AACTATATCCGTAACCTGGATAGTGAAACAGGGGCAATGGTGCGC |
| | CTGCTGCAAGATGGCGATTAG |
| | (SEQ ID NO: 69) |
| nCre-nIntein | GSGATNFSLLKQAGDVEENPGPPKKKRKVTSDEVRKNLMDMFRDR |
| Construct: | QAFSEHTWKMLLSVCRSWAAWCKLNKFAEYCLSFGTEILTVEYGP |
| P2A-NLS-nCre- | LPIGKIVSEEINCSVYSVDPEGRVYTQAIAQWHDRGEQEVLEYEL |
| nIntein-T2A-GFP | EDGSVIRATSDHRFLTTDYQLLAIEEIFARQLDLLTLENIKQTEE |
| (artificial) | ALDNHRLPFPLLDAGTIKGSGEGRGSLLTCGDVEENPGPVSKGEE |
| | LFTGVVPILVELDGDVNGHKFSVSGEGEGDATYGKLTLKFICTTG |
| | KLPVPWPTLVTTLTYGVQCFSRYPDHMKQHDFFKSAMPEGYVQER |
| | TIFFKDDGNYKTRAEVKFEGDTLVNRIELKGIDFKEDGNILGHKL |
| | EYNYNSHNVYIMADKQKNGIKVNFKIRHNIEDGSVQLADHYQQNT |
| | PIGDGPVLLPDNHYLSTQSALSKDPNEKRDHMVLLEFVTAAGITL |
| | GMDELYK |
| | (SEQ ID NO: 78) |
| cCre-cIntein | GSGATNFSLLKQAGDVEENPGPVKVIGRRSLGVQRIFDIGLPQDH |
| Construct: | NFLLANGAIAANCFNKSNRKWFPAEPEDVRDYLLYLQARGLAVKT |
| P2A-cIntein-cCre- | IQQHLGQLNMLHRRSGLPRPSDSNAVSLVMRRIRKENVDAGERAK |
| T2A-GFP | QALAFERTDFDQVRSLMENSDRCQDIRNLAFLGIAYNTLLRIAEI |
| (artificial) | ARIRVKDISRTDGGRMLIHIGRTKTLVSTAGVEKALSLGVTKLVE |
| | RWISVSGVADDPNNYLFCRVRKNGVAAPSATSQLSTRALEGIFEA |
| | THRLIYGAKDDSGQRYLAWSGHSARVGAARDMARAGVSIPEIMQA |
| | GGWTNVNIVMNYIRNLDSETGAMVRLLQDGDGSGEGRGSLLTCGD |
| | VEENPGPVSKGEELFTGVVPILVELDGDVNGHKFSVSGEGEGDAT |
| | YGKLTLKFICTTGKLPVPWPTLVTTLTYGVQCFSRYPDHMKQHDF |
| | FKSAMPEGYVQERTIFFKDDGNYKTRAEVKFEGDTLVNRIELKGI |
| | DFKEDGNILGHKLEYNYNSHNVYIMADKQKNGIKVNFKIRHNIED |
| | GSVQLADHYQQNTPIGDGPVLLPDNHYLSTQSALSKDPNEKRDHM |
| | VLLEFVTAAGITLGMDELYK |
| | (SEQ ID NO: 79) |

### Example 17: Production of Sterile Avian Embryos Devoid of Functional Primordial Germ Cells (PGCs) while Maintaining Founder Populations Via a Cre-LoxP and Intein Reconstitution System

Objective: To produce a population of sterile avian embryos devoid of functional PGCs while maintaining founder populations using a Cre-LoxP and intein reconstitution system.

The Cre recombinase enzyme is a genomic modifier, which alters a DNA sequence flanked by two unique recognition sites known as LoxP (SEQ ID NO: 1). Using different strategies, the flanked DNA sequence can be inversed, replaced, or excised. If the two LoxP sites are orientated in tandem, the DNA sequence between them is excised from the genome, leaving one LoxP site as a scar in the genome. If the excised DNA fragment contains an essential part of a FRG, such as promoter or encoding exon, the expression of FRG will be silenced. The Cre recombinase enzyme can be split into two inactive halves, each in a different breed, and when these two halves are reunited in a progeny avian of the two breeds, the enzyme's activity is regained. The expression of the inactive elements can be regulated by a ubiquitous promoter, such as the pCAGG (SEQ ID NO: 2), which is active in many cell types, or by a tissue specific promoter, which is active only in PGCs, such as promoters of FRGs.

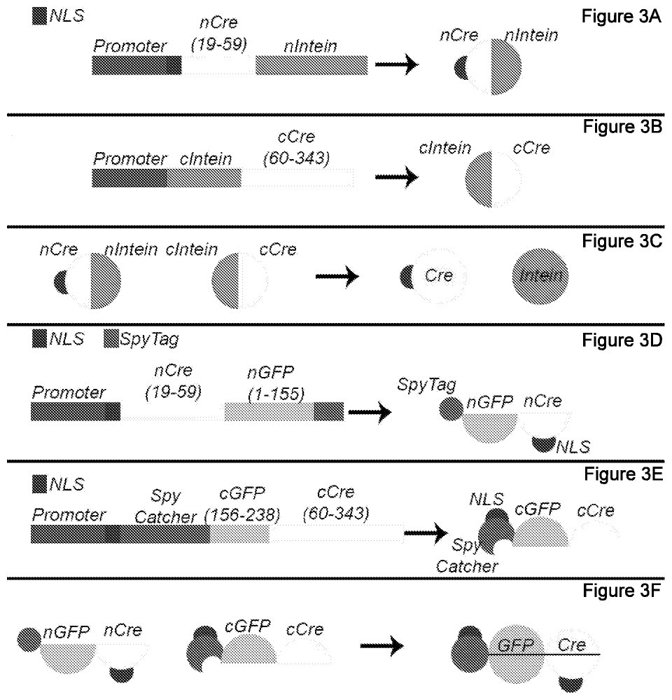

The concept behind this approach is similar to what is described above, the two inactive halves of Cre, each is linked to a different peptide, thus forming two SIEs. When the SIEs are co-expressed, they dimerize and create an active form of Cre. Notably, the system benefits from forming a covalent bond, which renders the Cre to be highly active following dimerization, as presented in FIGS. 3A-3C.

FIG. 3A is a schematic representation of the first SIE transgene on Breed A, comprising a promoter (blue), followed by a nuclear localization signal (NLS) (SEQ ID NO: 3, encoding SEQ ID NO: 4) (purple), followed by a portion of the N-terminal 19-59 amino acid residues of the Cre recombinase enzyme (nCre) (SEQ ID NO: 5, encoding SEQ ID NO: 6) (yellow), followed by the N-terminal part of the Intein (nIntein) (SEQ ID NO: 7, encoding SEQ ID NO: 8) (red). These elements are fused in an open reading frame.

FIG. 3B is a schematic representation of the second SIE transgene on Breed B, comprising a promoter (blue), followed by the C-terminal part of the Intein (cIntein) (SEQ ID NO: 9, encoding SEQ ID NO: 10) (red), followed by the C-terminal 60-343 amino acid residues of the Cre recombinase enzyme (cCre) (SEQ ID NO: 11, encoding SEQ ID NO: 12) (yellow).

FIG. 3C is a schematic representation of the outcomes of crossing Beed A with Breed B to yield Breed AB. When the nCre-nIntein and the cIntein-cCre are co-expressed, the Intein peptides dimerize, and the Intein self-cleaves itself out (red circle), leaving the NLS and the two portions of Cre (nCre/cCre) covalently bound and active.

### Example 18: Production of Sterile Avian Embryos Devoid of Functional Primordial Germ Cells (PGCs) while Maintaining Founder Populations Via a Cre-LoxP and SpyTag/SpyCatcher Binary System with a Marker

Objective: To produce a population of sterile avian embryos devoid of functional PGCs while maintaining founder populations via genomic modification using a Cre-LoxP recombinase system in combination with a SpyTag-SpyCatcher conjugating element system.

The Cre recombinase enzyme is a genomic modifier, which alters a DNA sequence flanked by two unique recognition sites known as LoxP (SEQ ID NO: 1). Using different strategies, the flanked DNA sequence can be inversed, replaced, or excised. If the two LoxP sites are orientated in tandem, the DNA sequence between them is excised from the genome, leaving one LoxP site as a scar in the genome. If the excised DNA fragment contains an essential part of a FRG, such as promoter or encoding exon, the expression of FRG will be silenced. The Cre recombinase enzyme can be split into two inactive halves, each in a different breed, and when these two halves are reunited in a progeny avian of the two breeds, the enzyme's activity is regained. The expression of the inactive elements can be regulated by a ubiquitous promoter, such as the pCAGG (SEQ ID NO: 2), which is active in many cell types, or by a tissue specific promoter, which is active only in PGCs, such as promoters of FRGs.

The concept behind this approach is similar to what is described above, the two inactive halves of Cre, each is linked to a different peptide, thus forming two SIEs. When the SIEs are co-expressed, they dimerize and create an active form of Cre. Notably, the system benefits from forming a covalent bond, which renders the Cre to be highly active following dimerization, as presented in FIGS. 3D-3E. These are the SpyTag plasmids.

FIG. 3D is a schematic representation of the first SIE transgene on Breed A, comprising a promoter (blue), followed by a nuclear localization signal (NLS) (purple), followed by an N-terminal fragment of Cre (nCre) (SEQ ID NO: 5, encoding SEQ ID NO: 6) (yellow), followed by the N-terminal amino acid residues 1-155 of Green Fluorescent Protein (nGFP) (green), followed by SpyTag sequence (bordeaux color). In some embodiments, SEQ ID NO: 82 codes for the nucleotide sequence of a SpyTag expression vector plasmid. SEQ ID NO: 82 comprises the expression vector of the pCAG promoter, which drives the expression of the following elements: Flag tag-NLS-nCre-nGFP-SpyTag, which are all fused in-frame.

FIG. 3E is a schematic representation of the second SIE transgene on Breed B, comprising a promoter (blue), followed by an NLS (purple), followed by the SpyCatcher sequence (Bordeaux color), followed by the C-terminal amino acid residues 156-238 of Green Fluorescent Protein (cGFP), followed by a C-terminal fragment of Cre (cCre) (SEQ ID NO: 11, encoding SEQ ID NO: 12) (yellow). In some embodiments, SEQ ID NO: 83 codes for the nucleotide sequence of a SpyCatcher expression vector plasmid. SEQ ID NO: 83 comprises the pCAG promoter which drives the expression of the following elements: Myc Tag-NLS-SpyCatcher-cGFP-cCre, which are all fused in-frame.

FIG. 3F is a schematic representation of the outcomes of crossing between Breed A and Breed B to yield Breed AB. When the NLS-nCre-nGFP-SpyTag and the NLC-SpyCather-cGFP-cCre are co-expressed, the SpyTag and SpyCatcher dimerize and covalently bind. This unites the two inactive parts of the GFP and of the Cre, rendering the fused protein to become fluorescent and the Cre enzyme to become active.

### Example 19: Production of Sterile Avian Embryos Devoid of Functional Primordial Germ Cells (PGCs) while Maintaining Founder Populations Via Inactivation of Deleted in AZoospermia-Like (DAZL) Using a Cre Recombinase-LoxP and an Intein Reconstitution System

Objective: To produce a population of sterile avian embryos devoid of functional PGCs while maintaining founder populations via inactivation of Deleted in AZoospermia-Like (DAZL) using a Cre recombinase-LoxP and an intein reconstitution system.

Deleted in AZoospermia-Like (DAZL) is an FRG. The following example unites the two elements described above, namely the binary regulatory mechanism and the strategy to inactivate FRGs. This example also describes a mechanism of expressing SIEs in a PGCs-specific context.

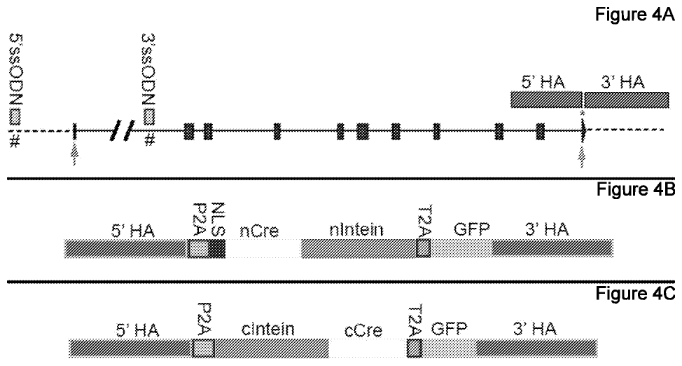

In this example, the Intein system elements are expressed under the expression regulation mechanism of the FRG DAZL (ch2:34,383,439.34,400,982; white leghorn layer GRCg7w), by fusing in-frame the Cre elements (nCre: SEQ ID NO: 5, encoding SEQ ID NO: 6; cCre: SEQ ID NO: 11, encoding SEQ ID NO: 12) Intein elements (nIntein: SEQ ID NO: 7, encoding SEQ ID NO: 8; cIntein (SEQ ID NO: 9, encoding SEQ ID NO: 10) downstream to the last coding exon of DAZL, with the self-cleaving peptide P2A (SEQ ID NO: 13, encoding SEQ ID NO: 14). The schematic representation of the genomic region of DAZL is shown in FIG. 4A. To facilitate the identification of the integration, the reporter gene GFP (SEQ ID NO: 15, encoding SEQ ID NO: 16) is also introduced in-frame.

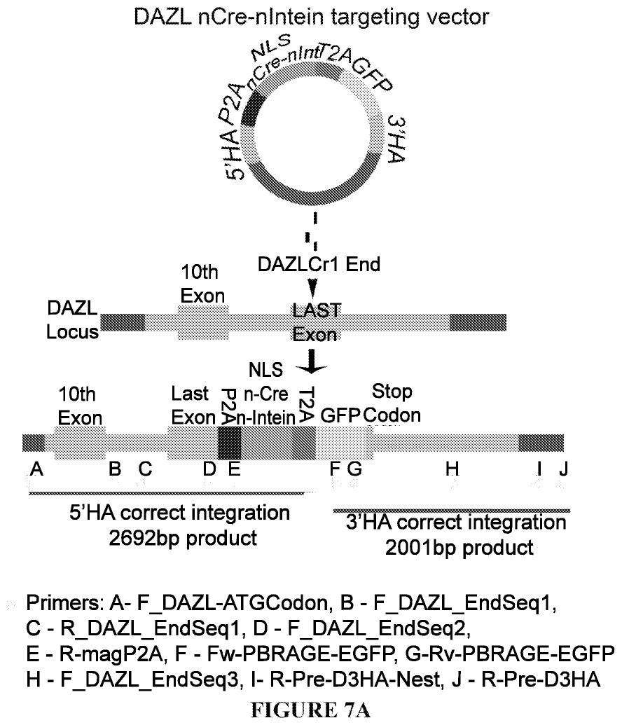

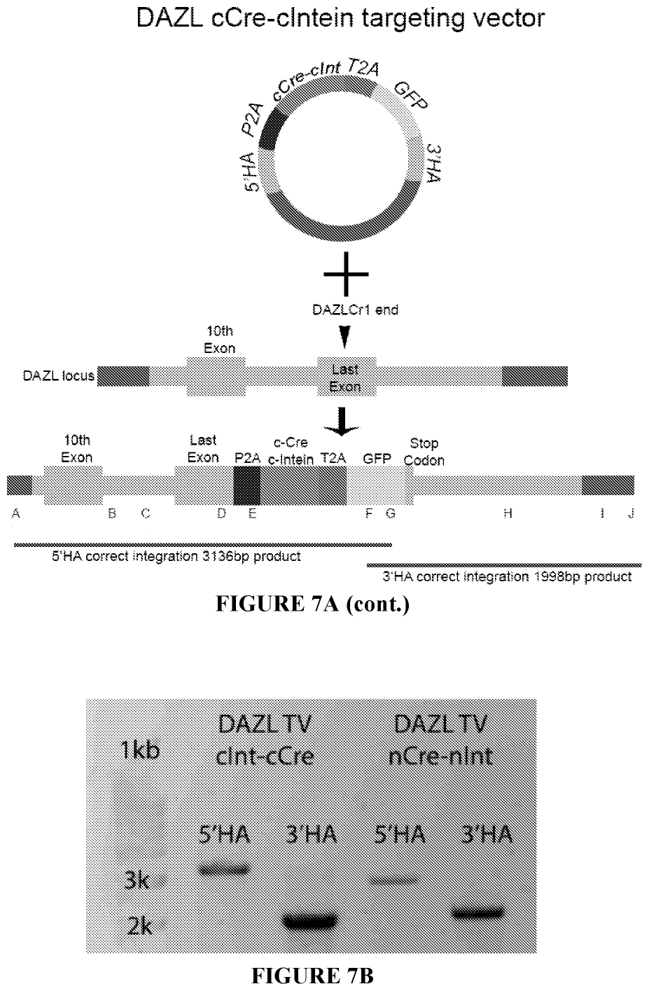

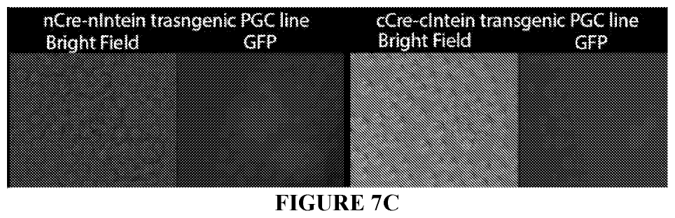

The GFP (SEQ ID NO: 15, encoding SEQ ID NO: 16) is connected by a self-cleaving peptide T2A (SEQ ID NO: 17, encoding SEQ ID NO: 18). The 5′ and 3′ homology arms (HA), which direct the recombination, and flank the Intein cassettes are SEQ ID NO: 23 & SEQ ID NO: 24, respectively, located on ch2:34,384,285.34,385,615 white leghorn layer GRCg7w, and ch2:34,382,718.3,4384,284 white leghorn layer GRCg7w, respectively (FIGS. 4A-4C, dark green boxes). The introduction of the Intein targeting vectors (TVs) is generated by homologous recombination, which is mediated by DNA double-strand break (DSB), generated by the CRISPR-Cas9 system. The location of single-guide ribonucleic acid (sgRNA) used for this DSB (TV-sgRNA, SEQ ID NO: 25; ch2:34,384,310.34,384,329 white leghorn layer GRCg7w) is shown in FIG. 4A (asterisk sign). The vectors described here were used to obtain the results in FIGS. 7A AND 7B.

In one embodiment, TV-sgRNA (SEQ ID NO: 25) codes for a RNA sequence. In one embodiment, TV-sgRNA (SEQ ID NO: 25) codes for a DNA sequence providing a template for the synthesis of the sgRNA molecule. One skilled in the art would appreciate that a DNA sequence may provide the template for a sgRNA sequence that is an RNA sequence, wherein the RNA sequence has the nucleobase uracil (U) while DNA contains thymine (T) and the RNA contains the sugar ribose, while DNA contains the slightly different sugar deoxyribose.

The schematic representation of the first Intein targeting vectors, comprising the 5′HA-P2A-NLS-nCre-nIntein-T2A-GFP-3′HA is shown in FIG. 4B. The schematic representation of the second Intein targeting vector, comprising the 5′HA-P2A-cIntein-cCre-T2A-GFP-3′HA is shown in FIG. 4C.

To deactivate the expression of DAZL, two LoxP (SEQ ID NO: 1) sites, flanking the first exon of gene, are introduced. Excising the first exon from the genome renders the DAZL null. The introduction of the LoxP sites is generated by homologous recombination of a single-strand oligodeoxynucleotide (ssODN), comprising a LoxP site (SEQ ID NO: 1), flanked by two short homology arms. For each of the two integrations of the LoxP, a specific ssODN is designed. The two ssODN molecules are termed 5′ssODN (with LoxP, SEQ ID NO: 19) and 3′ssODN (with LoxP, SEQ ID NO: 20) with respect to their position of the first DAZL exon. The introduction of the ssODN elements is generated by homologous recombination mediated by DSB, generated by the CRISPR-Cas9 system. The locations of two sgRNA (sgRNA-5′ssODN SEQ ID NO: 21; ch2:34,397,674.34,397,693 white leghorn layer GRCg7w, and sgRNA-3′ssODN SEQ ID NO: 22; ch2:34,392,740.34,392,759 white leghorn layer GRCg7w, respectively) used for this DSB are shown (sharp sign #) in FIG. 4A. The skilled artisan would appreciate that a DNA sequence may provide a template for the synthesis of the sgRNA molecule. One skilled in the art would appreciate that a DNA sequence may provide the template for a sgRNA sequence that is an RNA sequence, wherein the RNA sequence has the nucleobase uracil (U) while DNA contains thymine (T) and the RNA contains the sugar ribose, while DNA contains the slightly different sugar deoxyribose.

Collectively, for each breed, Breed A and Breed B, three genomic integrations are created. The first, the 5′ssODN upstream to the first DAZL exon, the second, 3′ssODN downstream to the first exon (in the first intron), and the third, the SIEs Intein-targeting vectors (for Breed A, P2A-NLS-nCre-nIntein-GFP, and for Breed B, P2A-cIntein-cCre-GFP) fused in-frame downstream to the last coding sequences of DAZL, in the last exon.

Crossing between the breeds results in Breed AB embryos harboring the two integrated SIEs; the first is 5′HA-P2A-NLS-nCre-nIntein-T2A-GFP-3′HA, and second is the 5′HA-P2A-cIntein-cCre-T2A-GFP-3′HA. Both are expressed under the endogenous promoter of DAZL. Upon dimerization of the two proteins, the two inactive halves of Cre form an active recombinase enzyme. The active Cre recombinase excise the first exon, which is flanked by the 5′ and the 3′ LoxP sites, which were introduced with the 5′ssODN, and the 3′ssODN, respectively. Additionally, GFP is expressed in all DAZL-expressing cells. This results in the inactivation of the FRG, DAZL.

### Example 20: Production of Sterile Avian Embryos Devoid of Functional Primordial Germ Cells (PGCs) while Maintaining Founder Populations Via Inactivation of Deleted in AZoospermia-Like (DAZL) Using a Cre Recombinase-LoxP and a Flp Recombinase-FRT

Objective: To produce a population of sterile avian embryos devoid of functional PGCs while maintaining founder populations via inactivation of Deleted in AZoospermia-Like (DAZL) using a Cre recombinase-LoxP and a Flp recombinase-FRT.

This example describes a binary system that results in embryos with null mutations in DAZL, an FRG. Two fertile breeds are generated, which, when crossed, give rise to sterile embryos, using two different DNA recombinase enzymes, Cre recombinase (with NLS, SEQ ID NO: 26, encoding SEQ ID NO: 27) and flippase (Flipase) recombinase (Flp; Flip) (with NLS, SEQ ID NO: 28, encoding SEQ ID NO: 29).

As used herein, the term “Flp” is used interchangeably with the term “Flip” having all the same meanings and qualities.

In this example, the two enzymes serve as units of the binary system that comprise SIE, and they are expressed under the expression regulation mechanism of the FRG DAZL.

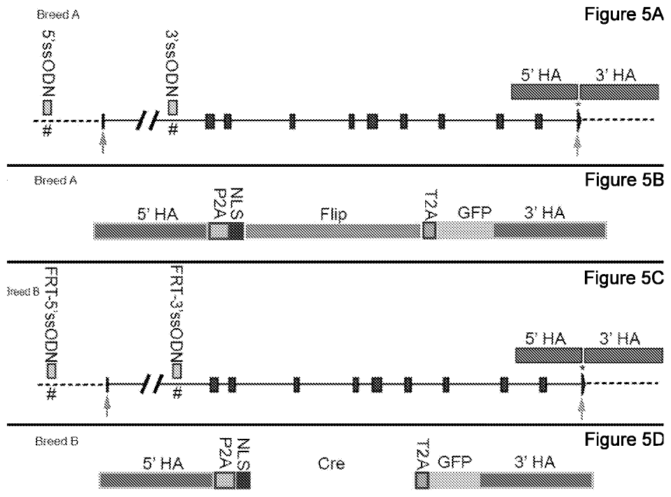

Two breeds are created, Breed A and Breed B. The genome of Breed A comprises insertions of LoxP sites flanking the first exon of DAZL. These are used to excise the first exon from the genome and render the DAZL null, as described in the previous example above. The introduction of the LoxP sites is generated by homologous recombination of a ssODN molecule, comprising a LoxP site (SEQ ID NO: 1), flanked by two short homology arms. For each of the two integrations of the LoxP, a specific ssODN is designed. The two single-stranded oligodeoxynucleotide (ssODN) molecules are termed 5′ssODN (SEQ ID NO: 19) and 3′ssODN (SEQ ID NO: 20) with respect to their position of the first DAZL exon. The introduction of the ssODN elements is generated by homologous recombination mediated by double-stranded breaks (DSB), generated by the CRISPR-Cas9 system. The locations of two sgRNA (sgRNA-5′ssODN SEQ ID NO: 21; ch2:34,397,674.34,397,693 white leghorn layer GRCg7w, and sgRNA-3′ssODN SEQ ID NO: 22; ch2:34,392,740.34,392,759 white leghorn layer GRCg7w, respectively) used for these DSB (sharp sign #) are shown in FIG. 5A.

In addition to the two ssODN integrations, the SIE, in this case Flp, is fused in-frame downstream to the last coding exon of DAZL, with the self-cleaving peptide P2A (SEQ ID NO: 13, encoding SEQ ID NO: 14), followed by a nuclear localization signal (NLS) (SEQ ID NO: 3, encoding SEQ ID NO: 4). To facilitate the identification of the genomic integration, the reporter gene green fluorescent protein (GFP) (SEQ ID NO: 15, encoding SEQ ID NO: 16) is also introduced in-frame. The GFP (SEQ ID NO: 15, encoding SEQ ID NO: 16) is connected by a self-cleaving peptide T2A (SEQ ID NO: 17, encoding SEQ ID NO: 18) to the 3′ end of Flp. To integrate these elements into the genome, a targeting vector (TV) is designed. This targeting vector consists of two 5′ and 3′ homology arms that direct specific integration by homologous recombination. These arms flank the P2A-NLS-FLP-T2A-GFP cassette, as shown in FIG. 5B. Altogether, this targeting vector consists of the following elements—5′HA-P2A-FLP-T2A-GFP-3′HA, all fused in-frame. A schematic illustration of this TV is shown in FIG. 5B.

The genome of Breed B contains insertions of FRT sites (SEQ ID NO: 30) flanking the first exon of DAZL. These are used to excise the first exon from the genome thus render the DAZL null. The introductions of the FRT sites are generated by homologous recombination of a ssODN molecule, comprising a FRT site (SEQ ID NO: 30), flanked by two short homology arms. For each of the two integrations of the FRT, a specific ssODN is designed. The two ssODN molecules are termed FRT-5′ssODN (SEQ ID NO: 31) and FRT-3′ssOND (SEQ ID NO: 32) with respect to their position of the first DAZL exon. The introduction of the ssODN elements is generated by homologous recombination mediated by DSB, generated by the CRISPR-Cas9 system. The locations of two sgRNA (sgRNA-5′ssODN SEQ ID NO: 21; ch2:34,397,674.34,397,693 white leghorn layer GRCg7w, and sgRNA-3′ssODN SEQ ID NO: 22; ch2:34,392,740.34,392,759 white leghorn layer GRCg7w, respectively) used for these DSB are shown (sharp sign #) in FIG. 5C.

In addition to the two ssODN integrations, the SIE, here Cre (SEQ ID NO: 26 and SEQ ID NO: 27), is fused in-frame downstream to the last coding exon of DAZL, with the self-cleaving peptide P2A (SEQ ID NO: 13, encoding SEQ ID NO: 14), followed by a NLS sequence (SEQ ID NO: 3, encoding SEQ ID NO: 4). To facilitate the identification of the genomic integration, the reporter gene GFP (SEQ ID NO: 15, encoding SEQ ID NO: 16) is also introduced in-frame. The GFP (SEQ ID NO: 15, encoding SEQ ID NO: 16) is connected by a self-cleaving peptide T2A (SEQ ID NO: 17, encoding SEQ ID NO: 18) to the 3′ end of Cre. To integrate these elements to the genome, a targeting vector (TV) is designed as shown in FIG. 5D. This targeting vector consists of two 5′ and 3′ homology arms that direct specific integration by homologous recombination. These arms flank the P2A-NLS-Cre-T2A-GFP cassette. Altogether, this targeting vector consists of the following elements—5′HA-P2A-NLS-Cre-T2A-GFP-3′HA, all fused in-frame. A schematic illustration of this TV is shown in FIG. 5D.

By crossing between to chickens, from Breed A and Breed B, which are homozygotes to the insertions, all the offspring embryos Breed AB will carry both the Flp and the Cre recombinases. The Flp, which originates from the allele received from Breed A, will excise the first exon of DAZL, flanked by the FRT sites on the allele received from Breed B; and the Cre, which originates from the allele received from Breed B, will excise the first exon of DAZL, flanked by the LoxP sites on the allele received from Breed A. Excising both first exons of DAZL in Breed AB embryos will render them DAZL null.

### Example 21: Production of Sterile Avian Embryos Devoid of Functional Primordial Germ Cells (PGCs) while Maintaining Founder Populations Via Inactivation of Deleted in AZoospermia-Like (DAZL) Using a Cre Recombinase-LoxP and an Intein Reconstitution System

Objective: To produce a population of sterile avian embryos devoid of functional PGCs while maintaining founder populations via inactivation of Deleted in AZoospermia-Like (DAZL) using a Cre recombinase-LoxP and an intein reconstitution system.

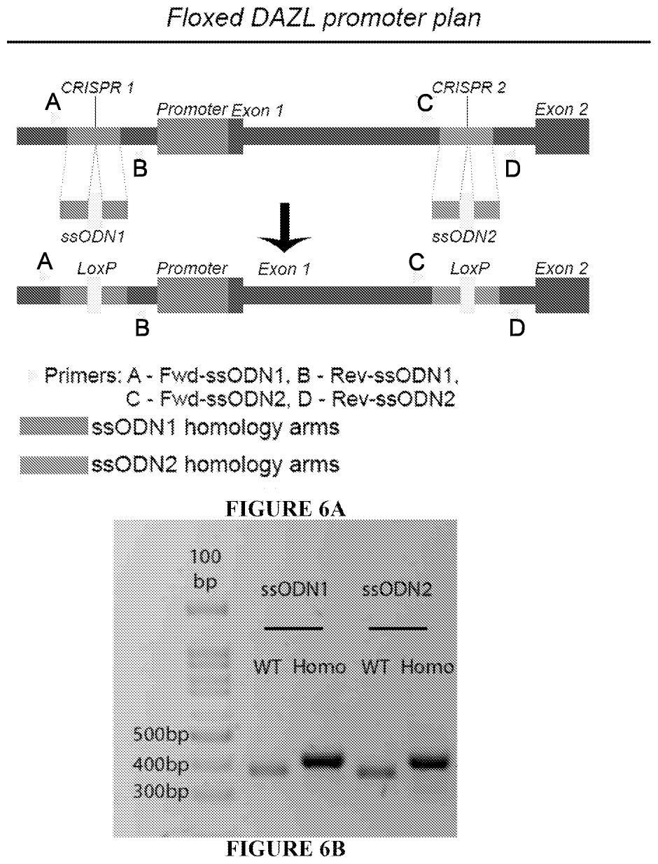

In this example, a strategy of generating two chicken breeds, each harboring an integration of two LoxP sites flanking the first exon of DAZL, resulting in a floxed DAZL promoter, is used. Excising the floxed DAZL promoter from the genome will result in the failure to form functional gametes (as depicted in FIG. 6A).

Two breeds are then produced. The design of the NLS-nCre-nIntein and cCre-cIntein targeting vectors and the mode of integration to PGCs genome are depicted in FIG. 7A.

Breed A expresses the fusion protein NLS-nCre-nIntein under the regulation of the DAZL promoter (FIG. 7A, left). This is achieved by in-frame fusion of the self-cleaving peptide P2A (SEQ ID NO: 53, encoding SEQ ID NO: 75) downstream of the coding sequence of DAZL, while omitting the stop codon. Following NLS-nCre-nIntein, the self-cleaving peptide T2A (SEQ ID NO: 54, encoding SEQ ID NO: 76) and the reporter gene GFP (SEQ ID NO: 55, encoding SEQ ID NO: 77) are also fused in-frame, as 5′HA-P2A-NLS-nCre-nIntein-T2A-GFP-3′HA. Thus, GFP is also expressed under the regulation of the DAZL promoter.

Breed B follows a similar design, but with the fusion protein cCre-cIntein fused in frame, as 5′HA-P2A-cCre-cIntein-T2A-GFP-3′HA. (FIG. 7A, right).

Through crossing between the two breeds, the two fusion proteins, NLS-nCre-nIntein and cCre-cIntein, dimerize and form an active form of the Cre enzyme, which excises the first exon of DAZL, rendering it null.

The process of generating pure PGC lines involves the following steps: electroporation, fluorescence-activated cell sorting (FACS) to isolate individual cells, and cultivation of these cells to form pure colonies. These colonies exhibit variation, with some being unmodified, while others show 1-4 integrations. Through PCR screening of each colony, their specific modifications are each identified.

PGCs were prepared with vectors as above. Vectors were prepared as follows:

FIG. 6A shows the strategy used to integrate the two LoxP sites, using CRISPR-mediated homologous recombination of ssODNs in order to knock out the DAZL gene via the insertion of two LoxP sites that flank the promoter region on exon 1 in primordial germ cells' (PGCs) genome. To ensure precise integration of the LoxP sites at the desired location, two single-stranded oligodeoxynucleotides (ssODN) were planned. ssODN1 (SEQ ID NO: 35) was designed for integration upstream, and ssODN2 (SEQ ID NO: 36) downstream, of exon 1. Each ssODN comprised 60 base pair (bp) homology arms on either side of the LoxP sequence, resulting in a total oligonucleotide sequence length of 156 bp. Homologous recombination was facilitated using CRISPR-mediated double-strand breaks (DSBs) at each site, achieved with CRISPR1 (SEQ ID NO: 33) and CRISPR2 (SEQ ID NO: 34) sgRNAs, respectively. In some embodiments, the sgRNA sequence may include a protospacer adjacent motif (PAM) sequence, which is a short nucleotide sequence (usually 2-6 bp) enabling the Cas nuclease in a CRISPR system to cut, e.g., added to the 3′-end of the sgRNA (e.g., “AGG”) at the 3′-end of the sgRNA.

Primers flanking each ssODN integration site (see FIG. 6A; A: F-Pre-D-ssODN1 [SEQ ID NO: 37]; B: R-Pre-D-ssODN1 [SEQ ID NO: 38]; C: F-Pre-D-ssODN2 [SEQ ID NO: 39]; D: R-Pre-D-ssODN2 [SEQ ID NO: 40]) were designed to validate the integration by performing PCR amplification of the region containing the inserted LoxP sequence.

FIG. 6B shows the results of PCR analysis, via gel electrophoresis (marker in left lane), of the LoxP integration. Extracted genomic DNA from PGCs was used as template for PCR amplification with the—following primers: A—Fwd-ssODN—(SEQ ID NO: 37), B—Rev-ssODN-(SEQ ID NO: 38), C—Fwd-ssODN-(SEQ ID NO: 39), D—Rev-ssODN2 (SEQ ID NO: 40), as depicted in FIG. 6A. Genomic DNA from unmodified (not modified with the LoxP inserts, as described herein) GMO PGCs was used as a positive control. The expected size of the unmodified product is 364 bp. Upon LoxP integration, the predicted size increases to 398 bp for both ssODN1 and ssODN2 integrations (see FIG. 6B). The PCR products of both ssODN1 and ssODN2 were sequenced-verified (SEQ ID NO: 42 and SEQ ID NO: 43, respectively). FIG. 6B depicts PCR analysis of a colony with four LoxP integrations, two on each allele, indicating a homozygous modification. Notably, the successful homologous recombination of both ssODNs occurred on both alleles, as confirmed by PCR, as evidenced by the appearance of a single band (Homo) and sequencing. As a result, the primordial germ cells (PGCs) generated in this process are homozygous for the insertion of the LoxP sites. The whole genomic sequence of the GMO bird (without the LoxP modifications described herein) is SEQ ID NO: 41, and the whole genomic sequence with LoxP insertions is SEQ ID NO: 44.

To demonstrate the ability of Cre to excise Exon 1, PGCs were prepared and screened as described above, and a heterozygote PGC line was selected and transiently transfected. This line differs from the one presented in FIG. 6B. On one allele, it carries two LoxP sites, while the other allele remains unmodified, resulting in a heterozygous state.

The pCAG-Cre recombinase construct (SEQ ID NO: 80, expressing SEQ ID NO: 81) was expressed in this line, and the excision of exon 1 was confirmed. Performing this experiment in heterozygous cells was necessary due to the essential role of DAZL in cell viability. If pCAG-Cre is expressed in homozygous cells, DAZL would be knocked out, leading to cell death. By expressing pCAG-Cre in heterozygous cells, the presence of the unmodified DAZL allele ensures cell survival, allowing for subsequent analysis.

To demonstrate the feasibility of excising exon 1, flanked by two loxP sites, the pCAGG-Cre plasmid (SEQ ID NO: 80 and SEQ ID NO: 81) was transiently expressed in double LoxP integration heterozygotes' PGCs. (For this experiment, heterozygous, rather than homozygous, expression was necessary, due to the fact that homozygous PGCs would die.) In this line, one allele contains an insertion of two loxP sites, while the other allele remains unmodified. Essentially, transient expression was performed on PGCs that were generated as described in FIG. 6A, but selected for heterozygotes, and the plasmid was expressed in heterozygotes' PGCs, with only one unmodified allele. For the experiment with pCAG-Cre plasmid, heterozygotic PGCs were used. Upon transiently expressing Cre, removal of exon 1 was verified. Thus, transiently expressing Cre resulted in the excision of exon 1 on one allele, leaving the other allele unmodified. This colony was injected into male surrogate chimera embryos, which hatched and, upon reaching sexual maturity, the sperm tested positive for the deletion.

Exemplary targeting vectors for integration of Cre-Intein fusion proteins into the reading frame of the DAZL gene are shown in FIGS. 7A-7C.

FIG. 7A depicts a schematic representation of the targeting vectors (TV; target vectors) designed and used according to the methods described herein.

The two vectors have the same 5′ and 3′ homology arms (HA; SEQ ID NO: 46 and SEQ ID NO: 47, respectively), directing the integration to the DAZL locus. The elements of the vector for Breed A comprise a NLS (SEQ ID NO: 48, encoding SEQ ID NO: 70), an N-terminal moiety of the Cre recombinase sequence (SEQ ID NO: 49, encoding SEQ ID NO: 71), and an N-terminal moiety of the Intein sequence (SEQ ID NO: 50, encoding SEQ ID NO: 72). The elements of the vector for Breed B comprise a C-terminal moiety of the Cre recombinase sequence (SEQ ID NO: 51, encoding SEQ ID NO: 73) and a C-terminal moiety of the Intein sequence (SEQ ID NO: 52, encoding SEQ ID NO: 74). The fusion proteins, NLS-nCre-nIntein (SEQ ID NOS: 70-72, encoded by SEQ ID NOS: 48-50, respectively; see FIG. 7A, left) or cCre-cIntein (SEQ ID NOS: 73-74, encoded by SEQ ID NOS: 51-52, respectively; see FIG. 7A, right) are fused in-frame to the C-terminus of the DAZL coding sequence by the P2A peptide (SEQ ID NO: 75, encoded by SEQ ID NO: 53). Following them is the T2A peptide (SEQ ID NO: 76, encoded by SEQ ID NO: 54) fused to the reporter gene GFP (SEQ ID NO: 77, encoded by SEQ ID NO: 55). Therefore, the nCre-nIntein targeting vector for Breed A comprises P2A-NLS-nCre-nIntein-T2A-GFP (SEQ ID NO: 78) and the cCre-cIntein targeting vector for Breed B comprises P2A-cIntein-cCre-T2A-GFP (SEQ ID NO: 79). The CRISPR-mediated homologous recombination is done using a specific sgRNA sequence (SEQ ID NO: 45). The last two nucleic acids in the DAZL NLS-nCre-nIntein TV (nCre recombinase) (SEQ ID NO: 49) are for creating an in-frame link with the first amino acid of the DAZL NLS-nCre-nIntein TV (nIntein) (SEQ ID NO: 50). This codon enters a linking lysine (K) amino acid. In some embodiments, the sgRNA sequence may include a protospacer adjacent motif (PAM) sequence, which is a short nucleotide sequence (usually 2-6 bp) enabling the Cas nuclease in a CRISPR system to cut, e.g., added to the 3′-end “of the sgRNA (e.g., “AGG”) at the 3′-end of the sgRNA. The primers that were used for sequencing and PCR analysis are depicted as A-J (SEQ ID NOS: 56-65, respectively). The targeting vectors are homologous recombination targeting vectors. They serve as templates for homology-directed repair (HDR) integration of Intein-GFP downstream to the DAZL coding sequence (DAZL CDS). Once HDR occurs, the expression of the Intein-GFP is regulated by the DAZL endogenous promoter.

On-site integration was validated with primers A and G (SEQ ID NOS: 56 and 62, respectively), as well as F and J (SEQ ID NOS: 61 and 65, respectively). Primers A and J (SEQ ID NOS: 56 and 65, respectively) are located outside of the homology arms, while primers G and F (SEQ ID NOS: 62 and 61, respectively) are located inside the insert. For the correct integration of the NLS-nCre-nIntein vector (see SEQ ID NO: 48, SEQ ID NO: 49, and SEQ ID NO: 50, encoding SEQ ID NO: 70, SEQ ID NO: 71, and SEQ ID NO: 72), the expected PCR product between primers A and G (SEQ ID NO: 56 and SEQ ID NO: 62, respectively) is 2692 bp, to confirm correct integration of the 5′HA (SEQ ID NO: 46). The expected PCR product, upon correct integration, between primers F and J (SEQ ID NO: 61 and SEQ ID NO: 65, respectively) is 2001 bp, to confirm correct integration of the 3′HA (SEQ ID NO: 47). For the correct integration of the cCre-cIntein vector (see SEQ ID NO: 51 and SEQ ID NO: 52, encoding SEQ ID NO: 73 and SEQ ID NO: 74), the expected PCR product between primers A and G (SEQ ID NO: 56 and SEQ ID NO: 62, respectively) is 3136 bp, to confirm correct integration of the 5′HA (SEQ ID NO: 46) and the expected PCR product, upon correct integration, between primers F and J (SEQ ID NO: 61 and SEQ ID NO: 65, respectively) is 1998 bp, to confirm correct integration of the 3′HA (SEQ ID NO: 47).

PCR analysis of transformed PGCs colonies was performed with the results shown in FIG. 7B. Either the NLS-nCre-nIntein targeting vector (depicted in short as cInt-cCre), or the targeting vector cCre-cIntein (depicted in short as cInt-cCre) were integrated to the genome of PGCs. Extracted genomic DNA for each colony was used as template for PCR as described above. The PCR analysis confirmed the predicted products length, confirming that both targeting vectors were correctly integrated. The primers A-J (SEQ ID NOS: 56-65, respectively) were used for sequencing of unmodified PGCs genome (SEQ ID NO: 66) for control, and to validate the correct integration of the NLS-nCre-nIntein TV and cCre-cIntein TV into DAZL to yield the NLS-nCre-nIntein knockin to DAZL (SEQ ID NO: 67) and cCre-cIntein knockin to DAZL (SEQ ID NO: 68) (see FIG. 7B).

FIG. 7C depicts two pure PGCs lines, which underwent homologous recombination—mediated integration with the targeting vectors shown in FIG. 7A. Each colony is presented in brightfield and fluorescence (detecting GFP), left to right, respectively. The colony on the left (the two left panels) has the nCre-nIntein integration. The colony on the right has the cCre-cIntein integration. These two colonies correspond to the targeting vectors in FIG. 7A.

While certain features of the sterile avian embryos, products and uses thereof have been illustrated and described herein, many modifications, substitutions, changes, and equivalents will now occur to those of ordinary skill in the art. It is, therefore, to be understood that the appended claims are intended to cover all such modifications and changes as fall within the true spirit of these embryos and uses thereof.

1. A deoxyribonucleic acid (DNA) editing system comprising:
(a) a first agent comprising a first exogenous polynucleotide, the first exogenous polynucleotide comprising:
(a) a first promoter; and
(b) a first element of interest operably linked to the promoter, the first element of interest encoding a first protein moiety of interest,
wherein a first genetically modified avian having a genome comprising the first agent retains fertility and viability while producing a primordial germ cell (PGC) having a genome comprising the first agent without impairing viability of the PGC; and
(b) a second agent comprising a second exogenous polynucleotide, the second exogenous polynucleotide comprising:
(a) a second promoter; and
(b) a second element of interest operably linked to the promoter, the second element of interest encoding a second protein moiety of interest,
wherein a second genetically modified avian having a genome comprising the second agent retains fertility and viability while producing a primordial germ cell (PGC) having a genome comprising the second agent without impairing viability of the PGC,
the first agent and the second agent, when co-expressed in a genetically modified progeny avian embryo or progeny avian, wherein the genetically modified progeny avian embryo or progeny avian is a progeny of a first genetically modified avian arising from the first genetically modified PGC comprising the first agent and a second genetically modified avian being opposite-gendered from the first genetically modified avian and arising from the second genetically modified PGC comprising the second agent, inducing sterility or inhibiting fertility in the genetically modified progeny avian embryo or progeny avian without impairing viability.

2. The deoxyribonucleic acid (DNA) editing system of claim 1, wherein the first protein moiety of interest is a functionally inactive first protein moiety of interest and the second protein moiety of interest is a functionally inactive second protein moiety of interest, wherein:
(a) the functionally inactive first protein moiety of interest and the functionally inactive second protein moiety of interest, dimerized or bound covalently, comprise at least one functionally active protein of interest or fragment thereof, wherein:
(a) the functionally active protein of interest or fragment thereof comprises a genomic modifier, the genomic modifier targeting a gene of interest (GOI) or fragment thereof on a chromosome, the GOI modified to introduce one or more target sites specific to the genomic modifier, and the GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active gene of interest (GOI), the modified trait inducing sterility or inhibiting fertility in the functionally modified and genetically modified progeny avian embryo or progeny avian without impairing viability;
(b) the functionally active protein of interest or fragment thereof comprises a toxin inducing cell death of a PGC, either without impairing viability of somatic cells or limited to benign somatic cell loss or both, in the functionally modified and genetically modified progeny avian embryo or progeny avian, and inducing sterility or inhibiting fertility in the functionally modified and genetically modified progeny avian embryo or progeny avian without impairing viability; or
(c) a combination thereof;
(b) the functionally inactive first protein moiety of interest and the functionally inactive second protein moiety of interest, when co-expressed, comprise at least one functionally active protein of interest or fragment thereof, the co-expression of which inducing cell death of a PGC without impairing viability of somatic cells in the functionally modified and genetically modified progeny avian embryo or progeny avian and inducing sterility or inhibiting fertility in the functionally modified and genetically modified avian without impairing viability; or
(c) a combination of any of the above.

3. The deoxyribonucleic acid (DNA) editing system of claim 2, wherein the first GOI and the second GOI are the same GOI and the first chromosome of interest and the second chromosome of interest are the same chromosome of interest.

4. The deoxyribonucleic acid (DNA) editing system of claim 2, wherein:
(i) the first exogenous polynucleotide further comprising a self-cleaving peptide moiety operably linked to the first promoter and to the first element of interest encoding the functionally inactive first protein moiety of interest; and the second exogenous polynucleotide further comprising a self-cleaving peptide moiety distinct from the first self-cleaving peptide moiety and operably linked to the second promoter and to the second element of interest encoding the functionally inactive second protein moiety of interest, wherein the first self-cleaving peptide moiety and the second self-cleaving peptide moiety, dimerized or bound covalently, comprise a functionally active self-cleaving peptide moiety or fragment thereof, the functionally active self-cleaving peptide moiety or fragment thereof splicing the functionally inactive first protein moiety of interest and the functionally inactive second protein moiety of interest to produce the functionally active protein of interest or fragment thereof;
(ii) the first and second exogenous polynucleotides are as defined in item (i) wherein the first self-cleaving peptide moiety is a functionally inactive intein moiety; and the second self-cleaving peptide moiety is a functionally inactive intein moiety, or wherein the functionally inactive first intein moiety has a sequence at least 95% identical to SEQ ID NO: 72, and the functionally inactive second intein moiety has a sequence at least 95% identical to SEQ ID NO: 74; or wherein the functionally inactive first intein moiety has a sequence at least 95% identical to SEQ ID NO: 8 or SEQ ID NO: 72, and the functionally inactive second intein moiety has a sequence at least 95% identical to SEQ ID NO: 10 or SEQ ID NO: 74;
(iii) the first exogenous polynucleotide further encodes a first conjugating element operably linked to the first promoter and to the first element of interest encoding the functionally inactive first protein moiety of interest; and the second exogenous polynucleotide further encodes a second conjugating element operably linked to the second promoter and to the second element of interest encoding the functionally inactive second protein moiety of interest, the first conjugating element conjugating to the second conjugating element to produce the functionally active protein of interest or fragment thereof;
(iv) the first and second exogenous polynucleotides are as defined in item (iii) wherein the first conjugating element comprises SpyTag and the second conjugating element comprises SpyCatcher;
(v) the first and second exogenous polynucleotides are as defined in item (iii) wherein the first conjugating element comprises the first element of interest and the second conjugating element comprises the second element of interest, wherein:
(a) the first conjugating element comprises a functionally inactive first moiety of a recombinase and the second conjugating element comprises a functionally inactive second moiety of the recombinase, the second moiety of the recombinase distinct from the first moiety of the recombinase, the first conjugating element conjugating to the second conjugating element to produce the functionally active recombinase or fragment thereof;
(b) the first conjugating element comprises a functionally inactive first moiety of a CRISPR protein and the second conjugating element comprises a functionally inactive second moiety of the CRISPR protein, the second moiety of the CRISPR protein distinct from the first moiety of the CRISPR protein, the first conjugating element conjugating to the second conjugating element to produce the functionally active CRISPR protein or fragment thereof; or
(c) a combination thereof;
or
(vi) the first exogenous polynucleotide further encodes a first conjugating element operably linked to the first promoter and to the first element of interest encoding the functionally inactive first protein moiety of interest; and the second exogenous polynucleotide further encodes a second conjugating element operably linked to the second promoter and to the second element of interest encoding the functionally inactive second protein moiety of interest, the first conjugating element conjugating to the second conjugating element to produce the functionally active protein of interest or fragment thereof.

5.-9. (canceled)

10. The deoxyribonucleic acid (DNA) editing system of claim 2, wherein the genomic modifier comprises;
(i) a site-specific recombinase comprising a Cre recombinase, and the functionally inactive first Cre protein moiety of interest has a sequence at least 95% identical to SEQ ID NO: 6 or SEQ ID NO: 71 and the functionally inactive second Cre protein moiety of interest has a sequence at least 95% identical to SEQ ID NO: 12 or SEQ ID NO: 73, and the recombinase recognition site comprises a locus of X-over P1 site (LoxP), or
(ii) a Cre recombinase (Cre) protein or functionally active fragment thereof; and the first exogenous polynucleotide further encoding a functionally inactive first intein moiety operably linked to the first promoter and to the first element of interest encoding a functionally inactive first Cre protein moiety of interest; and the second exogenous polynucleotide further encoding a functionally inactive second intein moiety distinct from the functionally inactive first intein moiety and operably linked to the second promoter and to the second element of interest encoding a functionally inactive second Cre protein moiety of interest, distinct from the first Cre protein moiety of interest, wherein the functionally inactive first CRE moiety or the functionally inactive second CRE moiety is fused in-frame to a nuclear localizing signal (NLS), wherein the functionally inactive first Cre moiety and the functionally inactive second Cre moiety, dimerized or bound covalently, comprise a functionally active Cre or functionally active fragment thereof, and wherein the functionally inactive first intein moiety and the functionally inactive second intein moiety, dimerized or bound covalently, comprise a functionally active intein or fragment thereof, the functionally active intein or fragment thereof splicing the functionally inactive first protein moiety of interest and the functionally inactive second protein moiety of interest to produce the functionally active protein of interest or fragment thereof, wherein the functionally inactive first Cre protein moiety of interest has a sequence at least 95% identical to SEQ ID NO: 6 or SEQ ID NO: 71, the functionally inactive second Cre protein moiety of interest has a sequence at least 95% identical to SEQ ID NO: 12 or SEQ ID NO: 73; wherein the functionally inactive first intein moiety has a sequence at least 95% identical to SEQ ID NO: 8 or SEQ ID NO: 72, and the functionally inactive second intein moiety has a sequence at least 95% identical to SEQ ID NO: 10 or SEQ ID NO: 74.

11. (canceled)

12. The deoxyribonucleic acid (DNA) editing system of claim 2, wherein the first agent has a sequence at least 95% identical to SEQ ID NO: 78 and the second agent has a sequence at least 95% identical to SEQ ID NO: 79.

13. The deoxyribonucleic acid (DNA) editing system of claim 1, the first protein moiety of interest comprising a functionally active first genomic modifier and the second protein moiety of interest comprising a functionally active second genomic modifier, wherein:
(i) the functionally active second genomic modifier targeting a first gene of interest (GOI) or fragment thereof on a chromosome, the first GOI or fragment thereof modified to introduce one or more target sites specific to the second genomic modifier, and the first GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active first gene of interest (GOI), the modified trait inducing sterility or inhibiting fertility in the functionally modified and genetically modified progeny avian embryo or progeny avian without impairing viability; and
(ii) the functionally active first genomic modifier targeting a second gene of interest (GOI) or fragment thereof on a chromosome, the second GOI or fragment thereof modified to introduce one or more target sites specific to the second genomic modifier, and the second GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active second gene of interest, the modified trait inducing sterility or inhibiting fertility in the functionally modified and genetically modified progeny avian embryo or progeny avian without impairing viability.

14. The deoxyribonucleic acid (DNA) editing system of claim 13, wherein:
(i) the first gene of interest (GOI) and the second GOI are the same GOI, wherein the first GOI, prior to modification with the one or more target sites or to the deletion, disruption, or functional modification of the GOI, and the second GOI, prior to genetic modification with the one or more target sites or to the deletion, disruption or functional modification of the GOI, are the same GOI and the first chromosome of interest and the second chromosome of interest are the same chromosome of interest;
(ii) the fragment of the first gene of interest (GOI) and the fragment of the second GOI are distinct fragments of the same GOI;
(iii) the first genomic modifier and the second genomic modifier are independently selected from a non-identical site-specific recombinase enzyme or functionally active fragment thereof;
or
(iv) (a) the first genomic modifier comprises a functionally active tyrosine recombinase comprising Flippase recombinase (Flp) moiety, operably linked to a nuclear localizing signal (NLS), and the first GOI comprises a recombinase recognition site, the recombinase recognition site comprising LoxP; and (b) the second genomic modifier comprises a functionally active tyrosine recombinase comprising a Cre recombinase moiety, operably linked to a nuclear localizing signal (NLS), and the second GOI comprises a recombinase recognition site, the recombinase recognition site comprising FRT,
wherein the first genomic modifier comprising the Flp moiety, operably linked to a NLS, has a sequence at least 95% identical to SEQ ID NO: 29; or wherein the second genomic modifier comprising the Cre moiety, operably linked to a NLS, has a sequence at least 95% identical to SEQ ID NO: 27.

15.-17. (canceled)

18. The deoxyribonucleic acid (DNA) editing system of claim 2, wherein the gene of interest (GOI) sequence or fragment thereof or the first GOI or fragment thereof of claim 13 or the second GOI or fragment thereof of claim 13 has
(i) an isolated function specific to a PGC; or
(ii) a function specific to gametogenesis, meiosis, gamete maturation, gamete function, or gamete fertilization in the genetically modified avian,
wherein deletion, disruption, or functional modification of the gene of interest (GOI), the first GOI, or the second GOI reduces or inhibits survival, maturation, or differentiation of a PGC or the specific gametogenesis, gamete maturation, or gamete functional modification reduces or inhibits gametogenesis, meiosis, gamete function, or gamete fertilization in an avian.

19. The deoxyribonucleic acid (DNA) editing system of claim 18, wherein the gene of interest (GOI) sequence or fragment thereof, the first GOI or fragment thereof, or the second GOI or fragment thereof comprises a deleted in azoospermia-like (DAZL) gene, a deleted in azoospermia 1 (DAZ1) gene, a zona pellucida binding protein 1/2 (ZPBP1/2) gene, a cyclin-dependent kinases regulatory subunit 2 (CKS2; CDC28 Protein Kinase Regulatory Subunit 2) gene, a spermatogenesis associated 16 (SPATA16) gene, a DEAD-box helicase 4 (DDX4) gene, a serine/threonine-protein phosphatase PP1-gamma catalytic subunit (PPP1CC) gene, an Izumo sperm-egg fusion 1 (IZUMO1) gene, a synaptonemal complex central element protein 1 (SYCE1) gene, a YTH domain-containing 2 (YTHDC2) gene, a Meiosis Specific With Coiled-Coil Domain (MEIOC) gene, a septin-4 (SEPT4) gene, a stromal antigen 3 (STAG3) gene, a Nanos C2HC-type zinc finger 3 (NANOS3) gene, or a combination of any of these.

20. The deoxyribonucleic acid (DNA) editing system of claim 2, wherein the first genomic modifier or the second genomic modifier or both comprises a site-specific recombinase enzyme or functionally active fragment thereof.

21. The deoxyribonucleic acid (DNA) editing system of claim 20, wherein the site-specific recombinase is a tyrosine recombinase or a serine recombinase.

22. The deoxyribonucleic acid (DNA) editing system of claim 21, wherein the site-specific recombinase comprises a tyrosine recombinase comprising a Cre recombinase (Cre), a Dre recombinase (Dre), a flippase recombinase (Flp), or a Vika recombinase (Vika); and the target site comprises a recombinase recognition site, the recombinase recognition site comprising, respectively, a Lox site (Lox), a Rox site (Rox), a FRT site (FRT), or a Vox site (Vox).

23. The deoxyribonucleic acid (DNA) editing system of claim 2, wherein:
(i) the first exogenous polynucleotide encodes a functionally active marker operably linked to the first protein moiety of interest; the second exogenous polynucleotide encodes a functionally active marker operably linked to the second protein moiety of interest; or a combination thereof;
(ii) the first exogenous polynucleotide encodes a nuclear localizing signal (NLS) operably linked to the first protein moiety of interest; the second exogenous polynucleotide encodes a nucleic localizing signal (NLS) operably linked to the second protein moiety of interest; or a combination thereof;
(iii) the first exogenous polynucleotide further comprises one or more self-cleaving peptides operably linked to the first protein moiety of interest; and wherein the second exogenous polynucleotide further comprises one or more self-cleaving peptides operably linked to the second protein moiety of interest;
(iv) the first exogenous polynucleotide further comprises a first 5′ homology arm (HA) and a first 3′ homology arm (HA), said first 5′ HA and said first 3′ HA specific for a first insertion site of interest on the avian genome; and wherein the second exogenous polynucleotide further comprises a second 5′ homology arm (HA) and a second 3′ homology arm (HA), said second 5′ HA and said second 3′ HA specific for a second insertion site of interest on the avian genome, wherein:
(a) the first 5′ HA has a nucleotide sequence that is substantially homologous to the 5′ region flanking a first gene of interest (GOI) in a first chromosome of interest and the first 3′ HA has a nucleotide sequence that is substantially homologous to the 3′ region flanking the first GOI in the first chromosome of interest:
(b) the second 5′ HA has a nucleotide sequence that is substantially homologous to the 5′ region flanking a second GOI in a second chromosome of interest and the second 3′ HA, or both has a nucleotide sequence that is substantially homologous to the 3′ region flanking the second GOI in the second chromosome of interest; or
(c) both; or
(v) the first and second exogenous polynucleotides are as defined in item (iv) wherein the gene of interest (GOI) or a fragment thereof comprises a Deleted in AZoospermia-Like (DAZL) gene or a fragment thereof; and wherein the first 5′ HA, the second 5′ HA, or both has a sequence at least 95% identical to SEQ ID NO: 46 or SEQ ID NO: 23, and the first 3′ HA, the second 3′ HA, or both has a sequence at least 95% identical to SEQ ID NO: 47 or SEQ ID NO: 24.

24.-27. (canceled)

28. The deoxyribonucleic acid (DNA) editing system of claim 1, wherein the first promoter, the second promoter, or both comprises:
(i) a promoter specific to a primordial germ cell (PGC);
(ii) a tissue-specific promoter; or
(iii) a ubiquitous promoter.

29. A primordial germ cell (PGC) system comprising a first genetically modified avian primordial germ cell (PGC) and a second genetically modified avian primordial germ cell (PGC):
(i) the first genetically modified avian PGC comprising a first agent, the first agent comprising a first exogenous polynucleotide, the first exogenous polynucleotide comprising:
(a) a first promoter; and
(b) a first element of interest operably linked to the promoter, the first element of interest encoding a first protein moiety of interest,
wherein a first genetically modified avian having a genome comprising the first agent retains fertility and viability while producing a primordial germ cell (PGC) having a genome comprising the first agent without impairing viability of the PGC; and
(ii) the second genetically modified avian PGC comprising a second agent, the second agent comprising a second exogenous polynucleotide, the second exogenous polynucleotide comprising:
(a) a second promoter; and
(b) a second element of interest operably linked to the promoter, the second element of interest encoding a second protein moiety of interest,
wherein a second genetically modified avian having a genome comprising the second agent retains fertility and viability while producing a primordial germ cell (PGC) having a genome comprising the second agent without impairing viability of the PGC, the first agent and the second agent, when co-expressed in a population of genetically modified progeny avian embryos or progeny avians, wherein the genetically modified progeny avian embryo or progeny avian is a progeny of a first genetically modified avian arising from the first genetically modified PGC comprising the first agent and an opposite-gendered second genetically modified avian arising from the second genetically modified PGC comprising the second agent, inducing sterility or inhibiting fertility in 100% of the population of genetically modified progeny avian embryo or progeny avian without impairing viability.

30. The primordial germ cell (PGC) system of claim 29, wherein:
(i) the first protein moiety of interest is a functionally inactive first protein moiety of interest and the second protein moiety of interest is a functionally inactive second protein moiety of interest, wherein:
(a) the functionally inactive first protein moiety of interest and the functionally inactive second protein moiety of interest, dimerized or bound covalently, comprise at least one functionally active protein of interest or fragment thereof, wherein:
(a) the functionally active protein of interest or fragment thereof comprises a genomic modifier, the genomic modifier targeting a gene of interest (GOI) or fragment thereof on a chromosome, the GOI modified to introduce one or more target sites specific to the genomic modifier, and the GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active gene of interest (GOI), the modified trait inducing sterility or inhibiting fertility in the functionally modified and genetically modified progeny avian embryo or progeny avian without impairing viability;
(b) the functionally active protein of interest or fragment thereof comprises a toxin inducing cell death of a PGC, either without impairing viability of somatic cells or limited to benign somatic cell loss or both, in the functionally modified and genetically modified progeny avian embryo or progeny avian, and inducing sterility or inhibiting fertility in the functionally modified and genetically modified progeny avian embryo or progeny avian without impairing viability; or
(c) a combination thereof;
(b) the functionally inactive first protein moiety of interest and the functionally inactive second protein moiety of interest, when co-expressed, comprise at least one functionally active protein of interest or fragment thereof, the co-expression of which inducing cell death of a PGC without impairing viability of somatic cells in the functionally modified and genetically modified progeny avian embryo or progeny avian and inducing sterility or inhibiting fertility in the functionally modified and genetically modified avian without impairing viability; or
(c) a combination of any of the above; or
(ii) the first protein moiety of interest comprises a functionally active first genomic modifier and the second protein moiety of interest comprising a functionally active second genomic modifier, wherein:
(a) the functionally active second genomic modifier targeting a first gene of interest (GOI) or fragment thereof on a chromosome, the first GOI or fragment thereof modified to introduce one or more target sites specific to the second genomic modifier, and the first GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active first gene of interest (GOI), the modified trait inducing sterility or inhibiting fertility in the functionally modified and genetically modified progeny avian embryo or progeny avian; and
(b) the functionally active first genomic modifier targeting a second gene of interest (GOI) or fragment thereof on a chromosome, the second GOI or fragment thereof modified to introduce one or more target sites specific to the second genomic modifier, and the second GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active second gene of interest, the modified trait inducing sterility or inhibiting fertility in the functionally modified and genetically modified progeny avian embryo or progeny avian without impairing viability.

31. (canceled)

32. A sterile avian breeding system to produce 100% sterility in a population of embryos, said system comprising a first genetically modified avian and a second genetically modified avian having an opposite sex to the first genetically modified avian:
(i) the first genetically modified avian comprising a first genetically modified avian PGC comprising a first agent, the first agent comprising a first exogenous polynucleotide, the first exogenous polynucleotide comprising:
(a) a first promoter; and
(b) a first element of interest operably linked to the promoter, the first element of interest encoding a first protein moiety of interest; and
(ii) the second genetically modified avian comprising a second genetically modified avian PGC comprising a second agent, the second agent comprising a second exogenous polynucleotide, the second exogenous polynucleotide comprising:
(a) a second promoter; and
(b) a second element of interest operably linked to the promoter, the second element of interest encoding a second protein moiety of interest,
the first agent and the second agent, when co-expressed in a genetically modified progeny avian embryo or progeny avian, wherein the genetically modified progeny avian embryo or progeny avian is a progeny of the first genetically modified avian arising from the first genetically modified PGC comprising the first agent and an opposite-gendered second genetically modified avian arising from the second genetically modified PGC comprising the second agent, inducing sterility or inhibiting fertility in the genetically modified progeny avian embryo or progeny avian without impairing viability, the first genetically modified avian having a genome comprising the first agent retains fertility and viability while producing a primordial germ cell (PGC) having a genome comprising the first agent without impairing viability of the PGC and the second genetically modified avian having a genome comprising the second agent retains fertility and viability while producing a primordial germ cell (PGC) having a genome comprising the second agent without impairing viability of the PGC.

33. The sterile avian breeding system of claim 32, wherein
(i) the first protein moiety of interest is a functionally inactive first protein moiety of interest and the second protein moiety of interest is a functionally inactive second protein moiety of interest, wherein:
(1) the functionally inactive first protein moiety of interest and the functionally inactive second protein moiety of interest, dimerized or bound covalently, comprise at least one functionally active protein of interest or a functionally active fragment thereof, wherein:
(a) the functionally active protein of interest or fragment thereof comprises a genomic modifier, the genomic modifier targeting a gene of interest (GOI) or fragment thereof on a chromosome, the GOI modified to introduce one or more target sites specific to the genomic modifier, and the GOI when deleted, disrupted, or functionally modified, modifying a trait in a functionally modified and genetically modified progeny primordial germ cell (PGC) or in the functionally modified and genetically modified progeny avian embryo or progeny avian produced by the functionally modified and genetically modified progeny PGC, when compared to an isogenic PGC or an isogenic avian comprising a functionally active gene of interest (GOI), the modified trait inducing sterility or inhibiting fertility in the functionally modified and genetically modified progeny avian embryo or progeny avian without impairing viability;
(b) the functionally active protein of interest or fragment thereof comprises a toxin inducing cell death of a PGC, either without impairing viability of somatic cells or limited to benign somatic cell loss or both, in the functionally modified and genetically modified progeny avian embryo or progeny avian, and inducing sterility or inhibiting fertility in the functionally modified and genetically modified progeny avian embryo or progeny avian without impairing viability; or
(c) a combination thereof;
(2) the functionally inactive first protein moiety of interest and the functionally inactive second protein moiety of interest, when co-expressed, comprise at least one functionally active protein of interest or fragment thereof, the co-expression of which inducing cell death of a PGC without impairing viability of somatic cells in the functionally modified and genetically modified progeny avian embryo or progeny avian and inducing sterility or inhibiting fertility in the functionally modified and genetically modified avian without impairing viability; or
(3) a combination of any of the above: or
(ii) the first protein moiety of interest comprises a functionally active first genomic modifier and the second protein moiety of interest comprises a functionally active second genomic modifier, wherein:
(a) the functionally active second genomic modifier targeting a first gene of interest (GOI) or fragment thereof on a chromosome, the first GOI or fragment thereof modified to introduce one or more target sites specific to the second genomic modifier, and the first GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active first gene of interest (GOI), the modified trait inducing sterility or inhibiting fertility in the functionally modified and genetically modified progeny avian embryo or progeny avian; and
(b) the functionally active first genomic modifier targeting a second gene of interest (GOI) or fragment thereof on a chromosome, the second GOI or fragment thereof modified to introduce one or more target sites specific to the second genomic modifier, and the second GOI or fragment thereof when deleted, disrupted, or functionally modified, modifying a trait in a functionally modified and genetically modified progeny primordial germ cell (PGC) or in a functionally modified and genetically modified progeny avian embryo or progeny avian produced by the functionally modified and genetically modified progeny PGC, when compared to an isogenic PGC or an isogenic avian comprising a functionally active second gene of interest, the modified trait inducing sterility or inhibiting fertility in the functionally modified and genetically modified progeny avian embryo or progeny avian without impairing viability.

34.-37. (canceled)
